SEQUENCE LISTING (37 sequences)

SEQ ID NO: 1 - protein, 18 aa, Thosea asigna virus
EGRGSLLTCGDVEENPGP

SEQ ID NO: 2 - protein, 19 aa, Porcine teschovirus-1
ATNFSLLKQAGDVEENPGP

SEQ ID NO: 3 - protein, 20 aa, Equine rhinitis A virus
QCTNYALLKLAGDVESNPGP

SEQ ID NO: 4 - protein, 22 aa, Foot-and-mouth disease virus
VKQTLNFDLLKLAGDVESNPGP

SEQ ID NO: 5 - protein, 20 aa, Artificial Sequence
  Description of Artificial Sequence Synthetic peptide
ETQRCTWHMGELVWCEREHN

SEQ ID NO: 6 - protein, 20 aa, Artificial Sequence
  Description of Artificial Sequence Synthetic peptide
KEASCSYWLGELVWCVAGVE

SEQ ID NO: 7 - protein, 13 aa, Artificial Sequence
  Description of Artificial Sequence Synthetic peptide
DCAWHLGELVWCT

SEQ ID NO: 8 - protein, 50 aa, Artificial Sequence
  Description of Artificial Sequence Synthetic polypeptide
  MISC_FEATURE (1)..(50): This sequence may encompass 3-50 residues
RRRRRRRRRRRRRRRRRRRRRRRRRRRRRRRRRRRRRRRRRRRRRRRRRR

SEQ ID NO: 9 - protein, 9 aa, Artificial Sequence
  Description of Artificial Sequence Synthetic peptide
RRRRRRRRR

SEQ ID NO: 10 - protein, 9 aa, Artificial Sequence
  Description of Artificial Sequence Synthetic peptide
EEEEEEEEE

SEQ ID NO: 11 - protein, 7 aa, Simian virus 40
PKKKRKV

SEQ ID NO: 12 - protein, 16 aa, Unknown
  Description of Unknown Nucleoplasmin bipartite NLS sequence
KRPAATKKAGQAKKKK

SEQ ID NO: 13 - protein, 9 aa, Unknown
  Description of Unknown C-myc NLS sequence
PAAKRVKLD

SEQ ID NO: 14 - protein, 11 aa, Unknown
  Description of Unknown C-myc NLS sequence
RQRRNELKRSP

SEQ ID NO: 15 - protein, 38 aa, Homo sapiens
NQSSNFGPMKGGNFGGRSSGPYGGGGQYFAKPRNQGGY

SEQ ID NO: 16 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 17 - protein, 8 aa, Unknown
  Description of Unknown Myoma T protein sequence
VSRKRPRP

SEQ ID NO: 18 - protein, 8 aa, Unknown
  Description of Unknown Myoma T protein sequence
PPKKARED

SEQ ID NO: 19 - protein, 8 aa, Homo sapiens
PQPKKKPL

SEQ ID NO: 20 - protein, 12 aa, Mus musculus
SALIKKKKKMAP

SEQ ID NO: 21 - protein, 5 aa, Influenza virus
DRLRR

SEQ ID NO: 22 - protein, 7 aa, Influenza virus
PKQKKRK

SEQ ID NO: 23 - protein, 10 aa, Hepatitis delta virus
RKLKKKIKKL

SEQ ID NO: 24 - protein, 10 aa, Mus musculus
REKKKFLKRR

SEQ ID NO: 25 - protein, 20 aa, Homo sapiens
KRKGDEVDGVDEVAKKKSKK

SEQ ID NO: 26 - protein, 17 aa, Homo sapiens
RKCLQAGMNLEARKTKK

SEQ ID NO: 27 - protein, 5 aa, Unknown
  Description of Unknown Dual acylation region sequence
  MOD_RES (4)..(4): Any amino acid
MGCXC

SEQ ID NO: 28 - protein, 5 aa, Unknown
  Description of Unknown Caspase-10 recognition sequence
  MOD_RES (5)..(5): Any amino acid
IEADX

SEQ ID NO: 29 - protein, 5 aa, Unknown
  Description of Unknown Caspase-2 recognition sequence
  MOD_RES (5)..(5): Any amino acid except Asp, Glu, Lys, Pro, Gln or Arg
DVADX

SEQ ID NO: 30 - protein, 5 aa, Unknown
  Description of Unknown Caspase-2 recognition sequence
  MOD_RES (5)..(5): Any amino acid except Asp, Glu, Lys, Pro, Gln or Arg
DEHDX

SEQ ID NO: 31 - protein, 5 aa, Unknown
  Description of Unknown Caspase-3 recognition sequence
  MOD_RES (5)..(5): Any amino acid except Asp, Glu, Lys, Pro, Gln or Arg
DMQDX

SEQ ID NO: 32 - protein, 5 aa, Unknown
  Description of Unknown Caspase-3 recognition sequence
  MOD_RES (5)..(5): Any amino acid except Asp, Glu, Lys, Pro, Gln or Arg
DEVDX

SEQ ID NO: 33 - protein, 5 aa, Unknown
  Description of Unknown Caspase-4 recognition sequence
  MOD_RES (5)..(5): Any amino acid except Asp, Glu, Lys, Pro, Gln or Arg
LEVDX

SEQ ID NO: 34 - protein, 5 aa, Unknown
  Description of Unknown Caspase-7 recognition sequence
  MOD_RES (5)..(5): Any amino acid except Asp, Glu, Lys, Pro, Gln or Arg
DEVDX

SEQ ID NO: 35 - protein, 5 aa, Unknown
  Description of Unknown Caspase-9 recognition sequence
  MOD_RES (5)..(5): Any amino acid
LEHDX

SEQ ID NO: 36 - protein, 5 aa, Unknown
  Description of Unknown Granzyme B recognition sequence
  MOD_RES (5)..(5): Any amino acid
IEPDX

SEQ ID NO: 37 - protein, 8 aa, Unknown
  Description of Unknown HRV3C protease recognition sequence
LEVLFQGP

(19) United States
(12) Patent Application Publication
(10) Pub. No.: US 2020/0157534 A1
(43) Pub. Date: May 21, 2020

(54) METHODS AND SYSTEMS FOR CONDITIONALLY REGULATING GENE EXPRESSION

(71) Applicant: Refuge Biotechnologies, Inc., Menlo Park, CA (US)

(72) Inventors: Jianbin WANG, Menlo Park, CA (US); Bing WANG, Menlo Park, CA (US); Pei-Qi LIU, Menlo Park, CA (US)

(21) Appl. No.: 16/739,400

(22) Filed: Jan. 10, 2020

Related U.S. Application Data
(63) Continuation of application No. PCT/US18/41704, filed on Jul. 11, 2018.
(60) Provisional application No. 62/587,668, filed on Nov. 17, 2017.
(60) Provisional application No. 62/531,752, filed on Jul. 12, 2017.

(51) Int. Cl. C12N 15/11; C12N 9/22

(52) U.S. Cl. CPC C12N 15/11; C12N 2800/80; C12N 9/22

(57) ABSTRACT

The present disclosure provides systems, methods, and compositions for conditionally regulating expression of a target gene. Aspects of the present disclosure utilize intracellular signal transduction pathways to regulate the expression of a gene (e.g., transgene, exogenous gene, endogenous gene).

### CROSS-REFERENCE

This application is a continuation application of International Application No. PCT/US18/41704, filed on Jul. 11, 2018, which claims the benefit of U.S. Provisional Application No. 62/531,752, filed on Jul. 12, 2017, and U.S. Provisional Application No. 62/587,668, filed on Nov. 17, 2017, which applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 21, 2018, is named 50489-707_601_SL.txt and is 9,729 bytes in size.

### BACKGROUND

A receptor is a protein molecule that can receive biochemical signals from outside a cell. In some cases, receptors are linked directly or indirectly to cellular biochemical pathways, and ligand binding to a receptor (e.g., biochemical signal) can activate or inhibit the receptor's associated biochemical pathways. Interaction of a cellular receptor with a ligand can play a central role in sensing environmental cues and translating extracellular stimulation into intracellular signaling. Intracellular signaling can result in the regulation of biochemical processes including transcriptional activation of gene expression and new protein synthesis to control cell behaviors.

Engineering cells with features that can be conditionally controlled by environmental cues can be useful for tuning cellular responses and also for gene and cell therapy applications. Conditional gene expression systems allow for conditional regulation of one or more target genes. Conditional gene expression systems such as drug-inducible gene expression systems allow for the activation and/or deactivation of gene expression in response to a stimulus, such as the presence of a drug. Currently available systems, however, can be limited due to imprecise control, insufficient levels of induction (e.g., activation and/or deactivation of gene expression), and lack of specificity.

### SUMMARY

In view of the foregoing, there exists a considerable need for alternative methods and systems to carry out conditional regulation of gene expression.

In an aspect, the present disclosure provides a system for regulating expression of a target gene in a cell comprising (a) a transmembrane receptor comprising a ligand binding domain and a signaling domain, wherein the signaling domain activates a signaling pathway of the cell upon binding of a ligand to the ligand binding domain; and (b) an expression cassette comprising a nucleic acid sequence encoding a gene modulating polypeptide (GMP) placed under control of a promoter, wherein the GMP comprises an actuator moiety, and wherein the promoter is activated to drive expression of the GMP upon binding of the ligand to the ligand binding domain, wherein the expressed GMP regulates expression of the target gene.

In some embodiments, the promoter comprises an endogenous promoter that is activated upon binding of the ligand to the ligand binding domain. In some embodiments, the nucleic acid encoding the GMP is operably linked to the endogenous promoter. In some embodiments, the expression cassette comprises a gene encoding an endogenous protein, wherein the gene is located upstream of the nucleic acid sequence encoding the GMP, and wherein expression of the endogenous protein is driven by the endogenous promoter. In some embodiments, the gene and the nucleic acid sequence encoding the GMP are joined by a nucleic acid sequence encoding a peptide linker. In some embodiments, the peptide linker comprises a protease recognition sequence. In some embodiments, the peptide linker comprises a self-cleaving segment. In some embodiments, the self-cleaving segment comprises a 2A peptide. In some embodiments, the 2A peptide is T2A, P2A, E2A, or F2A. In some embodiments, the gene and the nucleic acid sequence encoding the GMP are joined by a nucleic acid sequence comprising an internal ribosome entry site (IBES). In some embodiments, the promoter comprises an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter.

In some embodiments, the promoter comprises an exogenous promoter that is activated upon binding of the ligand to the ligand binding domain. In some embodiments, the exogenous promoter comprises a synthetic promoter sequence or a fragment thereof. In some embodiments, the nucleic acid sequencing encoding the GMP is operably linked to the exogenous promoter.

In some embodiments, the transmembrane receptor comprises an endogenous receptor, a synthetic receptor, or any fragment thereof. In some embodiments, the transmembrane receptor comprises a chimeric antigen receptor (CAR), a T cell receptor (TCR), a G-protein coupled receptor (GPCR), an integrin receptor, or a Notch receptor. In some embodiments, the transmembrane receptor comprises a GPCR or a variant thereof. In some embodiments, the transmembrane receptor comprises a chimeric antigen receptor (CAR). In some embodiments, the ligand binding domain of the CAR comprises at least one of a Fab, a single-chain Fv (scFv), an extracellular receptor domain, and an Fc binding domain. In some embodiments, the signaling domain of the CAR comprises an immunoreceptor tyrosine-based activation motif (ITAM). In some embodiments, the signaling domain of the CAR comprises an immunoreceptor tyrosine-based inhibition motif (ITIM). In some embodiments, the signaling domain of the CAR comprises a co-stimulatory domain.

In some embodiments, the actuator moiety is an RNA-guided actuator moiety, and the system further comprises a guide-RNA that complexes with the RNA-guided actuator moiety. In some embodiments, the RNA-guided actuator moiety is Cas9. In some embodiments, Cas9 is an S. pyogenes Cas9. In some embodiments, Cas9 is an S. aureus Cas9. In some embodiments, Cas9 substantially lacks nuclease activity. In some embodiments, the RNA-guided actuator moiety is Cpf1. In some embodiments, Cpf1 substantially lacks nuclease activity. In some embodiments, the GMP comprises at least one targeting peptide, such as a nuclear localization sequence (NLS). In some embodiments, the GMP comprises a transcription activator or repressor.

In some embodiments, the target gene encodes for a cytokine. In some embodiments, the target gene encodes for an immune checkpoint inhibitor. In some embodiments, the immune checkpoint inhibitor is PD-1, CTLA-4, LAG3, TIM-3, A2AR, B7-H3, B7-H4, BTLA, IDO, KIR, or VISTA.

In some embodiments, the target gene encodes for a T cell receptor (TCR) alpha, beta, gamma, and/or delta chain.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK) cell.

In an aspect, the present disclosure provides a system for regulating expression of a target gene in a cell, comprising (a) a first transmembrane receptor comprising a first ligand binding domain and a first signaling domain, wherein the first signaling domain activates a first signaling pathway of the cell upon binding of a first ligand to the first ligand binding domain; (b) a second transmembrane receptor comprising a second ligand binding domain and a second signaling domain, wherein the second signaling domain activates a second signaling pathway of the cell upon binding of a second ligand to the second ligand binding domain; and (c) an expression cassette comprising a nucleic acid sequence encoding a gene modulating polypeptide (GMP) placed under control of a promoter, wherein the GMP comprises an actuator moiety, and wherein the promoter is activated to drive expression of the GMP upon (i) binding of the first ligand to the first ligand binding domain, and/or (ii) binding of the second ligand to the second ligand binding domain, wherein the GMP regulates expression of the target gene.

In some embodiments, the promoter comprises an endogenous promoter that is activated upon binding of the first ligand to the first ligand binding domain. In some embodiments, the promoter comprises an endogenous promoter that is activated upon binding of the second ligand to the second ligand binding domain. In some embodiments, the nucleic acid sequence encoding the GMP is operably linked to the endogenous promoter. In some embodiments, the expression cassette comprises a gene encoding an endogenous protein, wherein the gene is located upstream of the nucleic acid sequencing encoding the GMP, and wherein expression of the endogenous protein is driven by the endogenous promoter. In some embodiments, the gene and the nucleic acid sequence encoding the GMP are joined by a nucleic acid sequence encoding a peptide linker. In some embodiments, the peptide linker comprises a protease recognition sequence. In some embodiments, the peptide linker comprises a self-cleaving segment. In some embodiments, the self-cleaving segment comprises a 2A peptide. In some embodiments, the 2A peptide is T2A, P2A, E2A, or F2A. In some embodiments, the gene and the nucleic acid sequence encoding the GMP are joined by a nucleic acid sequence comprising an internal ribosome entry site (IRES). In some embodiments, the promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter.

In some embodiments, the promoter is an exogenous promoter that is activated upon binding of the first ligand to the first ligand binding domain. In some embodiments, the promoter is an exogenous promoter that is activated upon binding of the second ligand to the second ligand binding domain. In some embodiments, the exogenous promoter comprises a synthetic promoter sequence or a fragment thereof. In some embodiments, the nucleic acid sequencing encoding the GMP is operably linked to the exogenous promoter.

In some embodiments, at least one of the first and second transmembrane receptors comprises an endogenous receptor, a synthetic receptor, or any fragment thereof. In some embodiments, at least one of the first and second transmembrane receptors comprises a chimeric antigen receptor (CAR), a T cell receptor (TCR), G-protein coupled receptor (GPCR), integrin receptor, or Notch receptor. In some embodiments, at least one of the first and second transmembrane receptors comprises a GPCR or a variant thereof. In some embodiments, at least one of the first and second transmembrane receptors comprises a chimeric antigen receptor (CAR). In some embodiments, the ligand binding domain of the CAR comprises at least one of a Fab, a single-chain Fv (scFv), an extracellular receptor domain, and an Fc binding domain. In some embodiments, the signaling domain of the CAR comprises an immunoreceptor tyrosine-based activation motif (ITAM). In some embodiments, the signaling domain of the CAR comprises an immunoreceptor tyrosine-based inhibition motif (ITIM). In some embodiments, the signaling domain of the CAR comprises a co-stimulatory domain.

In some embodiments, the actuator moiety is an RNA-guided actuator moiety, and the system further comprises a guide-RNA that complexes with the RNA-guided actuator moiety. In some embodiments, the RNA-guided actuator moiety is Cas9. In some embodiments, Cas9 is an S. pyogenes Cas9. In some embodiments, Cas9 is an S. aureus Cas9. In some embodiments, Cas9 substantially lacks nuclease activity. In some embodiments, the RNA-guided actuator moiety is Cpf1. In some embodiments, Cpf1 substantially lacks nuclease activity. In some embodiments, the GMP comprises at least one targeting peptide, such as a nuclear localization sequence (NLS). In some embodiments, the GMP comprises a transcription activator or repressor.

In some embodiments, the target gene encodes for a cytokine. In some embodiments, the target gene encodes for an immune checkpoint inhibitor. In some embodiments, the immune checkpoint inhibitor is PD-1, CTLA-4, LAG3, TIM-3, A2AR, B7-H3, B7-H4, BTLA, IDO, KIR, or VISTA.

In some embodiments, the target gene encodes for a T cell receptor (TCR) alpha, beta, gamma, and/or delta chain.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK) cell.

In an aspect, the present disclosure provides a system for regulating expression of two target genes in a cell, comprising (a) a first transmembrane receptor comprising a first ligand binding domain and a first signaling domain, wherein the first signaling domain activates a first signaling pathway of the cell upon binding of a first ligand to the first ligand binding domain; (b) a second transmembrane receptor comprising a second ligand binding domain and a second signaling domain, wherein the second signaling domain activates a second signaling pathway of the cell upon binding of a second ligand to the second ligand binding domain; (c) a first expression cassette comprising a nucleic acid sequence encoding a first gene modulating polypeptide (GMP) placed under the control of a first promoter, wherein the first GMP comprises a first actuator moiety, and wherein the first promoter is activated to drive expression of the first GMP upon binding of the first ligand to the first ligand binding domain; and (d) a second expression cassette comprising a nucleic acid sequence encoding a second gene modulating polypeptide (GMP) placed under the control of a second promoter, wherein the second GMP comprises a second actuator moiety, and wherein the second promoter is activated to drive expression of the second GMP upon binding of the second ligand to the second ligand binding domain, wherein (i) the first GMP regulates expression of a first target gene and (ii) the second GMP regulates expression of a second target gene.

In some embodiments, the first promoter comprises a first endogenous promoter that is activated upon binding of the first ligand to the first ligand binding domain. In some embodiments, the second promoter comprises a second endogenous promoter that is activated upon binding of the second ligand to the second ligand binding domain. In some embodiments, the nucleic acid sequence encoding the first GMP is operably linked to the first endogenous promoter. In some embodiments, the nucleic acid sequence encoding the second GMP is operably linked to the second endogenous promoter. In some embodiments, the first expression cassette comprises a first gene encoding a first endogenous protein, wherein the first gene is located upstream of the nucleic acid sequence encoding the first GMP, and wherein expression of the first endogenous protein is driven by the first endogenous promoter. In some embodiments, the second expression cassette comprises a second gene encoding a second endogenous protein, wherein the second gene is located upstream of the nucleic acid sequence encoding the second GMP, and wherein expression of the second endogenous protein is driven by the second endogenous promoter. In some embodiments, the first gene and the nucleic acid sequence encoding the first GMP are joined by a nucleic acid sequence encoding a peptide linker. In some embodiments, the second gene and the nucleic acid sequence encoding the second GMP are joined by a nucleic acid sequence encoding a peptide linker. In some embodiments, the peptide linker joining the first gene and the first GMP coding sequence and/or the peptide linker joining the second gene and the second GMP coding sequence comprises a protease recognition sequence. In some embodiments, the peptide linker joining the first gene and the first GMP coding sequence and/or the peptide linker joining the second gene and the second GMP coding sequence comprises a self-cleaving segment. In some embodiments, the self-cleaving segment comprises a 2A peptide. In some embodiments, the 2A peptide is T2A, P2A, E2A, or F2A. In some embodiments, the first gene and the nucleic acid sequence encoding the first GMP are joined by a nucleic acid sequence comprising a first internal ribosome entry site (IRES). In some embodiments, the second gene and the nucleic acid sequence encoding the second GMP are joined by a nucleic acid sequence comprising a second internal ribosome entry site (IRES). In some embodiments, the first promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter. In some embodiments, the second promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter.

In some embodiments, the first promoter comprises a first exogenous promoter that is activated upon binding of the first ligand to the first ligand binding domain. In some embodiments, the second promoter comprises a second exogenous promoter that is activated upon binding of the second ligand to the second ligand binding domain. In some embodiments, the first exogenous promoter comprises a synthetic promoter sequence or any fragment thereof. In some embodiments, the second exogenous promoter comprises a synthetic promoter sequence or any fragment thereof. In some embodiments, the nucleic acid sequencing encoding the first GMP is operably linked to the first exogenous promoter. In some embodiments, the nucleic acid sequencing encoding the second GMP is operably linked to the second exogenous promoter.

In some embodiments, at least one of the first and second transmembrane receptors comprises an endogenous receptor, a synthetic receptor, or a fragment thereof. In some embodiments, at least one of the first and second transmembrane receptors comprises a chimeric antigen receptor (CAR), a T cell receptor (TCR), a G-protein coupled receptor (GPCR), an integrin receptor, or a Notch receptor. In some embodiments, at least one of the first and second transmembrane receptors comprises a GPCR or a variant thereof. In some embodiments, at least one of the first and second transmembrane receptors comprises a chimeric antigen receptor (CAR). In some embodiments, the ligand binding domain of the CAR comprises at least one of a Fab, a single-chain Fv (scFv), an extracellular receptor domain, and an Fc binding domain. In some embodiments, the signaling domain of the CAR comprises an immunoreceptor tyrosine-based activation motif (ITAM). In some embodiments, the signaling domain of the CAR comprises an immunoreceptor tyrosine-based inhibition motif (ITIM). In some embodiments, the signaling domain of the CAR comprises a co-stimulatory domain.

In some embodiments, the actuator moiety of at least one of the first GMP and second GMP is an RNA-guided actuator moiety, and the system further comprises a guide-RNA that complexes with the RNA-guided actuator moiety. In some embodiments, the RNA-guided actuator moiety is Cas9. In some embodiments, Cas9 is an S. pyogenes Cas9. In some embodiments, Cas9 is an S. aureus Cas9. In some embodiments, Cas9 substantially lacks nuclease activity. In some embodiments, the RNA-guided actuator moiety is Cpf1. In some embodiments, Cpf1 substantially lacks nuclease activity. In some embodiments, at least one of the first GMP and second GMP comprises at least one targeting peptide, such as a nuclear localization sequence (NLS). In some embodiments, at least one of the first GMP and second GMP comprises to a transcription activator or repressor.

In some embodiments, the first and/or second target gene encodes for a cytokine. In some embodiments, the first and/or second target gene encodes for an immune checkpoint inhibitor. In some embodiments, the immune checkpoint inhibitor is PD-1, CTLA-4, LAG3, TIM-3, A2AR, B7-H3, B7-H4, BTLA, IDO, KIR, or VISTA.

In some embodiments, the target gene encodes for a T cell receptor (TCR) alpha, beta, gamma, and/or delta chain.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK) cell.

In an aspect, the present disclosure provides a system for regulating expression of a target gene in a cell, comprising (a) a first transmembrane receptor comprising a first ligand binding domain and a first signaling domain, wherein the first signaling domain activates a first signaling pathway of the cell upon binding of a first ligand to the first ligand binding domain; (b) a second transmembrane receptor comprising a second ligand binding domain and a second signaling domain, wherein the second signaling domain activates a second signaling pathway of the cell upon binding of a second ligand to the second ligand binding domain; (c) a first expression cassette comprising a nucleic acid encoding a first partial gene modulating polypeptide (GMP) placed under control of a first promoter, wherein the first partial GMP comprises a first portion of an actuator moiety, and wherein the first promoter is activated to drive expression of the first partial GMP upon binding of the first ligand to the first ligand binding domain; and (c) a second expression cassette comprising a nucleic acid encoding a second partial gene modulating polypeptide (GMP) placed under control of a second promoter, wherein the second partial GMP comprises a second portion of an actuator moiety, and wherein the second promoter is activated to drive expression of the second partial GMP upon binding of the second ligand to the second ligand binding domain, wherein the first and second portion of the actuator moiety complex to form a reconstituted GMP comprising a functional actuator moiety, wherein the reconstituted GMP regulates expression of the target gene.

In some embodiments, the first promoter comprises a first endogenous promoter that is activated upon binding of the first ligand to the first ligand binding domain. In some embodiments, the second promoter comprises a second endogenous promoter that is activated upon binding of the second ligand to the second ligand binding domain. In some embodiments, the nucleic acid sequence encoding the first partial GMP is operably linked to the first endogenous promoter. In some embodiments, the nucleic acid sequence encoding the second partial GMP is operably linked to the second endogenous promoter. In some embodiments, the first expression cassette comprises a first gene encoding a first endogenous protein, wherein the first gene is located upstream of the nucleic acid sequence encoding the first partial GMP, and wherein expression of the first endogenous protein is driven by the first endogenous promoter. In some embodiments, the second expression cassette comprises a second gene encoding a second endogenous protein, wherein the second gene is located upstream of the nucleic acid sequence encoding the second partial GMP, and wherein expression of the second endogenous protein is driven by the second endogenous promoter. In some embodiments, the first gene and the nucleic acid sequence encoding the first partial GMP are joined by a nucleic acid sequence encoding a peptide linker. In some embodiments, the second gene and the nucleic acid sequence encoding the second partial GMP are joined by a nucleic acid sequence encoding a peptide linker. In some embodiments, the peptide linker joining the first gene and the first partial GMP coding sequence and/or the peptide linker joining the second gene and the second partial GMP coding sequence comprises a protease recognition sequence. In some embodiments, the peptide linker joining the first gene and the first partial GMP coding sequence and/or the peptide linker joining the second gene and the second GMP coding sequence comprises a self-cleaving segment. In some embodiments, the self-cleaving segment comprises a 2A peptide. In some embodiments, the 2A peptide is T2A, P2A, E2A, or F2A. In some embodiments, the first gene and the nucleic acid sequence encoding the first partial GMP are joined by a nucleic acid sequence comprising a first internal ribosome entry site (IRES). In some embodiments, the second gene and the nucleic acid sequence encoding the second partial GMP are joined by a nucleic acid sequence comprising a second internal ribosome entry site (IRES). In some embodiments, the first promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter. In some embodiments, the second promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter.

In some embodiments, the first promoter comprises a first exogenous promoter that is activated upon binding of the first ligand to the first ligand binding domain. In some embodiments, the second promoter comprises a second exogenous promoter that is activated upon binding of the second ligand to the second ligand binding domain. In some embodiments, the first exogenous promoter comprises a synthetic promoter sequence or any fragment thereof. In some embodiments, the second exogenous promoter comprises a synthetic promoter sequence or any fragment thereof. In some embodiments, the nucleic acid sequencing encoding the first partial GMP is operably linked to the first exogenous promoter. In some embodiments, the nucleic acid sequencing encoding the second partial GMP is operably linked to the second exogenous promoter.

In some embodiments, at least one of the first and second transmembrane receptors comprises an endogenous receptor, a synthetic receptor, or a fragment thereof. In some embodiments, at least one of the first and second transmembrane receptors comprises a chimeric antigen receptor (CAR), a T cell receptor (TCR), a G-protein coupled receptor (GPCR), an integrin receptor, or a Notch receptor. In some embodiments, at least one of the first and second transmembrane receptors comprises a GPCR or a variant thereof. In some embodiments, at least one of the first and second transmembrane receptors comprises a chimeric antigen receptor (CAR). In some embodiments, the ligand binding domain of the CAR comprises at least one of a Fab, a single-chain Fv (scFv), an extracellular receptor domain, and an Fc binding domain. In some embodiments, the signaling domain of the CAR comprises an immunoreceptor tyrosine-based activation motif (ITAM). In some embodiments, the signaling domain of the CAR comprises an immunoreceptor tyrosine-based inhibition motif (ITIM). In some embodiments, the signaling domain of the CAR comprises a co-stimulatory domain.

In some embodiments, the functional actuator moiety comprises an RNA-guided actuator moiety, and the system further comprises a guide-RNA that complexes with the RNA-guided actuator moiety. In some embodiments, the RNA-guided actuator moiety is Cas9. In some embodiments, Cas9 is an S. pyogenes Cas9. In some embodiments, Cas9 is an S. aureus Cas9. In some embodiments, Cas9 substantially lacks nuclease activity. In some embodiments, the RNA-guided actuator moiety is Cpf1. In some embodiments, Cpf1 substantially lacks nuclease activity. In some embodiments, at least one of the first partial GMP and second partial GMP comprises at least one targeting peptide, such as a nuclear localization sequence (NLS). In some embodiments, at least one of the first partial GMP and second partial GMP comprises a transcription activator or repressor.

In some embodiments, the target gene encodes for a cytokine. In some embodiments, the target gene encodes for an immune checkpoint inhibitor. In some embodiments, the immune checkpoint inhibitor is PD-1, CTLA-4, LAG3, TIM-3, A2AR, B7-H3, B7-H4, BTLA, IDO, KIR, or VISTA.

In some embodiments, the target gene encodes for a T cell receptor (TCR) alpha, beta, gamma, and/or delta chain.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK) cell.

In an aspect, the present disclosure provides a method of inducing expression of a gene modulating polypeptide (GMP), comprising (a) providing a cell expressing a transmembrane receptor having a ligand binding domain and a signaling domain; (b) binding a ligand to the ligand binding domain of the transmembrane receptor, wherein the binding activates a signaling pathway of the cell such that a promoter operably linked to a nucleic acid sequence encoding the GMP is in turn activated; and (c) expressing the GMP upon activation of the promoter.

In an aspect, the present disclosure provides a method of regulating expression of a target gene in a cell, comprising (a) contacting a ligand to a transmembrane receptor comprising a ligand binding domain and a signaling domain, wherein upon the contacting, the signaling domain activates a signaling pathway of the cell; (b) expressing a gene modulating polypeptide (GMP) comprising an actuator moiety from an expression construct comprising a nucleic acid sequence encoding the GMP placed under control of a promoter, wherein the promoter is activated to drive expression of the GMP upon binding of the ligand to the ligand binding domain; and (c) increasing or decreasing expression of the target gene via binding of the expressed GMP, thereby regulating expression of the target gene.

In various embodiments of the methods disclosed herein, the transmembrane receptor comprises an endogenous receptor. In various embodiments of the methods disclosed herein, the transmembrane receptor comprises a synthetic receptor. In various embodiments of the methods disclosed herein, the transmembrane receptor comprises a chimeric antigen receptor (CAR), a T cell receptor (TCR), a G-protein coupled receptor (GPCR), an integrin receptor, or a Notch receptor.

In various embodiments of the methods disclosed herein, the transmembrane receptor comprises a GPCR or a variant thereof. In some embodiments, the transmembrane receptor comprises a natural or engineered TCR. In various embodiments of the methods disclosed herein, the transmembrane receptor comprises a TCR for an alpha-fetoprotein (AFP), melanoma-associated antigen 4 (MAGE-A4), melanoma-associated antigen 10 (MAGE-A10), or NY-ESO-1 protein-derived peptide in complex with a human leukocyte antigen (HLA) complex. In various embodiments of the methods disclosed herein, the transmembrane receptor comprises a chimeric antigen receptor (CAR). In some embodiments, the ligand binding domain of the CAR comprises at least one of a Fab, a single-chain Fv (scFv), an extracellular receptor domain, and an Fc binding domain. In some embodiments, the signaling domain of the CAR comprises an immunoreceptor tyrosine-based activation motif (ITAM). In some embodiments, the signaling domain of the CAR comprises an immunoreceptor tyrosine-based inhibition motif (ITIM). In some embodiments, the signaling domain of the CAR comprises a co-stimulatory domain.

In some embodiments, the actuator moiety is an RNA-guided actuator moiety. In some embodiments, the RNA-guided actuator moiety is Cas9. In some embodiments, Cas9 is an S. pyogenes Cas9. In some embodiments, Cas9 is an S. aureus Cas9. In some embodiments, Cas9 substantially lacks nuclease activity. In some embodiments, the RNA-guided actuator moiety is Cpf1. In some embodiments, Cpf1 substantially lacks nuclease activity. In some embodiments, the GMP comprises a nuclear localization sequence (NLS). In some embodiments, the GMP comprises a transcription activator or repressor.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK) cell.

In an aspect, the present disclosure provides an expression cassette comprising a promoter operably linked to a nucleic acid sequence encoding a gene modulating polypeptide (GMP) that comprises an actuator moiety, wherein the expression cassette is characterized in that the promoter is activated to drive expression of the GMP from the expression cassette when the expression cassette is present in a cell expressing a transmembrane receptor which has been activated by binding of a ligand to the transmembrane receptor.

In some embodiments, the transmembrane receptor comprises a signaling domain, and the signaling domain activates a signaling pathway of the cell when the transmembrane receptor is activated. In some embodiments, the signaling domain of the transmembrane receptor activates an immune cell signaling pathway.

In some embodiments, a transcription factor of the activated signaling pathway of the cell binds the promoter, thereby activating the promoter to drive expression of the GMP from the expression cassette. In some embodiments, the promoter comprises an endogenous promoter sequence. In some embodiments, the promoter comprises a synthetic promoter sequence. In some embodiments, the promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter. In some embodiments, the second promoter is an IL-2 promoter, an IFN-γ promoter, an IRF4 promoter, a NR4A1 promoter, a PRDM1 promoter, a TBX21 promoter, a CD69 promoter, a CD25 promoter, or a GZMB promoter.

In some embodiments, the actuator moiety is an RNA-guided actuator moiety. In some embodiments, the RNA-guided actuator moiety is Cas9. In some embodiments, Cas9 is an S. pyogenes Cas9. In some embodiments, Cas9 is an S. aureus Cas9. In some embodiments, Cas9 substantially lacks nuclease activity. In some embodiments, the RNA-guided actuator moiety is Cpf1. In some embodiments, Cpf1 substantially lacks nuclease activity. In some embodiments, the GMP comprises a nuclear localization sequence (NLS). In some embodiments, the GMP comprises a transcription activator or a transcription repressor.

In some embodiments, the expression cassette is integrated into the cell genome. In some embodiments, the expression cassette is integrated into the cell genome via lentivirus. In some embodiments, the expression cassette is integrated into the cell genome via a programmable nuclease. In some embodiments, the programmable nuclease is a RNA-guided nuclease, a zinc finger nuclease (ZNF), or a transcription activator-like effector nuclease (TALEN).

In some embodiments, the expression cassette is integrated into the cell genome at a region comprising a safe harbor site. In some embodiments, the expression cassette is integrated into the AAVS1 site of chromosome 19. In some embodiments, the expression cassette is integrated into the CCR5 site of chromosome 3.

In an aspect, the present disclosure provides an expression cassette comprising (i) a nucleic acid sequence encoding a gene modulating polypeptide (GMP), and (ii) at least one integration sequence which facilitates integration of the expression cassette into a cell genome, wherein the GMP comprises an actuator moiety, and wherein the expression cassette is characterized in that activation of a transmembrane receptor by binding of a ligand to the transmembrane receptor activates a promoter to drive expression of the GMP from the expression cassette when the expression cassette has been integrated into the cell genome via the at least one integration sequence.

In some embodiments, the at least one integration sequence facilitates integration of the expression cassette into a region of the cell genome such that the nucleic acid sequence encoding the GMP is operably linked to an endogenous promoter.

In some embodiments, the at least one integration sequence facilitates integration of the expression cassette into a region of the cell genome such that the nucleic acid sequence encoding the GMP is (i) operably linked to an endogenous promoter and (ii) located downstream of a gene encoding an endogenous protein, wherein expression of the endogenous protein in the cell is driven by the endogenous promoter.

In some embodiments, the nucleic acid sequence encoding the GMP is joined to the gene by a nucleic acid sequence encoding a peptide linker. In some embodiments, the nucleic acid sequence encoding the GMP is joined in-frame to the gene. In some embodiments, the peptide linker comprises a protease recognition sequence. In some embodiments, the peptide linker comprises a self-cleaving segment. In some embodiments, the self-cleaving segment comprises a 2A peptide. In some embodiments, the 2A peptide is T2A, P2A, E2A, or F2A. In some embodiments, the nucleic acid sequence encoding the GMP is joined to the gene by a nucleic acid sequence comprising an internal ribosome entry site (IRES).

In some embodiments, the at least one integration sequence comprises a homology sequence, and the expression cassette is integrated into the cell genome via homology-directed repair (HDR). In some embodiments, two integration sequences flank the nucleic acid sequence encoding a gene modulating polypeptide (GMP), each integration sequence of the two comprising a homology sequence. In some embodiments, the homology sequence facilitates integration of the expression cassette into a targeted region of the cell genome. In some embodiments, the nucleic acid sequence encoding a gene modulating polypeptide is located downstream of the promoter after integration of the expression cassette.

In an aspect, the present disclosure provides a cell comprising any system or expression cassette disclosed herein. In some embodiments, the cell is a hematopoietic cell, a hematopoietic progenitor cell, or a hematopoietic stem cell. In some embodiments, the cell is a hematopoietic cell, and wherein the hematopoietic cell is a lymphocyte, natural killer (NK) cell, monocyte, macrophage, or dendritic cell (DC).

In some embodiments, the expression cassette of the system is present in the cell as part of a plasmid. In some embodiments, the expression cassette of the system is integrated into the cell genome. In some embodiments, the expression cassette is integrated into the cell genome via a programmable nuclease. In some embodiments, the programmable nuclease is a RNA-guided nuclease, a zinc finger nuclease (ZNF), or a transcription activator-like effector nuclease (TALEN).

In some embodiments, the expression cassette of the system is integrated into the cell genome at a region comprising a genomic safe harbor site. In some embodiments, the expression cassette of the system is integrated into the AAVS1 site of chromosome 19. In some embodiments, the expression cassette of the system is integrated into the CCR5 site of chromosome 3.

In an aspect, the present disclosure provides a system for regulating expression of a target gene in a cell comprising (a) a transmembrane receptor comprising a ligand binding domain and a signaling domain, wherein the signaling domain activates a signaling pathway of the cell upon binding of a ligand to the ligand binding domain; and (b) an expression cassette comprising a promoter operably linked to a nucleic acid sequence encoding a gene modulating polypeptide (GMP), wherein the GMP comprises an actuator moiety, and wherein the promoter is activated to drive expression of the GMP upon binding of the ligand to the ligand binding domain, wherein the expressed GMP regulates expression of the target gene.

In an aspect, the present disclosure provides a system for regulating expression of a target gene in a cell. The system comprises (a) a transmembrane receptor comprising a ligand binding domain, a signaling domain, and a gene modulating polypeptide (GMP), wherein the GMP comprises an actuator moiety linked to a cleavage recognition site, and wherein the signaling domain activates a signaling pathway of the cell upon binding of a ligand to the ligand binding domain; and (b) an expression cassette comprising a nucleic acid sequence encoding a cleavage moiety, wherein the nucleic acid sequence is placed under the control of a promoter activated by the signaling pathway to drive expression of the cleavage moiety upon binding of the ligand to the ligand binding domain, wherein the expressed cleavage moiety cleaves the cleavage recognition site when in proximity to the cleavage recognition site to release the actuator moiety, and wherein the released actuator moiety regulates expression of a target gene.

In an aspect, the present disclosure provides a system for regulating expression of a target gene in a cell. The system comprises (a) a transmembrane receptor comprising a ligand binding domain, a signaling domain, and a cleavage moiety, wherein the signaling domain activates a signaling pathway of the cell upon binding of a ligand to the ligand binding domain; and (b) an expression cassette comprising a nucleic acid sequence encoding a fusion protein comprising a gene modulating polypeptide (GMP) linked to a nuclear export signal peptide, wherein the GMP comprises an actuator moiety linked to a cleavage recognition site, and wherein the nucleic acid sequence is placed under the control of a promoter activated by the signaling pathway to drive expression of the fusion protein upon binding of the ligand to the ligand binding domain, wherein the cleavage moiety cleaves the cleavage recognition site of the fusion protein when the fusion protein is in proximity to the cleavage moiety to release the actuator moiety, and wherein the released actuator moiety regulates expression of a target gene. In some embodiments, the cleavage moiety is linked to an intracellular region of the transmembrane receptor.

In an aspect, the present disclosure provides a system for regulating expression of a target gene in a cell. The system comprises (a) a transmembrane receptor comprising a ligand binding domain and a signaling domain, wherein the signaling domain activates a signaling pathway of the cell upon binding of a ligand to the ligand binding domain; and (b) an expression cassette comprising a nucleic acid sequence encoding a cleavage moiety, wherein the nucleic acid sequence is placed under the control of a promoter activated by the signaling pathway to drive expression of the cleavage moiety upon binding of the ligand to the ligand binding domain, wherein the expressed cleavage moiety cleaves a cleavage recognition site of a fusion protein comprising a gene modulating polypeptide (GMP) linked to a nuclear export signal peptide, the GMP comprising an actuator moiety linked to the cleavage recognition site, wherein cleavage of the cleavage recognition site releases the actuator moiety and the released actuator moiety regulates expression of a target gene. In some embodiments, the system comprises the fusion protein comprising the gene modulating polypeptide (GMP) linked to the nuclear export signal peptide.

In an aspect, the present disclosure provides a system for regulating expression of a target gene in a cell. The system comprises (a) a transmembrane receptor comprising a ligand binding domain and a signaling domain, wherein the signaling domain activates a signaling pathway of the cell upon binding of a ligand to the ligand binding domain; and (b) an expression cassette comprising a nucleic acid sequence encoding a fusion protein comprising a gene modulating polypeptide (GMP) linked to a nuclear export signal peptide, wherein the GMP comprises an actuator moiety linked to a cleavage recognition sequence, and wherein the nucleic acid sequence is placed under the control of a promoter activated by the signaling pathway to drive expression of the fusion protein upon binding of the ligand to the ligand binding domain, wherein upon release of the actuator moiety via cleavage by a cleavage moiety at the cleavage recognition site, the released actuator moiety regulates expression of a target gene. In some embodiments, the system further comprises a cleavage moiety. In some embodiments, the cleavage moiety cleaves the cleavage recognition site of the expressed fusion protein when in proximity to the cleavage recognition site.

In an aspect, the present disclosure provides a system for regulating expression of a target gene in a cell. The system comprises (a) transmembrane receptor comprising a ligand binding domain and a signaling domain, wherein the signaling domain activates a signaling pathway of the cell upon binding of a ligand to the ligand binding domain; (b) a first expression cassette comprising a first nucleic acid sequence encoding a fusion protein comprising a gene modulating polypeptide (GMP) linked to a nuclear export signal peptide, wherein the GMP comprises an actuator moiety linked to a cleavage recognition sequence, and wherein the first nucleic acid sequence is placed under the control of a first promoter activated by the signaling pathway to drive expression of the fusion protein upon binding of the ligand to the ligand binding domain; and (c) a second expression cassette comprising a second nucleic acid sequence encoding a cleavage moiety, wherein the second nucleic acid sequence is placed under the control of a second promoter activated by the signaling pathway to drive expression of the cleavage moiety upon binding of the ligand to the ligand binding domain, wherein the expressed cleavage moiety cleaves the cleavage recognition site of the expressed fusion protein when in proximity to the cleavage recognition site to release actuator moiety, and wherein the released actuator moiety regulates expression of a target gene.

In an aspect, the present disclosure provides a system for regulating expression of a target gene in a cell. The system comprises (a) a transmembrane receptor comprising a ligand binding domain and a signaling domain, wherein the signaling domain activates a signaling pathway of the cell upon binding of a ligand to the ligand binding domain; (b) a first expression cassette comprising a first nucleic acid sequence encoding a first partial gene modulating polypeptide (GMP), the first partial GMP comprising a first portion of an actuator moiety, wherein the first nucleic acid sequence is placed under the control of a first promoter activated by the signaling pathway to drive expression of the first partial GMP upon binding of the ligand to the ligand binding domain; and (c) a second expression cassette comprising a second nucleic acid sequence encoding a second partial gene modulating polypeptide (GMP), the second partial GMP comprising a second portion of an actuator moiety, wherein the second nucleic acid sequence is placed under the control of a second promoter activated by the signaling pathway to drive expression of the second partial GMP upon binding of the ligand to the ligand binding domain, and wherein the first partial GMP and second partial GMP complex to form a reconstituted actuator moiety, wherein the reconstituted actuator moiety regulates expression of the target gene.

In an aspect, the present disclosure provides a system for regulating expression of a target gene in a cell. The system comprises (a) a transmembrane receptor comprising a ligand binding domain and a signaling domain, wherein the signaling domain activates a signaling pathway of the cell upon binding of a ligand to the ligand binding domain; (b) a first expression cassette comprising a first nucleic acid sequence encoding a first partial cleavage moiety, wherein the first nucleic acid sequence is placed under the control of a first promoter activated by the signaling pathway to drive expression of the first partial cleavage moiety upon binding of the ligand to the ligand binding domain; and (c) a second expression cassette comprising a second nucleic acid sequence encoding a second partial cleavage moiety, wherein the second nucleic acid sequence is placed under control of a second promoter activated by the signaling pathway to drive expression of the second partial cleavage moiety upon binding of the ligand to the ligand binding domain, wherein the first partial cleavage moiety and the second partial cleavage moiety complex to form a reconstituted cleavage moiety, and upon cleavage by the reconstituted cleavage moiety at a cleavage recognition site to release an actuator moiety from a nuclear export signal peptide, the actuator moiety regulates expression of the target gene. In some embodiments, the system further comprises a fusion polypeptide comprising a nuclear export signal peptide linked to the actuator moiety via the cleavage recognition site.

In an aspect, the present disclosure provides a system for regulating expression of a target gene in a cell. The system comprises (a) a transmembrane receptor comprising a ligand binding domain and a signaling domain, wherein the signaling domain activates a signaling pathway of the cell upon binding of a ligand to the ligand binding domain; and (b) an expression cassette comprising a nucleic acid encoding one or both of (i) a cleavage moiety and (ii) a fusion protein comprising a gene modulating polypeptide (GMP) linked to a nuclear export signal peptide, the GMP comprising an actuator moiety linked to a cleavage recognition site, wherein expression of one or both of the cleavage moiety and the fusion protein is driven by a promoter activated by the signaling pathway upon binding of a ligand to the ligand binding domain, wherein the actuator moiety is released upon cleavage of the cleavage recognition site by the cleavage moiety, and wherein the released GMP regulates expression of a target polynucleotide.

In some embodiments, the transmembrane receptor comprises an endogenous receptor or a synthetic receptor. In some embodiments, the transmembrane receptor comprises a chimeric antigen receptor (CAR), a T cell receptor (TCR), a G-protein coupled receptor (GPCR), an integrin receptor, or a Notch receptor.

In some embodiments, the actuator moiety comprises polynucleotide-guided endonuclease. In some embodiments, the polynucleotide-guided endonuclease is an RNA-guided endonuclease. In some embodiments, the RNA-guided endonuclease is a Cas protein. In some embodiments, the Cas protein is Cas9. In some embodiments, Cas9 is an S. pyogenes Cas9. In some embodiments, Cas9 is an S. aureus Cas9. In some embodiments, the Cas protein substantially lacks nuclease activity. In some embodiments, the Cas protein is Cpf1. In some embodiments, Cpf1 substantially lacks nuclease activity.

In some embodiments, the actuator moiety is linked to a transcription activator. In some embodiments, the actuator moiety is linked to a transcription repressor.

In some embodiments, the promoter is selected from an IL-2, IFN-γ, IRF4, NR4A1, PRDM1, TBX21, CD69, CD25, and GZMB promoter.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK cell).

In an aspect, the present disclosure provides a method of regulating expression of a target gene in a cell. The method comprises (a) contacting a ligand to a transmembrane receptor comprising a ligand binding domain, a signaling domain, and a gene modulating polypeptide (GMP), the GMP comprising an actuator moiety linked to a cleavage recognition site, wherein upon contacting the ligand to the ligand binding domain, the signaling domain activates a signaling pathway of the cell; (b) expressing a cleavage moiety from an expression cassette comprising a nucleic acid sequence encoding the cleavage moiety, wherein the nucleic acid sequence is placed under the control of a promoter activated by the signaling pathway to drive expression of the cleavage moiety upon binding of the ligand to the ligand binding domain; and (c) cleaving, by the cleavage moiety, the cleavage recognition site to release the actuator moiety from the transmembrane receptor, wherein the released actuator moiety regulates expression of the target gene.

In an aspect, the present disclosure provides a method of regulating expression of a target gene in a cell. The method comprises (a) contacting a ligand to a transmembrane receptor comprising a ligand binding domain, a signaling domain, and a cleavage moiety, wherein upon contacting the ligand to the ligand binding domain, the signaling domain activates a signaling pathway of the cell; (b) expressing a fusion protein comprising a gene modulating polypeptide (GMP) linked to a nuclear export signal peptide, the GMP comprising an actuator moiety linked to a cleavage recognition site from an expression cassette comprising the nucleic acid sequence, wherein the nucleic acid sequence is placed under the control of a promoter activated by the signaling pathway to drive expression of the fusion protein upon binding of the ligand to the ligand binding domain; and (c) cleaving, by the cleavage moiety, the cleavage recognition site to release the actuator moiety, wherein the released actuator moiety regulates expression of a target gene. In some embodiments, the cleavage moiety is linked to an intracellular region of the transmembrane receptor.

In an aspect, the present disclosure provides a method of regulating expression of a target gene in a cell. The method comprises (a) contacting a ligand with a transmembrane receptor comprising a ligand binding domain and a signaling domain, wherein upon contacting the ligand to the ligand binding domain, the signaling domain activates a signaling pathway of the cell; (b) expressing a cleavage moiety from an expression cassette comprising a nucleic acid sequence encoding the cleavage moiety, wherein the nucleic acid sequence is placed under the control of a promoter activated by the signaling pathway to drive expression of the cleavage moiety upon binding of the ligand to the ligand binding domain; and (c) cleaving, by the cleavage moiety, a cleavage recognition site of a fusion protein comprising a gene modulating polypeptide (GMP) linked to a nuclear export signal peptide, wherein the GMP comprises an actuator moiety linked to the cleavage recognition site, wherein upon cleaving, the actuator moiety is released, wherein the released actuator moiety regulates expression of a target gene.

In an aspect, the present disclosure provides a method of regulating expression of a target gene in a cell. The method comprises (a) contacting a ligand to a transmembrane receptor comprising a ligand binding domain and a signaling domain, wherein upon contacting the ligand to the ligand binding domain, the signaling domain activates a signaling pathway of the cell; (b) expressing a fusion protein comprising a gene modulating polypeptide (GMP) linked to a nuclear export signal peptide from an expression cassette comprising a nucleic acid sequence encoding the fusion protein, the GMP comprising an actuator moiety linked to a cleavage recognition sequence, wherein the nucleic acid sequence is placed under the control of a promoter activated by the signaling pathway to drive expression of the fusion protein upon binding of the ligand to the ligand binding domain; and (c) cleaving, by a cleavage moiety, the cleavage recognition site of the fusion protein to release the actuator moiety, wherein the released actuator moiety regulates expression of a target gene. In some embodiments, the cleavage moiety cleaves the cleavage recognition site of the expressed fusion protein when in proximity to the cleavage recognition site.

In an aspect, the present disclosure provides a method of regulating expression of a target gene in a cell. The method comprises (a) contacting a ligand to a transmembrane receptor comprising a ligand binding domain and a signaling domain, wherein upon contacting the ligand to the ligand binding domain, the signaling domain activates a signaling pathway of the cell; (b) expressing a fusion protein comprising a gene modulating polypeptide (GMP) linked to a nuclear export signal peptide from a first expression cassette comprising a first nucleic acid sequence encoding the fusion protein, the GMP comprising an actuator moiety linked to a cleavage recognition sequence, wherein the nucleic acid sequence is placed under the control of a first promoter activated by the signaling pathway to drive expression of the fusion protein upon binding of the ligand to the ligand binding domain; (c) expressing a cleavage moiety from a second expression cassette comprising a nucleic acid sequence encoding the cleavage moiety, wherein the nucleic acid is placed under the control of a second promoter activated by the signaling pathway to drive expression of the cleavage moiety upon binding of the ligand to the ligand binding domain; and (d) cleaving the cleavage recognition site of the expressed fusion protein using the expressed cleavage moiety to release the actuator moiety, wherein the released actuator moiety regulates expression of a target gene.

In an aspect, the present disclosure provides a method of regulating expression of a target gene in a cell. The method comprises (a) contacting a ligand to a transmembrane receptor comprising a ligand binding domain and a signaling domain, wherein upon contacting the ligand to the ligand binding domain, the signaling domain activates a signaling pathway of the cell; (b) expressing a first partial gene modulating polypeptide (GMP) from a first expression cassette comprising a first nucleic acid sequence encoding the first partial GMP, the first partial GMP comprising a first portion of an actuator moiety, wherein the first nucleic acid sequence is placed under the control of a first promoter activated by the signaling pathway to drive expression of the first partial GMP upon binding of the ligand to the ligand binding domain; (c) expressing a second partial gene modulating polypeptide (GMP) from a second expression cassette comprising a second nucleic acid sequence encoding the second partial GMP, the second partial GMP comprising a second portion of an actuator moiety, wherein the second nucleic acid sequence is placed under the control of a second promoter activated by the signaling pathway to drive expression of the second partial GMP upon binding of the ligand to the ligand binding domain; and (d) forming a complex of the first partial GMP and second partial GMP to form a reconstituted actuator moiety, wherein the reconstituted actuator moiety regulates expression of the target gene.

In an aspect, the present disclosure provides a method of regulating expression of a target gene in a cell. The method comprises (a) contacting a ligand to a transmembrane receptor comprising a ligand binding domain and a signaling domain, wherein upon binding of the ligand to the ligand binding domain, the signaling domain activates a signaling pathway of the cell; (b) expressing a first partial cleavage moiety from a first expression cassette comprising a first nucleic acid sequence encoding the first partial cleavage moiety, wherein the first nucleic acid sequence is placed under the control of a first promoter activated by the signaling pathway to drive expression of the first partial cleavage moiety upon binding of the ligand to the ligand binding domain; (c) expressing a second partial cleavage moiety from a second expression cassette comprising a second nucleic acid sequence encoding the second partial cleavage moiety, wherein the second nucleic acid sequence is placed under the control of a second promoter activated by the signaling pathway to drive expression of the second partial cleavage moiety upon binding of the ligand to the ligand binding domain; (d) forming a complex of the first and second partial cleavage moiety to yield a reconstituted cleavage moiety; and (e) cleaving, by the reconstituted cleavage moiety, a cleavage recognition site to release an actuator moiety from a nuclear export signal peptide using the reconstituted cleavage moiety, wherein the released actuator moiety regulates expression of the target gene.

In an aspect, the present disclosure provides a method of regulating expression of a target gene in a cell. The method comprises (a) contacting a ligand to a transmembrane receptor comprising a ligand binding domain and a signaling domain, wherein upon contacting the ligand to the ligand binding domain, the signaling domain activates a signaling pathway of the cell; (b) expressing one or both of (i) a cleavage moiety and (ii) a fusion protein comprising a gene modulating polypeptide (GMP) linked to a nuclear export signal peptide, the GMP comprising an actuator moiety linked to a cleavage recognition site, from an expression cassette comprising a nucleic acid sequence encoding one or both of (i) and (ii), wherein the nucleic acid sequence is placed under the control of a promoter activated by the signaling pathway upon binding of a ligand to the ligand binding domain; and (c) releasing the actuator moiety upon cleavage of the cleavage recognition site by the cleavage moiety, wherein the released actuator moiety regulates expression of a target polynucleotide.

In some embodiments, the transmembrane receptor comprises an endogenous receptor or a synthetic receptor. In some embodiments, the transmembrane receptor comprises a chimeric antigen receptor (CAR), a T cell receptor (TCR), a G-protein coupled receptor (GPCR), an integrin receptor, or a Notch receptor.

In some embodiments, the actuator moiety comprises a polynucleotide-guided endonuclease. In some embodiments, the polynucleotide-guided endonuclease is an RNA-guided endonuclease. In some embodiments, the RNA-guided endonuclease is a Cas protein. In some embodiments, the Cas protein is Cas9. In some embodiments, Cas9 is an S. pyogenes Cas9. In some embodiments, Cas9 is an S. aureus Cas9. In some embodiments, the Cas protein substantially lacks nuclease activity. In some embodiments, the Cas protein is Cpf1. In some embodiments, Cpf1 substantially lacks nuclease activity.

In some embodiments, the actuator moiety is linked to a transcription activator. In some embodiments, the actuator moiety is linked to a transcription repressor.

In some embodiments, the promoter is selected from an IL-2, IFN-γ, IRF4, NR4A1, PRDM1, TBX21, CD69, CD25, and GZMB promoter.

In some embodiments, the cell is an immune cell, a hematopoietic progenitor cell, or a hematopoietic stem cell. In some embodiments, the cell is an immune cell. In some embodiments, the immune cell is a lymphocyte. In some embodiments, the lymphocyte is a T cell. In some embodiments, the lymphocyte is a natural killer (NK cell). In some embodiments, the target gene encodes for a cytokine. In some embodiments, the target gene encodes for an immune checkpoint inhibitor. In some embodiments, the immune checkpoint inhibitor is PD-1, CTLA-4, LAG3, TIM-3, A2AR, B7-H3, B7-H4, BTLA4, IDO, KIR, or VISTA. In some embodiments, the target gene encodes for a T cell receptor (TCR) alpha, beta, delta, or gamma chain.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 provides an illustrative schematic of a system provided herein comprising one transmembrane receptor.

FIG. 2 provides an illustrative schematic of a system provided herein comprising two transmembrane receptors.

FIG. 3A illustrates schematically the regulation of reporter gene expression in a Jurkat-derived cell line using a system disclosed herein. FIG. 3B-FIG. 3E 3B-E show conditional expression of a GFP reporter gene by a ligand-dependent signal cascade. FIG. 3B provides histograms of GFP expression which is indirectly driven by various promoters through dCas9-VPR and sgRNA. FIG. 3C and FIG. 3D. 3C and 3D quantify the results of FIG. 3B. FIG. 3E demonstrates ligand-receptor interaction dependent induction of GFP expression (e.g., presence or absence of CAR).

FIG. 4A and FIG. 4B show conditional expression of a GFP reporter gene by a ligand-dependent signal cascade in stable cell lines. FIG. 4A shows induction of GFP reporter expression by various promoters in stable cell lines. FIG. 4B shows activation of GZMB promoter in a ligand or receptor-specific manner using sorted stable cell lines.

FIG. 5A and FIG. 5B shows simultaneous induction of expression of multiple genes, including an endogeneous gene, by an inducible synthetic promoter through the CAR signaling pathway. FIG. 5A shows up-regulation of GFP reporter gene expression. FIG. 5B shows up-regulation of CD95 endogenous gene expression.

FIG. 6 provides an illustrative schematic of a system provided herein comprising a transmembrane receptor linked to a gene modulating polypeptide (GMP).

FIG. 7 provides an illustrative schematic of a system provided herein comprising a transmembrane receptor linked to a cleavage moiety.

FIG. 8 provides an illustrative schematic of a system provided herein in which a cleavage moiety can be expressed from an expression cassette.

FIG. 9 provides an illustrative schematic of a system provided herein in which a fusion polypeptide comprising a gene modulating polypeptide (GMP) linked to a nuclear export signal peptide (NES) can be expressed from an expression cassette.

FIG. 10 provides an illustrative schematic of a system provided herein in which both a cleavage moiety and a fusion polypeptide comprising a gene modulating polypeptide (GMP) linked to a nuclear export signal peptide (NES) can be expressed from one or more expression cassettes.

FIG. 11A and FIG. 11B show that CMV can be induced through the CAR signaling pathway.

FIG. 12 shows conditional expression of a GFP reporter gene by ligand-dependent signal cascade in a system disclosed herein.

FIG. 13 shows conditional expression of a GFP reporter gene by ligand-dependent signal cascade in a system disclosed herein.

FIG. 14 shows suppression of PD-1 expression with dCas9-KRAB controlled by inducible promoters NFATRE and GZMB.

FIG. 15 provides an illustrative schematic of a system provided herein in which the activation of a combination of multiple receptors and signal transduction pathways can conditionally up-regulate or down-regulate the expression of different target genes simultaneously, utilizing the RNA-binding capacity of bacteriophage proteins MCP and PCP.

FIG. 16 provides an illustrative schematic of a system provided herein in which the activation of a combination of multiple receptors and signal transduction pathways can conditionally up-regulate or down-regulate the expression of different target genes simultaneously, utilizing the RNA-binding capacity of PUF proteins.

### DETAILED DESCRIPTION

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used in the specification and claims, the singular forms “a,” “an,” and “the” include plural references unless the context clearly dictates otherwise. For example, the term “a transmembrane receptor” can include a plurality of transmembrane receptors.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not orginating from a natural organism (e.g. a cell can be a synthetically made, sometimes termed an artificial cell).

The term “antigen,” as used herein, refers to a molecule or a fragment thereof (e.g., ligand) capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

The term “antibody,” as used herein, refers to a proteinaceous binding molecule with immunoglobulin-like functions. The term antibody includes antibodies (e.g., monoclonal and polyclonal antibodies), as well as variants thereof. Antibodies include, but are not limited to, immunoglobulins (Ig's) of different classes (i.e. IgA, IgG, IgM, IgD and IgE) and subclasses (such as IgG1, IgG2, etc.). A variant can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The terms “Fc receptor” or “FcR,” as used herein, generally refers to a receptor, or any variant thereof, that can bind to the Fc region of an antibody. In certain embodiments, the FcR is one which binds an IgG antibody (a gamma receptor, Fcgamma R) and includes receptors of the Fcgamma RI (CD64), Fcgamma RII (CD32), and Fcgamma RIII (CD16) subclasses, including allelic variants and alternatively spliced forms of these receptors. Fcgamma RII receptors include Fcgamma RIIA (an “activating receptor”) and Fcgamma RIM (an “inhibiting receptor”), which have similar amino acid sequences that differ primarily in the cytoplasmic domains thereof. The term “FcR” also includes the neonatal receptor, FcRn, which is responsible for the transfer of maternal IgGs to the fetus.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif.; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer (e.g., transgene). A non-native gene can also refer to a naturally occurring nucleic acid or polypeptide sequence that comprises mutations, insertions and/or deletions (e.g., non-native sequence).

The terms “upstream” and “downstream,” as used herein, refer to positions defined in terms relative to the forward strand of a double stranded (ds) DNA molecule. Sequences “upstream” are found at positions nearer the 5′ end of the forward strand (and therefore nearer the 3′ end of the reverse strand) than are “downstream” sequences, which are nearer the 3′ end of the forward strand (and therefore also nearer the 5′ end of the reverse strand).

The terms “target polynucleotide” and “target nucleic acid,” as used herein, refer to a nucleic acid or polynucleotide which is targeted by an actuator moiety of the present disclosure. A target polynucleotide can be DNA (e.g., endogenous or exogenous). DNA can refer to template to generate mRNA transcripts and/or the various regulatory regions which regulate transcription of mRNA from a DNA template. A target polynucleotide can be a portion of a larger polynucleotide, for example a chromosome or a region of a chromosome. A target polynucleotide can refer to an extrachromosomal sequence (e.g., an episomal sequence, a minicircle sequence, a mitochondrial sequence, a chloroplast sequence, etc.) or a region of an extrachromosomal sequence. A target polynucleotide can be RNA. RNA can be, for example, mRNA which can serve as template encoding for proteins. A target polynucleotide comprising RNA can include the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression of a gene product. In general, the term “target sequence” refers to a nucleic acid sequence on a single strand of a target nucleic acid. The target sequence can be a portion of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by an actuator moiety, can result in altered gene expression and/or activity. A target polynucleotide, when targeted by an actuator moiety, can result in an edited nucleic acid sequence. A target nucleic acid can comprise a nucleic acid sequence that may not be related to any other sequence in a nucleic acid sample by a single nucleotide substitution. A target nucleic acid can comprise a nucleic acid sequence that may not be related to any other sequence in a nucleic acid sample by a 2, 3, 4, 5, 6, 7, 8, 9, or 10 nucleotide substitutions. In some embodiments, the substitution may not occur within 5, 10, 15, 20, 25, 30, or 35 nucleotides of the 5′ end of a target nucleic acid. In some embodiments, the substitution may not occur within 5, 10, 15, 20, 25, 30, 35 nucleotides of the 3′ end of a target nucleic acid.

The terms “transfection” or “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The term “expression cassette,” as used herein, refers to a nucleic acid that includes a nucleotide sequence such as a coding sequence and sequences necessary for expression of the coding sequence. The term expression cassette includes regions of the genome, including that which has been edited by genome editing techniques. The term expression cassette also includes nucleic acids that are separate from the genome of a cell (e.g., existing as a plasmid or linear polypeptide). An expression cassette can comprise genomic sequences, such as natural genomic sequences (e.g., endogenous promoter sequences, endogenous genes, etc.) and non-natural sequences (e.g., GMP coding sequence, synthetic promoter sequences, etc.). An expression cassette can be viral or non-viral. An expression cassette includes a nucleic acid construct which, when introduced into a host cell, results in transcription and/or translation of a RNA or polypeptide, respectively. Antisense constructs or sense constructs that are not or cannot be translated are expressly included by this definition. One of skill will recognize that the inserted polynucleotide sequence need not be identical, but may be only substantially similar to a sequence of the gene from which it was derived.

A “plasmid,” as used herein, generally refers to a non-viral expression vector, e.g., a nucleic acid molecule that encodes for genes and/or regulatory elements necessary for the expression of genes. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “promoter,” as used herein, refers to a polynucleotide sequence capable of driving transcription of a coding sequence in a cell. Thus, promoters of the disclosure include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” generally refers to a promoter capable of initiating transcription in nearly all tissue types and under a variety of cellular conditions. An “inducible promoter” generally refers to a promoter that initiates transcription only under particular cellular conditions, environmental conditions, developmental conditions, or drug or chemical conditions. A “tissue-specific promoter” refers to a promoter which initiates transcription only in one or a few particular tissue types.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “regulating” with reference to expression or activity, as used herein, refers to altering the level of expression or activity. Regulation can occur at the transcriptional level, post-transcriptional level, translational level, and/or post-translational level.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “variant,” when used herein with reference to a polypeptide, refers to a polypeptide related, but not identical, to a wild type polypeptide, for example either by amino acid sequence, structure (e.g., secondary and/or tertiary), activity (e.g., enzymatic activity) and/or function. Variants include polypeptides comprising one or more amino acid variations (e.g., mutations, insertions, and deletions), truncations, modifications, or combinations thereof compared to a wild type polypeptide. Variants also include derivatives of the wild type polypeptide and fragments of the wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “gene modulating polypeptide” or “GMP,” as used herein, refers to a polypeptide comprising at least an actuator moiety capable of regulating expression or activity of a gene and/or editing a nucleic acid sequence. A GMP can comprise additional peptide sequences which are not directly involved in modulating gene expression, for example targeting sequences, polypeptide folding domains, etc.

The term “actuator moiety,” as used herein, refers to a moiety which can regulate expression or activity of a gene and/or edit a nucleic acid sequence, whether exogenous or endogenous. An actuator moiety can regulate expression of a gene at the transcriptional level, post-transcriptional level, translational level, and/or post-translation level. An actuator moiety can regulate gene expression at the transcription level, for example, by regulating the production of mRNA from DNA, such as chromosomal DNA or cDNA. In some embodiments, an actuator moiety recruits at least one transcription factor that binds to a specific DNA sequence, thereby controlling the rate of transcription of genetic information from DNA to mRNA. An actuator moiety can itself bind to DNA and regulate transcription by physical obstruction, for example preventing proteins such as RNA polymerase and other associated proteins from assembling on a DNA template. An actuator moiety can regulate expression of a gene at the translation level, for example, by regulating the production of protein from mRNA template. In some embodiments, an actuator moiety regulates gene expression at a post-transcriptional level by affecting the stability of an mRNA transcript. In some embodiments, an actuator moiety regulates gene expression at a post-translational level by altering the polypeptide modification, such as glycosylation of newly synthesized protein. In some embodiments, an actuator moiety regulates expression of a gene by editing a nucleic acid sequence (e.g., a region of a genome). In some embodiments, an actuator moiety regulates expression of a gene by editing an mRNA template. Editing a nucleic acid sequence can, in some cases, alter the underlying template for gene expression.

A Cas protein referred to herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive, partially active, constitutively active, fully active, inducible active and/or more active, (e.g. more than the wild type homologue of the protein or polypeptide). A Cas protein can be Cas9. A Cas protein can be Cpf1. A Cas protein can be C2c2. A Cas protein can be Cas 13a. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA.

The term “crRNA,” as used herein, can generally refer to a nucleic acid with at least about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can generally refer to a nucleic acid with at most about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can refer to a modified form of a crRNA that can comprise a nucleotide change such as a deletion, insertion, or substitution, variant, mutation, or chimera. A crRNA can be a nucleic acid having at least about 60% sequence identity to a wild type exemplary crRNA (e.g., a crRNA from S. pyogenes, S. aureus, etc) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary crRNA sequence (e.g., a crRNA from S. pyogenes S. aureus, etc) over a stretch of at least 6 contiguous nucleotides.

The term “tracrRNA,” as used herein, can generally refer to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc) sequence over a stretch of at least 6 contiguous nucleotides.

As used herein, a “guide nucleic acid” can refer to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A guide nucleic acid can comprise two polynucleotide chains and can be called a “double guide nucleic acid.” If not otherwise specified, the term “guide nucleic acid” can be inclusive, referring to both single guide nucleic acids and double guide nucleic acids.

A guide nucleic acid can comprise a segment that can be referred to as a “nucleic acid-targeting segment” or a “nucleic acid-targeting sequence.” A nucleic acid-targeting segment can comprise a sub-segment that can be referred to as a “protein binding segment” or “protein binding sequence” or “Cas protein binding segment”.

The terms “cleavage recognition sequence” and “cleavage recognition site,” as used herein, with reference to peptides, refers to a site of a peptide at which a chemical bond, such as a peptide bond or disulfide bond, can be cleaved. Cleavage can be achieved by various methods. Cleavage of peptide bonds can be facilitated, for example, by an enzyme such as a protease

The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a GMP) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. A fusion protein can be a non-native sequence in a Cas protein.

As used herein, the “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

In an aspect, the present disclosure provides a system for regulating expression of a target gene in a cell. The system comprises (a) a transmembrane receptor comprising a ligand binding domain and a signaling domain, wherein the signaling domain activates a signaling pathway of the cell upon binding of a ligand to the ligand binding domain and (b) an expression cassette comprising a nucleic acid sequence encoding a gene modulating polypeptide (GMP) placed under control of a promoter, wherein the GMP comprises an actuator moiety, and wherein the promoter is activated to drive expression of the GMP upon binding of the ligand to the ligand binding domain, wherein the expressed GMP regulates expression of the target gene. In some embodiments, the promoter is activated to drive expression of the GMP preferentially upon binding of the ligand to the ligand binding domain. In some embodiments, the promoter is activated to drive expression of the GMP primarily upon binding of the ligand to the ligand binding domain. In some embodiments, the promoter is activated to drive expression of the GMP only upon binding of the ligand to the ligand binding domain.

The transmembrane receptor of a subject system can comprise an extracellular region, a transmembrane region, and an intracellular region. The extracellular region can comprise a ligand binding domain suitable for binding a ligand. The intracellular region can comprise a signaling domain which activates a signaling pathway of the cell upon binding of a ligand to the ligand binding domain. The transmembrane region, or a region of the receptor that spans a cell membrane, can link or join the extracellular region to the intracellular region.

The transmembrane receptor of a subject system can comprise an endogenous receptor, a synthetic receptor, or variant thereof. Endogenous receptors include those which are naturally found in a cell. Exogenous receptors include receptors exogenously introduced into a cell. An exogenous receptor may contain sequences naturally found in a cell. In another example, an exogenous receptor may be a receptor of a different organism or species. Exogenous receptors also include synthetic receptors which are not naturally occurring in any organism. Exogenous receptors include chimeric receptors, which refer to receptors constructed by joining regions (e.g., extracellular, transmembrane, intracellular, etc.) of different molecules (e.g., different proteins, homologous proteins, orthologous proteins, etc).

A synthetic transmembrane receptor of a subject system can comprise a chimeric receptor having at least an extracellular region, a transmembrane region, and an intracellular region. The extracellular region can comprise a ligand binding domain capable of binding a ligand. In some cases, the ligand binding domain is that of an endogenous receptor. In some cases, the ligand binding domain is a synthetic or artificial ligand binding domain which has been engineered in vitro to have certain properties, such as, but not limited to, binding specificity and binding affinity for a particular ligand. The transmembrane region may form any of a variety of three-dimensional structures, including alpha helices and beta barrels. The intracellular region can comprise a signaling domain capable of activating a signaling pathway of the cell. The extracellular, transmembrane, and intracellular regions of a synthetic transmembrane receptor can be selected so as to create a chimeric receptor with desired properties. For example, a synthetic transmembrane receptor can be constructed to as to generate a receptor with binding specificity and affinity for a particular ligand. For further example, the synthetic receptor can be constructed so as to generate a receptor which activates one or multiple signaling pathways of the cell. In some embodiments, a transmembrane receptor has a minimal or no intracellular region and the transmembrane and/or transmembrane-proximal region functions as the signaling domain.

A synthetic transmembrane receptor resulting from the joining of various regions, or domains, from different molecules can be different from the molecules from which the domains originated, for example structurally and functionally. However, the individual domains can, in some cases, retain the native structure and/or activity. For example, the individual domains may retain at least 5%, 10%, 15%, 20%, 25%, 30%, 35%, 40%, 45%, 50%, 55%, 60%, 65%, 70%, 75%, 80%, 85%, 90%, or 95% of the native structure and/or activity. For example, an extracellular region comprising a ligand binding domain can retain at least 5%, 10%, 15%, 20%, 25%, 30%, 35%, 40%, 45%, 50%, 55%, 60%, 65%, 70%, 75%, 80%, 85%, 90%, or 95% of the binding affinity of the molecule from which the extracellular region was derived. For further example, an intracellular region comprising a signaling domain can retain at least 5%, 10%, 15%, 20%, 25%, 30%, 35%, 40%, 45%, 50%, 55%, 60%, 65%, 70%, 75%, 80%, 85%, 90%, or 95% of the ability to activate a signaling pathway of the cell compared to the molecule from which the intracellular region was derived.

In some embodiments, the transmembrane receptor comprises an endogenous receptor. Any suitable endogenous receptor can be used in a subject system for regulating expression of a target gene in a cell. The transmembrane receptor can comprise a Notch receptor; a G-protein coupled receptor (GPCR); an integrin receptor; a cadherin receptor; a catalytic receptor, including receptors possessing enzymatic activity and receptors which, rather than possessing intrinsic enzymatic activity, act by stimulating non-covalently associated enzymes (e.g., kinases); death receptors such as members of the tumor necrosis factor receptor (TNFR) superfamily; immune receptors, such as a T cell receptor (TCR); or any variant thereof. In some embodiments, the transmembrane receptor of the system comprises a GPCR. In some embodiments, the transmembrane receptor of the system comprises an integrin subunit.

In some embodiments, the transmembrane receptor of a subject system comprises an exogenous receptor. In some embodiments, the exogenous receptor is a synthetic receptor. In some embodiments, the synthetic receptor is a chimeric receptor. The transmembrane receptor can comprise a chimeric antigen receptor (CAR), a synthetic integrin receptor, a synthetic Notch receptor, or a synthetic GPCR receptor.

In some embodiments, the transmembrane receptor comprises a chimeric antigen receptor (CAR). The ligand binding domain (e.g., extracellular region) of the CAR can comprise a Fab, a single-chain variable fragment (scFv), the extracellular region of an endogenous receptor (e.g., GPCR, integrin receptor, T-cell receptor, B-cell receptor, etc), or an Fc binding domain. The CAR can comprise a transmembrane domain which situates the receptor in a cell membrane (e.g., plasma membrane, organelle membrane, etc). In some embodiments, the signaling domain (e.g., intracellular region) of the CAR comprises at least one immunoreceptor tyrosine-based activation motif (ITAM). In some embodiments, the signaling domain (e.g., intracellular region) of the CAR comprises at least one immunoreceptor tyrosine-based inhibition motif (ITIM). In some embodiments, the CAR comprises both an ITAM motif and an ITIM motif. In some embodiments, the CAR comprises at least one co-stimulatory domain.

Upon binding of a ligand to the ligand binding domain of a transmembrane receptor, whether an endogenous transmembrane receptor or an exogenous transmembrane receptor (e.g., a synthetic receptor, e.g., chimeric receptor), the signaling domain of the receptor can activate at least one signaling pathway of the cell. The signaling pathway and its associated proteins can be involved in regulating (e.g., activating and/or de-activating) a cellular response such as programmed changes in gene expression via translational regulation; transcriptional regulation; and epigenetic modification including the regulation of methylation, acetylation, phosphorylation, ubiquitylation, sumoylation, ribosylation, and citrullination.

In some cases, the cellular response resulting from activation of the signaling pathway includes changes in gene expression via transcriptional regulation. The cellular response resulting from activation of the signaling pathway may be an increase in expression of a gene via transcriptional regulation. Alternatively, the cellular response resulting from activation of the signaling pathway may be a decrease in expression of a gene via transcriptional regulation. Activation of a single signaling pathway can, in some cases, result in changes in expression levels of multiple genes. The changes may be increases in expression, decreases in expression, or a combination of increase and decrease for different genes. In some cases, at least one transcription factor is recruited to a promoter where it can increase or decrease expression of a gene. In some cases, multiple signaling pathways can regulate the expression levels of one gene.

Transcriptional regulation in response to signaling pathway activation can be utilized in systems provided herein to express a gene modulating polypeptide (GMP). A nucleic acid sequence encoding a GMP, or GMP coding sequence, can be placed under control of a promoter that is responsive to the signaling pathway activated in the cell in response to ligand-receptor binding.

In some cases, the promoter is an endogenous promoter that is activated upon binding of a ligand to the ligand binding domain (e.g., activation of a signaling pathway of the cell). Endogenous promoters include promoter sequences naturally found in a cell genome. Endogenous promoters also include endogenous promoter sequences which are found naturally in a cell genome but which are not at their natural location in the genome. In some cases, the promoter of a system is an endogenous promoter which regulates expression of a gene and is specifically activated by an interaction between a given ligand and receptor pair. For example, expression of the gene can be detected when a given ligand-receptor pair interact (e.g., bind). In some cases, the promoter of a system is preferentially activated by an interaction between a given ligand and receptor pair. In some cases, the promoter of a system is primarily activated by an interaction between a given ligand and receptor pair. For example, expression of the gene is primarily detected when a given ligand-receptor pair interact (e.g., bind). In some cases, the promoter of a system is only activated by an interaction between a given ligand and receptor pair. For example, expression of the gene is only detected when a given ligand-receptor pair interact (e.g., bind).

In some embodiments, the signaling pathway activated in the cell is the PI3K/AKT pathway. In some embodiments, the transmembrane receptor comprises a receptor tyrosine kinase, integrin, B cell receptor, T cell receptor, cytokine receptor, or G-protein coupled receptor and the promoter regulates expression of PRKCE, ITGAM, ITGA5, IRAK1, PRKAA2, EIF2AK2, PTEN, EIF4E, PRKCZ, GRK6, MAPK1, TSC1, PLK1, AKT2, IKBKB, PIK3CA, CDK8, CDKN1B, NFKB2, BCL2, PIK3CB, PPP2R1A, MAPK8, BCL2L1, MAPK3, TSC2, ITGA1, KRAS, EIF4EBP1, RELA, PRKCD, NOS3, PRKAA1, MAPK9, CDK2, PPP2CA, PIM1, ITGB7, YWHAZ, ILK, TP53, RAF1, IKBKG, RELB, DYRK1A, CDKN1A, ITGB1, MAP2K2, JAK1, AKT1, JAK2, PIK3R1, CHUK, PDPK1, PPP2R5C, CTNNB1, MAP2K1, NFKB1, PAK3, ITGB3, CCND1, GSK3A, FRAP1, SFN, ITGA2, TTK, CSNK1A1, BRAF, GSK3B, AKT3, FOXO1, SGK, HSP90AA1, or RPS6KB1.

In some embodiments, the signaling pathway activated in the cell is the ERK/MAPK pathway. In some embodiments, the transmembrane receptor comprises EGFR, Trk A/B, fibroblast growth factor receptor (FGFR) or platelet-derived growth factor receptor (PDGFR) and the promoter regulates expression of PRKCE, ITGAM, ITGA5, HSPB1, IRAK1, PRKAA2, EIF2AK2, RAC1, RAP1A, TLN1, EIF4E, ELK1, GRK6, MAPK1, RAC2, PLK1, AKT2, PIK3CA, CDK8, CREB1, PRKCI, PTK2, FOS, RPS6KA4, PIK3CB, PPP2R1A, PIK3C3, MAPK8, MAPK3, ITGA1, ETS1, KRAS, MYCN, EIF4EBP1, PPARG, PRKCD, PRKAA1, MAPK9, SRC, CDK2, PPP2CA, PIM1, PIK3C2A, ITGB7, YWHAZ, PPP1CC, KSR1, PXN, RAF1, FYN, DYRK1A, ITGB1, MAP2K2, PAK4, PIK3R1, STAT3, PPP2R5C, MAP2K1, PAK3, ITGB3, ESR1, ITGA2, MYC, TTK, CSNK1A1, CRKL, BRAF, ATF4, PRKCA, SRF, STAT1, or SGK.

In some embodiments, the signaling pathway activated in the cell is a glucocorticoid receptor signaling pathway. In some embodiments, the transmembrane receptor comprises glucocorticoid receptor and the promoter regulates expression of RAC1, TAF4B, EP300, SMAD2, TRAF6, PCAF, ELK1, MAPK1, SMAD3, AKT2, IKBKB, NCOR2, UBE2I, PIK3CA, CREB1, FOS, HSPA5, NFKB2, BCL2, MAP3K14, STAT5B, PIK3CB, PIK3C3, MAPK8, BCL2L1, MAPK3, TSC22D3, MAPK10, NRIP1, KRAS, MAPK13, RELA, STAT5A, MAPK9, NOS2A, PBX1, NR3C1, PIK3C2A, CDKN1C, TRAF2, SERPINEL NCOA3, MAPK14, TNF, RAF1, IKBKG, MAP3K7, CREBBP, CDKN1A, MAP2K2, JAK1, IL8, NCOA2, AKT1, JAK2, PIK3R1, CHUK, STAT3, MAP2K1, NFKB1, TGFBR1, ESR1, SMAD4, CEBPB, JUN, AR, AKT3, CCL2, MMP1, STAT1, IL6, or HSP90AA1.

In some embodiments, the signaling pathway activated in the cell is a B cell receptor signaling pathway. In some embodiments, the transmembrane receptor comprises a B cell receptor and the promoter regulates expression of RAC1, PTEN, LYN, ELK1, MAPK1, RAC2, PTPN11, AKT2, IKBKB, PIK3CA, CREB1, SYK, NFKB2, CAMK2A, MAP3K14, PIK3CB, PIK3C3, MAPK8, BCL2L1, ABL1, MAPK3, ETS1, KRAS, MAPK13, RELA, PTPN6, MAPK9, EGR1, PIK3C2A, BTK, MAPK14, RAF1, IKBKG, RELB, MAP3K7, MAP2K2, AKT1, PIK3R1, CHUK, MAP2K1, NFKB1, CDC42, GSK3A, FRAP1, BCL6, BCL10, JUN, GSK3B, ATF4, AKT3, VAV3, or RPS6KB1.

In some embodiments, the signaling pathway activated in the cell is an integrin signaling pathway. In some embodiments, the transmembrane receptor comprises an integrin or integrin subunit and the promoter regulates expression of ACTN4, ITGAM, ROCK1, ITGA5, RAC1, PTEN, RAP1A, TLN1, ARHGEF7, MAPK1, RAC2, CAPNS1, AKT2, CAPN2, PIK3CA, PTK2, PIK3CB, PIK3C3, MAPK8, CAV1, CAPN1, ABL1, MAPK3, ITGA1, KRAS, RHOA, SRC, PIK3C2A, ITGB7, PPP1CC, ILK, PXN, VASP, RAF1, FYN, ITGB1, MAP2K2, PAK4, AKT1, PIK3R1, TNK2, MAP2K1, PAK3, ITGB3, CDC42, RND3, ITGA2, CRKL, BRAF, GSK3B, or AKT3.

In some embodiments, the signaling pathway activated in the cell is an insulin receptor signaling pathway. In some embodiments, the transmembrane receptor comprises an insulin receptor and the promoter regulates expression of PTEN, INS, EIF4E, PTPN1, PRKCZ, MAPK1, TSC1, PTPN11, AKT2, CBL, PIK3CA, PRKCI, PIK3CB, PIK3C3, MAPK8, IRS1, MAPK3, TSC2, KRAS, EIF4, EBP1, SLC2A4, PIK3C2A, PPP1CC, INSR, RAF1, FYN, MAP2K2, JAK1, AKT1, JAK2, PIK3R1, PDPK1, MAP2K1, GSK3A, FRAP1, CRKL, GSK3B, AKT3, FOXO1, SGK, or RPS6KB1.

In some embodiments, the signaling pathway activated in the cell is a T cell receptor signaling pathway. In some embodiments, the transmembrane receptor comprises a T cell receptor and the promoter regulates expression of RAC1, ELK1, MAPK1, IKBKB, CBL, PIK3CA, FOS, NFKB2, PIK3CB, PIK3C3, MAPK8, MAPK3, KRAS, RELA, PIK3C2A, BTK, LCK, RAF1, IKBKG, RELB, FYN, MAP2K2, PIK3R1, CHUK, MAP2K1, NFKB1, ITK, BCL10, JUN, or VAV3.

In some embodiments, the signaling pathway activated in the cell is a G-protein coupled receptor (GPCR) signaling pathway. In some embodiments, the transmembrane receptor comprises a GPCR and the promoter regulates expression of PRKCE, RAP1A, RGS16, MAPK1, GNAS, AKT2, IKBKB, PIK3CA, CREB1, GNAQ, NFKB2, CAMK2A, PIK3CB, PIK3C3, MAPK3, KRAS, RELA, SRC, PIK3C2A, RAF1, IKBKG, RELB, FYN, MAP2K2, AKT1, PIK3R1, CHUK, PDPK1, STAT3, MAP2K1, NFKB1, BRAF, ATF4, AKT3, or PRKCA.

In some cases, the promoter comprises a fragment of an endogenous promoter sequence which drives a desired level of expression. For example, minimal promoter elements which are smaller in size compared to full-length counterparts but still maintain a certain level of activity (e.g., at least 10%, 20%, 30%, 40%, 50%, 60%, 70%, 80%, or 90% activity) can be used.

In some embodiments, the promoter is an interleukin 2 (IL-2) promoter sequence, an interferon gamma (IFN-γ) promoter sequence, an interferon regulatory factor 4 (IRF4) promoter sequence, an nuclear receptor subfamily 4 group A member 1 (NR4A1, also known as nerve growth factor D3 (NGFIB)) promoter sequence, a PR domain zinc finger protein 1 (PRDM1) promoter sequence, a T-box transcription factor (TBX21) promoter sequence, a CD69 promoter sequence, a CD25 promoter sequence, or a granzyme B (GZMB) promoter sequence.

The expression cassette can comprise a GMP coding sequence operably linked to an endogenous promoter sequence. The expression cassette is, in some cases, not integrated into the cell genome. The expression cassette can be supplied to a cell as part of a non-integrating plasmid. The expression cassette is, in some cases, integrated into the cell genome. Integration into the cell genome may be targeted or non-targeted (e.g., random integration). In some embodiments, the expression cassette is integrated into the cell genome by lentivirus.

In some cases, the GMP coding sequence can be integrated into the genome such that the GMP coding sequence replaces an endogenous gene controlled by the promoter in the cell. In some cases, the GMP coding sequence does not replace an endogenous gene. The GMP coding sequence can be integrated into the genome such that the sequence encoding the GMP is located upstream of the endogenous gene. The GMP coding sequence can be integrated into the genome such that the GMP coding sequence is located downstream of the endogenous gene.

In some cases where the endogenous gene is located upstream of the GMP coding sequence, the GMP coding sequence and the endogenous gene may be joined by a nucleic acid sequence encoding a peptide linker. The sequence encoding the GMP may be joined in-frame to the endogenous gene such that the translated peptide sequence has the proper amino acid sequence. In some cases, the linker has a cleavage recognition site, such as a protease recognition site, allowing the protein encoded by the endogenous gene and the GMP can be separated by cleavage, e.g., protease cleavage, of the peptide linker. In some cases, the linker has a “self-cleaving” segment, such as a 2A peptide. 2A peptides, first discovered in picornaviruses, refer to peptide sequences, usually about 20 amino acids in length that allow multiple genes (e.g., at least two genes) to be expressed from the same mRNA. 2A peptides are thought to function by making the ribosome skip the synthesis of a peptide bond at the C-terminus of a 2A element, leading to separation between the end of the 2A sequence and the next peptide downstream. The “cleavage” typically occurs between the glycine and proline residues found on the C-terminus. In general, the upstream gene, or cistron, will have a few additional residues added to the end, while the downstream gene, or cistron, will start with the proline residue. Exemplary 2A peptides include T2A (EGRGSLLTCGDVEENPGP (SEQ ID NO: 1)), P2A (ATNFSLLKQAGDVEENPGP (SEQ ID NO: 2)), E2A (QCTNYALLKLAGDVESNPGP (SEQ ID NO: 3)), and F2A (VKQTLNFDLLKLAGDVESNPGP (SEQ ID NO: 4)).

In some cases where the endogenous gene is located upstream of the GMP coding sequence, the GMP coding sequence and the endogenous gene may be joined by a nucleic acid sequence which is non-coding. The non-coding nucleic acid sequence joining the endogenous gene and the GMP coding sequence can comprise an internal ribosome entry site (IRES), which allows for initiation of translation from an internal region of an mRNA. An IRES element can act as another ribosome recruitment site, thereby resulting in co-expression of two proteins from a single mRNA. The IRES elements may be between about 300-1000 bp in length (e.g., between about 400-900 bp, 500-800 bp, or 600-700 bp in length).

In some cases, the promoter is an exogenous promoter that is activated upon binding of a ligand of the ligand binding domain (e.g., activation of a signaling pathway of the cell). Exogenous promoter sequences include promoter sequences not naturally found in a cell genome, for example promoter sequences from a different species. In another example, an exogenous promoter can comprise a synthetic promoter sequence which does not naturally occur in any organism. In some cases, an exogenous promoter can comprise multiple copies of an endogenous promoter sequence, a synthetic promoter sequence, and combinations thereof.

In some cases, the promoter comprises a fragment of a synthetic promoter sequence which drives a desired level of expression. For example, minimal promoter elements which are smaller in size compared to full-length counterparts but still maintain a certain level of activity (e.g., at least 10%, 20%, 30%, 40%, 50%, 60%, 70%, 80%, or 90% activity) can be used.

The expression cassette can comprise a GMP coding sequence operably linked to the exogenous promoter. The expression cassette is, in some cases, integrated into the cell genome. In some embodiments, the expression cassette is integrated into the cell genome by lentivirus. The integration may be targeted or non-targeted (e.g., random integration). The expression cassette is, in some cases, not integrated into the cell genome. The expression cassette can be supplied to a cell as part of a non-integrating plasmid.

The level of expression of the GMP can depend on the promoter and/or the signaling pathway utilized in the system. In some cases, the GMP can be expressed at high levels relative to the endogenous gene(s) controlled by the promoter. In some cases, the GMP can be expressed at moderate levels relative to the endogenous gene(s) controlled by the promoter. In some cases, the GMP can be expressed at low levels relative to the endogenous gene(s) controlled by the promoter. In some cases, the GMP can be expressed at levels similar to the endogenous gene(s) controlled by the promoter. The specificity of GMP expression can also depend on the promoter and/or the signaling pathways utilized in the system. In some cases, the GMP is preferentially expressed when the transmembrane receptor binds a ligand. In some cases, the GMP is primarily expressed when the transmembrane receptor binds a ligand. In some cases, the GMP is only expressed when the transmembrane receptor binds a ligand.

The resulting expressed GMP comprises an actuator moiety and can regulate expression of the target gene in the cell. The actuator moiety can bind to a target polynucleotide to regulate expression and/or activity of the target gene. In some embodiments, the target polynucleotide comprises genomic DNA. In some embodiments, the target polynucleotide comprises a region of a plasmid, for example a plasmid carrying an exogenous gene. In some embodiments, the target polynucleotide comprises RNA, for example mRNA. In some embodiments, the target polynucleotide comprises an endogenous gene or gene product.

The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease or a variant thereof. The actuator moiety can regulate expression or activity of a gene and/or edit the sequence of a nucleic acid (e.g., a gene and/or gene product). In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity. An actuator moiety having reduced or minimal nuclease activity can regulate expression and/or activity of a gene by physical obstruction of a target polynucleotide or recruitment of additional factors effective to suppress or enhance expression of the target polynucleotide. The actuator moiety can physically obstruct the target polynucleotide or recruit additional factors effective to suppress or enhance expression of the target polynucleotide. In some embodiments, the actuator moiety comprises a transcriptional activator effective to increase expression of the target polynucleotide. In some embodiments, the actuator moiety comprises a transcriptional repressor effective to decrease expression of the target polynucleotide. In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. In some embodiments, the actuator moiety is a DNA-guided actuator moiety. In some embodiments, the actuator moiety is an RNA-guided actuator moiety. An actuator moiety can regulate expression or activity of a gene and/or edit a nucleic acid sequence, whether exogenous or endogenous.

Any suitable nuclease can be used. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); and any variant thereof.

Any target gene can be regulated by the GMP. It is contemplated that genetic homologues of a gene described herein are covered. For example, a gene can exhibit a certain identity and/or homology to genes disclosed herein. Therefore, it is contemplated that the expression of a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level) can be regulated. It is also contemplated that the expression of a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be regulated.

In some embodiments, the target gene encodes for a cytokine. Non-limiting examples of cytokines include 4-1BBL, activin βA, activin βB, activin βC, activin βE, artemin (ARTN), BAFF/BLyS/TNFSF138, BMP10, BMP15, BMP2, BMP3, BMP4, BMP5, BMP6, BMP7, BMP8a, BMP8b, bone morphogenetic protein 1 (BMP1), CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CD153/CD30L/TNFSF8, CD40L/CD154/TNFSF5, CD40LG, CD70, CD70/CD27L/TNFSF7, CLCF1, c-MPL/CD110/TPOR, CNTF, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, EDA-A1, FAM19A1, FAM19A2, FAM19A3, FAM19A4, FAM19A5, Fas Ligand/FASLG/CD95L/CD178, GDF10, GDF11, GDF15, GDF2, GDF3, GDF4, GDF5, GDF6, GDF7, GDF8, GDF9, glial cell line-derived neurotrophic factor (GDNF), growth differentiation factor 1 (GDF1), IFNA1, IFNA10, IFNA13, IFNA14, IFNA2, IFNA4, IFNA5/IFNaG, IFNA7, IFNA8, IFNB1, IFNE, IFNG, IFNZ, IFNω/IFNW1, IL11, IL18, IL18BP, IL1A, IL1B, IL1F10, IL1F3/IL1RA, IL1F5, IL1F6, IL1F7, IL1F8, IL1F9, IL1RL2, IL31, IL33, IL6, IL8/CXCL8, inhibin-A, inhibin-B, Leptin, LIF, LTA/TNFB/TNFSF1, LTB/TNFC, neurturin (NRTN), OSM, OX-40L/TNFSF4/CD252, persephin (PSPN), RANKL/OPGL/TNFSF11 (CD254), TL1A/TNFSF15, TNFA, TNF-alpha/TNFA, TNFSF10/TRAIL/APO-2L (CD253), TNFSF12, TNFSF13, TNFSF14/LIGHT/CD258, XCL1, and XCL2. In some embodiments, the target gene encodes for an immune checkpoint inhibitor. Non-limiting examples of such immune checkpoint inhibitors include PD-1, CTLA-4, LAG3, TIM-3, A2AR, B7-H3, B7-H4, BTLA, IDO, KIR, and VISTA. In some embodiments, the target gene encodes for a T cell receptor (TCR) alpha, beta, gamma, and/or delta chain.

In an aspect, the present disclosure provides a system for regulating expression of a target gene in a cell comprising two transmembrane receptors. The system comprises (a) a first transmembrane receptor comprising a first ligand binding domain and a first signaling domain, wherein the first signaling domain activates a first signaling pathway of the cell upon binding of a first ligand to the first ligand binding domain; (b) a second transmembrane receptor comprising a second ligand binding domain and a second signaling domain, wherein the second signaling domain activates a second signaling pathway of the cell upon binding of a second ligand to the second ligand binding domain; and (c) and expression cassette comprising a nucleic acid sequence encoding a gene modulating polypeptide (GMP) placed under control of a promoter, wherein the GMP comprises an actuator moiety, and wherein the promoter is activated to drive expression of the GMP upon (i) binding of the first ligand to the first ligand binding domain, and/or (ii) binding of the second ligand to the second ligand binding domain.

The first and second transmembrane receptors can each individually comprise an endogenous receptor, a synthetic receptor, or any variant thereof. Each of the first and second transmembrane receptors can comprise a Notch receptor; a G-protein coupled receptor (GPCR); an integrin receptor; a cadherin receptor; a catalytic receptor, including receptors possessing enzymatic activity and receptors which, rather than possessing intrinsic enzymatic activity, act by stimulating non-covalently associated enzymes (e.g., kinases); death receptors such as members of the tumor necrosis factor receptor (TNFR) superfamily; immune receptors, such as T cell receptors (TCR); or any variant thereof. In some embodiments, the transmembrane receptor of the system comprises a GPCR. Each of the first and second transmembrane receptors can comprise an exogenous receptor, such a synthetic receptor comprising a chimeric antigen receptor (CAR), a synthetic integrin receptor, a synthetic Notch receptor, or a synthetic GPCR receptor. In some cases, the first and the second transmembrane receptors may be the same type of receptor (e.g., both GPCR, synthetic GPCR, integrin, synthetic integrin, etc). In some cases, the first and second transmembrane receptors are different types of receptors. For example, the first receptor may comprise a GPCR while the second comprises a CAR. For further example, the first receptor may comprise an integrin subunit while the second comprises a Notch. Any desired combination of receptors can be used.

The first and second transmembrane receptors can bind different ligands. The first and second transmembrane receptors can bind different ligands with different affinities. In some cases, the first and second transmembrane receptors bind different ligands with similar binding affinities. The first and second transmembrane receptors can activate different signaling pathways of the cell when bound to ligand. In some cases, the two signaling pathways overlap. In some cases, the two signaling pathways do not overlap.

In some cases, at least one of the first and second transmembrane receptors comprises a GPCR. In some embodiments, at least one of the first and second transmembrane receptors comprises a chimeric antigen receptor (CAR). The ligand binding domain (e.g., extracellular region) of the CAR, as previously described herein, can comprise a Fab, a single-chain variable fragment (scFv), the extracellular region of an endogenous receptor (e.g., GPCR, integrin receptor, T-cell receptor, B-cell receptor, etc), or an Fc binding domain. The CAR can comprise a transmembrane domain which situates the receptor in a cell membrane (e.g., plasma membrane, organelle membrane, etc). In some embodiments, the signaling domain (e.g., intracellular region) of the CAR comprises an immunoreceptor tyrosine-based activation motif (ITAM). In some embodiments, the signaling domain (e.g., intracellular region) of the CAR comprises an immunoreceptor tyrosine-based inhibition motif (ITIM). In some embodiments, the CAR comprises both an ITAM motif and an ITIM motif. In some embodiments, the CAR comprises at least one co-stimulatory domain.

Upon binding of a first ligand to the first ligand binding domain, binding of a second ligand to the second ligand binding domain, or binding of both ligand binding domains to ligands, the signaling domain(s) of the receptor(s) can activate at least one signaling pathway of the cell. The signaling pathway and its associated proteins can be involved in regulating (e.g., activating and/or de-activating) a cellular response such as programmed changes in gene expression via translational regulation; transcriptional regulation; and epigenetic modification including the regulation of methylation, acetylation, phosphorylation, ubiquitylation, sumoylation, ribosylation, and citrullination.

As described in the system comprising one transmembrane receptor, transcriptional regulation in response to signaling pathway activation can be utilized to express a gene modulating polypeptide (GMP). A nucleic acid sequence encoding a GMP, or GMP coding sequence, can be placed under control of a promoter that is responsive to the first signaling pathway, second signaling pathway, or both first and second signaling pathways activated in the cell in response to ligand-receptor binding.

In some cases, the promoter is an endogenous promoter that is activated upon binding of a ligand to the ligand binding domain (e.g., activation of a signaling pathway of the cell). In some cases, the promoter comprises a fragment of an endogenous promoter sequence which drives a desired level of expression. For example, minimal promoter elements which are smaller in size compared to full-length counterparts but still maintain a certain level of activity (e.g., at least 10%, 20%, 30%, 40%, 50%, 60%, 70%, 80%, or 90% activity) can be used.

In some embodiments, the promoter is an interleukin 2 (IL-2) promoter sequence, an interferon gamma (IFN-γ) promoter sequence, an interferon regulatory factor 4 (IRF4) promoter sequence, an nuclear receptor subfamily 4 group A member 1 (NR4A1, also known as nerve growth factor D3 NGFIB) promoter sequence, a PR domain zinc finger protein 1 (PRDM1) promoter sequence, a T-box transcription factor (TBX21) promoter sequence, a CD69 promoter sequence, a CD25 promoter sequence, or a granzyme B (GZMB) promoter sequence.

The expression cassette can comprise a GMP coding sequence operably linked to an endogenous promoter sequence. The expression cassette is, in some cases, not integrated into the cell genome. The expression cassette can be supplied to a cell as part of a non-integrating plasmid. The expression cassette is, in some cases, integrated into the cell genome. Integration may be targeted or non-targeted (e.g., random integration). In some embodiments, the expression cassette is integrated into the cell genome by lentivirus.

In some cases where the endogenous gene is located upstream of the GMP coding sequence, the GMP coding sequence and the endogenous gene may be joined by a nucleic acid sequence encoding a peptide linker. The sequence encoding the GMP may be joined in-frame to the endogenous gene such that the translated peptide sequence has the proper amino acid sequence. In some cases, the linker has a cleavage recognition site, such as a protease recognition site, allowing the protein encoded by the endogenous gene and the GMP can be separated by cleavage, e.g., protease cleavage, of the peptide linker. In some cases, the linker has a “self-cleaving” segment, such as a 2A peptide. Exemplary 2A peptides include T2A (EGRGSLLTCGDVEENPGP (SEQ ID NO: 1)), P2A (ATNFSLLKQAGDVEENPGP (SEQ ID NO: 2)), E2A (QCTNYALLKLAGDVESNPGP (SEQ ID NO: 3)), and F2A (VKQTLNFDLLKLAGDVESNPGP (SEQ ID NO: 4)).

In some cases where the endogenous gene is located upstream of the GMP coding sequence, the GMP coding sequence and the endogenous gene may be joined by a nucleic acid sequence which is non-coding. The non-coding nucleic acid sequence joining the endogenous gene and the GMP coding sequence can comprise an internal ribosome entry site (IRES), which allows for initiation of translation from an internal region of an mRNA.

In some cases, the promoter is an exogenous promoter that is activated upon binding of a ligand of the ligand binding domain (e.g., activation of a signaling pathway of the cell). Exogenous promoter sequences include promoter sequences not naturally found in a cell genome, for example promoter sequences from a different species. An exogenous promoter can comprise a synthetic promoter sequence which does not naturally occur in any organism. In some cases, an exogenous promoter can comprise multiple copies of an endogenous promoter sequence, a synthetic promoter sequence, and combinations thereof.

In some cases, the promoter comprises a fragment of a synthetic promoter sequence which drives a desired level of expression. For example, minimal promoter elements which are smaller in size compared to full-length counterparts but still maintain a certain level of activity (e.g., at least 10%, 20%, 30%, 40%, 50%, 60%, 70%, 80%, or 90% activity) can be used.

The expression cassette can comprise a GMP coding sequence operably linked to the exogenous promoter. The expression cassette is, in some cases, integrated into the cell genome. In some embodiments, the expression cassette is integrated into the cell genome by lentivirus. The integration may be targeted or non-targeted (e.g., random integration). The expression cassette is, in some cases, not integrated into the cell genome. The expression cassette can be supplied to a cell as part of a non-integrating plasmid.

The resulting expressed GMP comprises an actuator moiety and can regulate expression of the target gene in the cell. The actuator moiety can bind to a target polynucleotide to regulate expression and/or activity of the target gene. In some embodiments, the target polynucleotide comprises genomic DNA. In some embodiments, the target polynucleotide comprises a region of a plasmid, for example a plasmid carrying an exogenous gene. In some embodiments, the target polynucleotide comprises RNA, for example mRNA. In some embodiments, the target polynucleotide comprises an endogenous gene or gene product.

The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease, or a variant thereof. The actuator moiety can regulate expression or activity of a gene and/or edit the sequence of a nucleic acid (e.g., a gene and/or gene product). In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity. An actuator moiety having reduced or minimal nuclease activity can regulate expression and/or activity of a gene by physical obstruction of a target polynucleotide or recruitment of additional factors effective to suppress or enhance expression of the target polynucleotide. The actuator moiety can physically obstruct the target polynucleotide or recruit additional factors effective to suppress or enhance expression of the target polynucleotide. In some embodiments, the actuator moiety comprises a transcriptional activator effective to increase expression of the target polynucleotide. In some embodiments, the actuator moiety comprises a transcriptional repressor effective to decrease expression of the target polynucleotide. In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. In some embodiments, the actuator moiety is a DNA-guided actuator moiety. In some embodiments, the actuator moiety is an RNA-guided actuator moiety. An actuator moiety can regulate expression or activity of a gene and/or edit a nucleic acid sequence, whether exogenous or endogenous.

Any suitable nuclease can be used in a two receptor system. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); and any variant thereof.

Any target gene can be regulated by the GMP of a two receptor system. It is contemplated that genetic homologues of a gene described herein are covered. For example, a gene can exhibit a certain identity and/or homology to genes disclosed herein. Therefore, it is contemplated that the expression of a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level) can be regulated. It is also contemplated that the expression of a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be regulated.

In some embodiments, the target gene encodes for a cytokine. Non-limiting examples of cytokines include 4-1BBL, activin βA, activin βB, activin βC, activin βE, artemin (ARTN), BAFF/BLyS/TNFSF138, BMP10, BMP15, BMP2, BMP3, BMP4, BMP5, BMP6, BMP7, BMP8a, BMP8b, bone morphogenetic protein 1 (BMP1), CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CD153/CD30L/TNFSF8, CD40L/CD154/TNFSF5, CD40LG, CD70, CD70/CD27L/TNFSF7, CLCF1, c-MPL/CD110/TPOR, CNTF, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, EDA-A1, FAM19A1, FAM19A2, FAM19A3, FAM19A4, FAM19A5, Fas Ligand/FASLG/CD95L/CD178, GDF10, GDF11, GDF15, GDF2, GDF3, GDF4, GDF5, GDF6, GDF7, GDF8, GDF9, glial cell line-derived neurotrophic factor (GDNF), growth differentiation factor 1 (GDF1), IFNA1, IFNA10, IFNA13, IFNA14, IFNA2, IFNA4, IFNA5/IFNaG, IFNA7, IFNA8, IFNB1, IFNE, IFNG, IFNZ, IFNω/IFNW1, IL11, IL18, IL18BP, IL1A, IL1B, IL1F10, IL1F3/IL1RA, IL1F5, IL1F6, IL1F7, IL1F8, IL1F9, IL1RL2, IL31, IL33, IL6, IL8/CXCL8, inhibin-A, inhibin-B, Leptin, LIF, LTA/TNFB/TNFSF1, LTB/TNFC, neurturin (NRTN), OSM, OX-40L/TNFSF4/CD252, persephin (PSPN), RANKL/OPGL/TNFSF11 (CD254), TL1A/TNFSF15, TNFA, TNF-alpha/TNFA, TNFSF10/TRAIL/APO-2L (CD253), TNFSF12, TNFSF13, TNFSF14/LIGHT/CD258, XCL1, and XCL2. In some embodiments, the target gene encodes for an immune checkpoint inhibitor. Non-limiting examples of such immune checkpoint inhibitors include PD-1, CTLA-4, LAG3, TIM-3, A2AR, B7-H3, B7-H4, BTLA, IDO, KIR, and VISTA. In some embodiments, the target gene encodes for a T cell receptor (TCR) alpha, beta, gamma, and/or delta chain.

In addition to regulating expression of a target gene in a cell, systems comprising two transmembrane receptors can be utilized to regulate expression of two target genes in a cell. In an aspect, the present disclosure provides a system for regulating expression of two target genes in a cell comprising two transmembrane receptors. The system comprises (a) a first transmembrane receptor comprising a first ligand binding domain and a first signaling domain, wherein the first signaling domain activates a first signaling pathway of the cell upon binding of a first ligand to the first ligand binding domain; (b) a second transmembrane receptor comprising a second ligand binding domain and a second signaling domain, wherein the second signaling domain activates a second signaling pathway of the cell upon binding of a second ligand to the second ligand binding domain; (c) a first expression cassette comprising a nucleic acid sequence encoding a first gene modulating polypeptide (GMP), wherein the first GMP comprises a first actuator moiety, and wherein the first promoter is activated to drive expression of the first GMP upon binding of the first ligand to the first ligand binding domain; and (d) a second expression cassette comprising a nucleic acid sequence encoding a second gene modulating polypeptide (GMP), wherein the second GMP comprises a second actuator moiety, and wherein the second promoter is activated to drive expression of the second GMP upon binding of the second ligand to the second ligand binding domain, wherein (i) the first GMP regulates expression of a first target gene and (ii) the second GMP regulates expression of a second target gene. Systems comprising two transmembrane receptors and two expression cassettes can allow for the orthogonal regulation of two target genes.

As previously described herein, the first and second transmembrane receptors can each individually comprise an endogenous receptor, a synthetic receptor, or any variant thereof. Each of the first and second transmembrane receptors can comprise a Notch receptor; a G-protein coupled receptor (GPCR); a T cell receptor (TCR), an integrin receptor; a cadherin receptor; a catalytic receptor, including receptors possessing enzymatic activity and receptors which, rather than possessing intrinsic enzymatic activity, act by stimulating non-covalently associated enzymes (e.g., kinases); death receptors such as members of the tumor necrosis factor receptor (TNFR) superfamily; immune receptors; or any variant thereof. In some embodiments, the transmembrane receptor of the system comprises a GPCR. Each of the first and second transmembrane receptors can comprise an exogenous receptor, such a synthetic receptor comprising a chimeric antigen receptor (CAR), a synthetic integrin receptor, a synthetic Notch receptor, or a synthetic GPCR receptor. In some cases, the first and the second transmembrane receptors may be the same type of receptor (e.g., both GPCR, synthetic GPCR, integrin, synthetic integrin, etc). In some cases, the first and second transmembrane receptors are different types of receptors. For example, the first receptor may comprise a GPCR while the second comprises a CAR. For further example, the first receptor may comprise an integrin subunit while the second comprises a Notch. Any desired combination of receptors can be used.

The first and second transmembrane receptors can bind different ligands. The first and second transmembrane receptors can activate different signaling pathways of the cell when bound to ligand. In some cases, the two signaling pathways overlap. In some cases, the two signaling pathways do not overlap.

The first and second GMPs can each individually comprise an actuator moiety comprising a nuclease. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); and any variant thereof.

The actuator moieties of the first and second GMPs may be any suitable actuator moiety disclosed herein. In some cases, the actuator moieties of the first and second GMP are the same. For example, both first and second GMPs comprise a Cas protein, such as a Cas9 protein. In some cases, both of the first and second GMPs comprise Cpf1. However, the actuator moieties of the first and second GMP can be different.

In some embodiments, the first target gene and the second target gene are both up-regulated. In some embodiments, the first target gene and the second target gene are both down-regulated. In some embodiments, the first target gene is up-regulated and the second target gene is down-regulated. In some embodiments, the first target gene is down-regulated and the second target gene is up-regulated.

In some cases, an actuator moiety can be split into two or more portions. The two or more portions of the actuator moiety, when expressed, can complex to form a functional actuator moiety. A system comprising two transmembrane receptors can be used, in some cases, to express two portions of a split actuator moiety. In an aspect, the present disclosure provides a system for regulating expression of a target gene in a cell comprising (a) a first transmembrane receptor comprising a first ligand binding domain and a first signaling domain, wherein the first signaling domain activates a first signaling pathway of the cell upon binding of a first ligand to the first ligand binding domain; (b) a second transmembrane receptor comprising a second ligand binding domain and a second signaling domain, wherein the second signaling domain activates a second signaling pathway of the cell upon binding of a second ligand to the second ligand binding domain; (c) a first expression cassette comprising a nucleic acid sequence encoding a first partial gene modulating polypeptide (GMP) placed under control of a first promoter, wherein the first partial GMP comprises a first portion of an actuator moiety, and wherein the first promoter is activated to drive expression of the first partial GMP upon binding of the first ligand to the first ligand binding domain; and (d) a second expression cassette comprising a nucleic acid sequence encoding a second partial gene modulating polypeptide (GMP) placed under control of a second promoter, wherein the second partial GMP comprises a second portion of an actuator moiety, and wherein the second promoter is activated to drive expression of the second partial GMP upon binding of the second ligand to the second ligand binding domain; wherein the first and second portion of the actuator moiety complex to form a reconstituted GMP comprising a functional actuator moiety, wherein the reconstituted GMP regulates expression of the target gene.

Any one of the actuator moieties provided herein can be split into two or more portions. The split position of an actuator moiety may be selected using ordinary skill in the art, for instance based on crystal structure data. In some cases, an optimal split position is determined by generating a library of actuator moieties split at different positions of the protein and screening. These split actuator moieties may be screened for characteristics such as the ability of two or more portions to reconstitute, retention of binding affinity, retention of binding specificity, enzymatic activity, etc. Unstructured regions may be preferred as split positions when generating partial actuator moieties.

The two or more portions can reconstitute a functional actuator moiety by complexing spontaneously when the two or more portions are in proximity. In some cases, complexing of the two or more portions occurs with the assistance of a dimerizing agent.

A functional actuator moiety formed by complexing two or more portions of a split actuator moiety may retain a portion of the activity of the unsplit moiety. For example, the functional actuator moiety comprising two or more portions of the actuator moiety may have at least 30%, 40%, 50%, 60%, 70%, 80%, 90%, or 100% of the activity of the unsplit (single-portion) actuator moiety. Activity can refer to any naturally occurring property of the actuator moiety, for example binding affinity, binding specificity, enzymatic activity etc. Activity includes the ability to target and/or bind a target polynucleotide.

In some cases, the reconstituted GMP comprising the functional actuator moiety can be a complex of at least two different GMPs. The at least two different GMPs can complex spontaneously into the reconstituted GMP when the at least two different GMPs are in proximity. In some cases, complexing of the at least two different GMPs into the reconstituted GMP occurs with the assistance of a complexing agent (e.g., an oligonucleotide).

In some cases, the first partial GMP of the reconstituted GMP is at least a portion and/or variant of a first GMP, and the second partial GMP of the reconstituted GMP is at least a portion and/or variant of a second GMP, wherein the first GMP and the second GMP are different. In some cases, a guide-RNA (e.g., sgRNA) can complex with the first partial GMP and the second partial GMP to form the reconstituted GMP comprising the functional actuator moiety. The complex comprising the first partial GMP, the second partial GMP and the guide-RNA can be a gene modulating unit (GMU). The guide-RNA can comprise (i) at least one binding sequence for the first partial GMP and (ii) at least one binding sequence for the second partial GMP. The guide-RNA can comprise (i) at least 1, 2, 3, 4, 5 or more binding sequences for the first partial GMP and (ii) at least 1, 2, 3, 4, 5 or more binding sequences for the second partial GMP. Thus, the guide-RNA can complex with (i) at least one of the first partial GMP and (ii) at least one of the second partial GMP to form the GMU. The guide-RNA can complex with (i) at least 1, 2, 3, 4, 5 or more of the first partial GMP and (ii) at least 1, 2, 3, 4, 5 or more of the second partial GMP to form the GMU.

In some cases, the first partial GMP is a Cas protein. The Cas protein can be mutated and/or modified to yield a nuclease deficient protein or a protein with decreased nuclease activity relative to a wild-type Cas protein. In some cases, the second partial GMP is a fusion protein comprising a RNA-binding protein and a transcription regulator (e.g., an actiator or a repressor). The fusin protein can comprise a peptide linker between the RNA-binding protein and the transcription regulator. In some cases, the RNA-binding protein of the fusion protein is at least a portion of a protein from a virus (e.g., a coat protein). In some cases, the virus is a RNA virus. In some cases, the RNA virus is a RNA bacteriophage. Examples of the RNA bacteriophage include f2, MS2, R17, fr, M12, Qβ, and PP7. Examples of a protein from the RNA bacteriophage include MCP (from MS2) and PCP (from PP7). In some cases, the RNA-binding protein of the fusion protein is at least a portion of a non-viral protein. In some cases, the non-viral protein is an RNA-regulatory protein. In some cases, the non-viral protein is from a PUF protein family (Pumilio and fem-3 binding factor (FBF)). Examples of a PUF protein include wild type PUF, PUFa, PUFb, PUFc, PUFw, PUF (3-2), and PUF (6-2/7-2).

In some embodiments of systems herein for regulating expression of a target gene, the actuator moiety is temporarily unable to access a target polynucleotide. For example, the actuator moiety may be linked to a peptide localization sequence which sequesters the actuator moiety in a location of the cell different from that of a target polynucleotide corresponding to the target gene. In some cases, the actuator moiety may be linked to an inhibitory peptide sequence or other modification which prevents the actuator moiety from acting on the target polynucleotide. A cleavage moiety present in the system can cleave a cleavage recognition site to release the actuator moiety from the peptide localization sequence or inhibitory sequence, thus enabling the actuator moiety to act on the target polynucleotide.

In an aspect, the present disclosure provides a system for regulating expression of a target gene in a cell, the system comprising a transmembrane receptor comprising a ligand binding domain, a signaling domain, and a gene modulating polypeptide (GMP), the GMP comprising an actuator moiety linked to a cleavage recognition site, wherein the signaling domain activates a signaling pathway of the cell upon binding of a ligand to the ligand binding domain; and an expression cassette comprising a nucleic acid sequence encoding a cleavage moiety, wherein the nucleic acid sequence is placed under the control of a promoter activated by the signaling pathway to drive expression of the cleavage moiety upon binding of the ligand to the ligand binding domain, wherein the expressed cleavage moiety cleaves the cleavage recognition site to release the actuator moiety, and wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, the signaling domain, the cleavage recognition site, and the actuator moiety. The ligand binding domain can be located in the extracellular region of the cell. The signaling domain, the cleavage recognition site, and the actuator moiety can be located in the intracellular region of the cell.

With reference to FIG. 6, a transmembrane receptor can comprise a chimeric antigen receptor (CAR). The chimeric transmembrane receptor can have an extracellular ligand binding domain comprising a single-chain Fv (scFv), a transmembrane region, at least one signaling domain in the intracellular region, and a gene modulating polypeptide (GMP). In some cases, the GMP comprises an actuator moiety (e.g., dCas9) linked to a cleavage recognition sequence (e.g., TEV cleavage sequence, TCS). The actuator moiety can, in some cases, be linked to a transcription activator (e.g., VP64-p65-Rta (VPR)) or repressor (e.g., Kruppel associated box (KRAB)). The signaling domain can activate an intrinsic signaling pathway of the cell upon binding of a ligand to the ligand binding domain. The signaling pathway can drive expression of a cleavage moiety from an expression cassette present in the cell. In some cases, the cleavage moiety is a TEV protease. The expressed TEV protease can cleave the TEV cleavage sequence (TCS) and release the actuator moiety from the receptor. One or more guide nucleic acids (e.g., sgRNAs) can complex with the dCas9 which can then regulate expression of a target gene. FIG. 6 provides a non-limiting example system and various combinations of receptor, gene modulating polypeptide, actuator moiety, cleavage moiety, cleavage recognition sequence, expression cassette, promoter, etc are contemplated in the present disclosure. For example, in some cases, the transmembrane receptor may comprise a T cell receptor (TCR).

In an aspect, the present disclosure provides a system for regulating expression of a target gene in a cell, comprising a transmembrane receptor comprising a ligand binding domain, a signaling domain, and a cleavage moiety, wherein the signaling domain activates a signaling pathway of the cell upon binding of a ligand to the ligand binding domain; and an expression cassette comprising a nucleic acid sequence encoding a fusion protein comprising a gene modulating polypeptide (GMP) linked to a nuclear export signal peptide, the GMP comprising an actuator moiety linked to a cleavage recognition site, wherein the nucleic acid sequence is placed under the control of a promoter activated by the signaling pathway to drive expression of the fusion protein upon binding of the ligand to the ligand binding domain, wherein the cleavage moiety cleaves the cleavage recognition site of the fusion protein to release the actuator moiety, wherein the released actuator moiety regulates expression of a target polynucleotide, for example a target gene. In some embodiments, the cleavage moiety cleaves the cleavage recognition site when in proximity to the cleavage recognition site. In some embodiments, the cleavage moiety is linked to the intracellular region of the transmembrane receptor. In some cases, the transmembrane receptor comprises, from the N-terminus to the C-terminus, the ligand binding domain, a transmembrane region, the signaling domain, and the cleavage moiety. The ligand binding domain can be located in the extracellular region of the cell. The signaling domain and the cleavage moiety can be located in the intracellular region of the cell.

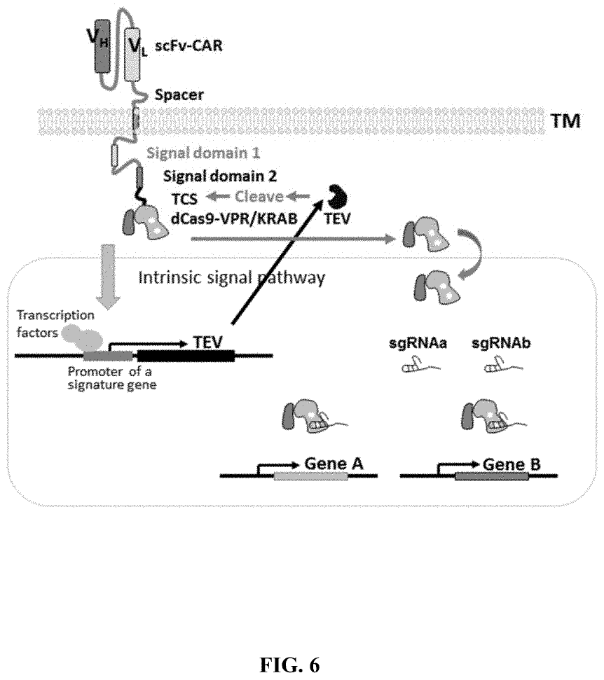

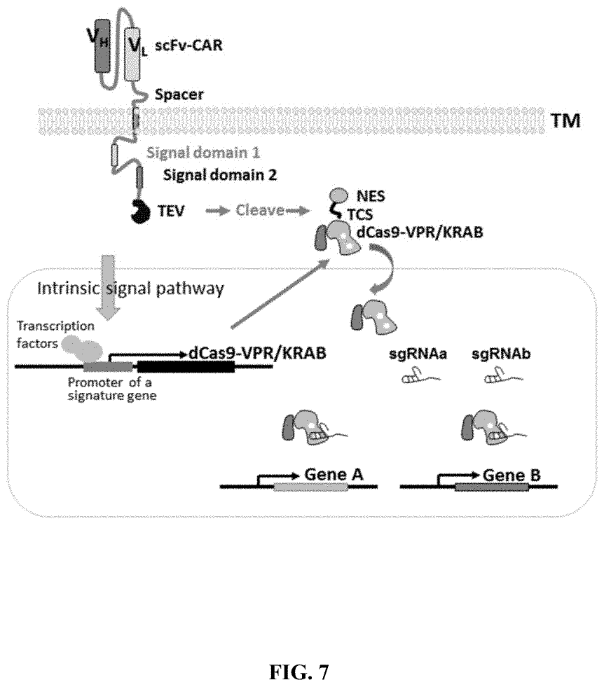

With reference to FIG. 7, a transmembrane receptor can comprise a chimeric antigen receptor (CAR). The chimeric transmembrane receptor can have an extracellular ligand binding domain comprising a single-chain Fv (scFv), a transmembrane region, at least one signaling domain in the intracellular region, and a cleavage moiety. In some cases, the cleavage moiety is a TEV protease. The signaling domain can activate an intrinsic signaling pathway of the cell upon binding of a ligand to the ligand binding domain. The signaling pathway can drive expression of a fusion polypeptide comprising a GMP linked to a nuclear export signal peptide (NES) from an expression cassette present in the cell. The GMP can comprise an actuator moiety, for example a dCas9, linked to a cleavage recognition sequence (e.g., TEV cleavage sequence, TCS). The actuator moiety can, in some cases, be linked to a transcription activator (e.g., VPR) or repressor (e.g., KRAB). The TEV protease can cleave the TEV cleavage sequence (TCS) and release the actuator moiety from the NES. One or more guide nucleic acids (e.g., sgRNAs) can complex with the released dCas9 which can then regulate expression of a target gene. FIG. 7 provides a non-limiting example system and various combinations of receptor, gene modulating polypeptide, actuator moiety, cleavage moiety, cleavage recognition sequence, expression cassette, promoter, etc are contemplated in the present disclosure. For example, in some cases, the transmembrane receptor may comprise a T cell receptor (TCR).

In some aspects, the present disclosure provides a system for regulating expression of a target gene in a cell comprising a transmembrane receptor comprising a ligand binding domain and a signaling domain, wherein the signaling domain activates a signaling pathway of the cell upon binding of a ligand to the ligand binding domain; an expression cassette comprising a nucleic acid sequence encoding a cleavage moiety, wherein the nucleic acid sequence is placed under the control of a promoter activated by the signaling pathway to drive expression of the cleavage moiety upon binding of the ligand to the ligand binding domain, wherein the expressed cleavage moiety cleaves a cleavage recognition site of a fusion protein comprising a gene modulating polypeptide (GMP) linked to a nuclear export signal peptide, the GMP comprising an actuator moiety linked to the cleavage recognition site. Cleavage of the cleavage recognition site can release the actuator moiety, and the released actuator moiety can regulate expression of a target polynucleotide, for example a target gene. In some embodiments, the cleavage moiety cleaves the cleavage recognition site when in proximity to the cleavage recognition site. In some embodiments, the system comprises the fusion protein comprising the GMP linked to the nuclear export signal peptide. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell. In some cases, the nuclear export signal peptide is linked at its C-terminus to the cleavage recognition site, which in turn is linked at its C-terminus to the actuator moiety.

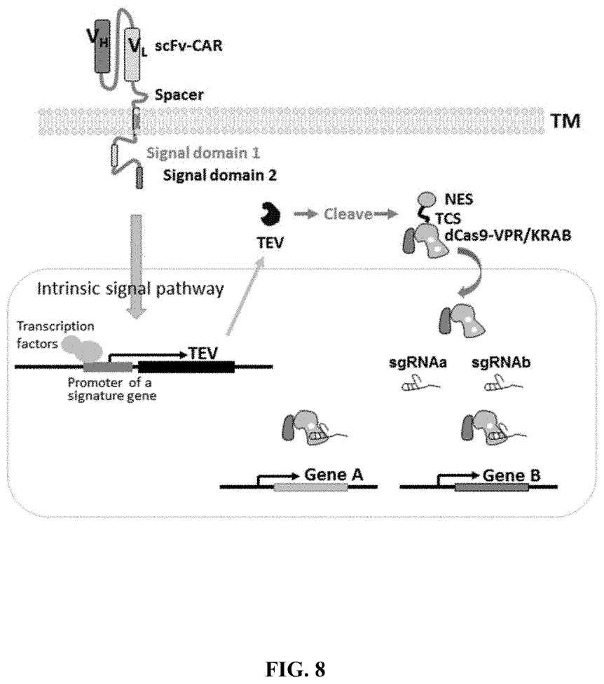

With reference to FIG. 8, a transmembrane receptor can comprise a chimeric antigen receptor (CAR). The chimeric transmembrane receptor can have an extracellular ligand binding domain comprising a single-chain Fv (scFv), a transmembrane region, and at least one signaling domain in the intracellular region. The signaling domain can activate an intrinsic signaling pathway of the cell upon binding of a ligand to the ligand binding domain. The signaling pathway can drive expression of a cleavage moiety from an expression cassette present in the cell. In some cases, the cleavage moiety is a TEV protease. A fusion polypeptide comprising a GMP linked to a nuclear export signal peptide (NES) may also be present in the system. The GMP can comprise an actuator moiety, for example dCas9, linked to a cleavage recognition sequence (e.g., TEV cleavage sequence, TCS). The actuator moiety can, in some cases, be linked to a transcription activator (e.g., VPR) or repressor (e.g., KRAB). The expressed TEV protease can cleave the TEV cleavage sequence (TCS) and release the actuator moiety from the NES. One or more guide nucleic acids (e.g., sgRNAs) can complex with the dCas9 which can then regulate expression of a target gene. FIG. 8 provides a non-limiting example system and various combinations of receptor, gene modulating polypeptide, actuator moiety, cleavage moiety, cleavage recognition sequence, expression cassette, promoter, etc are contemplated in the present disclosure. For example, in some cases, the transmembrane receptor can comprise a T cell receptor (TCR).

In an aspect, the present disclosure provides a system for regulating expression of a target gene in a cell, comprising a transmembrane receptor comprising a ligand binding domain and a signaling domain, wherein the signaling domain activates a signaling pathway of the cell upon binding of a ligand to the ligand binding domain; and an expression cassette comprising a nucleic acid sequence encoding a fusion protein comprising a gene modulating polypeptide (GMP) linked to a nuclear export signal peptide, the GMP comprising an actuator moiety linked to a cleavage recognition sequence, wherein the nucleic acid sequence is placed under the control of a promoter activated by the signaling pathway to drive expression of the fusion protein upon binding of the ligand to the ligand binding domain, wherein upon release of the actuator moiety via cleavage by a cleavage moiety at the cleavage recognition site, the released actuator moiety regulates expression of a target polynucleotide, for example a target gene. In some embodiments, the cleavage moiety cleaves the cleavage recognition site when in proximity to the cleavage recognition site. In some embodiments, the system comprises the cleavage moiety. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell. In some cases, the nuclear export signal peptide is linked at its C-terminus to the cleavage recognition site, which in turn is linked at its C-terminus to the actuator moiety.

With reference to FIG. 9, a transmembrane receptor can comprise a chimeric antigen receptor (CAR). The chimeric transmembrane receptor can have an extracellular ligand binding domain comprising a single-chain Fv (scFv), a transmembrane region, and at least one signaling domain in the intracellular region. The signaling domain can activate an intrinsic signaling pathway of the cell upon binding of a ligand to the ligand binding domain. The signaling pathway can drive expression of a fusion polypeptide comprising a GMP linked to a nuclear export signal peptide (NES) from an expression cassette present in the cell. The GMP can comprise an actuator moiety, for example dCas9, linked to a cleavage recognition sequence (e.g., TEV cleavage sequence, TCS). The actuator moiety can, in some cases, be linked to a transcription activator (e.g., VPR) or repressor (e.g., KRAB). A cleavage moiety, such as a TEV protease, may also be present in the system. The TEV protease can cleave the TEV cleavage sequence (TCS) and release the actuator moiety from the NES. One or more guide nucleic acids (e.g., sgRNAs) can complex with the dCas9 which can then regulate expression of a target gene. FIG. 9 provides a non-limiting example system and various combinations of receptor, gene modulating polypeptide, actuator moiety, cleavage moiety, cleavage recognition sequence, expression cassette, promoter, etc are contemplated in the present disclosure. For example, in some cases, the transmembrane receptor can comprise a T cell receptor (TCR).

In an aspect, the present disclosure provides a system for regulating expression of a target gene in a cell comprising a transmembrane receptor comprising a ligand binding domain and a signaling domain, wherein the signaling domain activates a signaling pathway of the cell upon binding of a ligand to the ligand binding domain; a first expression cassette comprising a first nucleic acid sequence encoding a fusion protein comprising a gene modulating polypeptide (GMP) linked to a nuclear export signal peptide, the GMP comprising an actuator moiety linked to a cleavage recognition sequence, wherein the first nucleic acid sequence is placed under the control of a first promoter activated by the signaling pathway to drive expression of the fusion protein upon binding of the ligand to the ligand binding domain; and a second expression cassette comprising a nucleic acid sequence encoding a cleavage moiety, wherein the second nucleic acid sequence is placed under the control of a second promoter activated by the signaling pathway to drive expression of the cleavage moiety upon binding of the ligand to the ligand binding domain, wherein the expressed cleavage moiety cleaves the cleavage recognition site to release actuator moiety, wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell. In some cases, the nuclear export signal peptide is linked at its C-terminus to the cleavage recognition site, which in turn is linked at its C-terminus to the actuator moiety.

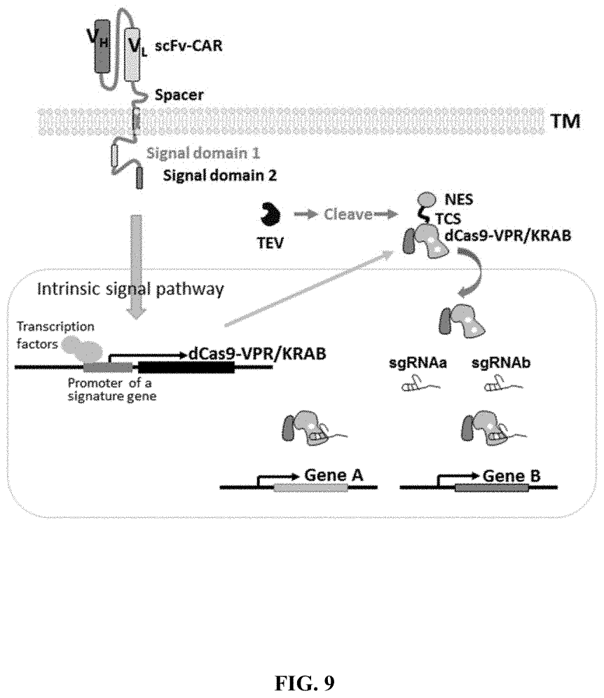

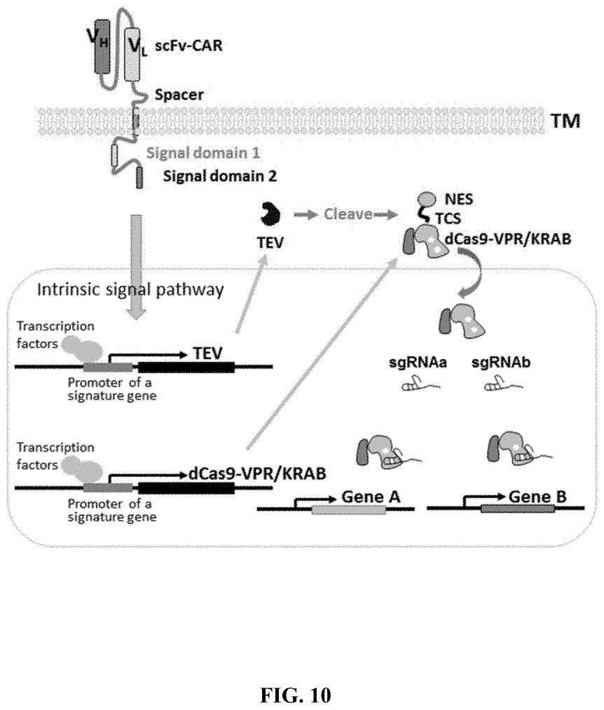

With reference to FIG. 10, a transmembrane receptor can comprise a chimeric antigen receptor (CAR). The chimeric transmembrane receptor can have an extracellular ligand binding domain comprising a single-chain F (scFv), a transmembrane region, and at least one signaling domain in the intracellular region. The signaling domain can activate an intrinsic signaling pathway of the cell upon binding of a ligand to the ligand binding domain. The signaling pathway can drive expression of a fusion polypeptide comprising a GMP linked to a nuclear export signal peptide (NES) from an expression cassette present in the cell. In some cases, the GMP comprises an actuator moiety, for example dCas9, linked to a cleavage recognition sequence (e.g., TEV cleavage sequence, TCS). The actuator moiety can, in some cases, be linked to a transcription activator (e.g., VPR) or repressor (e.g., KRAB). The signaling pathway can drive expression of a cleavage moiety from an expression cassette present in the cell. The cleavage moiety can be a TEV protease. The fusion polypeptide and the cleavage moiety may be on the same or different expression cassettes. The TEV protease can cleave the TEV cleavage sequence (TCS) and release the actuator moiety from the NES. One or more guide nucleic acids (e.g., sgRNAs) can complex with the dCas9 which can then regulate expression of a target gene. FIG. 10 provides a non-limiting example system and various combinations of receptor, gene modulating polypeptide, actuator moiety, cleavage moiety, cleavage recognition sequence, expression cassette, promoter, etc are contemplated in the present disclosure. For example, in some cases, the transmembrane receptor can comprise a T cell receptor (TCR).

In an aspect, the present disclosure provides a system for regulating expression of a target gene in a cell, comprising a transmembrane receptor comprising a ligand binding domain and a signaling domain, wherein the signaling domain activates a signaling pathway of the cell upon binding of a ligand to the ligand binding domain; a first expression cassette comprising a first nucleic acid sequence encoding a first partial gene modulating (GMP), the first partial GMP comprising a first portion of an actuator moiety, wherein the first nucleic acid sequence is placed under the control of a first promoter activated by the signaling pathway to drive expression of the first partial GMP upon binding of the ligand to the ligand binding domain; a second expression cassette comprising a second nucleic acid sequence encoding a second partial gene modulating polypeptide (GMP), the second partial GMP comprising a second portion of an actuator moiety, wherein the second nucleic acid sequence is placed under the control of a second promoter activated by the signaling pathway to drive expression of the second partial GMP upon binding of the ligand to the ligand binding domain; and wherein the first partial GMP and the second partial GMP complex to form a reconstituted actuator moiety, wherein the reconstituted actuator moiety regulates expression of the target gene. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell.

In an aspect, the present disclosure provides a system for regulating expression of a target gene in a cell, comprising a transmembrane receptor comprising a ligand binding domain and a signaling domain, wherein the signaling domain activates a signaling pathway of the cell upon binding of a ligand to the ligand binding domain; a first expression cassette comprising a first nucleic acid sequence encoding a first partial cleavage moiety, wherein the first nucleic acid sequence is placed under the control of a first promoter activated by the signaling pathway to drive expression of the first partial cleavage moiety upon binding of the ligand to the ligand binding domain; and a second expression cassette comprising a second nucleic acid sequence encoding a second partial cleavage moiety, wherein the second nucleic acid sequence is placed under control of a second promoter activated by the signaling pathway to drive expression of the second partial cleavage moiety upon binding of the ligand to the ligand binding domain; and wherein the first partial cleavage moiety and the second partial cleavage moiety complex to form a reconstituted cleavage moiety, and upon cleavage by the reconstituted cleavage moiety at a cleavage recognition site to release an actuator moiety from a nuclear export signal peptide, the actuator moiety regulates expression of a target polynucleotide, for example a target gene. In some embodiments, the system comprises a fusion polypeptide comprising the nuclear export signal peptide linked to the actuator moiety via the cleavage recognition site. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell. In some cases, the nuclear export signal peptide is linked at its C-terminus to the cleavage recognition site, which in turn is linked at its C-terminus to the actuator moiety.

In an aspect, the present disclosure provides a system for regulating expression of a target gene in a cell, comprising a transmembrane receptor comprising a ligand binding domain and a signaling domain, wherein the signaling domain activates a signaling pathway of the cell upon binding of a ligand to the ligand binding domain; and an expression cassette comprising a nucleic acid encoding one or both of (i) a cleavage moiety and (ii) a fusion protein comprising a gene modulating polypeptide (GMP) linked to a nuclear export signal peptide, the GMP comprising an actuator moiety linked to a cleavage recognition site, wherein expression of one or both of the cleavage moiety and the fusion protein is driven by a promoter activated by the signaling pathway upon binding of a ligand to the ligand binding domain, wherein the actuator moiety is released upon cleavage of the cleavage recognition site by the cleavage moiety, and wherein the released GMP regulates expression of a target polynucleotide.

The actuator moiety of embodiments herein can be any suitable actuator moiety, non-limiting examples of which are provided herein. In various embodiments of the aspects herein, the actuator moiety can be a polynucleotide-guided endonuclease. The endonuclease may be a wild-type protein or a mutant thereof. The mutant thereof can have different properties compared to the wild-type protein, for example, the mutant may have decreased nuclease activity. In some cases, the polynucleotide-guided endonuclease is an RNA-guided endonuclease, and the system further comprises a guide RNA.

In various embodiments of the aspects herein, a transmembrane receptor comprises an endogenous receptor. Non-limiting examples of endogenous receptors include Notch receptors; G-protein coupled receptors (GPCRs); integrin receptors; cadherin receptors; catalytic receptors including receptors possessing enzymatic activity and receptors which, rather than possessing intrinsic enzymatic activity, act by stimulating non-covalently associated enzymes (e.g., kinases); death receptors such as members of the tumor necrosis factor receptor (TNFR) superfamily; and immune receptors, such as T cell receptors (TCR).

In various embodiments of the aspects herein, a transmembrane receptor comprises an exogenous receptor. An exogenous receptor, in some cases, is a receptor of a different organism or species. An exogenous receptor, in some cases, can comprise a synthetic receptor which is not naturally found in a cell. A synthetic transmembrane receptor, in some embodiments, is a chimeric receptor constructed by joining multiple domains (e.g., extracellular, transmembrane, intracellular, etc.) from different molecules (e.g., different proteins, homologous proteins, orthologous proteins, etc).

A chimeric transmembrane of a subject system can comprise an endogenous receptor, or any variant thereof. A chimeric transmembrane receptor can bind specifically to at least one ligand, for example via a ligand binding domain. The ligand binding domain generally forms a part of the extracellular region of a transmembrane receptor and can sense extracellular ligands. In response to ligand binding, the intracellular region of the chimeric transmembrane receptor can activate a signaling pathway of the cell. In some cases, a signaling domain of the receptor activates the signaling pathway of the cell.

In some embodiments, a transmembrane receptor comprises a Notch receptor, or any variant thereof (e.g., synthetic or chimeric receptor). Notch receptors are transmembrane proteins that mediate cell-cell contact signaling and play a central role in development and other aspects of cell-to-cell communication, e.g. communication between two contacting cells (receiver cell and sending cell). Notch receptors expressed in a receiver cell recognize their ligands (the delta family of proteins), expressed on a sending cell. The engagement of notch and delta on these contacting cells leads to two-step proteolysis of the notch receptor that ultimately causes the release of the intracellular portion of the receptor from the membrane into the cytoplasm.

In some embodiments, a transmembrane receptor comprises a Notch receptor selected from Notch1, Notch2, Notch3, and Notch4, any homolog thereof, and any variant thereof. In some embodiments, a chimeric receptor comprises at least an extracellular region (e.g., ligand binding domain) of a Notch receptor, or any variant thereof. In some embodiments, a chimeric receptor comprises at least a membrane spanning region of a Notch, or any variant thereof. In some embodiments, a chimeric receptor comprises at least an intracellular region (e.g., cytoplasmic domain) of a Notch, or any variant thereof. A chimeric receptor polypeptide comprising a Notch, or any variant thereof, can bind a Notch ligand. In some embodiments, ligand binding to a chimeric receptor comprising a Notch, or any variant thereof, results in activation of a Notch signaling pathway.

In some embodiments, a transmembrane receptor comprises a G-protein coupled receptor (GPCR), or any variant thereof (e.g., synthetic or chimeric receptor). GPCRs are generally characterized by seven membrane-spanning a helices and can be arranged in a tertiary structure resembling a barrel, with the seven transmembrane helices forming a cavity within the plasma membrane that serves as a ligand-binding domain. Ligands can also bind elsewhere to a GPCR, for example to the extracellular loops and/or the N-terminal tail. Ligand binding can activate an associated G protein, which then functions in various signaling pathways. To de-activate this signaling, a GPCR can first be chemically modified by phosphorylation. Phosphorylation can then recruit co-adaptor proteins (e.g., arrestin proteins) for additional signaling.

In some embodiments, a transmembrane receptor comprises a GPCR selected from Class A Orphans; Class B Orphans; Class C Orphans; taste receptors, type 1; taste receptors, type 2; 5-hydroxytryptamine receptors; acetylcholine receptors (muscarinic); adenosine receptors; adhesion class GPCRs; adrenoceptors; angiotensin receptors; apelin receptor; bile acid receptor; bombesin receptors; bradykinin receptors; calcitonin receptors; calcium-sensing receptors; cannabinoid receptors; chemerin receptor; chemokine receptors; cholecystokinin receptors; class Frizzled GPCRs (e.g., Wnt receptors); complement peptide receptors; corticotropin-releasing factor receptors; dopamine receptors; endothelin receptors; G protein-coupled estrogen receptor; formylpeptide receptors; free fatty acid receptors; GABAB receptors; galanin receptors; ghrelin receptor; glucagon receptor family; glycoprotein hormone receptors; gonadotrophin-releasing hormone receptors; GPR18, GPR55 and GPR119; histamine receptors; hydroxycarboxylic acid receptors; kisspeptin receptor; leukotriene receptors; lysophospholipid (LPA) receptors; lysophospholipid (S1P) receptors; melanin-concentrating hormone receptors; melanocortin receptors; melatonin receptors; metabotropic glutamate receptors; motilin receptor; neuromedin U receptors; neuropeptide FF/neuropeptide AF receptors; neuropeptide S receptor; neuropeptide W/neuropeptide B receptors; neuropeptide Y receptors; neurotensin receptors; opioid receptors; orexin receptors; oxoglutarate receptor; P2Y receptors; parathyroid hormone receptors; platelet-activating factor receptor; prokineticin receptors; prolactin-releasing peptide receptor; prostanoid receptors; proteinase-activated receptors; QRFP receptor; relaxin family peptide receptors; somatostatin receptors; succinate receptor; tachykinin receptors; thyrotropin-releasing hormone receptors; trace amine receptor; urotensin receptor; vasopressin and oxytocin receptors; VIP and PACAP receptors.

In some embodiments, a transmembrane receptor comprises a GPCR selected from the group consisting of: 5-hydroxytryptamine (serotonin) receptor 1A (HTR1A), 5-hydroxytryptamine (serotonin) receptor 1B (HTR1B), 5-hydroxytryptamine (serotonin) receptor 1D (HTR1D), 5-hydroxytryptamine (serotonin) receptor 1E (HTR1E), 5-hydroxytryptamine (serotonin) receptor 1F (HTR1F), 5-hydroxytryptamine (serotonin) receptor 2A (HTR2A), 5-hydroxytryptamine (serotonin) receptor 2B (HTR2B), 5-hydroxytryptamine (serotonin) receptor 2C (HTR2C), 5-hydroxytryptamine (serotonin) receptor 4 (HTR4), 5-hydroxytryptamine (serotonin) receptor 5A (HTR5A), 5-hydroxytryptamine (serotonin) receptor 5B (HTR5BP), 5-hydroxytryptamine (serotonin) receptor 6 (HTR6), 5-hydroxytryptamine (serotonin) receptor 7, adenylate cyclase-coupled (HTR7), cholinergic receptor, muscarinic 1 (CHRM1), cholinergic receptor, muscarinic 2 (CHRM2), cholinergic receptor, muscarinic 3 (CHRM3), cholinergic receptor, muscarinic 4 (CHRM4), cholinergic receptor, muscarinic 5 (CHRM5), adenosine A1 receptor (ADORA1), adenosine A2a receptor (ADORA2A), adenosine A2b receptor (ADORA2B), adenosine A3 receptor (ADORA3), adhesion G protein-coupled receptor A1 (ADGRA1), adhesion G protein-coupled receptor A2 (ADGRA2), adhesion G protein-coupled receptor A3 (ADGRA3), adhesion G protein-coupled receptor B1 (ADGRB1), adhesion G protein-coupled receptor B2 (ADGRB2), adhesion G protein-coupled receptor B3 (ADGRB3), cadherin EGF LAG seven-pass G-type receptor 1 (CELSR1), cadherin EGF LAG seven-pass G-type receptor 2 (CELSR2), cadherin EGF LAG seven-pass G-type receptor 3 (CELSR3), adhesion G protein-coupled receptor D1 (ADGRD1), adhesion G protein-coupled receptor D2 (ADGRD2), adhesion G protein-coupled receptor E1 (ADGRE1), adhesion G protein-coupled receptor E2 (ADGRE2), adhesion G protein-coupled receptor E3 (ADGRE3), adhesion G protein-coupled receptor E4 (ADGRE4P), adhesion G protein-coupled receptor E5 (ADGRE5), adhesion G protein-coupled receptor F1 (ADGRF1), adhesion G protein-coupled receptor F2 (ADGRF2), adhesion G protein-coupled receptor F3 (ADGRF3), adhesion G protein-coupled receptor F4 (ADGRF4), adhesion G protein-coupled receptor F5 (ADGRF5), adhesion G protein-coupled receptor G1 (ADGRG1), adhesion G protein-coupled receptor G2 (ADGRG2), adhesion G protein-coupled receptor G3 (ADGRG3), adhesion G protein-coupled receptor G4 (ADGRG4), adhesion G protein-coupled receptor G5 (ADGRG5), adhesion G protein-coupled receptor G6 (ADGRG6), adhesion G protein-coupled receptor G7 (ADGRG7), adhesion G protein-coupled receptor L1 (ADGRL1), adhesion G protein-coupled receptor L2 (ADGRL2), adhesion G protein-coupled receptor L3 (ADGRL3), adhesion G protein-coupled receptor L4 (ADGRL4), adhesion G protein-coupled receptor V1 (ADGRV1), adrenoceptor alpha 1A (ADRA1A), adrenoceptor alpha 1B (ADRA1B), adrenoceptor alpha 1D (ADRA1D), adrenoceptor alpha 2A (ADRA2A), adrenoceptor alpha 2B (ADRA2B), adrenoceptor alpha 2C (ADRA2C), adrenoceptor beta 1 (ADRB1), adrenoceptor beta 2 (ADRB2), adrenoceptor beta 3 (ADRB3), angiotensin II receptor type 1 (AGTR1), angiotensin II receptor type 2 (AGTR2), apelin receptor (APLNR), G protein-coupled bile acid receptor 1 (GPBAR1), neuromedin B receptor (NMBR), gastrin releasing peptide receptor (GRPR), bombesin like receptor 3 (BRS3), bradykinin receptor B1 (BDKRB1), bradykinin receptor B2 (BDKRB2), calcitonin receptor (CALCR), calcitonin receptor like receptor (CALCRL), calcium sensing receptor (CASR), G protein-coupled receptor, class C (GPRC6A), cannabinoid receptor 1 (brain) (CNR1), cannabinoid receptor 2 (CNR2), chemerin chemokine-like receptor 1 (CMKLR1), chemokine (C—C motif) receptor 1 (CCR1), chemokine (C—C motif) receptor 2 (CCR2), chemokine (C—C motif) receptor 3 (CCR3), chemokine (C—C motif) receptor 4 (CCR4), chemokine (C—C motif) receptor 5 (gene/pseudogene) (CCR5), chemokine (C—C motif) receptor 6 (CCR6), chemokine (C—C motif) receptor 7 (CCR7), chemokine (C—C motif) receptor 8 (CCR8), chemokine (C—C motif) receptor 9 (CCR9), chemokine (C—C motif) receptor 10 (CCR10), chemokine (C—X—C motif) receptor 1 (CXCR1), chemokine (C—X—C motif) receptor 2 (CXCR2), chemokine (C—X—C motif) receptor 3 (CXCR3), chemokine (C—X—C motif) receptor 4 (CXCR4), chemokine (C—X—C motif) receptor 5 (CXCR5), chemokine (C—X—C motif) receptor 6 (CXCR6), chemokine (C—X3-C motif) receptor 1 (CX3CR1), chemokine (C motif) receptor 1 (XCR1), atypical chemokine receptor 1 (Duffy blood group) (ACKR1), atypical chemokine receptor 2 (ACKR2), atypical chemokine receptor 3 (ACKR3), atypical chemokine receptor 4 (ACKR4), chemokine (C—C motif) receptor-like 2 (CCRL2), cholecystokinin A receptor (CCKAR), cholecystokinin B receptor (CCKBR), G protein-coupled receptor 1 (GPR1), bombesin like receptor 3 (BRS3), G protein-coupled receptor 3 (GPR3), G protein-coupled receptor 4 (GPR4), G protein-coupled receptor 6 (GPR6), G protein-coupled receptor 12 (GPR12), G protein-coupled receptor 15 (GPR15), G protein-coupled receptor 17 (GPR17), G protein-coupled receptor 18 (GPR18), G protein-coupled receptor 19 (GPR19), G protein-coupled receptor 20 (GPR20), G protein-coupled receptor 21 (GPR21), G protein-coupled receptor 22 (GPR22), G protein-coupled receptor 25 (GPR25), G protein-coupled receptor 26 (GPR26), G protein-coupled receptor 27 (GPR27), G protein-coupled receptor 31 (GPR31), G protein-coupled receptor 32 (GPR32), G protein-coupled receptor 33 (gene/pseudogene) (GPR33), G protein-coupled receptor 34 (GPR34), G protein-coupled receptor 35 (GPR35), G protein-coupled receptor 37 (endothelin receptor type B-like) (GPR37), G protein-coupled receptor 37 like 1 (GPR37L1), G protein-coupled receptor 39 (GPR39), G protein-coupled receptor 42 (gene/pseudogene) (GPR42), G protein-coupled receptor 45 (GPR45), G protein-coupled receptor 50 (GPR50), G protein-coupled receptor 52 (GPR52), G protein-coupled receptor 55 (GPR55), G protein-coupled receptor 61 (GPR61), G protein-coupled receptor 62 (GPR62), G protein-coupled receptor 63 (GPR63), G protein-coupled receptor 65 (GPR65), G protein-coupled receptor 68 (GPR68), G protein-coupled receptor 75 (GPR75), G protein-coupled receptor 78 (GPR78), G protein-coupled receptor 79 (GPR79), G protein-coupled receptor 82 (GPR82), G protein-coupled receptor 83 (GPR83), G protein-coupled receptor 84 (GPR84), G protein-coupled receptor 85 (GPR85), G protein-coupled receptor 87 (GPR87), G protein-coupled receptor 88 (GPR88), G protein-coupled receptor 101 (GPR101), G protein-coupled receptor 119 (GPR119), G protein-coupled receptor 132 (GPR132), G protein-coupled receptor 135 (GPR135), G protein-coupled receptor 139 (GPR139), G protein-coupled receptor 141 (GPR141), G protein-coupled receptor 142 (GPR142), G protein-coupled receptor 146 (GPR146), G protein-coupled receptor 148 (GPR148), G protein-coupled receptor 149 (GPR149), G protein-coupled receptor 150 (GPR150), G protein-coupled receptor 151 (GPR151), G protein-coupled receptor 152 (GPR152), G protein-coupled receptor 153 (GPR153), G protein-coupled receptor 160 (GPR160), G protein-coupled receptor 161 (GPR161), G protein-coupled receptor 162 (GPR162), G protein-coupled receptor 171 (GPR171), G protein-coupled receptor 173 (GPR173), G protein-coupled receptor 174 (GPR174), G protein-coupled receptor 176 (GPR176), G protein-coupled receptor 182 (GPR182), G protein-coupled receptor 183 (GPR183), leucine-rich repeat containing G protein-coupled receptor 4 (LGR4), leucine-rich repeat containing G protein-coupled receptor 5 (LGR5), leucine-rich repeat containing G protein-coupled receptor 6 (LGR6), MAS1 proto-oncogene (MAS1), MAS1 proto-oncogene like (MAS1L), MAS related GPR family member D (MRGPRD), MAS related GPR family member E (MRGPRE), MAS related GPR family member F (MRGPRF), MAS related GPR family member G (MRGPRG), MAS related GPR family member X1 (MRGPRX1), MAS related GPR family member X2 (MRGPRX2), MAS related GPR family member X3 (MRGPRX3), MAS related GPR family member X4 (MRGPRX4), opsin 3 (OPN3), opsin 4 (OPN4), opsin 5 (OPN5), purinergic receptor P2Y (P2RY8), purinergic receptor P2Y (P2RY10), trace amine associated receptor 2 (TAAR2), trace amine associated receptor 3 (gene/pseudogene) (TAAR3), trace amine associated receptor 4 (TAAR4P), trace amine associated receptor 5 (TAAR5), trace amine associated receptor 6 (TAAR6), trace amine associated receptor 8 (TAAR8), trace amine associated receptor 9 (gene/pseudogene) (TAAR9), G protein-coupled receptor 156 (GPR156), G protein-coupled receptor 158 (GPR158), G protein-coupled receptor 179 (GPR179), G protein-coupled receptor, class C (GPRC5A), G protein-coupled receptor, class C (GPRC5B), G protein-coupled receptor, class C (GPRC5C), G protein-coupled receptor, class C (GPRC5D), frizzled class receptor 1 (FZD1), frizzled class receptor 2 (FZD2), frizzled class receptor 3 (FZD3), frizzled class receptor 4 (FZD4), frizzled class receptor 5 (FZD5), frizzled class receptor 6 (FZD6), frizzled class receptor 7 (FZD7), frizzled class receptor 8 (FZD8), frizzled class receptor 9 (FZD9), frizzled class receptor 10 (FZD10), smoothened, frizzled class receptor (SMO), complement component 3a receptor 1 (C3AR1), complement component 5a receptor 1 (C5AR1), complement component 5a receptor 2 (C5AR2), corticotropin releasing hormone receptor 1 (CRHR1), corticotropin releasing hormone receptor 2 (CRHR2), dopamine receptor D1 (DRD1), dopamine receptor D2 (DRD2), dopamine receptor D3 (DRD3), dopamine receptor D4 (DRD4), dopamine receptor D5 (DRD5), endothelin receptor type A (EDNRA), endothelin receptor type B (EDNRB), formyl peptide receptor 1 (FPR1), formyl peptide receptor 2 (FPR2), formyl peptide receptor 3 (FPR3), free fatty acid receptor 1 (FFAR1), free fatty acid receptor 2 (FFAR2), free fatty acid receptor 3 (FFAR3), free fatty acid receptor 4 (FFAR4), G protein-coupled receptor 42 (gene/pseudogene) (GPR42), gamma-aminobutyric acid (GABA) B receptor, 1 (GABBR1), gamma-aminobutyric acid (GABA) B receptor, 2 (GABBR2), galanin receptor 1 (GALR1), galanin receptor 2 (GALR2), galanin receptor 3 (GALR3), growth hormone secretagogue receptor (GHSR), growth hormone releasing hormone receptor (GHRHR), gastric inhibitory polypeptide receptor (GIPR), glucagon like peptide 1 receptor (GLP1R), glucagon-like peptide 2 receptor (GLP2R), glucagon receptor (GCGR), secretin receptor (SCTR), follicle stimulating hormone receptor (FSHR), luteinizing hormone/choriogonadotropin receptor (LHCGR), thyroid stimulating hormone receptor (TSHR), gonadotropin releasing hormone receptor (GNRHR), gonadotropin releasing hormone receptor 2 (pseudogene) (GNRHR2), G protein-coupled receptor 18 (GPR18), G protein-coupled receptor 55 (GPR55), G protein-coupled receptor 119 (GPR119), G protein-coupled estrogen receptor 1 (GPER1), histamine receptor H1 (HRH1), histamine receptor H2 (HRH2), histamine receptor H3 (HRH3), histamine receptor H4 (HRH4), hydroxycarboxylic acid receptor 1 (HCAR1), hydroxycarboxylic acid receptor 2 (HCAR2), hydroxycarboxylic acid receptor 3 (HCAR3), KISS1 receptor (KISS1R), leukotriene B4 receptor (LTB4R), leukotriene B4 receptor 2 (LTB4R2), cysteinyl leukotriene receptor 1 (CYSLTR1), cysteinyl leukotriene receptor 2 (CYSLTR2), oxoeicosanoid (OXE) receptor 1 (OXER1), formyl peptide receptor 2 (FPR2), lysophosphatidic acid receptor 1 (LPAR1), lysophosphatidic acid receptor 2 (LPAR2), lysophosphatidic acid receptor 3 (LPAR3), lysophosphatidic acid receptor 4 (LPAR4), lysophosphatidic acid receptor 5 (LPAR5), lysophosphatidic acid receptor 6 (LPAR6), sphingosine-1-phosphate receptor 1 (S1PR1), sphingosine-1-phosphate receptor 2 (S1PR2), sphingosine-1-phosphate receptor 3 (S1PR3), sphingosine-1-phosphate receptor 4 (S1PR4), sphingosine-1-phosphate receptor 5 (S1PR5), melanin concentrating hormone receptor 1 (MCHR1), melanin concentrating hormone receptor 2 (MCHR2), melanocortin 1 receptor (alpha melanocyte stimulating hormone receptor) (MC1R), melanocortin 2 receptor (adrenocorticotropic hormone) (MC2R), melanocortin 3 receptor (MC3R), melanocortin 4 receptor (MC4R), melanocortin 5 receptor (MC5R), melatonin receptor 1A (MTNR1A), melatonin receptor 1B (MTNR1B), glutamate receptor, metabotropic 1 (GRM1), glutamate receptor, metabotropic 2 (GRM2), glutamate receptor, metabotropic 3 (GRM3), glutamate receptor, metabotropic 4 (GRM4), glutamate receptor, metabotropic 5 (GRM5), glutamate receptor, metabotropic 6 (GRM6), glutamate receptor, metabotropic 7 (GRM7), glutamate receptor, metabotropic 8 (GRM8), motilin receptor (MLNR), neuromedin U receptor 1 (NMUR1), neuromedin U receptor 2 (NMUR2), neuropeptide FF receptor 1 (NPFFR1), neuropeptide FF receptor 2 (NPFFR2), neuropeptide S receptor 1 (NPSR1), neuropeptides B/W receptor 1 (NPBWR1), neuropeptides B/W receptor 2 (NPBWR2), neuropeptide Y receptor Y1 (NPY1R), neuropeptide Y receptor Y2 (NPY2R), neuropeptide Y receptor Y4 (NPY4R), neuropeptide Y receptor Y5 (NPY5R), neuropeptide Y receptor Y6 (pseudogene) (NPY6R), neurotensin receptor 1 (high affinity) (NTSR1), neurotensin receptor 2 (NTSR2), opioid receptor, delta 1 (OPRD1), opioid receptor, kappa 1 (OPRK1), opioid receptor, mu 1 (OPRM1), opiate receptor-like 1 (OPRL1), hypocretin (orexin) receptor 1 (HCRTR1), hypocretin (orexin) receptor 2 (HCRTR2), G protein-coupled receptor 107 (GPR107), G protein-coupled receptor 137 (GPR137), olfactory receptor family 51 subfamily E member 1 (OR51E1), transmembrane protein, adipocyte associated 1 (TPRA1), G protein-coupled receptor 143 (GPR143), G protein-coupled receptor 157 (GPR157), oxoglutarate (alpha-ketoglutarate) receptor 1 (OXGR1), purinergic receptor P2Y (P2RY1), purinergic receptor P2Y (P2RY2), pyrimidinergic receptor P2Y (P2RY4), pyrimidinergic receptor P2Y (P2RY6), purinergic receptor P2Y (P2RY11), purinergic receptor P2Y (P2RY12), purinergic receptor P2Y (P2RY13), purinergic receptor P2Y (P2RY14), parathyroid hormone 1 receptor (PTH1R), parathyroid hormone 2 receptor (PTH2R), platelet-activating factor receptor (PTAFR), prokineticin receptor 1 (PROKR1), prokineticin receptor 2 (PROKR2), prolactin releasing hormone receptor (PRLHR), prostaglandin D2 receptor (DP) (PTGDR), prostaglandin D2 receptor 2 (PTGDR2), prostaglandin E receptor 1 (PTGER1), prostaglandin E receptor 2 (PTGER2), prostaglandin E receptor 3 (PTGER3), prostaglandin E receptor 4 (PTGER4), prostaglandin F receptor (PTGFR), prostaglandin 12 (prostacyclin) receptor (IP) (PTGIR), thromboxane A2 receptor (TBXA2R), coagulation factor II thrombin receptor (F2R), F2R like trypsin receptor 1 (F2RL1), coagulation factor II thrombin receptor like 2 (F2RL2), F2R like thrombin/trypsin receptor 3 (F2RL3), pyroglutamylated RFamide peptide receptor (QRFPR), relaxin/insulin-like family peptide receptor 1 (RXFP1), relaxin/insulin-like family peptide receptor 2 (RXFP2), relaxin/insulin-like family peptide receptor 3 (RXFP3), relaxin/insulin-like family peptide receptor 4 (RXFP4), somatostatin receptor 1 (SSTR1), somatostatin receptor 2 (SSTR2), somatostatin receptor 3 (SSTR3), somatostatin receptor 4 (SSTR4), somatostatin receptor 5 (SSTR5), succinate receptor 1 (SUCNR1), tachykinin receptor 1 (TACR1), tachykinin receptor 2 (TACR2), tachykinin receptor 3 (TACR3), taste 1 receptor member 1 (TAS1R1), taste 1 receptor member 2 (TAS1R2), taste 1 receptor member 3 (TAS1R3), taste 2 receptor member 1 (TAS2R1), taste 2 receptor member 3 (TAS2R3), taste 2 receptor member 4 (TAS2R4), taste 2 receptor member 5 (TAS2R5), taste 2 receptor member 7 (TAS2R7), taste 2 receptor member 8 (TAS2R8), taste 2 receptor member 9 (TAS2R9), taste 2 receptor member 10 (TAS2R10), taste 2 receptor member 13 (TAS2R13), taste 2 receptor member 14 (TAS2R14), taste 2 receptor member 16 (TAS2R16), taste 2 receptor member 19 (TAS2R19), taste 2 receptor member 20 (TAS2R20), taste 2 receptor member 30 (TAS2R30), taste 2 receptor member 31 (TAS2R31), taste 2 receptor member 38 (TAS2R38), taste 2 receptor member 39 (TAS2R39), taste 2 receptor member 40 (TAS2R40), taste 2 receptor member 41 (TAS2R41), taste 2 receptor member 42 (TAS2R42), taste 2 receptor member 43 (TAS2R43), taste 2 receptor member 45 (TAS2R45), taste 2 receptor member 46 (TAS2R46), taste 2 receptor member 50 (TAS2R50), taste 2 receptor member 60 (TAS2R60), thyrotropin-releasing hormone receptor (TRHR), trace amine associated receptor 1 (TAAR1), urotensin 2 receptor (UTS2R), arginine vasopressin receptor 1A (AVPR1A), arginine vasopressin receptor 1B (AVPR1B), arginine vasopressin receptor 2 (AVPR2), oxytocin receptor (OXTR), adenylate cyclase activating polypeptide 1 (pituitary) receptor type I (ADCYAP1R1), vasoactive intestinal peptide receptor 1 (VIPR1), vasoactive intestinal peptide receptor 2 (VIPR2), and any variant thereof.

In some embodiments, a chimeric receptor comprises a G-protein coupled receptor (GPCR), or any variant thereof. In some embodiments, a chimeric receptor comprises at least an extracellular region (e.g., ligand binding domain) of a GPCR, or any variant thereof. In some embodiments, a chimeric receptor comprises at least a membrane spanning region of a GPCR, or any variant thereof. In some embodiments, a chimeric receptor comprises at least an intracellular region (e.g., cytoplasmic domain) of a GPCR, or any variant thereof. A chimeric receptor comprising a GPCR, or any variant thereof, can bind a GPCR ligand. In some embodiments, ligand binding to a chimeric receptor comprising a GPCR, or any variant thereof, results in activation of a GPCR signaling pathway.

In some embodiments, a transmembrane receptor comprises an integrin receptor, an integrin receptor subunit, or any variant thereof (e.g., synthetic or chimeric receptor). Integrin receptors are transmembrane receptors that can function as bridges for cell-cell and cell-extracellular matrix (ECM) interactions. Integrin receptors are generally formed as heterodimers consisting of an α subunit and a β subunit which associate non-covalently. There exist at least 18 α subunits and at least 8 β subunits. Each subunit generally comprises an extracellular region (e.g., ligand binding domain), a region spanning a membrane, and an intracellular region (e.g., cytoplasmic domain).

In some embodiments, a transmembrane receptor comprises an integrin receptor a subunit, or any variant thereof, selected from the group consisting of: α1, α2, α3, α4, α5, α6, α7, α8, α9, α10, α11, αV, αL, αM, αX, αD, αE, and cub. In some embodiments, a transmembrane receptor comprises an integrin receptor β subunit, or any variant thereof, selected from the group consisting of: β1, β2, β3, β4, β5, β6, β7, and β8. A transmembrane receptor of a subject system comprising an a subunit, a β subunit, or any variant thereof, can heterodimerize (e.g., α subunit dimerizing with a β subunit) to form an integrin receptor, or any variant thereof. Non-limiting examples of integrin receptors include an α1β1, α2β1, α3β1, α4β1, α5β1, α6β1, α7β1, α8β1, α9β1, α10β1, αVβ1, αLβ1, αMβ1, αXβ1, αDβ1, αIIbβ1, αEβ1, α1β2, α2β2, α3β2, α4β2, α5β2, α6β2, α7β2, α8β2, α9β2, α10β2, αVβ2, αLβ2, αMβ2, αXβ2, αDβ2, αIIbβ2, αEβ2, α1β3, α2β3, α3β3, α4β3, α5β3, α6β3, α7β3, α8β3, α9β3, α10β3, αVβ3, αLβ3, αMβ3, αXβ3, αDβ3, αIIbβ3, αEβ3, α1β4, α2β4, α3β4, α4β4, α5β4, α6β4, α7β4, α8β4, α9β4, α10β4, αVβ4, αLβ4, αMβ4, αXβ4, αDβ4, αIIbβ4, αEβ4, α1β5, α2β5, α3β5, α4β5, α5β5, α6β5, α7β5, α8β5, α9β5, α10β5, αVβ5, αLβ5, αMβ5, αXβ5, αDβ5, αIIbβ5, αEβ5, α1β6, α2β6, α3β6, α4β6, α5β6, α6β6, α7β6, α8β6, α9β6, α10β6, αVβ6, αLβ6, αMβ6, αXβ6, αDβ6, αIIbβ6, αEβ6, α1β7, α2β7, α3β7, α4β7, α5β7, α6β7, α7β7, α8β7, α9β7, α10β7, αVβ7, αLβ7, αMβ7, αXβ7, αDβ7, αIIbβ7, αEβ7, α1β8, α2β8, α3β8, α4β8, α5β8, α6β8, α7β8, α8β8, α9β8, α10β8, αVβ8, αLβ8, αMβ8, αXβ8, αDβ8, 0%08, and αEβ8 receptor.

In some embodiments, a chimeric receptor comprises at least an extracellular region (e.g., ligand binding domain) of an integrin subunit (e.g., α subunit or β subunit), or any variant thereof. In some embodiments, a chimeric receptor comprises at least a region spanning a membrane of an integrin subunit (e.g., α subunit or β subunit), or any variant thereof. In some embodiments, a chimeric receptor comprises at least an intracellular region (e.g., cytoplasmic domain) of an integrin subunit (e.g., α subunit or β subunit), or any variant thereof. A chimeric receptor comprising an integrin subunit, or any variant thereof, can bind an integrin ligand. In some embodiments, ligand binding to a chimeric receptor comprising an integrin subunit, or any variant thereof, results in activation of an integrin signaling pathway.

In some embodiments, a transmembrane receptor comprises a cadherin molecule, or any variant thereof (e.g., synthetic or chimeric receptor). Cadherin molecules, which can function as both ligands and receptors, refer to certain proteins involved in mediating cell adhesion. Cadherin molecules generally consist of five tandem repeated extracellular domains, a single membrane-spanning segment and a cytoplasmic region. E-cadherin, or CDH1, for example, consists of 5 repeats in the extracellular domain, one transmembrane domain, and an intracellular domain. When E-cadherin is phosphorylated at a region of the intracellular domain, adaptor proteins such as beta-catenin and p120-catenin can bind to the receptor.

In some embodiments, a transmembrane receptor comprises a cadherin, or any variant thereof, selected from a classical cadherin, a desmosoma cadherin, a protocadherin, and an unconventional cadherin. In some embodiments, a transmembrane receptor comprises a classical cadherin, or any variant thereof, selected from CDH1 (E-cadherin, epithelial), CDH2 (N-cadherin, neural), CDH12 (cadherin 12, type 2, N-cadherin 2), and CDH3 (P-cadherin, placental). In some embodiments, a transmembrane receptor comprises a desmosoma cadherin, or any variant thereof, selected from desmoglein (DSG1, DSG2, DSG3, DSG4) and desmocollin (DSC1, DSC2, DSC3). In some embodiments, a transmembrane receptor comprises a protocadherin, or any variant thereof, selected from PCDH1, PCDH10, PCDH11X, PCDH11Y, PCDH12, PCDH15, PCDH17, PCDH18, PCDH19, PCDH20, PCDH7, PCDH8, PCDH9, PCDHA1, PCDHA10, PCDHA11, PCDHA12, PCDHA13, PCDHA2, PCDHA3, PCDHA4, PCDHA5, PCDHA6, PCDHA7, PCDHA8, PCDHA9, PCDHAC1, PCDHAC2, PCDHB1, PCDHB10, PCDHB11, PCDHB12, PCDHB13, PCDHB14, PCDHB15, PCDHB16, PCDHB17, PCDHB18, PCDHB2, PCDHB3, PCDHB4, PCDHB5, PCDHB6, PCDHB7, PCDHB8, PCDHB9, PCDHGA1, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB4, PCDHGB5, PCDHGB6, PCDHGB7, PCDHGC3, PCDHGC4, PCDHGC5, FAT, FAT2, and FAT). In some embodiments, a transmembrane receptor comprises an unconventional cadherin selected from CDH4 (R-cadherin, retinal), CDH5 (VE-cadherin, vascular endothelial), CDH6 (K-cadherin, kidney), CDH7 (cadherin 7, type 2), CDH8 (cadherin 8, type 2), CDH9 (cadherin 9, type 2, T1-cadherin), CDH10 (cadherin 10, type 2, T2-cadherin), CDH11 (OB-cadherin, osteoblast), CDH13 (T-cadherin, H-cadherin, heart), CDH15 (M-cadherin, myotubule), CDH16 (KSP-cadherin), CDH17 (LI cadherin, liver-intestine), CDH18 (cadherin 18, type 2), CDH19 (cadherin 19, type 2), CDH20 (cadherin 20, type 2), CDH23 (cadherin 23, neurosensory epithelium), CDH24, CDH26, CDH28, CELSR1, CELSR2, CELSR3, CLSTN1, CLSTN2, CLSTN3, DCHS1, DCHS2, LOC389118, PCLKC, RESDA1, and RET.

In some embodiments, a chimeric receptor comprises a cadherin molecule, or any variant thereof. In some embodiments, a chimeric receptor comprises at least an extracellular region of a cadherin, or any variant thereof. In some embodiments, a chimeric receptor comprises at least a region spanning a membrane of a cadherin, or any variant thereof. In some embodiments, a chimeric receptor comprises at least an intracellular region (e.g., cytoplasmic domain) of a cadherin, or any variant thereof. A chimeric receptor polypeptide comprising a cadherin, or any variant thereof, can bind a cadherin ligand. In some embodiments, ligand binding to a chimeric receptor comprising a cadherin, or any variant thereof, results in activation of a cadherin signaling pathway.

In some embodiments, a transmembrane receptor comprises a catalytic receptor, or any variant thereof (e.g., synthetic or chimeric receptor). Examples of catalytic receptors include, but are not limited to, receptor tyrosine kinases (RTKs) and receptor threonine/serine kinases (RTSKs). Catalytic receptors such as RTKs and RTSKs possess certain enzymatic activities. RTKs, for example, can phosphorylate substrate proteins on tyrosine residues which can then act as binding sites for adaptor proteins. RTKs generally comprise an N-terminal extracellular ligand-binding domain, a single transmembrane α helix, and a cytosolic C-terminal domain with protein-tyrosine kinase activity. Some RTKs consist of single polypeptides while some are dimers consisting of two pairs of polypeptide chains, for example the insulin receptor and some related receptors. The binding of ligands to the extracellular domains of these receptors can activate the cytosolic kinase domains, resulting in phosphorylation of both the receptors themselves and intracellular target proteins that propagate the signal initiated by ligand binding. In some RTKs, ligand binding induces receptor dimerization. Some ligands (e.g., growth factors such as PDGF and NGF) are themselves dimers consisting of two identical polypeptide chains. These growth factors can directly induce dimerization by simultaneously binding to two different receptor molecules. Other growth factors (e.g., such as EGF) are monomers but have two distinct receptor binding sites that can crosslink receptors. Ligand-induced dimerization can result in autophosphorylation of the receptor, wherein the dimerized polypeptide chains cross-phosphorylate one another. Some receptors can multimerize.

In some embodiments, a transmembrane receptor comprises a class I RTK (e.g., the epidermal growth factor (EGF) receptor family including EGFR; the ErbB family including ErbB-2, ErbB-3, and ErbB-4), a class II RTK (e.g., the insulin receptor family including INSR, IGF-1R, and IRR), a class III RTK (e.g., the platelet-derived growth factor (PDGF) receptor family including PDGFR-α, PDGFR-β, CSF-1R, KIT/SCFR, and FLK2/FLT3), a class IV RTK (e.g., the fibroblast growth factor (FGF) receptor family including FGFR-1, FGFR-2, FGFR-3, and FGFR-4), a class V RTK (e.g., the vascular endothelial growth factor (VEGF) receptor family including VEGFR1, VEGFR2, and VEGFR3), a class VI RTK (e.g., the hepatocyte growth factor (HGF) receptor family including hepatocyte growth factor receptor (HGFR/MET) and RON), a class VII RTK (e.g., the tropomyosin receptor kinase (Trk) receptor family including TRKA, TRKB, and TRKC), a class VIII RTK (e.g., the ephrin (Eph) receptor family including EPHA1, EPHA2, EPHA3, EPHA4, EPHA5, EPHA6, EPHA7, EPHA8, EPHB1, EPHB2, EPHB3, EPHB4, EPHB5, and EPHB6), a class IX RTK (e.g., AXL receptor family such as AXL, MER, and TRYO3), a class X RTK (e.g., LTK receptor family such as LTK and ALK), a class XI RTK (e.g., TIE receptor family such as TIE and TEK), a class XII RTK (e.g., ROR receptor family ROR1 and ROR2), a class XIII RTK (e.g., the discoidin domain receptor (DDR) family such as DDR1 and DDR2), a class XIV RTK (e.g., RET receptor family such as RET), a class XV RTK (e.g., KLG receptor family including PTK7), a class XVI RTK (e.g., RYK receptor family including Ryk), a class XVII RTK (e.g., MuSK receptor family such as MuSK), or any variant thereof.

In some embodiments, a chimeric receptor comprises at least an extracellular region (e.g., ligand binding domain) of a catalytic receptor such as a RTK, or any variant thereof. In some embodiments, a chimeric receptor comprises at least a membrane spanning region of a catalytic receptor such as a RTK, or any variant thereof. In some embodiments, a chimeric receptor comprises at least an intracellular region (e.g., cytosolic domain) of a catalytic receptor such as a RTK, or any variant thereof. A chimeric receptor comprising an RTK, or any variant thereof, can bind a RTK ligand. In some embodiments, ligand binding to a chimeric receptor comprising an RTK, or any variant thereof, results in activation of a RTK signaling pathway.

In some embodiments, a chimeric receptor comprises at least an extracellular region (e.g., ligand binding domain) of a catalytic receptor such as an RTSK, or any variant thereof. In some embodiments, a chimeric receptor comprises at least a membrane spanning region of a catalytic receptor such as an RTSK, or any variant thereof. In some embodiments, a chimeric receptor comprises at least an intracellular region (e.g., cytosolic domain) of a catalytic receptor such as an RTSK, or any variant thereof. A chimeric receptor comprising an RTSK, or any variant thereof, can bind a RTSK ligand. In some embodiments, ligand binding to a chimeric receptor comprising an RTSK, or any variant thereof, results in activation of a RTSK signaling pathway.

In some embodiments, a transmembrane receptor comprising an RTSK, or any variant thereof, can phosphorylate a substrate at serine and/or threonine residues, and may select specific residues based on a consensus sequence. A transmembrane receptor can comprise a type I RTSK, type II RTSK, or any variant thereof. In some embodiments, a transmembrane receptor comprising a type I receptor serine/threonine kinase is inactive unless complexed with a type II receptor. In some embodiments, a transmembrane receptor comprising a type II receptor serine/threonine comprises a constitutively active kinase domain that can phosphorylate and activate a type I receptor when complexed with the type I receptor. A type II receptor serine/threonine kinase can phosphorylate the kinase domain of the type I partner, causing displacement of protein partners.

Displacement of protein partners can allow binding and phosphorylation of other proteins, for example certain members of the SMAD family. A transmembrane receptor can comprise a type I receptor, or any variant thereof, selected from the group consisting of: ALK1 (ACVRL1), ALK2 (ACVR1A), ALK3 (BMPR1A), ALK4 (ACVR1B), ALK5 (TGFβR1), ALK6 (BMPR1B), and ALK7 (ACVR1C). A transmembrane receptor can comprise a type II receptor, or any variant thereof, selected from the group consisting of: TGFβR2, BMPR2, ACVR2A, ACVR2B, and AMHR2 (AMHR).

In some embodiments, a transmembrane receptor comprises a receptor which stimulates non-covalently associated intracellular kinases, such as a Src kinase (e.g., c-Src, Yes, Fyn, Fgr, Lck, Hck, Blk, Lyn, and Frk) or a JAK kinase (e.g., JAK1, JAK2, JAK3, and TYK2) rather than possessing intrinsic enzymatic activity, or any variant thereof. These include the cytokine receptor superfamily such as receptors for cytokines and polypeptide hormones. Cytokine receptors generally contain an N-terminal extracellular ligand-binding domain, transmembrane α helices, and a C-terminal cytosolic domain. The cytosolic domains of cytokine receptors are generally devoid of any known catalytic activity. Cytokine receptors instead can function in association with non-receptor kinases (e.g., tyrosine kinases or threonine/serine kinases), which can be activated as a result of ligand binding to the receptor.

In some embodiments, a chimeric receptor comprises at least an extracellular region (e.g., ligand binding domain) of a catalytic receptor that non-covalently associates with an intracellular kinase (e.g., a cytokine receptor), or any variant thereof. In some embodiments, a chimeric receptor comprises at least a membrane spanning region of a catalytic receptor that non-covalently associates with an intracellular kinase (e.g., a cytokine receptor), or any variant thereof. In some embodiments, a chimeric receptor comprises at least an intracellular region (e.g., cytosolic domain) of a catalytic receptor that non-covalently associates with an intracellular kinase (e.g., a cytokine receptor), or any variant thereof. A chimeric receptor comprising a catalytic receptor that non-covalently associates with an intracellular kinase, or any variant thereof, can bind a ligand. In some embodiments, ligand binding to a chimeric receptor comprising a catalytic receptor that non-covalently associates with an intracellular kinase, or any variant thereof, results in activation of a signaling pathway.

Cytokine receptors generally contain an N-terminal extracellular ligand-binding domain, transmembrane α helices, and a C-terminal cytosolic domain. The cytosolic domains of cytokine receptors are generally devoid of any known catalytic activity. Cytokine receptors instead can function in association with non-receptor kinases (e.g., tyrosine kinases or threonine/serine kinases), which can be activated as a result of ligand binding to the receptor.

In some embodiments, a transmembrane receptor comprises a cytokine receptor, for example a type I cytokine receptor or a type II cytokine receptor, or any variant thereof. In some embodiments, a transmembrane receptor comprises an interleukin receptor (e.g., IL-2R, IL-3R, IL-4R, IL-5R, IL-6R, IL-7R, IL-9R, IL-11R, IL-12R, IL-13R, IL-15R, IL-21R, IL-23R, IL-27R, and IL-31R), a colony stimulating factor receptor (e.g., erythropoietin receptor, CSF-1R, CSF-2R, GM-CSFR, and G-CSFR), a hormone receptor/neuropeptide receptor (e.g., growth hormone receptor, prolactin receptor, and leptin receptor), or any variant thereof. In some embodiments, a transmembrane receptor comprises a type II cytokine receptor, or any variant thereof. In some embodiments, a transmembrane receptor comprises an interferon receptor (e.g., IFNAR1, IFNAR2, and IFNGR), an interleukin receptor (e.g., IL-10R, IL-20R, IL-22R, and IL-28R), a tissue factor receptor (also called platelet tissue factor), or any variant thereof.

In some embodiments, a transmembrane receptor comprises a death receptor, a receptor containing a death domain, or any variant thereof. Death receptors are often involved in regulating apoptosis and inflammation. Death receptors include members of the TNF receptor family such as TNFR1, Fas receptor, DR4 (also known as TRAIL receptor 1 or TRAILR1) and DR5 (also known as TRAIL receptor 2 or TRAILR2).

In some embodiments, a chimeric receptor comprises at least an extracellular region (e.g., ligand binding domain) of a death receptor, or any variant thereof. In some embodiments, a chimeric receptor comprises at least a membrane spanning region of a death receptor, or any variant thereof. In some embodiments, a chimeric receptor comprises at least an intracellular region (e.g., cytosolic) domain of a death receptor, or any variant thereof. A chimeric receptor comprising a death receptor, or any variant thereof, can undergo receptor oligomerization in response to ligand binding, which in turn can result in the recruitment of specialized adaptor proteins and activation of signaling cascades, such as caspase cascades.

In some embodiments, a transmembrane receptor comprises an immune receptor, or any variant thereof. Immune receptors include members of the immunoglobulin superfamily (IgSF) which share structural features with immunoglobulins, e.g., a domain known as an immunoglobulin domain or fold. IgSF members include, but are not limited to, cell surface antigen receptors, co-receptors and costimulatory molecules of the immune system, and molecules involved in antigen presentation to lymphocytes.

In some embodiments, a chimeric receptor comprises an immune receptor, or any variant thereof. In some embodiments, a chimeric receptor comprises at least an extracellular region (e.g., ligand binding domain) of an immune receptor, or any variant thereof. In some embodiments, a chimeric receptor comprises at least a region spanning a membrane of an immune receptor, or any variant thereof. In some embodiments, a chimeric receptor comprises at least an intracellular region (e.g., cytoplasmic domain) of an immune receptor, or any variant thereof. A chimeric receptor comprising an immune receptor, or any variant thereof, can recruit a binding partner. In some embodiments, ligand binding to a chimeric receptor comprising an immune receptor, or any variant thereof, results in activation of an immune cell signaling pathway.

In some embodiments, a transmembrane receptor comprises a cell surface antigen receptor such as a T cell receptor (TCR), a B cell receptor (BCR), or any variant thereof. T cell receptors generally comprise two chains, either the TCR-alpha and -beta chains or the TCR-delta and -gamma chains. A transmembrane receptor comprising a TCR, or any variant thereof, can bind a major histocompatibility complex (MHC) protein. B cell receptors generally comprises a membrane bound immunoglobulin and a signal transduction moiety. A transmembrane receptor comprising a BCR, or any variant thereof, can bind a cognate BCR antigen.

In some embodiments, a transmembrane receptor comprises a chimeric antigen receptor (CAR). The ligand binding domain of the CAR can bind any ligand. In some cases, the ligand is referred to as an antigen. The ligand binding domain can comprise a monoclonal antibody, a polyclonal antibody, a recombinant antibody, a human antibody, a humanized antibody, or a functional variant thereof, including, but not limited to, a Fab, a Fab′, a F(ab′)2, an Fv, a single-chain Fv (scFv), minibody, a diabody, and a single-domain antibody such as a heavy chain variable domain (VH), a light chain variable domain (VL) and a variable domain (VHH) of camelid derived Nanobody. In some embodiments, the ligand binding domain comprises at least one of a Fab, a Fab′, a F(ab′)2, an Fv, and a scFv. In some embodiments, the ligand binding domain comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, the ligand binding domain of the CAR domain comprises a transmembrane receptor, or any variant thereof. For example, the ligand binding domain can comprise at least a ligand binding domain of a transmembrane receptor.

In some embodiments, the ligand binding domain comprises a humanized antibody. A humanized antibody can be produced using a variety of techniques including, but not limited to, CDR-grafting, veneering or resurfacing, chain shuffling, and other techniques. Human variable domains, including light and heavy chains, can be selected to reduce the immunogenicity of humanized antibodies. In some embodiments, the ligand binding domain of a chimeric transmembrane receptor comprises a fragment of a humanized antibody which binds an antigen with high affinity and possesses other favorable biological properties, such as reduced and/or minimal immunogenicity. A humanized antibody or antibody fragment can retain a similar antigenic specificity as the corresponding non-humanized antibody.

In some embodiments, the ligand binding domain comprises a single-chain variable fragment (scFv). scFv molecules can be produced by linking the heavy chain (VH) and light chain (VL) regions of immunoglobulins together using flexible linkers, such as polypeptide linkers. scFvs can be prepared according to various methods.

In some embodiments, the ligand binding domain is engineered to bind a specific target antigen. For example, the ligand binding domain can be an engineered scFv. A ligand binding domain comprising a scFv can be engineered using a variety of methods, including but not limited to display libraries such as phage display libraries, yeast display libraries, cell based display libraries (e.g., mammalian cells), protein-nucleic acid fusions, ribosome display libraries, and/or an E. coli periplasmic display libraries. In some embodiments, a ligand binding domain which is engineered may bind to an antigen with a higher affinity than an analogous antibody or an antibody which has not undergone engineering.

In some embodiments, the ligand binding domain binds multiple ligands (e.g., antigens), e.g., at least 2, 3, 4, 5, 6, 7, 8, 9, or 10 antigens. A ligand binding domain can bind two related antigens, such as two subtypes of botulin toxin (e.g., botulinum neurotoxin subtype A1 and subtype A2). A ligand binding domain can bind two unrelated proteins, such as receptor tyrosine kinase erbB-2 (also referred to as Neu, ERBB2, and HER2) and vascular endothelial growth factor (VEGF). A ligand binding domain capable of binding two antigens can comprise an antibody engineered to bind two unrelated protein targets at distinct but overlapping sites of the antibody. In some embodiments, a ligand binding domain which binds multiple antigens comprises a bispecific antibody molecule. A bispecific antibody molecule can have a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). The first and second epitopes can overlap. In some embodiments, the first and second epitopes do not overlap. In some embodiments, the first and second epitopes are on different antigens, e.g., different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope.

In some embodiments, the extracellular region of a chimeric transmembrane receptor comprises multiple ligand binding domains, for example at least 2 ligand binding domains (e.g., at least 3, 4, 5, 6, 7, 8, 9, or 10 ligand binding domains). The multiple ligand binding domains can exhibit binding to the same or different antigen. In some embodiments, the extracellular region comprises at least two ligand binding domains, for example at least two scFvs linked in tandem. In some embodiments, two scFv fragments are linked by a peptide linker.

The ligand binding domain of an extracellular region of a chimeric transmembrane receptor can bind a membrane bound antigen, for example an antigen at the extracellular surface of a cell (e.g., a target cell). In some embodiments, the ligand binding domain binds an antigen that is not membrane bound (e.g., non-membrane-bound), for example an extracellular antigen that is secreted by a cell (e.g., a target cell) or an antigen located in the cytoplasm of a cell (e.g., a target cell). Antigens (e.g., membrane bound and non-membrane bound) can be associated with a disease such as a viral, bacterial, and/or parasitic infection; inflammatory and/or autoimmune disease; or neoplasm such as a cancer and/or tumor. Non-limiting examples of antigens which can be bound by a ligand binding domain of a chimeric transmembrane receptor polypeptide of a subject system include, but are not limited to, 1-40-β-amyloid, 4-1BB, SAC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β3 (ADRB3), AGS-22M6, a folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (a chain of IL-2 receptor), CD27, CD274, CD28, CD3, CD3ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C—X—C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRCSD), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-Rα), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAIVIE, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, f3 Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR I′ Alternate Reading Frame Protein (TARP), telomerase, TEM1, tenascin C, TGF-β (e.g., TGF-β1, TGF-β2, TGF-β3), thyroid stimulating hormone receptor (TSHR), tissue factor pathway inhibitor (TFPI), Tn antigen ((Tn Ag) or (GalNAca-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, and κ-light chain.

In some embodiments, the ligand binding domain binds an antigen selected from the group consisting of: 707-AP, a biotinylated molecule, a-Actinin-4, abl-bcr alb-b3 (b2α2), abl-bcr alb-b4 (b3α2), adipophilin, AFP, AIM-2, Annexin II, ART-4, BAGE, b-Catenin, bcr-abl, bcr-abl p190 (e1α2), bcr-abl p210 (b2α2), bcr-abl p210 (b3α2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2M, HST-2 (FGF6), HST-2/neu, hTERT, iCE, IL-11Ra, IL-13Ra2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAIVIE, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain. In some embodiments, the ligand binding domain binds to a tumor associated antigen.

In some embodiments, the ligand binding domain binds an antigen comprising an antibody e.g., an antibody bound to a cell surface protein or polypeptide. The protein or polypeptide on the cell surface bound by an antibody can comprise an antigen associated with a disease such as a viral, bacterial, and/or parasitic infection; inflammatory and/or autoimmune disease; or neoplasm such as a cancer and/or tumor. In some embodiments, the antibody binds a tumor associated antigen (e.g., protein or polypeptide). In some embodiments, a ligand binding domain of a chimeric transmembrane receptor disclosed herein can bind a monoclonal antibody, a polyclonal antibody, a recombinant antibody, a human antibody, a humanized antibody, or a functional variant thereof, including, but not limited to, a Fab, a Fab′, a F(ab′)2, an Fc, an Fv, a scFv, minibody, a diabody, and a single-domain antibody such as a heavy chain variable domain (VH), a light chain variable domain (VL) and a variable domain (VHH) of camelid derived Nanobody. In some embodiments, a ligand binding domain can bind at least one of a Fab, a Fab′, a F(ab′)2, an Fc, an Fv, and a scFv. In some embodiments, the ligand binding domain binds an Fc domain of an antibody.

In some embodiments, the ligand binding domain binds an antibody selected from the group consisting of: 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, ABP 494 (cetuximab biosimilar), abrilumab, ABT-700, ABT-806, Actimab-A (actinium Ac-225 lintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine 1-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imciromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDI0680, MEDI-3617, MEDI-551 (inebilizumab), MEDI-565, MEDI6469, mepolizumab, metelimumab, MGB453, MGD006/S80880, MGD007, MGD009, MGD011, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, R07009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox. In certain embodiments, the ligand binding domain binds an Fc domain of an aforementioned antibody.

In some embodiments, the ligand binding domain binds an antibody which in turn binds an antigen selected from the group consisting of: 1-40-β-amyloid, 4-1BB, SAC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (a chain of IL-2 receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSF1R, CSF2, CTLA-4, C—X—C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-γ, IgE, IgE Fc region, IGF-1 receptor, IGF-1, IGHE, IL 17A, IL 17F, IL 20, IL-12, IL-13, IL-17, IL-1β, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7 (37, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-Rα, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β1, TGF-β2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRP1 (glycoprotein 75), VEGFA, VEGFR1, VEGFR2, vimentin, and VWF.

In some embodiments, a ligand binding domain can bind an antibody mimetic. Antibody mimetics, as described elsewhere herein, can bind a target molecule with an affinity comparable to an antibody. In some embodiments, the ligand binding domain can bind a humanized antibody which is described elsewhere herein. In some embodiments, the ligand binding domain of a chimeric transmembrane receptor can bind a fragment of a humanized antibody. In some embodiments, the ligand binding domain can bind a single-chain variable fragment (scFv).

In some embodiments, the ligand binding domain binds an Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A and protein G). Additionally, Fc binding domains may be synthetic polypeptides engineered specifically to bind the Fc portion of any of the Ig molecules described herein with desired affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, and a nanobody. Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide library for binding activities to Fc.

In some embodiments, the ligand binding domain comprises an Fc binding domain comprising an extracellular ligand-binding domain of a mammalian Fc receptor. Fc receptors are generally cell surface receptors expressed on the surface of many immune cells (including B cells, dendritic cells, natural killer (NK) cells, macrophages, neutorphils, mast cells, and eosinophils) and exhibit binding specificity to the Fc domain of an antibody. In some cases, binding of an Fc receptor to an Fc portion of the antibody can trigger antibody dependent cell-mediated cytotoxicity (ADCC) effects. The Fc receptor used for constructing a chimeric transmembrane receptor polypeptide described herein may be a naturally-occurring polymorphism variant, such as a variant which may have altered (e.g., increased or decreased) affinity to an Fc domain as compared to a wild-type counterpart. Alternatively, the Fc receptor may be a functional variant of a wild-type counterpart, carrying one or more mutations (e.g., up to 10 amino acid residue substitutions) that alters the binding affinity to the Fc portion of an Ig molecule. In some embodiments, the mutation may alter the glycosylation pattern of the Fc receptor and thus the binding affinity to an Fc domain.

Table 1 lists a number of exemplary polymorphisms in Fc receptor extracellular domains (see, e.g., Kim et al., J. Mol. Evol. 53:1-9, 2001).

TABLE 1
Exemplary Polymorphisms in Fc Receptors

| Amino Acid | | | | | | | | | | |
|---|---|---|---|---|---|---|---|---|---|---|
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcγRII) | N | N | N | S | E | S | S | S | I | I |
| M31932 (FcγRI) | S | T | N | R | E | A | F | T | I | G |
| X06948 (FcαεI) | R | S | E | S | Q | S | E | S | I | V |

Fc receptors can generally be classified based on the isotype of the antibody to which it is able to bind. For example, Fc-gamma receptors (FcγR) generally bind to IgG antibodies (e.g., IgG1, IgG2, IgG3, and IgG4); Fc-alpha receptors (FcαR) generally bind to IgA antibodies; and Fc-epsilon receptors (FcεR) generally bind to IgE antibodies. In some embodiments, the ligand binding domain comprises an Fcγ receptor or any variant thereof. In some embodiments, the ligand binding domain comprises an Fc binding domain comprising an FcR selected from FcγRI (CD64), FcγRIa, FcγRIb, FcγRIc, FcγRIIA (CD32) including allotypes H131 and R131, FcγRIIB (CD32) including FcγRIIB-1 and FcγRIIB-2, FcγRIIIA (CD16a) including allotypes V158 and F158, FcγRIIIB (CD16b) including allotypes FcγRIIIb-NA1 and FcγRIIIb-NA2, and any variant thereof. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2). In some embodiments, the ligand binding domain comprises an FCC receptor or any variant thereof. In some embodiments, the ligand binding domain comprises a FcR selected from FcεRI, FcεRII (CD23), and any variant thereof. In some embodiments, the ligand binding domain comprises an Fcα receptor or any variant thereof. In some embodiments, the ligand binding domain comprises an FcR selected from FcαRI (CD89), Fcα/μR, and any variant thereof. In some embodiments, the ligand binding domain comprises an FcR selected from FcRn, and any variant thereof. Selection of the ligand binding domain of an Fc receptor for use in the chimeric transmembrane receptor may depend on various factors such as the isotype of the antibody to which binding of the Fc binding domain is desired and the desired affinity of the binding interaction.

In some embodiments, the ligand binding domain comprises the extracellular ligand-binding domain of CD16, which may incorporate a naturally occurring polymorphism that can modulate affinity for an Fc domain. In some embodiments, the ligand binding domain comprises the extracellular ligand-binding domain of CD16 incorporating a polymorphism at position 158 (e.g., valine or phenylalanine). In some embodiments, the ligand binding domain is produced under conditions that alter its glycosylation state and its affinity for an Fc domain. In some embodiments, the ligand binding domain comprises the extracellular ligand-binding domain of CD16 incorporating modifications that render the chimeric transmembrane receptor polypeptide incorporating it specific for a subset of IgG antibodies.

For example, mutations that increase or decrease the affinity for an IgG subtype (e.g., IgG1) may be incorporated. In some embodiments, the ligand binding domain comprises the extracellular ligand-binding domain of CD32, which may incorporate a naturally occurring polymorphism that may modulate affinity for an Fc domain. In some embodiments, the ligand binding domain comprises the extracellular ligand-binding domain of CD32 incorporating modifications that render the chimeric transmembrane receptor polypeptide incorporating it specific for a subset of IgG antibodies. For example, mutations that increase or decrease the affinity for an IgG subtype (e.g., IgG1) may be incorporated.

In some embodiments, the ligand binding domain comprises the extracellular ligand-binding domain of CD64, which may incorporate a naturally occurring polymorphism that may modulate affinity for an Fc domain. In some embodiments, the ligand binding domain is produced under conditions that alter its glycosylation state and its affinity for an Fc domain. In some embodiments, the ligand binding domain comprises the extracellular ligand-binding domain of CD64 incorporating modifications that render the chimeric transmembrane receptor polypeptide incorporating it specific for a subset of IgG antibodies. For example, mutations that increase or decrease the affinity for an IgG subtype (e.g., IgG1) may be incorporated.

In other embodiments, the ligand binding domain comprises a naturally occurring bacterial protein that is capable of binding to the Fc portion of an IgG molecule, or any variant thereof (e.g., protein A, protein G). In some embodiments, the ligand binding domain comprises protein A, or any variant thereof. Protein A refers to a 42 kDa surface protein originally found in the cell wall of the bacterium Staphylococcus aureus. It is composed of five domains that each fold into a three-helix bundle and are able to bind IgG through interactions with the Fc region of most antibodies as well as the Fab region of human VH3 family antibodies. In some embodiments, the ligand binding domain comprises protein G, or any variant thereof. Protein G refers to an approximately 60-kDa protein expressed in group C and G Streptococcal bacteria that binds to both the Fab and Fc region of mammalian IgGs. While native protein G also binds albumin, recombinant variants have been engineered that eliminate albumin binding.

Ligand binding domains can also be created de novo using combinatorial biology or directed evolution methods. Starting with a protein scaffold (e.g., an scFv derived from IgG, a Kunitz domain derived from a Kunitz-type protease inhibitor, an ankyrin repeat, the Z domain from protein A, a lipocalin, a fibronectin type III domain, an SH3 domain from Fyn, or others), amino acid side chains for a set of residues on the surface may be randomly substituted in order to create a large library of variant scaffolds. From large libraries, it is possible to isolate variants with affinity for a target like the Fc domain by first selecting for binding, followed by amplification by phage, ribosome or cell display. Repeated rounds of selection and amplification can be used to isolate those proteins with the highest affinity for the target. Exemplary Fc-binding peptides may comprise the amino acid sequence of ETQRCTWHMGELVWCEREHN (SEQ ID NO: 5), KEASCSYWLGELVWCVAGVE (SEQ ID NO: 6), or DCAWHLGELVWCT (SEQ ID NO: 7).

Any of the Fc binders described herein may have a suitable binding affinity for the Fc domain of an antibody. Binding affinity refers to the apparent association constant or KA. The KA is the reciprocal of the dissociation constant, KD. The extracellular ligand-binding domain of an Fc receptor domain of the chimeric transmembrane receptor polypeptides described herein may have a binding affinity KD of at least 10-5, 10-6, 10-7, 10-8, 10-9, 10-10 M or lower for the Fc portion of an antibody. In some embodiments, the ligand binding domain which binds an Fc portion of an antibody has a high binding affinity for antibody, isotype of antibodies, or subtype(s) thereof, as compared to the binding affinity of the ligand binding domain to another antibody, isotype of antibodies or subtypes thereof.

In some embodiments, the extracellular ligand-binding domain of an Fc receptor has specificity for an antibody, isotype of antibodies, or subtype(s) thereof, as compared to binding of the extracellular ligand-binding domain of an Fc receptor to another antibody, isotype of antibodies, or subtypes thereof. Fcγ receptors with relatively high affinity binding include CD64A, CD64B, and CD64C. Fcγ receptors with relatively low affinity binding include CD32A, CD32B, CD16A, and CD16B. An Fcε receptor with relatively high affinity binding includes FcεRI, and an Fcε receptor with relatively low affinity binding includes FcεRII/CD23.

The binding affinity or binding specificity for an Fc receptor, or any variant thereof or for a chimeric transmembrane receptor comprising an Fc binding domain can be determined by a variety of methods including equilibrium dialysis, equilibrium binding, gel filtration, ELISA, surface plasmon resonance, and spectroscopy.

In some embodiments, a ligand binding domain comprising the extracellular ligand-binding domain of an Fc receptor comprises an amino acid sequence that is at least 90% (e.g., 91%, 92%, 93%, 94%, 95%, 96%, 97%, 98%, 99% or greater) identical to the amino acid sequence of the extracellular ligand-binding domain of a naturally-occurring Fcγ receptor, an Fcα receptor, an Fcε receptor, or FcRn. The“percent identity” or “% identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the ligand binding domain comprises an Fc binding domain comprising a variant of an extracellular ligand-binding domain of an Fc receptor. In some embodiments, the variant extracellular ligand-binding domain of an Fc receptor may comprise up to 10 amino acid residue variations (e.g., 1, 2, 3, 4, 5, 6, 7, 8, 9 or 10) relative to the amino acid sequence of the reference extracellular ligand-binding domain. In some embodiments, the variant can be a naturally-occurring variant due to gene polymorphism. In other embodiments, the variant can be a non-naturally occurring modified molecule. For example, mutations can be introduced into the extracellular ligand-binding domain of an Fc receptor to alter its glycosylation pattern and thus its binding affinity to the corresponding Fc domain.

In some examples, the ligand binding domain comprises a Fc binding comprising an Fc receptor selected from CD16A, CD16B, CD32A, CD32B, CD32C, CD64A, CD64B, CD64C, or a variant thereof as described herein. The extracellular ligand-binding domain of an Fc receptor may comprise up to 10 amino acid residue variations (e.g., 1, 2, 3, 4, 5, 6, 7, 8, 9 or 10) relative to the amino acid sequence of the extracellular ligand-binding domain of CD16A, CD16B, CD32A, CD32B, CD32C, CD64A, CD64B, CD64C as described herein. Mutation of amino acid residues of the extracellular ligand-binding domain of an Fc receptor may result in an increase in binding affinity for the Fc receptor domain to bind to an antibody, isotype of antibodies, or subtype(s) thereof relative to Fc receptor domains that do not comprise the mutation. For example, mutation of residue 158 of the Fc-gamma receptor CD16A may result in an increase in binding affinity of the Fc receptor to an Fc portion of an antibody. In some embodiments, the mutation is a substitution of a phenylalanine to a valine at residue 158 of the Fcγ receptor CD16A. Various suitable alternative or additional mutations can be made in the extracellular ligand-binding domain of an Fc receptor that may enhance or reduce the binding affinity to an Fc portion of a molecule such as an antibody.

The extracellular region comprising a ligand binding domain can be linked to the intracellular region, for example by a membrane spanning segment. In some embodiments, the membrane spanning segment comprises a polypeptide. The membrane spanning polypeptide linking the extracellular region and the intracellular region of the chimeric transmembrane receptor can have any suitable polypeptide sequence. In some cases, the membrane spanning polypeptide comprises a polypeptide sequence of a membrane spanning portion of an endogenous or wild-type membrane spanning protein. In some embodiments, the membrane spanning polypeptide comprises a polypeptide sequence having at least 1 (e.g., at least 2, 3, 4, 5, 6, 7, 8, 9, 10 or greater) of an amino acid substitution, deletion, and insertion compared to a membrane spanning portion of an endogenous or wild-type membrane spanning protein. In some embodiments, the membrane spanning polypeptide comprises a non-natural polypeptide sequence, such as the sequence of a polypeptide linker. The polypeptide linker may be flexible or rigid. The polypeptide linker can be structured or unstructured. In some embodiments, the membrane spanning polypeptide transmits a signal from the extracellular region to the intracellular region of the receptor, for example a signal indicating ligand-binding.

The signaling domain of a CAR can comprise an immune cell signaling domain. The immune cell signaling domain can comprise any signaling domain, or variant thereof, involved in immune cell signaling. For example, a signaling domain is involved in regulating primary activation of the TCR complex either in a stimulatory way or in an inhibitory way. An primary signaling domain can comprise a signaling domain of an Fey receptor (FcγR), an FCC receptor (FcεR), an Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lck, MAPK, MHC complex, NFAT, NF-κB, PLC-γ, iC3b, C3dg, C3d, and Zap70. In some embodiments, the signaling domain comprises an immunoreceptor tyrosine-based activation motif or ITAM. A primary signaling domain comprising an ITAM can comprise two repeats of the amino acid sequence YxxL/I separated by 6-8 amino acids, wherein each x is independently any amino acid, producing the conserved motif YxxL/Ix(6-8)YxxL/I. A primary signaling domain comprising an ITAM can be modified, for example, by phosphorylation when the ligand binding domain is bound to an antigen. A phosphorylated ITAM can function as a docking site for other proteins, for example proteins involved in various signaling pathways. In some embodiments, the signaling domain comprises a modified ITAM domain, e.g., a mutated, truncated, and/or optimized ITAM domain, which has altered (e.g., increased or decreased) activity compared to the native ITAM domain.

In some embodiments, the signaling domain comprises an FcγR signaling domain (e.g., ITAM). The FcγR signaling domain can be selected from FcγRI (CD64), FcγRIIA (CD32), FcγRIIB (CD32), FcγRIIIA (CD16a), and FcγRIIIB (CD16b). In some embodiments, the signaling domain comprises an FcεR signaling domain (e.g., ITAM). The FcεR signaling domain can be selected from FcεRI and FcεRII (CD23). In some embodiments, the signaling domain comprises an FcαR signaling domain (e.g., ITAM). The FcαR signaling domain can be selected from FcαRI (CD89) and Fcα/μR. In some embodiments, the signaling domain comprises a CD3ζ signaling domain. In some embodiments, the signaling domain comprises an ITAM of CD3ζ.

In some embodiments, a signaling domain comprises an immunoreceptor tyrosine-based inhibition motif or ITIM. A signaling domain comprising an ITIM can comprise a conserved sequence of amino acids (S/I/V/LxYxxI/V/L) that is found in the cytoplasmic tails of some inhibitory receptors of the immune system. A signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1 (IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIRE), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some embodiments, the signaling domain comprises a modified ITIM domain, e.g., a mutated, truncated, and/or optimized ITIM domain, which has altered (e.g., increased or decreased) activity compared to the native ITIM domain.

In some embodiments, the signaling domain comprises at least 2 ITAM domains (e.g., at least 3, 4, 5, 6, 7, 8, 9, or 10 ITAM domains). In some embodiments, the signaling domain comprises at least 2 ITIM domains (e.g., at least 3, 4, 5, 6, 7, 8, 9, or 10 ITIM domains) (e.g., at least 2 primary signaling domains). In some embodiments, the signaling domain comprises both ITAM and ITIM domains. The signaling domain of an intracellular region of a chimeric transmembrane receptor can include a co-stimulatory domain. In some embodiments, a co-stimulatory domain, for example from co-stimulatory molecule, can provide co-stimulatory signals for immune cell signaling, such as signaling from ITAM and/or ITIM domains, e.g., for the activation and/or deactivation of immune cells. In some embodiments, a costimulatory domain is operable to regulate a proliferative and/or survival signal in the immune cell. In some embodiments, a co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the co-stimulatory domain comprises a signaling domain of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8α, CD8β, CD82/Kai-1, CD84/SLAMF5, CD90/Thy1, CD96, CD5, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In some embodiments, the signaling domain comprises multiple co-stimulatory domains, for example at least two, e.g., at least 3, 4, or 5 co-stimulatory domains.

A transmembrane receptor comprising a GPCR, or any variant thereof (e.g., synthetic or chimeric receptor comprising at least one of a GPCR extracellular, transmembrane, and intracellular domain) can bind a ligand comprising any suitable GPCR ligand, or any variant thereof. Non-limiting examples of ligands which can be bound by a GPCR include (−)-adrenaline, (−)-noradrenaline, (lyso)phospholipid mediators, [des-Arg10]kallidin, [des-Arg9]bradykinin, [des-Gln14]ghrelin, [Hyp3]bradykinin, [Leu]enkephalin, [Met]enkephalin, 12-hydroxyheptadecatrienoic acid, 12R-HETE, 12S-HETE, 12S-HPETE, 15S-HETE, 17β-estradiol, 20-hydroxy-LTB4, 2-arachidonoylglycerol, 2-oleoyl-LPA, 3-hydroxyoctanoic acid, 5-hydroxytryptamine, 5-oxo-15-HETE, 5-oxo-ETE, 5-oxo-ETrE, 5-oxo-ODE, 5S-HETE, 5S-HPETE, 7α,25-dihydroxycholesterol, acetylcholine, ACTH, adenosine diphosphate, adenosine, adrenomedullin 2/intermedin, adrenomedullin, amylin, anandamide, angiotensin II, angiotensin III, annexin I, apelin receptor early endogenous ligand, apelin-13, apelin-17, apelin-36, aspirin triggered lipoxin A4, aspirin-triggered resolvin D1, ATP, beta-defensin 4A, big dynorphin, bovine adrenal medulla peptide 8-22, bradykinin, C3a, C5a, Ca2+, calcitonin gene related peptide, calcitonin, cathepsin G, CCK-33, CCK-4, CCK-8, CCL1, CCL11, CCL13, CCL14, CCL15, CCL16, CCL17, CCL19, CCL2, CCL20, CCL21, CCL22, CCL23, CCL24, CCL25, CCL26, CCL27, CCL28, CCL3, CCL4, CCL5, CCL7, CCL8, chemerin, chenodeoxycholic acid, cholic acid, corticotrophin-releasing hormone, CST-17, CX3CL1, CXCL1, CXCL10, CXCL11, CXCL12a, CXCL12f3, CXCL13, CXCL16, CXCL2, CXCL3, CXCL5, CXCL6, CXCL7, CXCL8, CXCL9, cysteinyl-leukotrienes (CysLTs), uracil nucleotides, deoxycholic acid, dihydrosphingosine-1-phosphate, dioleoylphosphatidic acid, dopamine, dynorphin A, dynorphin A-(1-13), dynorphin A-(1-8), dynorphin B, endomorphin-1, endothelin-1, endothelin-2, endothelin-3, F2L, Free fatty acids, FSH, GABA, galanin, galanin-like peptide, gastric inhibitory polypeptide, gastrin-17, gastrin-releasing peptide, ghrelin, GHRH, glucagon, glucagon-like peptide 1-(7-36) amide, glucagon-like peptide 1-(7-37), glucagon-like peptide 2, glucagon-like peptide 2-(3-33), GnRH I, GnRH II, GRP-(18-27), hCG, histamine, humanin, INSL3, INSL5, kallidin, kisspeptin-10, kisspeptin-13, kisspeptin-14, kisspeptin-54, kynurenic acid, large neuromedin N, large neurotensin, L-glutamic acid, LH, lithocholic acid, L-lactic acid, long chain carboxylic acids, LPA, LTB4, LTC4, LTD4, LTE4, LXA4, Lys-[Hyp3]-bradykinin, lysophosphatidylinositol, lysophosphatidylserine, Medium-chain-length fatty acids, melanin-concentrating hormone, melatonin, methylcarbamyl PAF, Mg2+, motilin, N-arachidonoylglycine, neurokinin A, neurokinin B, neuromedin B, neuromedin N, neuromedin S-33, neuromedin U-25, neuronostatin, neuropeptide AF, neuropeptide B-23, neuropeptide B-29, neuropeptide FF, neuropeptide S, neuropeptide SF, neuropeptide W-23, neuropeptide W-30, neuropeptide Y, neuropeptide Y-(3-36), neurotensin, nociceptin/orphanin FQ, N-oleoylethanolamide, obestatin, octopamine, orexin-A, orexin-B, Oxysterols, oxytocin, PACAP-27, PACAP-38, PAF, pancreatic polypeptide, peptide YY, PGD2, PGE2, PGF2α, PGI2, PGJ2, PHM, phosphatidylserine, PHV, prokineticin-1, prokineticin-2, prokineticin-2β, prosaposin, PrRP-20, PrRP-31, PTH, PTHrP, PTHrP-(1-36), QRFP43, relaxin, relaxin-1, relaxin-3, resolvin D1, resolvin E1, RFRP-1, RFRP-3, R-spondins, secretin, serine proteases, sphingosine 1-phosphate, sphingosylphosphorylcholine, SRIF-14, SRIF-28, substance P, succinic acid, thrombin, thromboxane A2, TIP39, T-kinin, TRH, TSH, tyramine, UDP-glucose, uridine diphosphate, urocortin 1, urocortin 2, urocortin 3, urotensin II-related peptide, urotensin-II, vasopressin, VIP, Wnt, Wnt-1, Wnt-10a, Wnt-10b, Wnt-11, Wnt-16, Wnt-2, Wnt-2b, Wnt-3, Wnt-3a, Wnt-4, Wnt-5a, Wnt-5b, Wnt-6, Wnt-7a, Wnt-7b, Wnt-8a, Wnt-8b, Wnt-9a, Wnt-9b, XCL1, XCL2, Zn2+, α-CGRP, α-ketoglutaric acid, α-MSH, α-neoendorphin, β-alanine, β-CGRP, β-D-hydroxybutyric acid, β-endorphin, β-MSH, β-neoendorphin, β-phenylethylamine, and γ-MSH.

A transmembrane receptor comprising an integrin subunit, or any variant thereof (e.g., a synthetic or chimeric receptor comprising at least one of an integrin extracellular, transmembrane, and intracellular domain), can bind a ligand comprising any suitable integrin ligand, or any variant thereof. Non-limiting examples of ligands which can be bound by an integrin receptor include adenovirus penton base protein, beta-glucan, bone sialoprotein (BSP), Borrelia burgdorferi, Candida albicans, collagens (CN, e.g., CNI-IV), cytotactin/tenascin-C, decorsin, denatured collagen, disintegrins, E-cadherin, echovirus 1 receptor, epiligrin, Factor X, Fc epsilon RH (CD23), fibrin (Fb), fibrinogen (Fg), fibronectin (Fn), heparin, HIV Tat protein, iC3b, intercellular adhesion molecule (e.g., ICAM-1,2,3,4,5), invasin, L1 cell adhesion molecule (L1-CAM), laminin, lipopolysaccharide (LPS), MAdCAM-1, matrix metalloproteinase-2 (MMPe), neutrophil inhibitory factor (NIF), osteopontin (OP or OPN), plasminogen, prothrombin, sperm fertilin, thrombospondin (TSP), vascular cell adhesion molecule 1 (VCAM-1), vitronectin (VN or VTN), and von Willebrand factor (vWF).

A transmembrane receptor comprising a cadherin, or any variant thereof (e.g., a synthetic or chimeric receptor comprising at least one of a cadherin extracellular, transmembrane, and intracellular domain), can bind a ligand comprising any suitable cadherin ligand, or any variant thereof. A cadherin ligand can comprise, for example, another cadherin receptor (e.g., a cadherin receptor of a cell).

A transmembrane receptor comprising a RTK, or any variant thereof (e.g., a synthetic or chimeric receptor comprising at least one of a RTK extracellular, transmembrane, and intracellular domain), can bind a ligand comprising any suitable RTK ligand, or any variant thereof. Non limiting examples of RTK ligands include growth factors, cytokines, and hormones. Growth factors include, for example, members of the epidermal growth factor family (e.g., epidermal growth factor or EGF, heparin-binding EGF-like growth factor or HB-EGF, transforming growth factor-α or TGF-α, amphiregulin or AR, epiregulin or EPR, epigen, betacellulin or BTC, neuregulin-1 or NRG1, neuregulin-2 or NRG2, neuregulin-3 or NRG3, and neuregulin-4 or NRG4), the fibroblast growth factor family (e.g., FGF1, FGF2, FGF3, FGF4, FGF5, FGF6, FGF7, FGF8, FGF9, FGF10, FGF11, FGF12, FGF13, FGF14, FGF15/19, FGF16, FGF17, FGF18, FGF20, FGF21, and FGF23), the vascular endothelial growth factor family (e.g., VEGF-A, VEGF-B, VEGF-C, VEGF-D, and PIGF), and the platelet-derived growth factor family (e.g., PDGFA, PDGFB, PDGFC, and PDGFD). Hormones include, for example, members of the insulin/IGF/relaxin family (e.g., insulin, insulin-like growth factors, relaxin family peptides including relaxin1, relaxin2, relaxin3, Leydig cell-specific insulin-like peptide (gene INSL3), early placenta insulin-like peptide (ELIP) (gene INSL4), insulin-like peptide 5 (gene INSL5), and insulin-like peptide 6).

A transmembrane receptor comprising a cytokine receptor, or any variant thereof (e.g., a synthetic or chimeric receptor comprising at least one of a cytokine receptor extracellular, transmembrane, and intracellular domain) can bind a ligand comprising any suitable cytokine receptor ligand, or any variant thereof. Non-limiting examples of cytokine receptor ligands include interleukins (e.g., IL-2, IL-3, IL-4, IL-5, IL-6, IL-7, IL-9, IL-10, IL-11, IL-12, IL-13, IL-15, IL-20, IL-21, IL-22, IL-23, IL-27, IL-28, and IL-31), interferons (e.g., IFN-α, IFN-β, IFN-γ), colony stimulating factors (e.g., erythropoietin, macrophage colony-stimulating factor, granulocyte macrophage colony-stimulating factors or GM-CSFs, and granulocyte colony-stimulating factors or G-CSFs), and hormones (e.g., prolactin and leptin).

A transmembrane receptor comprising a death receptor, or any variant thereof (e.g., a synthetic or chimeric receptor comprising at least one of a death receptor extracellular, transmembrane, and intracellular domain) can bind a ligand comprising any suitable ligand of a death receptor, or any variant thereof. Non-limiting examples of ligands bound by death receptors include TNFα, Fas ligand, and TNF-related apoptosis-inducing ligand (TRAIL).

A transmembrane receptor comprising a chimeric antigen receptor can bind a ligand comprising a membrane bound ligand (e.g., antigen), for example a ligand bound to the extracellular surface of a cell (e.g., a target cell). In some embodiments, the ligand is not non-membrane bound, for example an extracellular ligand that is secreted by a cell (e.g., a target cell). Ligands (e.g., membrane bound and non-membrane bound) can be antigenic (e.g., eliciting an immune response) and associated with a disease such as a viral, bacterial, and/or parasitic infection; inflammatory and/or autoimmune disease; or neoplasm such as a cancer and/or tumor. Cancer antigens, for example, are proteins produced by tumor cells that can elicit an immune response, particularly a T-cell mediated immune response. The selection of the antigen binding portions of a chimeric receptor polypeptide can depend on the particular type of cancer antigen to be targeted. In some embodiments, the tumor antigen comprises one or more antigenic cancer epitopes associated with a malignant tumor. Malignant tumors can express a number of proteins that can serve as target antigens for an immune attack. The antigen interaction domains can bind to cell surface signals, extracellular matrix (ECM), paracrine signals, juxtacrine signals, endocrine signals, autocrine signals, signals that can trigger or control genetic programs in cells, or any combination thereof. In some embodiments, interactions between the cell signals that bind to the recombinant chimeric receptor polypeptides involve a cell-cell interaction, cell-soluble chemical interaction, and cell-matrix or microenvironment interaction.

In various embodiments of the aspects herein, binding of a ligand to a transmembrane receptor activates a signaling pathway of the cell. Activation of the signaling pathway can result in recruitment of a transcription factor or multiple transcription factors to promoter sequences and subsequent increases or decreases in gene expression levels.

A variety of signaling pathways of a cell are available. Table 2 provides exemplary signaling pathways and genes associated with the signaling pathway. A signaling pathway activated by ligand binding to a transmembrane receptor in embodiments provided herein can be any one of those provided in Table 2. A promoter activated to drive expression of the GMP upon binding of a ligand to the ligand binding domain of a transmembrane receptor in embodiments provided can comprise the promoter sequence driving any of the genes provided in Table 2, any variant of the promoter sequence, or any partial promoter sequence (e.g., a minimal promoter sequence).

TABLE 2

| CELLULAR | |
|---|---|
| FUNCTION | GENES |
| PI3K/AKT | PRKCE; ITGAM; ITGA5; IRAK1; PRKAA2; |
| Signaling | EIF2AK2; PTEN; EIF4E; PRKCZ; |
| | GRK6; MAPK1; TSC1; PLK1; AKT2; IKBKB; |
| | PIK3CA; CDK8; CDKN1B; NFKB2; BCL2; |
| | PIK3CB; PPP2R1A; MAPK8; |
| | BCL2L1; MAPK3; TSC2; ITGA1; KRAS; |
| | EIF4EBP1; RELA; PRKCD; NOS3; PRKAA1; |
| | MAPK9; CDK2; PPP2CA; PIM1; ITGB7; |
| | YWHAZ; ILK; TP53; RAF1; IKBKG; RELB; |
| | DYRK1A; CDKN1A; ITGB1; MAP2K2; |
| | JAK1; AKT1; JAK2; PIK3R1; |
| | CHUK; PDPK1; PPP2R5C; CTNNB1; MAP |
| | 2K1; NFKB1; PAK3; ITGB3; CCND1; |
| | GSK3A; FRAP1; SFN; ITGA2; |
| | TTK; CSNK1A1; BRAF; GSK3B; AKT3; |
| | FOXO1; SGK; HSP90AA1; RP S6KB1 |
| ERK/MAPK | PRKCE; ITGAM; ITGA5; HSPB1; |
| Signaling | IRAK1; PRKAA2; EIF2AK2; RAC1; |
| | RAP1A; TLN1; EIF4E; ELK1; GRK6; |
| | MAPK1; RAC2; PLK1; AKT2; PIK3CA; |
| | CDK8; CREB1; PRKCI; PTK2; FOS; |
| | RPS6KA4; PIK3CB; PPP2R1A; |
| | PIK3C3; MAPK8; MAPK3; ITGA1; |
| | ETS1; KRAS; MYCN; EIF4EBP1; |
| | PPARG; PRKCD; PRKAA1; MAPK9; SRC; |
| | CDK2; PPP2CA; PIM1; PIK3C2A; |
| | ITGB7; YWHAZ; PPP1CC; KSR1; |
| | PXN; RAF1; FYN; DYRK1A; ITGB1; |
| | MAP2K2; PAK4; PIK3R1; STAT3; |
| | PPP2R5C; MAP2K1; PAK3; ITGB3; |
| | ESR1; ITGA2; MYC; TTK; CSNK1A1; |
| | CRKL; BRAF; ATF4; PRKCA; SRF; |
| | STAT1; SGK |
| Glucocorticoid | RAC1; TAF4B; EP300; SMAD2; |
| Receptor | TRAF6; PCAF; ELK1; MAPK1; SMAD3; |
| Signaling | AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; |
| | MAPK8; BCL2L1; MAPK3; TSC22D3; |
| | MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; |
| | CREBBP; CDKN1A; MAP2K2; JAK1; IL8; |
| | NCOA2; AKT1; JAK2; PIK3R1; CHUK; |
| | STAT3; MAP2K1; NFKB1; TGFBR1; |
| | ESR1; SMAD4; CEBPB; JUN; AR; AKT3; |
| | CCL2; MMP1; STAT1; IL6; HSP90AA1 |
| Axonal | PRKCE; ITGAM; ROCK1; ITGA5; |
| Guidance | CXCR4; ADAM12; IGF1; RAC1; RAP1A; |
| Signaling | EIF4E; PRKCZ; NRP1; NTRK2; |
| | ARHGEF7; SMO; ROCK2; MAPK1; |
| | PGF; RAC2; PTPN11; GNAS; AKT2; |
| | PIK3CA; ERBB2; PRKCI; PTK2; CFL1; |
| | GNAQ; PIK3CB; CXCL12; PIK3C3; WNT11; |
| | PRKD1; GNB2L1; ABL1; MAPK3; |
| | ITGA1; KRAS; RHOA; PRKCD; |
| | PIK3C2A; ITGB7; GLI2; PXN; VASP; RAF1; |
| | FYN; ITGB1; MAP2K2; PAK4; ADAM17; |
| | AKT1; PIK3R1; GLI1; WNT5A; |
| | ADAM10; MAP2K1; PAK3; ITGB3; |
| | CDC42; VEGFA; ITGA2; EPHA8; CRKL; |
| | RND1; GSK3B; AKT3; PRKCA |
| Ephrin | PRKCE; ITGAM; ROCK1; ITGA5; |
| Receptor | CXCR4; IRAK1; PRKAA2; EIF2AK2; |
| Signaling | RAC1; RAP1A; GRK6; ROCK2; MAPK1; |
| | PGF; RAC2; PTPN11; GNAS; PLK1; AKT2; |
| | DOK1; CDK8; CREB1; PTK2; CFL1; |
| | GNAQ; MAP3K14; CXCL12; |
| | MAPK8; GNB2L1; ABL1; MAPK3; ITGA1; |
| | KRAS; RHOA; PRKCD; PRKAA1; |
| | MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; |
| | PTPN13; ATF4; AKT3; SGK |
| Actin | ACTN4; PRKCE; ITGAM; ROCK1; ITGA5; IRAK1; |
| Cytoskeleton | PRKAA2; EIF2AK2; RAC1; INS; ARHGEF7; GRK6; |
| Signaling | ROCK2; MAPK1; RAC2; PLK1; AKT2; |
| | PIK3CA; CDK8; PTK2; CFL1; PIK3CB; |
| | MYH9; DIAPH1; PIK3C3; MAPK8; |
| | F2R; MAPK3; SLC9A1; ITGA1; |
| | KRAS; RHOA; PRKCD; PRKAA1; MAPK9; |
| | CDK2; PIM1; PIK3C2A; ITGB7; PPP1CC; PXN; |
| | VIL2; RAF1; GSN; DYRK1A; ITGB1; |
| | MAP2K2; PAK4; PIP5K1A; PIK3R1; MAP2K1; |
| | PAK3; ITGB3; CDC42; APC; ITGA2; TTK; |
| | CSNK1A1; CRKL; BRAF; VAV3; SGK |
| Huntington's | PRKCE; IGF1; EP300; RCOR1; PRKCZ; |
| Disease | HDAC4; TGM2; MAPK1; CAPNS1; |
| Signaling | AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; |
| | MAPK3; CASP8; HDAC2; |
| | HDAC7A; PRKCD; HDAC11; |
| | MAPK9; HDAC9; PIK3C2A; HDAC3; TP53; |
| | CASP9; CREBBP; AKT1; PIK3R1; PDPK1; |
| | CASP1; APAF1; FRAP1; CASP2; JUN; BAX; |
| | ATF4; AKT3; PRKCA; CLTC; SGK; |
| | HDAC6; CASP3 |
| Apoptosis | PRKCE; ROCK1; BID; IRAK1; |
| Signaling | PRKAA2; EIF2AK2; BAK1; |
| | BIRC4; GRK6; MAPK1; CAPNS1; PLK1; |
| | AKT2; IKBKB; CAPN2; CDK8; FAS; |
| | NFKB2; BCL2; MAP3K14; MAPK8; |
| | BCL2L1; CAPN1; MAPK3; CASP8; KRAS; RELA; |
| | PRKCD; PRKAA1; MAPK9; CDK2; |
| | PIM1; TP53; TNF; |
| | RAF1; IKBKG; RELB; CASP9; DYRK1A; MAP2K2; |
| | CHUK; APAF1; MAP2K1; NFKB1; |
| | PAK3; LMNA; CASP2; BIRC2; TTK; |
| | CSNK1A1; BRAF; BAX; PRKCA; SGK; |
| | CASP3; BIRC3; PARP1 |
| B Cell | RAC1; PTEN; LYN; ELK1; MAPK1; RAC2; |
| Receptor | PTPN11; AKT2; IKBKB; PIK3CA; CREB1; SYK; |
| Signaling | NFKB2; CAMK2A; MAP3K14; PIK3CB; |
| | PIK3C3; MAPK8; BCL2L1; ABL1; |
| | MAPK3; ETS1; KRAS; MAPK13; RELA; |
| | PTPN6; MAPK9; EGR1; PIK3C2A; |
| | BTK; MAPK14; RAF1; IKBKG; RELB; |
| | MAP3K7; MAP2K2; AKT1; PIK3R1; |
| | CHUK; MAP2K1; NFKB1; CDC42; |
| | GSK3A; FRAP1; BCL6; BCL10; JUN; |
| | GSK3B; ATF4; AKT3; VAV3; RPS6KB1 |
| Leukocyte | ACTN4; CD44; PRKCE; ITGAM; |
| Extravasation | ROCK1; CXCR4; CYBA; |
| Signaling | RAC1; RAP1A; PRKCZ; ROCK2; RAC2; PTPN11; |
| | MIMP14; PIK3CA; PRKCI; PTK2; PIK3CB; |
| | CXCL12; PIK3C3; MAPK8; PRKD1; ABL1; |
| | MAPK10; CYBB; MAPK13; RHOA; PRKCD; |
| | MAPK9; SRC; PIK3C2A; BTK; |
| | MAPK14; NOX1; PXN; VIL2; VASP; ITGB1; |
| | MAP2K2; CTNND1; PIK3R1; CTNNB1; |
| | CLDN1; CDC42; F11R; ITK; |
| | CRKL; VAV3; CTTN; PRKCA; MMP1; MMP9 |
| Integrin | ACTN4; ITGAM; ROCK1; ITGA5; |
| Signaling | RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; |
| | ITGA1; KRAS; RHOA; |
| | SRC; PIK3C2A; ITGB7; PPP1CC; ILK; PXN; VASP; |
| | RAF1; FYN; ITGB1; MAP2K2; |
| | PAK4; AKT1; PIK3R1; |
| | TNK2; MAP2K1; PAK3; ITGB3; CDC42; RND3; |
| | ITGA2; CRKL; BRAF; GSK3B; AKT3 |
| Acute Phase | IRAK1; SOD2; MYD88; TRAF6; |
| Response | ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; |
| | MAPK9; FTL; NR3C1; |
| | TRAF2; SERPINE1; MAPK14; TNF; RAF1; PDK1; |
| | IKBKG; RELB; MAP3K7; MAP2K2; |
| | AKT1; JAK2; PIK3R1; CHUK; STAT3; |
| | MAP2K1; NFKB1; FRAP1; CEBPB; JUN; |
| | AKT3; IL1R1; IL6 |
| PTEN | ITGAM; ITGA5; RAC1; PTEN; PRKCZ; BCL2L11; |
| Signaling | MAPK1; RAC2; AKT2; EGFR; |
| | IKBKB; CBL; PIK3CA; CDKN1B; PTK2; |
| | NFKB2; BCL2; PIK3CB; BCL2L1; MAPK3; |
| | ITGA1; KRAS; ITGB7; ILK; PDGFRB; INSR; |
| | RAF1; IKBKG; CASP9; CDKN1A; |
| | ITGB1; MAP2K2; AKT1; PIK3R1; |
| | CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; |
| | BRCA1; GADD45A; BIRC5; AKT2; |
| | PIK3CA; CHEK1; TP53INP1; BCL2; |
| | PIK3CB; PIK3C3; MAPK8; THBS1; |
| | ATR; BCL2L1; E2F1; PMAIP1; CHEK2; |
| | TNFRSF10B; TP73; RB1; HDAC9; CDK2; |
| | PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; |
| | JUN; SNAI2; GSK3B; BAX; AKT3 |
| Aryl | HSPB1; EP300; FASN; TGM2; |
| Hydrocarbon | RXRA; MAPK1; NQO1; |
| Receptor | NCOR2; SP1; ARNT; CDKN1B; FOS; CHEK1; |
| Signaling | SMARCA4; NFKB2; MAPK8; |
| | ALDH1A1; ATR; E2F1; MAPK3; |
| | NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; |
| | CCND1; ATM; ESR1; |
| | CDKN2A; MYC; JUN; ESR2; BAX; IL6; CYP1B1; |
| | HSP90AA1 |
| Xenobiotic | PRKCE; EP300; PRKCZ; RXRA; MAPK1; NQO1; |
| Metabolism | NCOR2; PIK3CA; ARNT; PRKCI; NFKB2; |
| Signaling | CAMK2A; PIK3CB; PPP2R1A; PIK3C3; MAPK8; |
| | PRKD1; ALDH1A1; MAPK3; NRIP1; KRAS; |
| | MAPK13; PRKCD; GSTP1; MAPK9; NOS2A; |
| | ABCB1; AHR; PPP2CA; FTL; NFE2L2; |
| | PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK | PRKCE; IRAK1; PRKAA2; EIF2AK2; RAC 1; ELK1; |
| Signaling | GRK6; MAPK1; GADD45A; RAC2; |
| | PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; |
| | PIK3C2A; TRAF2; TP53; LCK; MAP3K7; DYRK1A; |
| | MAP2K2; PIK3R1; MAP2K1; PAK3; CDC42; |
| | JUN; TTK; CSNK1A1; |
| | CRKL; BRAF; SGK |
| PPAr/RXR | PRKAA2; EP300; INS; SMAD2; |
| Signaling | TRAF6; PPARA; FASN; |
| | RXRA; MAPK1; SMAD3; GNAS; IKBKB; NCOR2; |
| | ABCA1; GNAQ; NFKB2; MAP3K14; |
| | STAT5B; MAPK8; IRS1; MAPK3; |
| | KRAS; RELA; PRKAA1; PPARGC1A; |
| | NCOA3; MAPK14; INSR; RAF1; |
| | IKBKG; RELB; MAP3K7; CREBBP; |
| | MAP2K2; JAK2; CHUK; MAP2K1; NFKB1; |
| | TGFBR1; SMAD4; JUN; IL1R1; |
| | PRKCA; IL6; HSP90AA1; ADIPOQ |
| NF-KB | IRAK1; EIF2AK2; EP300; INS; MYD88; |
| Signaling | PRKCZ; TRAF6; TBK1; AKT2; EGFR; |
| | IKBKB; PIK3CA; BTRC; NFKB2; |
| | MAP3K14; PIK3CB; PIK3C3; MAPK8; |
| | RIPK1; HDAC2; KRAS; RELA; |
| | PIK3C2A; TRAF2; TLR4; PDGFRB; TNF; |
| | INSR; LCK; IKBKG; RELB; |
| | MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin | ERBB4; PRKCE; ITGAM; ITGA5; |
| Signaling | PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; |
| | KRAS; PRKCD; STAT5A; SRC; ITGB7; |
| | RAF1; ITGB1; MAP2K2; ADAM17; AKT1; |
| | PIK3R1; PDPK1; MAP2K1; ITGB3; EREG; |
| | FRAP1; PSEN1; ITGA2; MYC; NRG1; CRKL; |
| | AKT3; PRKCA; HSP90AA1; RPS6KB1 |
| Wnt & | CD44; EP300; LRP6; DVL3; CSNK1E; GJA1; SMO; |
| Beta catenin | AKT2; PIN1; CDH1; BTRC; |
| Signaling | GNAQ; MARK2; PPP2R1A; |
| | WNT11; SRC; DKK1; PPP2CA; SOX6; SFRP2; ILK; |
| | LEF1; SOX9; TP53; MAP3K7; |
| | CREBBP; TCF7L2; AKT1; PPP2R5C; |
| | WNT5A; LRP5; CTNNB1; TGFBR1; CCND1; |
| | GSK3A; DVL1; APC; CDKN2A; MYC; |
| | CSNK1A1; GSK3B; AKT3; SOX2 |
| Insulin | PTEN; INS; EIF4E; PTPN1; PRKCZ; MAPK1; TSC1; |
| Receptor | PTPN11; AKT2; CBL; PIK3CA; |
| Signaling | PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; |
| | MAP2K1; GSK3A; FRAP1; CRKL; GSK3B; |
| | AKT3; FOXO1; SGK; RPS6KB1 |
| IL-6 | HSPB1; TRAF6; MAPKAPK2; ELK1; |
| Signaling | MAPK1; PTPN11; IKBKB; FOS; NFKB2; |
| | MAP3K14; MAPK8; MAPK3; |
| | MAPK10; IL6ST; KRAS; MAPK13; IL6R; |
| | RELA; SOCS1; MAPK9; ABCB1; |
| | TRAF2; MAPK14; TNF; RAF1; IKBKG; |
| | RELB; MAP3K7; MAP2K2; IL8; |
| | JAK2; CHUK; STAT3; |
| | MAP2K1; NFKB1; CEBPB; JUN; IL1R1; SRF; IL6 |
| Hepatic | PRKCE; IRAK1; INS; MYD88; |
| Cholestasis | PRKCZ; TRAF6; PPARA; RXRA; IKBKB; |
| | PRKCI; NFKB2; MAP3K14; MAPK8; PRKD1; |
| | MAPK10; RELA; PRKCD; MAPK9; ABCB1; |
| | TRAF2; TLR4; TNF; INSR; IKBKG; |
| | RELB; MAP3K7; IL8; |
| | CHUK; NR1H2; TJP2; NFKB1; ESR1; |
| | SREBF1; FGFR4; JUN; IL1R1; PRKCA; IL6 |
| IGF-1 | IGF1; PRKCZ; ELK1; MAPK1; PTPN11; |
| Signaling | NEDD4; AKT2; PIK3CA; PRKCI; |
| | PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; |
| | MAP2K2; AKT1; PIK3R1; PDPK1; |
| | MAP2K1; IGFBP2; SFN; JUN; CYR61; AKT3; |
| | FOXO1; SRF; CTGF; RPS6KB1 |
| NRF2- | PRKCE; EP300; SOD2; PRKCZ; MAPK1; SQSTM1; |
| mediated | NQO1; PIK3CA; PRKCI; FOS; PIK3CB; |
| Oxidative | PIK3C3; MAPK8; PRKD1; MAPK3; |
| Stress | KRAS; PRKCD; GSTP1; MAPK9; FTL; |
| Response | NFE2L2; PIK3C2A; MAPK14; RAF1; |
| | MAP3K7; CREBBP; MAP2K2; AKT1; |
| | PIK3R1; MAP2K1; PPM; JUN; KEAP1; |
| | GSK3B; ATF4; PRKCA; EIF2AK3; HSP90AA1 |
| Hepatic | EDN1; IGF1; KDR; FLT1; SMAD2; |
| Fibrosis/ | FGFR1; MET; PGF; SMAD3; EGFR; |
| Hepatic | FAS; CSF1; NFKB2; BCL2; MYH9; IGF1R; IL6R; |
| Stellate | RELA; TLR4; PDGFRB; TNF; RELB; IL8; |
| Cell | PDGFRA; NFKB1; TGFBR1; SMAD4; |
| Activation | VEGFA; BAX; IL1R1; CCL2; HGF; MMP1; |
| | STAT1; IL6; CTGF; MMP9 |
| PPAR | EP300; INS; TRAF6; PPARA; RXRA; |
| Signaling | MAPK1; IKBKB; NCOR2; FOS; |
| | NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; |
| | MAP2K2; CHUK; PDGFRA; |
| | MAP2K1; NFKB1; JUN; IL1R1; HSP90AA1 |
| Fc Epsilon | PRKCE; RAC1; PRKCZ; LYN; MAPK1; |
| RI Signaling | RAC2; PTPN11; AKT2; PIK3CA; |
| | SYK; PRKCI; PIK3CB; PIK3C3; MAPK8; |
| | PRKD1; MAPK3; MAPK10; KRAS; |
| | MAPK13; PRKCD; MAPK9; PIK3C2A; |
| | BTK; MAPK14; TNF; RAF1; FYN; |
| | MAP2K2; AKT1; PIK3R1; PDPK1; MAP2K1; |
| | AKT3; VAV3; PRKCA |
| G-Protein | PRKCE; RAP1A; RGS16; MAPK1; |
| Coupled | GNAS; AKT2; IKBKB; PIK3CA; CREB1; |
| Receptor | GNAQ; NFKB2; CAMK2A; PIK3CB; |
| Signaling | PIK3C3; MAPK3; KRAS; RELA; SRC; |
| | PIK3C2A; RAF1; IKBKG; RELB; |
| | FYN; MAP2K2; AKT1; PIK3R1; CHUK; |
| | PDPK1; STAT3; MAP2K1; NFKB1; BRAF; |
| | ATF4; AKT3; PRKCA |
| Inositol | PRKCE; IRAK1; PRKAA2; EIF2AK2; PTEN; |
| Phosphate | GRK6; MAPK1; PLK1; AKT2; PIK3CA; |
| Metabolism | CDK8; PIK3CB; PIK3C3; MAPK8; MAPK3; |
| | PRKCD; PRKAA1; MAPK9; CDK2; |
| | PIM1; PIK3C2A; DYRK1A; MAP2K2; |
| | PIP5K1A; PIK3R1; MAP2K1; PAK3; |
| | ATM; TTK; CSNK1A1; BRAF; SGK |
| PDGF | EIF2AK2; ELK1; ABL2; MAPK1; |
| Signaling | PIK3CA; FOS; PIK3CB; PIK3C3; MAPK8; |
| | CAV1; ABL1; MAPK3; KRAS; SRC; |
| | PIK3C2A; PDGFRB; RAF1; |
| | MAP2K2; JAK1; JAK2; PIK3R1; |
| | PDGFRA; STAT3; SPHK1; MAP2K1; MYC; |
| | JUN; CRKL; PRKCA; SRF; STAT1; SPHK2 |
| VEGF | ACTN4; ROCK1; KDR; FLT1; ROCK2; |
| Signaling | MAPK1; PGF; AKT2; PIK3CA; |
| | ARNT; PTK2; BCL2; PIK3CB; PIK3C3; |
| | BCL2L1; MAPK3; KRAS; HIF1A; NOS3; |
| | PIK3C2A; PXN; RAF1; MAP2K2; |
| | ELAVL1; AKT1; PIK3R1; MAP2K1; SFN; |
| | VEGFA; AKT3; FOXO1; PRKCA |
| Natural | PRKCE; RAC1; PRKCZ; MAPK1; RAC2; PTPN11; |
| Killer Cell | KIR2DL3; AKT2; PIK3CA; SYK; PRKCI; PIK3CB; |
| Signaling | PIK3C3; PRKD1; MAPK3; KRAS; PRKCD; PTPN6; |
| | PIK3C2A; LCK; RAF1; FYN; |
| | MAP2K2; PAK4; AKT1; |
| | PIK3R1; MAP2K1; PAK3; AKT3; VAV3; PRKCA |
| Cell Cycle: | HDAC4; SMAD3; SUV39H1; HDAC5; CDKN1B; |
| G1/S | BTRC; ATR; ABL1; E2F1; HDAC2; HDAC7A; |
| Checkpoint | RB1; HDAC11; HDAC9; CDK2; E2F2; HDAC3; |
| Regulation | TP53; CDKN1A; CCND1; |
| | E2F4; ATM; RBL2; SMAD4; CDKN2A; |
| | MYC; NRG1; GSK3B; RBL1; HDAC6 |
| T Cell | RAC1; ELK1; MAPK1; IKBKB; CBL; PIK3CA; |
| Receptor | FOS; NFKB2; PIK3CB; PIK3C3; MAPK8; MAPK3; |
| Signaling | KRAS; RELA; PIK3C2A; BTK; LCK; RAF1; |
| | IKBKG; RELB; FYN; MAP2K2; PIK3R1; |
| | CHUK; MAP2K1; NFKB1; ITK; BCL10; |
| | JUN; VAV3 |
| Death | CRADD; HSPB1; BID; BIRC4; |
| Receptor | TBK1; IKBKB; FADD; |
| Signaling | FAS; NFKB2; BCL2; MAP3K14; |
| | MAPK8; RIPK1; CASP8; DAXX; TNFRSF10B; |
| | RELA; TRAF2; TNF; IKBKG; RELB; |
| | CASP9; CHUK; APAF1; NFKB1; CASP2; |
| | BIRC2; CASP3; BIRC3 |
| FGF | RAC1; FGFR1; MET; MAPKAPK2; |
| Signaling | MAPK1; PTPN11; |
| | AKT2; PIK3CA; CREB1; PIK3CB; PIK3C3; |
| | MAPK8; MAPK3; MAPK13; PTPN6; PIK3C2A; |
| | MAPK14; RAF1; AKT1; PIK3R1; STAT3; MAP2K1; |
| | AFGFR4; CRKL; ATF4; KT3; PRKCA; HGF |
| GM-CSF | LYN; ELK1; MAPK1; PTPN11; AKT2; |
| Signaling | PIK3CA; CAMK2A; STAT5B; PIK3CB; |
| | PIK3C3; GNB2L1; BCL2L1; MAPK3; |
| | ETS1; KRAS; RUNX1; PIM1; PIK3C2A; RAF1; |
| | MAP2K2; AKT1; JAK2; PIK3R1; STAT3; |
| | MAP2K1; CCND1; AKT3; STAT1 |
| Amyotrophic | BID; IGF1; RAC1; BIRC4; PGF; CAPNS1; CAPN2; |
| Lateral | PIK3CA; BCL2; PIK3CB; PIK3C3; BCL2L1; |
| Sclerosis | CAPN1; PIK3C2A; TP53; CASP9; PIK3R1; |
| Signaling | RAB5A; CASP1; APAF1; VEGFA; BIRC2; |
| | BAX; AKT3; CASP3; BIRC3 |
| JAK/Stat | PTPN1; MAPK1; PTPN11; |
| Signaling | AKT2; PIK3CA; STAT5B; |
| | PIK3CB; PIK3C3; MAPK3; KRAS; SOCS1; |
| | STAT5A; PTPN6; PIK3C2A; RAF1; CDKN1A; |
| | MAP2K2; JAK1; AKT1; JAK2; PIK3R1; |
| | STAT3; MAP2K1; FRAP1; AKT3; STAT1 |
| Nicotinate | PRKCE; IRAK1; PRKAA2; EIF2AK2; GRK6; |
| and | MAPK1; PLK1; AKT2; CDK8; MAPK8; MAPK3; |
| Nicotinamide | PRKCD; PRKAA1; PBEF1; MAPK9; CDK2; PIM1; |
| Metabolism | DYRK1A; MAP2K2; MAP2K1; PAK3; |
| | NT5E; TTK; CSNK1A1; BRAF; SGK |
| Chemokine | CXCR4; ROCK2; MAPK1; PTK2; FOS; |
| Signaling | CFL1; GNAQ; CAMK2A; CXCL12; |
| | MAPK8; MAPK3; KRAS; MAPK13; |
| | RHOA; CCR3; SRC; PPP1CC; |
| | MAPK14; NOX1; RAF1; |
| | MAP2K2; MAP2K1; JUN; CCL2; PRKCA |
| IL-2 | ELK1; MAPK1; PTPN11; AKT2; PIK3CA; |
| Signaling | SYK; FOS; STAT5B; PIK3CB; |
| | PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic | PRKCE; IGF1; PRKCZ; PRDX6; |
| Long | LYN; MAPK1; GNAS; |
| Term | PRKCI; GNAQ; PPP2R1A; IGF1R; PRKD1; MAPK3; |
| Depression | KRAS; GRN; PRKCD; NOS3; NOS2A; PPP2CA; |
| | YWHAZ; RAF1; MAP2K2; PPP2R5C; |
| | MAP2K1; PRKCA |
| Estrogen | TAF4B; EP300; CARM1; PCAF; MAPK1; NCOR2; |
| Receptor | SMARCA4; MAPK3; NRIP1; KRAS; SRC; NR3C1; |
| Signaling | HDAC3; PPARGC1A; RBM9; NCOA3; |
| | RAF1; CREBBP; MAP2K2; |
| | NCOA2; MAP2K1; PRKDC; ESR1; ESR2 |
| Protein | TRAF6; SMURF1; BIRC4; BRCA1; UCHL1; NEDD4; |
| Ubiquitination | CBL; UBE2I; BTRC; HSPA5; USP7; USP10; FBW7; |
| Pathway | USP9X; STUB1; USP22; B2M; BIRC2; PARK2; |
| | USP8; USP1; VHL; HSP90AA1; BIRC3 |
| IL-10 | TRAF6; CCR1; ELK1; IKBKB; SP1; FOS; NFKB2; |
| Signaling | MAP3K14; MAPK8; MAPK13; RELA; |
| | MAPK14; TNF; IKBKG; RELB; MAP3K7; JAK1; |
| | CHUK; STAT3; NFKB1; JUN; IL1R1; IL6 |
| VDR/RXR | PRKCE; EP300; PRKCZ; RXRA; GADD45A; HES1; |
| Activation | NCOR2; SP1; PRKCI; CDKN1B; PRKD1; PRKCD; |
| | RUNX2; KLF4; YY1; NCOA3; CDKN1A; |
| | NCOA2; SPP1; LRP5; CEBPB; FOXO1; PRKCA |
| TGF-beta | EP300; SMAD2; SMURF1; MAPK1; |
| Signaling | SMAD3; SMAD1; FOS; MAPK8; |
| | MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like | IRAK1; EIF2AK2; MYD88; TRAF6; PPARA; ELK1; |
| Receptor | IKBKB; FOS; NFKB2; MAP3K14; MAPK8; |
| Signaling | MAPK13; RELA; TLR4; MAPK14; IKBKG; RELB; |
| | MAP3K7; CHUK; NFKB1; TLR2; JUN |
| p38 MAPK | HSPB1; IRAK1; TRAF6; MAPKAPK2; |
| Signaling | ELK1; FADD; FAS; CREB1; DDIT3; |
| | RPS6KA4; DAXX; MAPK13; TRAF2; |
| | MAPK14; TNF; MAP3K7; TGFBR1; MYC; |
| | ATF4; IL1R1; SRF; STAT1 |
| Neurotrophin/ | NTRK2; MAPK1; PTPN11; PIK3CA; CREB1; FOS; |
| TRK | PIK3CB; PIK3C3; MAPK8; MAPK3; KRAS; |
| Signaling | PIK3C2A; RAF1; MAP2K2; AKT1; PIK3R1; |
| | PDPK1; MAP2K1; CDC42; JUN; ATF4 |
| FXR/RXR | INS; PPARA; FASN; RXRA; AKT2; SDC1; MAPK8; |
| Activation | APOB; MAPK10; PPARG; MTTP; |
| | MAPK9; PPARGC1A; TNF; CREBBP; |
| | AKT1; SREBF1; FGFR4; AKT3; FOXO1 |
| Synaptic | PRKCE; RAP1A; EP300; PRKCZ; MAPK1; CREB1; |
| Long Term | PRKCI; GNAQ; CAMK2A; PRKD1; MAPK3; KRAS; |
| Potentiation | PRKCD; PPP1CC; RAF1; CREBBP; |
| | MAP2K2; MAP2K1; ATF4; PRKCA |
| Calcium | RAP1A; EP300; HDAC4; MAPK1; HDAC5; CREB1; |
| Signaling | CAMK2A; MYH9; MAPK3; |
| | HDAC2; HDAC7A; HDAC11; |
| | HDAC9; HDAC3; CREBBP; CALR; |
| | CAMKK2; ATF4; HDAC6 |
| EGF | ELK1; MAPK1; EGFR; PIK3CA; FOS; |
| Signaling | PIK3CB; PIK3C3; MAPK8; MAPK3; |
| | PIK3C2A; RAF1; JAK1; PIK3R1; |
| | STAT3; MAP2K1; JUN; PRKCA; SRF; STAT1 |
| Hypoxia | EDN1; PTEN; EP300; NQO1; UBE2I; |
| Signaling | CREB1; ARNT; HIF1A; SLC2A4; NOS3; |
| in the | TP53; LDHA; AKT1; ATM; |
| Cardiovascular | VEGFA; JUN; ATF4; VHL; HSP90AA1 |
| System | |
| LPS/IL-1 | IRAK1; MYD88; TRAF6; PPARA; RXRA; ABCA1; |
| Mediated | MAPK8; ALDH1A1; GSTP1; MAPK9; |
| Inhibition | ABCB1; TRAF2; TLR4; TNF; |
| of RXR | MAP3K7; NR1H2; SREBF1; JUN; IL1R1 |
| Function | |
| LXR/RXR | FASN; RXRA; NCOR2; ABCA1; NFKB2; |
| Activation | IRF3; RELA; NOS2A; TLR4; TNF; |
| | RELB; LDLR; NR1H2; NFKB1; |
| | SREBF1; IL1R1; CCL2; IL6; MMP9 |
| Amyloid | PRKCE; CSNK1E; MAPK1; CAPNS1; |
| Processing | AKT2; CAPN2; CAPN1; MAPK3; |
| | MAPK13; MAPT; MAPK14; AKT1; |
| | PSEN1; CSNK1A1; GSK3B; AKT3; APP |
| IL-4 | AKT2; PIK3CA; PIK3CB; PIK3C3; IRS1; |
| Signaling | KRAS; SOCS1; PTPN6; NR3C1; |
| | PIK3C2A; JAK1; AKT1; JAK2; PIK3R1; |
| | FRAP1; AKT3; RPS6KB1 |
| Cell Cycle: | EP300; PCAF; BRCA1; GADD45A; PLK1; BTRC; |
| G2/M DNA | CHEK1; ATR; CHEK2; YWHAZ; TP53; CDKN1A; |
| Damage | PRKDC; ATM; SFN; CDKN2A |
| Checkpoint | |
| Regulation | |
| Nitric Oxide | KDR; FLT1; PGF; AKT2; PIK3CA; |
| Signaling | PIK3CB; PIK3C3; CAV1; PRKCD; |
| in the Car- | NOS3; PIK3C2A; AKT1; PIK3R1; |
| diovascular | VEGFA; AKT3; HSP90AA1 |
| System | |
| Purine | NME2; SMARCA4; MYH9; RRM2; ADAR; |
| Metabolism | EIF2AK4; PKM2; ENTPD1; RAD51; RRM2B; |
| | TJP2; RAD51C; NT5E; POLD1; NME1 |
| cAMP- | RAP1A; MAPK1; GNAS; CREB1; CAMK2A; |
| mediated | MAPK3; SRC; RAF1; MAP2K2; STAT3; MAP2K1; |
| Signaling | BRAF; ATF4 |
| Mitochondrial | SOD2; MAPK8; CASP8; MAPK10; MAPK9; |
| Dysfunction | CASP9; PARK7; PSEN1; PARK2; APP; CASP3 |
| Notch | HES1; JAG1; NUMB; NOTCH4; ADAM17; |
| Signaling | NOTCH2; PSEN1; NOTCH3; NOTCH1; DLL4 |
| Endoplasmic | HSPA5; MAPK8; XBP1; TRAF2; ATF6; |
| Reticulum | CASP9; ATF4; EIF2AK3; CASP3 |
| Stress Pathway | |
| Pyrimidine | NME2; AICDA; RRM2; EIF2AK4; |
| Metabolism | ENTPD1; RRM2B; NT5E; POLD1; NME1 |
| Parkinson's | UCHL1; MAPK8; MAPK13; MAPK14; |
| Signaling | CASP9; PARK7; PARK2; CASP3 |
| Cardiac & Beta | GNAS; GNAQ; PPP2R1A; GNB2L1; |
| Adrenergic | PPP2CA; PPP1CC; PPP2R5C |
| Signaling | |
| Glycolysis/ | HK2; GCK; GPI; ALDH1A1; PKM2; LDHA; HK1 |
| Gluco- | |
| neogenesis | |
| Interferon | IRF1; SOCS1; JAK1; JAK2; IFITM1; STAT1; IFIT3 |
| Signaling | |
| Sonic | ARRB2; SMO; GLI2; DYRK1A; GL11; |
| Hedgehog | GSK3B; DYRK1B |
| Signaling | |
| Glycero- | PLD1; GRN; GPAM; YWHAZ; SPHK1; SPHK2 |
| phospholipid | |
| Metabolism | |
| Phospholipid | PRDX6; PLD1; GRN; YWHAZ; SPHK1; SPHK2 |
| Degradation | |
| Tryptophan | SIAH2; PRMT5; NEDD4; ALDH1A1; |
| Metabolism | CYP1B1; SIAH1 |
| Lysine | SUV39H1; EHMT2; NSD1; SETD7; PPP2R5C |
| Degradation | |
| Nucleotide | ERCC5; ERCC4; XPA; XPC; ERCC1 |
| Excision | |
| Repair | |
| Pathway | |
| Starch and | UCHL1; HK2; GCK; GPI; HK1 |
| Sucrose | |
| Metabolism | |
| Aminosugars | NQO1; HK2; GCK; HK1 |
| Metabolism | |
| Arachidonic | PRDX6; GRN; YWHAZ; CYP1B1 |
| Acid | |
| Metabolism | |
| Circadian | CSNK1E; CREB1; ATF4; NR1D1 |
| Rhythm | |
| Signaling | |
| Coagulation | BDKRB1; F2R; SERPINE1; F3 |
| System | |
| Dopamine | PPP2R1A; PPP2CA; PPP1CC; PPP2R5C |
| Receptor | |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine | DNMT1; DNMT3B; AHCY; DNMT3A |
| Metabolism | |
| Pyruvate | GLO1; ALDH1A1; PKM2; LDHA |
| Metabolism | |
| Arginine and | ALDH1A1; NOS3; NOS2A |
| Proline | |
| Metabolism | |
| Eicosanoid | PRDX6; GRN; YWHAZ |
| Signaling | |
| Fructose and | HK2; GCK; HK1 |
| Mannose | |
| Metabolism | |
| Galactose | HK2; GCK; HK1 |
| Metabolism | |
| Stilbene, | PRDX6; PRDX1; TYR |
| Coumarine and | |
| Lignin | |
| Biosynthesis | |
| Antigen | CALR; B2M |
| Presentation | |
| Pathway | |
| Biosynthesis | NQO1; DHCR7 |
| of Steroids | |
| Butanoate | ALDH1A1; NLGN1 |
| Metabolism | |
| Citrate Cycle | IDH2; IDH1 |
| Fatty Acid | ALDH1A1; CYP1B1 |
| Metabolism | |
| Glycero- | PRDX6; CHKA |
| phospholipid | |
| Metabolism | |
| Histidine | PRMT5; ALDH1A1 |
| Metabolism | |
| Inositol | ERO1L; APEX1 |
| Metabolism | |
| Metabolism | GSTP1; CYP1B1 |
| of Xenobiotics | |
| by Cytochrome | |
| p450 | |
| Methane | PRDX6; PRDX1 |
| Metabolism | |
| Phenylalanine | PRDX6; PRDX1 |
| Metabolism | |
| Propanoate | ALDH1A1; LDHA |
| Metabolism | |
| Selenoamino | PRMT5; AHCY |
| Acid | |
| Metabolism | |
| Sphingolipid | SPHK1; SPHK2 |
| Metabolism | |
| Amino- | PRMT5 |
| phosphonate | |
| Metabolism | |
| Androgen | PRMT5 |
| and Estrogen | |
| Metabolism | |
| Ascorbate | ALDH1A1 |
| and Aldarate | |
| Metabolism | |
| Bile Acid | ALDH1A1 |
| Biosynthesis | |
| Cysteine | LDHA |
| Metabolism | |
| Fatty Acid | FASN |
| Biosynthesis | |
| Glutamate | GNB2L1 |
| Receptor | |
| Signaling | |
| NRF2- | PRDX1 |
| mediated | |
| Oxidative | |
| Stress | |
| Response | |
| Pentose | GPI |
| Phosphate | |
| Pathway | |
| Pentose and | UCHL1 |
| Glucuronate | |
| Inter- | |
| conversions | |
| Retinol | ALDH1A1 |
| Metabolism | |
| Riboflavin | TYR |
| Metabolism | |
| Tyrosine | PRMT5 |
| Metabolism | |
| Tyrosine | TYR |
| Metabolism | |
| Ubiquinone | PRMT5 |
| Biosynthesis | |
| Valine, | ALDH1A1 |
| Leucine and | |
| Isoleucine | |
| Degradation | |
| Glycine, | CHKA |
| Serine and | |
| Threonine | |
| Metabolism | |
| Lysine | ALDH1A1 |
| Degradation | |
| Pain/Taste | TRPM5; TRPA1 |
| Pain | TRPM7; TRPC5; TRPC6; TRPC1; Cnr1; |
| | cnr2; Grk2; Trpa1; Pomc; Cgrp; Crf; Pka; Era; |
| | Nr2b; TRPM5; Prkaca; |
| | Prkacb; Prkar1a; Prkar2a |
| Mitochondrial | AIF; CytC; SMAC (Diablo); Aifm-1; Aifm-2 |
| Function | |
| Developmental | BMP-4; Chordin (Chrd); Noggin (Nog); WNT |
| Neurology | (Wnt2; Wnt2b; Wnt3a; Wnt4; Wnt5a; Wnt6; Wnt7b; |
| | Wnt8b; Wnt9a; Wnt9b; Wnt10a; Wnt10b; |
| | Wnt16); beta-catenin; Dkk-1; Frizzled |
| | related proteins; Otx-2; Gbx2; FGF-8; |
| | Reelin; Dab1; unc-86 (Pou4f1 or Brn3a); Numb; Reln |

In some embodiments, the promoter comprises at least one of an interleukin 2 (IL-2) promoter sequence, an interferon gamma (IFN-γ) promoter sequence, an interferon regulatory factor 4 (IRF4) promoter sequence, an nuclear receptor subfamily 4 group A member 1 (NR4A1, also known as nerve growth factor D3 NGFIB) promoter sequence, a PR domain zinc finger protein 1 (PRDM1) promoter sequence, a T-box transcription factor (TBX21) promoter sequence, a CD69 promoter sequence, a CD25 promoter sequence, or a granzyme B (GZMB) promoter sequence.

Promoters that can be used with the methods and compositions of the disclosure include, for example, promoters active in a eukaryotic, mammalian, non-human mammalian or human cell. The promoter can be an inducible or constitutively active promoter. Alternatively or additionally, the promoter can be tissue or cell specific.

Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression.

In some embodiments of the aspects herein, the actuator moiety comprises a CRISPR-associated (Cas) protein or a Cas nuclease which functions in a non-naturally occurring CRISPR (Clustered Regularly Interspaced Short Palindromic Repeats)/Cas (CRISPR-associated) system. In bacteria, this system can provide adaptive immunity against foreign DNA (Barrangou, R., et al, “CRISPR provides acquired resistance against viruses in prokaryotes,” Science (2007) 315: 1709-1712; Makarova, K. S., et al, “Evolution and classification of the CRISPR-Cas systems,” Nat Rev Microbiol (2011) 9:467-477; Garneau, J. E., et al, “The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39: 9275-9282).

In a wide variety of organisms including diverse mammals, animals, plants, and yeast, a CRISPR/Cas system (e.g., modified and/or unmodified) can be utilized as a genome engineering tool. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E2 86; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62; Deltcheva, E., et al, “CRISPR RNA maturation by trans-encoded small RNA and host factor RNase III,” Nature (2011) 471:602-607), and has been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31: 233-239; Sander, J. D. & Joung, J. K, “CRISPR-Cas systems for editing, regulating and targeting genomes,” Nature Biotechnol. (2014) 32:347-355).

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity. An actuator moiety comprising a Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products.

In some embodiments, the actuator moiety comprises a Cas protein that forms a complex with a guide nucleic acid, such as a guide RNA. In some embodiments, the actuator moiety comprises a Cas protein that forms a complex with a single guide nucleic acid, such as a single guide RNA (sgRNA). In some embodiments, the actuator moiety comprises a RNA-binding protein (RBP) optionally complexed with a guide nucleic acid, such as a guide RNA (e.g., sgRNA), which is able to form a complex with a Cas protein.

In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. For example, an actuator moiety can comprise a Cas protein which lacks cleavage activity.

Any suitable CRISPR/Cas system can be used. A CRISPR/Cas system can be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

An actuator moiety comprising a Cas protein can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas13a, CaslO, CaslOd, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csxl7, Csxl4, Csxl0, Csxl6, CsaX, Csx3, Csxl, Csx15, Csf1, Csf2, Csf3, Csf4, and Cul966, and homologs or modified versions thereof.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, Leptotrichia wadeii, Leptotrichia wadeii F0279, Rhodobacter capsulatus SB1003, Rhodobacter capsulatus R121, Rhodobacter capsulatus DE442, Lachnospiraceae bacterium NK4A179, Lachnospiraceae bacterium MA2020, Clostridium aminophilum DSM 10710, Paludibacter propionicigenes WB4, Carnobacterium gallinarum DMS4847, Carnobacterium gallinarum DSM4847, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Listeria seeligeri, Listeria weihenstephanensis FSL R90317, Listeria weihenstephanensis FSL M60635, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like nuclease domain. The RuvC and HNH domains can each cut a different strand of double-stranded DNA to make a double-stranded break in the DNA. A Cas protein can comprise only one nuclease domain (e.g., Cpf1 comprises RuvC domain but lacks HNH domain).

A Cas protein can comprise an amino acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

A Cas protein can be a fusion protein. For example, a Cas protein can be fused to a cleavage domain, an epigenetic modification domain, a transcriptional activation domain, or a transcriptional repressor domain. A Cas protein can also be fused to a heterologous polypeptide providing increased or decreased stability. The fused domain or heterologous polypeptide can be located at the N-terminus, the C-terminus, or internally within the Cas protein.

In some embodiments, a Cas protein is a dead Cas protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

A dCas9 polypeptide can associate with a single guide RNA (sgRNA) to activate or repress transcription of target DNA. sgRNAs can be introduced into cells expressing a system disclosed herein. In some cases, such cells contain one or more different sgRNAs that target the same nucleic acid. In other cases, the sgRNAs target different nucleic acids in the cell. The nucleic acids targeted by the guide RNA can be any that are expressed in a cell such as an immune cell. The nucleic acids targeted may be a gene involved in immune cell regulation. In some embodiments, the nucleic acid is associated with cancer. The nucleic acid associated with cancer can be a cell cycle gene, cell response gene, apoptosis gene, or phagocytosis gene. The recombinant guide RNA can be recognized by a CRISPR protein, a nuclease-null CRISPR protein, and variants thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type Cas9 activity).

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A dead Cas protein can comprise one or more mutations relative to a wild-type version of the protein. The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

As non-limiting examples, residues D10, G12, G17, E762, H840, N854, N863, H982, H983, A984, D986, and/or A987 (or the corresponding mutations of any of the Cas proteins) can be mutated. For example, e.g., D10A, G12A, G17A, E762A, H840A, N854A, N863A, H982A, H983A, A984A, and/or D986A. Mutations other than alanine substitutions can be suitable.

A D10A mutation can be combined with one or more of H840A, N854A, or N856A mutations to produce a Cas9 protein substantially lacking DNA cleavage activity (e.g., a dead Cas9 protein). A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. aureus. In some embodiments, the Cas9 protein is derived from a Cas9 protein from S. pyogenes or S. aureus. For example, a S. pyogenes or S. aureus Cas9 protein lacking cleavage activity. In some embodiments, the Cas protein is Cpf1.

Cas9 can generally refer to a polypeptide with at least about 5%, 10%, 20%, 30%, 40%, 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most about 5%, 10%, 20%, 30%, 40%, 50%, 60%, 70%, 80%, 90%, 100% sequence identity and/or sequence similarity to a wild type exemplary Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

In various embodiments of the aspects herein, the disclosure provides a guide nucleic acid for use in a CRISPR/Cas system. A guide nucleic acid (e.g., guide RNA) can bind to a Cas protein and target the Cas protein to a specific location within a target polynucleotide. A guide nucleic acid can comprise a nucleic acid-targeting segment and a Cas protein binding segment.

A guide nucleic acid can refer to a nucleic acid that can hybridize to another nucleic acid, for example, the target polynucleotide in the genome of a cell. A guide nucleic acid can be RNA, for example, a guide RNA. A guide nucleic acid can be DNA. A guide nucleic acid can comprise DNA and RNA. A guide nucleic acid can be single stranded. A guide nucleic acid can be double-stranded. A guide nucleic acid can comprise a nucleotide analog. A guide nucleic acid can comprise a modified nucleotide. The guide nucleic acid can be programmed or designed to bind to a sequence of nucleic acid site-specifically.

A guide nucleic acid can comprise one or more modifications to provide the nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

The guide nucleic acid can comprise a nucleic acid-targeting region (e.g., a spacer region), for example, at or near the 5′ end or 3′ end, that is complementary to a protospacer sequence in a target polynucleotide. The spacer of a guide nucleic acid can interact with a protospacer in a sequence-specific manner via hybridization (i.e., base pairing). The protospacer sequence can be located 5′ or 3′ of protospacer adjacent motif (PAM) in the target polynucleotide. The nucleotide sequence of a spacer region can vary and determines the location within the target nucleic acid with which the guide nucleic acid can interact. The spacer region of a guide nucleic acid can be designed or modified to hybridize to any desired sequence within a target nucleic acid.

A guide nucleic acid can comprise two separate nucleic acid molecules, which can be referred to as a double guide nucleic acid. A guide nucleic acid can comprise a single nucleic acid molecule, which can be referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like and tracrRNA-like molecules.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence can be identified by identifying a PAM within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The percent complementarity between the nucleic acid-targeting sequence (e.g., spacer sequence) and the target nucleic acid (e.g., protospacer) can be at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and the crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp. In some embodiments, the dsRNA duplex of the Cas protein-binding segment can has a length of 36 base pairs. The percent complementarity between the nucleotide sequences that hybridize to form the dsRNA duplex of the protein-binding segment can be at least about 60%. For example, the percent complementarity between the nucleotide sequences that hybridize to form the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The linker (e.g., that links a crRNA and a tracrRNA in a single guide nucleic acid) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a DNA-targeting RNA is 4 nt.

Guide nucleic acids can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof.

A guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within guide nucleic acids, the phosphate groups can commonly be referred to as forming the internucleoside backbone of the guide nucleic acid. The linkage or backbone of the guide nucleic acid can be a 3′ to 5′ phosphodiester linkage.

A guide nucleic acid can comprise a modified backbone and/or modified internucleoside linkages. Modified backbones can include those that retain a phosphorus atom in the backbone and those that do not have a phosphorus atom in the backbone.

Suitable modified guide nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

A guide nucleic acid can comprise one or more phosphorothioate and/or heteroatom internucleoside linkages, in particular —CH2-NH—O—CH2-, —CH2-N(CH3)-O—CH2- (i.e. a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-).

A guide nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage replaces a phosphodiester linkage.

A guide nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A guide nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A guide nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be non-ionic mimics of guide nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A guide nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH2)nO) mCH3, O(CH2)nOCH3, O(CH2)nNH2, O(CH2)nCH3, O(CH2)nONH2, and O(CH2)nON((CH2)nCH3)2, where n and m are from 1 to about 10. A sugar substituent group can be selected from: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O-CH2 CH2OCH3, also known as 2′-O-(2-methoxyethyl) or 2′-MOE i.e., an alkoxyalkoxy group). A further suitable modification can include 2′-dimethylaminooxyethoxy, (i.e., a O(CH2)2ON(CH3)2 group, also known as 2′-DMAOE), and 2′-dimethylaminoethoxyethoxy (also known as 2′-O-dimethyl-amino-ethoxy-ethyl or 2′-DMAEOE), i.e., 2′-O-CH2-O-CH2-N(CH3)2.

Other suitable sugar substituent groups can include methoxy (—O—CH3), aminopropoxy CH2 CH2 CH2NH2), allyl (—CH2-CH═CH2), —O-allyl (—O—CH2-CH═CH2) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

A guide nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a guide nucleic acid can comprise chemically linking to the guide nucleic acid one or more moieties or conjugates that can enhance the activity, cellular distribution or cellular uptake of the guide nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

A modification may include a “Protein Transduction Domain” or PTD (i.e. a cell penetrating peptide (CPP)). The PTD can refer to a polypeptide, polynucleotide, carbohydrate, or organic or inorganic compound that facilitates traversing a lipid bilayer, micelle, cell membrane, organelle membrane, or vesicle membrane. A PTD can be attached to another molecule, which can range from a small polar molecule to a large macromolecule and/or a nanoparticle, and can facilitate the molecule traversing a membrane, for example going from extracellular space to intracellular space, or cytosol to within an organelle. A PTD can be covalently linked to the amino terminus of a polypeptide. A PTD can be covalently linked to the carboxyl terminus of a polypeptide. A PTD can be covalently linked to a nucleic acid. Exemplary PTDs can include, but are not limited to, a minimal peptide protein transduction domain; a polyarginine sequence comprising a number of arginines sufficient to direct entry into a cell (e.g., 3, 4, 5, 6, 7, 8, 9, 10, or 10-50 arginines), a VP22 domain, a Drosophila antennapedia protein transduction domain, a truncated human calcitonin peptide, polylysine, and transportan, arginine homopolymer of from 3 arginine residues to 50 arginine residues (SEQ ID NO: 8). The PTD can be an activatable CPP (ACPP). ACPPs can comprise a polycationic CPP (e.g., Arg9 (SEQ ID NO: 9) or “R9” (SEQ ID NO: 9)) connected via a cleavable linker to a matching polyanion (e.g., Glu9 (SEQ ID NO: 10) or “E9” (SEQ ID NO: 10)), which can reduce the net charge to nearly zero and thereby inhibits adhesion and uptake into cells. Upon cleavage of the linker, the polyanion can be released, locally unmasking the polyarginine and its inherent adhesiveness, thus “activating” the ACPP to traverse the membrane.

Guide nucleic acids can be provided in any form. For example, the guide nucleic acid can be provided in the form of RNA, either as two molecules (e.g., separate crRNA and tracrRNA) or as one molecule (e.g., sgRNA). The guide nucleic acid can be provided in the form of a complex with a Cas protein. The guide nucleic acid can also be provided in the form of DNA encoding the RNA. The DNA encoding the guide nucleic acid can encode a single guide nucleic acid (e.g., sgRNA) or separate RNA molecules (e.g., separate crRNA and tracrRNA). In the latter case, the DNA encoding the guide nucleic acid can be provided as separate DNA molecules encoding the crRNA and tracrRNA, respectively.

DNAs encoding guide nucleic acid can be stably integrated in the genome of the cell and, optionally, operably linked to a promoter active in the cell. DNAs encoding guide nucleic acids can be operably linked to a promoter in an expression construct.

Guide nucleic acids can be prepared by any suitable method. For example, guide nucleic acids can be prepared by in vitro transcription using, for example, T7 RNA polymerase. Guide nucleic acids can also be a synthetically produced molecule prepared by chemical synthesis.

A guide nucleic acid can comprise a sequence for increasing stability. For example, a guide nucleic acid can comprise a transcriptional terminator segment (i.e., a transcription termination sequence). A transcriptional terminator segment can have a total length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the transcriptional terminator segment can have a length of from about 15 nucleotides (nt) to about 80 nt, from about 15 nt to about 50 nt, from about 15 nt to about 40 nt, from about 15 nt to about 30 nt or from about 15 nt to about 25 nt. The transcription termination sequence can be functional in a eukaryotic cell or a prokaryotic cell.

In some embodiments, an actuator moiety comprises a “zinc finger nuclease” or “ZFN.” ZFNs refer to a fusion between a cleavage domain, such as a cleavage domain of FokI, and at least one zinc finger motif (e.g., at least 2, 3, 4, or 5 zinc finger motifs) which can bind polynucleotides such as DNA and RNA. The heterodimerization at certain positions in a polynucleotide of two individual ZFNs in certain orientation and spacing can lead to cleavage of the polynucleotide. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain. Exemplary ZFNs include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

In some embodiments, an actuator moiety comprising a ZFN can generate a double-strand break in a target polynucleotide, such as DNA. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template that contains homology arms flanking sites of the target DNA can be provided. In some embodiments, a ZFN is a zinc finger nickase which induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33. In some embodiments, a ZFN binds a polynucleotide (e.g., DNA and/or RNA) but is unable to cleave the polynucleotide.

In some embodiments, the cleavage domain of an actuator moiety comprising a ZFN comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type cleavage domain. The modified form of the cleavage domain can have no substantial nucleic acid-cleaving activity. In some embodiments, the cleavage domain is enzymatically inactive.

In some embodiments, an actuator moiety comprises a “TALEN” or “TAL-effector nuclease.” TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR. In some cases, a single-stranded donor DNA repair template is provided to promote HDR. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type nuclease domain. The modified form of the nuclease domain can have no substantial nucleic acid-cleaving activity. In some embodiments, the nuclease domain is enzymatically inactive.

In some embodiments, the transcription activator-like effector (TALE) protein is fused to a domain that can modulate transcription and does not comprise a nuclease. In some embodiments, the transcription activator-like effector (TALE) protein is designed to function as a transcriptional activator. In some embodiments, the transcription activator-like effector (TALE) protein is designed to function as a transcriptional repressor. For example, the DNA-binding domain of the transcription activator-like effector (TALE) protein can be fused (e.g., linked) to one or more transcriptional activation domains, or to one or more transcriptional repression domains. Non-limiting examples of a transcriptional activation domain include a herpes simplex VP16 activation domain and a tetrameric repeat of the VP16 activation domain, e.g., a VP64 activation domain. A non-limiting example of a transcriptional repression domain includes a Kruppel-associated box domain.

In some embodiments, an actuator moiety comprises a meganuclease. Meganucleases generally refer to rare-cutting endonucleases or homing endonucleases that can be highly specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-MsoI meganuclease, I-SceI meganuclease, and variants thereof. Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

In some embodiments, the nuclease domain of a meganuclease comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA.

In some embodiments, the actuator moiety comprises at least one targeting sequence which directs transport of the actuator moiety to a specific region of a cell. A targeting sequence can be used to direct transport of a polypeptide to which the targeting sequence is linked to a specific region of a cell. For example, a targeting sequence can direct the actuator moiety to a cell nucleus utilizing a nuclear localization signal (NLS), outside of the nucleus (e.g., the cytoplasm) utilizing a nuclear export signal (NES), the mitochondria, the endoplasmic reticulum (ER), the Golgi, chloroplasts, apoplasts, peroxisomes, plasma membrane, or membrane of various organelles of a cell. In some embodiments, a targeting sequence comprises a nuclear export signal (NES) and directs the actuator moiety outside of a nucleus, for example to the cytoplasm of a cell. A targeting sequence can direct the actuator moiety to the cytoplasm utilizing various nuclear export signals. Nuclear export signals are generally short amino acid sequences of hydrophobic residues (e.g., at least about 2, 3, 4, or 5 hydrophobic residues) that target a protein for export from the cell nucleus to the cytoplasm through the nuclear pore complex using nuclear transport. Not all NES substrates can be constitutively exported from the nucleus. In some embodiments, a targeting sequence comprises a nuclear localization signal (NLS, e.g., a SV40 NLS) and directs a polypeptide to a cell nucleus. A targeting sequence can direct the actuator moiety to a cell nucleus utilizing various nuclear localization signals (NLS). An NLS can be a monopartite sequence or a bipartite sequence.

Non-limiting examples of NLSs include and NLS sequence derived from: the NLS of the SV40 virus large T-antigen, having the amino acid sequence PKKKRKV (SEQ ID NO: 11); the NLS from nucleoplasmin (e.g. the nucleoplasmin bipartite NLS with the sequence KRPAATKKAGQAKKKK (SEQ ID NO: 12)); the c-myc NLS having the amino acid sequence PAAKRVKLD (SEQ ID NO: 13) or RQRRNELKRSP (SEQ ID NO: 14); the hRNPA1 M9 NLS having the sequence NQSSNFGPMKGGNFGGRSSGPYGGGGQYFAKPRNQGGY (SEQ ID NO: 15); the sequence RMRIZFKNKGKDTAELRRRRVEVSVELRKAKKDEQILKRRNV (SEQ ID NO: 16) of the IBB domain from importin-alpha; the sequences VSRKRPRP (SEQ ID NO: 17) and PPKKARED (SEQ ID NO: 18) of the myoma T protein; the sequence PQPKKKPL (SEQ ID NO: 19) of human p53; the sequence SALIKKKKKMAP (SEQ ID NO: 20) of mouse c-abl IV; the sequences DRLRR (SEQ ID NO: 21) and PKQKKRK (SEQ ID NO: 22) of the influenza virus NS1; the sequence RKLKKKIKKL (SEQ ID NO: 23) of the Hepatitis virus delta antigen; the sequence REKKKFLKRR (SEQ ID NO: 24) of the mouse Mx1 protein; the sequence KRKGDEVDGVDEVAKKKSKK (SEQ ID NO: 25) of the human poly(ADP-ribose) polymerase; and the sequence RKCLQAGMNLEARKTKK (SEQ ID NO: 26) of the steroid hormone receptors (human) glucocorticoid.

In some embodiments, the actuator moiety comprises a membrane targeting peptide and directs the actuator moiety to a plasma membrane or membrane of a cellular organelle. A membrane-targeting sequence can provide for transport of the actuator moiety to a cell surface membrane or other cellular membrane. Molecules in association with cell membranes contain certain regions that facilitate membrane association, and such regions can be incorporated into a membrane targeting sequence. For example, some proteins contain sequences at the N-terminus or C-terminus that are acylated, and these acyl moieties facilitate membrane association. Such sequences can be recognized by acyltransferases and often conform to a particular sequence motif. Certain acylation motifs are capable of being modified with a single acyl moiety (often followed by several positively charged residues (e.g. human c-Src) to improve association with anionic lipid head groups) and others are capable of being modified with multiple acyl moieties. For example the N-terminal sequence of the protein tyrosine kinase Src can comprise a single myristoyl moiety. Dual acylation regions are located within the N-terminal regions of certain protein kinases, such as a subset of Src family members (e.g., Yes, Fyn, Lck) and G-protein alpha subunits. Such dual acylation regions often are located within the first eighteen amino acids of such proteins, and conform to the sequence motif Met-Gly-Cys-Xaa-Cys (SEQ ID NO: 27), where the Met is cleaved, the Gly is N-acylated and one of the Cys residues is S-acylated. The Gly often is myristoylated and a Cys can be palmitoylated. Acylation regions conforming to the sequence motif Cys-Ala-Ala-Xaa (so called “CAAX boxes”), which can modified with C15 or C10 isoprenyl moieties, from the C-terminus of G-protein gamma subunits and other proteins also can be utilized. These and other acylation motifs include, for example, those discussed in Gauthier-Campbell et al., Molecular Biology of the Cell 15: 2205-2217 (2004); Glabati et al., Biochem. J. 303: 697-700 (1994) and Zlakine et al., J. Cell Science 110: 673-679 (1997), and can be incorporated in a targeting sequence to induce membrane localization.

In certain embodiments, a native sequence from a protein containing an acylation motif is incorporated into a targeting sequence. For example, in some embodiments, an N-terminal portion of Lck, Fyn or Yes or a G-protein alpha subunit, such as the first twenty-five N-terminal amino acids or fewer from such proteins (e.g., about 5 to about 20 amino acids, about 10 to about 19 amino acids, or about 15 to about 19 amino acids of the native sequence with optional mutations), may be incorporated within the N-terminus of a chimeric polypeptide. In certain embodiments, a C-terminal sequence of about 25 amino acids or less from a G-protein gamma subunit containing a CAAX box motif sequence (e.g., about 5 to about 20 amino acids, about 10 to about 18 amino acids, or about 15 to about 18 amino acids of the native sequence with optional mutations) can be linked to the C-terminus of a chimeric polypeptide.

Any membrane-targeting sequence can be employed. In some embodiments, such sequences include, but are not limited to myristoylation-targeting sequence, palmitoylation-targeting sequence, prenylation sequences (i.e., farnesylation, geranyl-geranylation, CAAX Box), protein-protein interaction motifs or transmembrane sequences (utilizing signal peptides) from receptors. Examples include those discussed in, for example, ten Klooster, J. P. et al, Biology of the Cell (2007) 99, 1-12; Vincent, S., et al., Nature Biotechnology 21:936-40, 1098 (2003).

Additional protein domains exist that can increase protein retention at various membranes. For example, an ˜120 amino acid pleckstrin homology (PH) domain is found in over 200 human proteins that are typically involved in intracellular signaling. PH domains can bind various phosphatidylinositol (PI) lipids within membranes (e.g. PI (3,4,5)-P3, PI (3,4)-P2, PI (4,5)-P2) and thus can play a key role in recruiting proteins to different membrane or cellular compartments. Often the phosphorylation state of PI lipids is regulated, such as by PI-3 kinase or PTEN, and thus, interaction of membranes with PH domains may not be as stable as by acyl lipids.

In some embodiments, a targeting sequence directing the actuator moiety to a cellular membrane can utilize a membrane anchoring signal sequence. Various membrane-anchoring sequences are available. For example, membrane anchoring signal sequences of various membrane bound proteins can be used. Sequences can include those from: 1) class I integral membrane proteins such as IL-2 receptor beta-chain and insulin receptor beta chain; 2) class II integral membrane proteins such as neutral endopeptidase; 3) type III proteins such as human cytochrome P450 NF25; and 4) type IV proteins such as human P-glycoprotein.

In some embodiments, the actuator moiety is linked to a polypeptide folding domain which can assist in protein folding. In some embodiments, an actuator moiety is linked to a cell-penetrating domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. The cell-penetrating domain can be located at the N-terminus, the C-terminus, or anywhere within the actuator moiety.

In some embodiments, the actuator moiety is fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the enzyme. In other cases, the actuator moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-κB p65 subunit, Epstein-Barr virus R transactivator (Rta) and are described in Chavez et al., Nat Methods, 2015, 12(4):326-328 and U.S. Patent App. Publ. No. 20140068797. Non-limiting exemplary repression domains include the KRAB (Kruppel-associated box) domain of Kox1, the Mad mSIN3 interaction domain (SID), ERF repressor domain (ERD), and are described in Chavez et al., Nat Methods, 2015, 12(4):326-328 and U.S. Patent App. Publ. No. 20140068797. An actuator moiety can also be fused to a heterologous polypeptide providing increased or decreased stability. The fused domain or heterologous polypeptide can be located at the N-terminus, the C-terminus, or internally within the actuator moiety.

An actuator moiety can comprise a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g. eBFP, eBFP2, Azurite, mKalamal, GFPuv, Sapphire, T-sapphire), cyan fluorescent proteins (e.g. eCFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

In some embodiments, the GMP is linked to another protein when expressed. The peptide linker joining the GMP and the other protein can contain a cleavage recognition sequence, for example a protease recognition sequence. Various proteases and their corresponding protease recognition sequences can be used. Some proteases can be highly promiscuous such that a wide range of protein substrates are hydrolysed. Some proteases can be highly specific and only cleave substrates with a certain sequence, e.g., a cleavage recognition sequence or peptide cleavage domain. In some embodiments, the cleavage recognitions sequence comprises multiple cleavage recognition sequences, and each cleavage recognition sequence can be recognized by the same or different cleavage moiety. Sequence-specific proteases that can be used as cleavage moieties include, but are not limited to, superfamily CA proteases, e.g., families C1, C2, C6, C10, C12, C16, C19, C28, C31, C32, C33, C39, C47, C51, C54, C58, C64, C65, C66, C67, C70, C71, C76, C78, C83, C85, C86, C87, C93, C96, C98, and C101, including papain (Carica papaya), bromelain (Ananas comosus), cathepsin K (liverwort) and calpain (Homo sapiens); superfamily CD proteases, e.g., family C11, C13, C14, C25, C50, C80, and C84: such as caspase-1 (Rattus norvegicus) and separase (Saccharomyces cerevisiae); superfamily CE protease, e.g., family C5, C48, C55, C57, C63, and C79 including adenain (human adenovirus type 2); superfamily CF proteases, e.g., family C15 including pyroglutamyl-peptidase I (Bacillus amyloliquefaciens); superfamily CL proteases, e.g., family C60 and C82 including sortase A (Staphylococcus aureus); superfamily CM proteases, e.g. family C18 including hepatitis C virus peptidase 2 (hepatitis C virus); superfamily CN proteases, e.g., family C9 including sindbis virus-type nsP2 peptidase (sindbis virus); superfamily CO proteases, e.g., family C40 including dipeptidyl-peptidase VI (Lysinibacillus sphaericus); superfamily CP proteases, e.g., family C97 including DeSI-1 peptidase (Mus musculus); superfamily PA proteases, e.g., family C3, C4, C24, C30, C37, C62, C74, and C99 including TEV protease (Tobacco etch virus); superfamily PB proteases, e.g., family C44, C45, C59, C69, C89, and C95 including amidophosphoribosyltransferase precursor (Homo sapiens); superfamily PC proteases, families C26, and C56 including γ-glutamyl hydrolase (Rattus norvegicus); superfamily PD proteases, e.g., family C46 including Hedgehog protein (Drosophila melanogaster); superfamily PE proteases, e.g., family P1 including DmpA aminopeptidase (Ochrobactrum anthropi); others proteases, e.g., family C7, C8, C21, C23, C27, C36, C42, C53 and C75. Additional proteases include serine proteases, e.g., those of superfamily SB, e.g., families S8 and S53 including subtilisin (Bacillus licheniformis); those of superfamily SC, e.g., families S9, S10, S15, S28, S33, and S37 including prolyl oligopeptidase (Sus scrofa); those of superfamily SE, e.g., families S11, S12, and S13 including D-Ala-D-Ala peptidase C (Escherichia coli); those of superfamily SF, e.g., families S24 and S26 including signal peptidase I (Escherichia coli); those of Superfamily SJ, e.g., families S16, S50, and S69 including lon-A peptidase (Escherichia coli); those of Superfamily SK, e.g., families S14, S41, and S49 including Clp protease (Escherichia coli); those of Superfamily SO, e.g., families S74 including Phage K1F endosialidase CIMCD self-cleaving protein (Enterobacteria phage K1F); those of superfamily SP, e.g., family S59 including nucleoporin 145 (Homo sapiens); those of superfamily SR, e.g., family S60 including Lactoferrin (Homo sapiens); those of superfamily SS, families S66 including murein tetrapeptidase LD-carboxypeptidase (Pseudomonas aeruginosa); those of superfamily ST, e.g., families S54 including rhomboid-1 (Drosophila melanogaster); those of superfamily PA, e.g., families S1, S3, S6, S7, S29, S30, 531, S32, S39, S46, S55, S64, S65, and S75 including Chymotrypsin A (Bos taurus); those of superfamily PB, e.g., families S45 and S63 including penicillin G acylase precursor (Escherichia coli); those of superfamily PC, e.g., families S51 including dipeptidase E (Escherichia coli); those of superfamily PE, e.g., families P1 including DmpA aminopeptidase (Ochrobactrum anthropi); those unassigned, e.g., families S48, S62, S68, S71, S72, S79, and S81 threonine proteases, e.g., those of superfamily PB clan, e.g., families T1, T2, T3, and T6 including archaean proteasome, β component (Thermoplasma acidophilum); and those of superfamily PE clan, e.g., family T5 including ornithine acetyltransferase (Saccharomyces cerevisiae); aspartic proteases, e.g., BACE1, BACE2; cathepsin D; cathepsin E; chymosin; napsin-A; nepenthesin; pepsin; plasmepsin; presenilin; renin; and HIV-1 protease, and metalloproteinases, e.g., exopeptidases, metalloexopeptidases; endopeptidases, and metalloendopeptidases. A cleavage recognition sequence (e.g., polypeptide sequence) can be recognized by any of the proteases disclosed herein.

In some embodiments, the cleavage recognition sequence is a substrate of a protease selected from the group consisting of: achromopeptidase, aminopeptidase, ancrod, angiotensin converting enzyme, bromelain, calpain, calpain I, calpain II, carboxypeptidase A, carboxypeptidase B, carboxypeptidase G, carboxypeptidase P, carboxypeptidase W, carboxypeptidase Y, caspase 1, caspase 2, caspase 3, caspase 4, caspase 5, caspase 6, caspase 7, caspase 8, caspase 9, caspase 10, caspase 11, caspase 12, caspase 13, cathepsin B, cathepsin C, cathepsin D, cathepsin E, cathepsin G, cathepsin H, cathepsin L, chymopapain, chymase, chymotrypsin, clostripain, collagenase, complement Clr, complement Cls, complement Factor D, complement factor I, cucumisin, dipeptidyl peptidase IV, elastase (leukocyte), elastase (pancreatic), endoproteinase Arg-C, endoproteinase Asp-N, endoproteinase Glu-C, endoproteinase Lys-C, enterokinase, factor Xa, ficin, furin, granzyme A, granzyme B, HIV Protease, IGase, kallikrein tissue, leucine aminopeptidase (general), leucine aminopeptidase (cytosol), leucine aminopeptidase (microsomal), matrix metalloprotease, methionine aminopeptidase, neutrase, papain, pepsin, plasmin, prolidase, pronase E, prostate specific antigen, protease alkalophilic from Streptomyces griseus, protease from Aspergillus, protease from Aspergillus saitoi, protease from Aspergillus sojae, protease (B. licheniformis) (alkaline or alcalase), protease from Bacillus polymyxa, protease from Bacillus sp, protease from Rhizopus sp., protease S, proteasomes, proteinase from Aspergillus oryzae, proteinase 3, proteinase A, proteinase K, protein C, pyroglutamate aminopeptidase, rennin, rennin, streptokinase, subtilisin, thermolysin, thrombin, tissue plasminogen activator, trypsin, tryptase and urokinase.

Table 3 lists exemplary proteases and associated recognition sequences that can be used in systems of the disclosure.

TABLE 3
Exemplary proteases and associated recognition sequences

| Protease | | Recognition |
|---|---|---|
| name | Synonyms | sequence |
| Arg-C | Arginyl peptidase, | R-x |
| | Endoproteinase | |
| | Arg-C, Tissue kallikrein | |
| Asp-N | Endoproteinase Asp-N, | x-D |
| | Peptidyl-Asp | |
| | metalloendopeptidase | |
| Asp-N (N- | Endoproteinase Asp-N, | x-[DE] |
| terminal | Peptidyl-Asp | |
| Glu) | metalloendopeptidase | |
| BNPS or | 3-Bromo-3-methy1-2- | W-x |
| NCS/urea | (2-nitrophenylthio)-3H- | |
| | indole, BNPS-skatol, | |
| | N-chlorosuccinimide/urea | |
| Caspase-1 | ICE, Interleukin-1β- | [FLWY]-x-[AHT]- |
| | converting Enzyme | D-{DEKPQR} |
| Caspase-10 | Flice2, Mch4 | I-E-A-D-x (SEQ ID |
| | | NO: 28) |
| Caspase-2 | Ich-1, Nedd2 | D-V-A-D- |
| | | {DEKPQR} (SEQ |
| | | ID NO: 29) or D-E- |
| | | H-D-{DEKPQR} |
| | | (SEQ ID NO: 30) |
| Caspase-3 | Apopain, CPP32, Yama | D-M-Q-D- |
| | | {DEKPQR} (SEQ |
| | | ID NO: 31) or D-E- |
| | | V-D-{DEKPQR} |
| | | (SEQ ID NO: 32) |
| Caspase-4 | ICE(rel)II, Ich-2, TX | L-E-V-D- |
| | | {DEKPQR} (SEQ |
| | | ID NO: 33) or |
| | | [LW]-E-H-D- |
| | | {DEKPQR} |
| Caspase-5 | ICE(rel)III, TY | [LW]-E-H-D-x |
| Caspase-6 | Mch2 | V-E-[HI]-D- |
| | | {DEKPQR} |
| Caspase-7 | CMH-1, ICE-LAP3, | D-E-V-D- |
| | Mch-3 | {DEKPQR} (SEQ |
| | | ID NO: 34) |
| Caspase-8 | FLICE, MASH, Mch5 | [IL]-E-T-D- |
| | | {DEKPQR} |
| Caspase-9 | ICE-Lap6, Mch6 | L-E-H-D-x (SEQ |
| | | ID NO: 35) |
| Chymotrypsin | | [FY]-{13} or W- |
| | | {MP} |
| Chymotrypsin | | [FLY]-{P} or W- |
| (low | | {MP} or M-{PY} |
| specificity) | | or H-{DMPW} |
| Clostripain | Clostridiopeptidase B | R-x |
| CNBr | Cyanogen bromide | M-x |
| CNBr | Cyanogen bromide | M-x or x-C |
| (methyl-Cys) | | |
| CNBr (with | Cyanogen bromide | [MW]-x |
| acids) | | |
| Enterokinase | Enteropeptidase | [DE](4)-K-x |
| Factor Xa | Coagulation factor Xa | [AFGILTVM]- |
| | | [DE]-G-R-x |
| Formic acid | | D-x |
| Glu-C (AmAc | Endoproteinase Glu-C, | E-x |
| buffer) | V8 protease, Glutamyl | |
| | endopeptidase | |
| Glu-C (Phos | Endoproteinase Glu-C, | [DE]-x |
| buffer) | V8 protease, Glutamyl | |
| | endopeptidase | |
| Granzyme B | Cytotoxic T-lymphocyte | I-E-P-D-x (SEQ ID |
| | proteinase 2, Granzyme-2, | NO: 36) |
| | GranzymeB, Lymphocyte | |
| | protease, SECT, T-cell | |
| | serine protease 1-3E | |
| HRV3C | Human rhinovirus 3C | L-E-V-L-F-Q-G-P |
| protease | protease, Picornain 3C, | (SEQ ID NO: 37) |
| | Protease 3C | |
| Hydroxylamine | Hydroxylammonium | N-G |
| Iodosobenzoic | 2-Iodosobenzoic acid | W-x |
| acid | | |
| Lys-C | Endoproteinase Lys-C, | K-x |
| | Lysyl endopeptidase | |
| Lys-N | Endoproteinase Lys-N, | x-K |
| | Peptidyl-Lys | |
| | metalloendopeptidase, | |
| | Armillaria mellea neutral | |
| | proteinase | |
| Lys-N (Cys | Endoproteinase Lys-N, | x-[CK] |
| modified) | Peptidyl-Lys | |
| | metalloendopeptidase, | |
| | Armillaria mellea neutral | |
| | proteinase | |
| Mild acid | | D-P |
| hydrolysis | | |
| NBS (long | N-Bromosuccinimide | [HWY]-x |
| exposure) | | |
| NBS (short | N-Bromosuccinimide | [WY]-x |
| exposure) | | |
| NTCB | 2-Nitro-5-thiocyanatobenzoic | x-C |
| | acid, 2-Nitro-5- | |
| | thiocyanobenzoic acid | |
| Pancreatic | Pancreatopeptidase E, | [AGSV]-x |
| elastase | Elastase-1 | |
| Pepsin A | Pepsin | {HKR}-{P}-{R}- |
| | | [FLWY]-{P} or |
| | | {HKR}-{P}- |
| | | [FLWY]-x-{P} |
| Pepsin A (low | Pepsin | {HKR}-{P}-{R}- |
| specificity) | | [FL]-{P} or |
| | | {HKR}-{P}-[FL]- |
| | | x-{P} |
| Prolyl | Prolyl oligopeptidase, | [HKR]-P-{P} |
| endopeptidase | Post-proline cleaving | |
| | enzyme | |
| Proteinase K | Endopeptidase K, | [AEFILTVWY]-x |
| | Peptidase K | |
| TEV protease | Tobacco etch virus protease, | E-x-x-Y-x-Q-[GS] |
| | Nuclear-inclusion-a | |
| | endopeptidase | |
| Thermolysin | Thermophilic-bacterial | {DE}-[AFILMV]- |
| | protease | {P} |
| Thrombin | Factor IIa | x-x-G-R-G-x or |
| | | [AFGILTVW]- |
| | | [AFGILTVW]-P- |
| | | R-{DE}-{DE} |
| Trypsin | Trypsin-1 | x-[KR]-{P} or W- |
| | | K-P or M-R-P |
| | | But not: |
| | | [CD]-K-D or C-K- |
| | | [HY] or C-R-K or |
| | | R-R-[HR] |
| Trypsin (Arg | | K-{P} |
| blocked) | | |
| Trypsin (Cys | | [RKC]-{P} |
| modified) | | |
| Trypsin (Lys | | R-{P} |
| blocked) | | |

Proteases selected for use as cleavage moieties can be selected based on desired characteristics such as peptide bond selectivity, activity at certain pHs, molecular mass, etc.

The expression of a variety of target genes can be regulated by a GMP expressed in the embodiments provided herein. Any target gene of a cell can be regulated by the GMP.

The actuator moiety of a subject system can bind to a target polynucleotide to regulate expression and/or activity of the target gene by physical obstruction of the target polynucleotide or recruitment of additional factors effective to suppress or enhance expression of the target polynucleotide. In some embodiments, the actuator moiety comprises a transcriptional activator effective to increase expression of the target polynucleotide. The actuator moiety can comprise a transcriptional repressor effective to decrease expression of the target polynucleotide.

A target polynucleotide of the various embodiments of the aspects herein can be DNA or RNA (e.g., mRNA). The target polynucleotide can be single-stranded or double-stranded. The target polynucleotide can be genomic DNA. The target polynucleotide can be any polynucleotide endogenous or exogenous to a cell. For example, the target polynucleotide can by a polynucleotide residing in the nucleus of a eukaryotic cell. The target polynucleotide can be a sequence coding a gene product (e.g., a protein) or a non-coding sequence (e.g., a regulatory polynucleotide). In some embodiments, the target polynucleotide comprises a region of a plasmid, for example a plasmid carrying an exogenous gene. In some embodiments, the target polynucleotide comprises RNA, for example mRNA. In some embodiments, the target polynucleotide comprises an endogenous gene or gene product.

The target polynucleotide may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissue compared with tissue(s) or cells of a non-disease control. In some embodiments, it is a gene that becomes expressed at an abnormally high level. In some embodiments, it is a gene that becomes expressed at an abnormally low level. The altered expression can correlate with the occurrence and/or progression of the disease. A disease-associated gene also refers to a gene possessing mutation(s) or genetic variation that is directly responsible or is in linkage disequilibrium with a gene(s) that is response for the etiology of a disease. The transcribed or translated products may be known or unknown, and may be at a normal or abnormal level.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web. Exemplary genes associated with certain diseases and disorders are provided in Tables 4 and 5.

Mutations in these genes and pathways can result in production of improper proteins or proteins in improper amounts which affect function.

TABLE 4

| DISEASE/DISORDERS | GENE(S) |
|---|---|
| Neoplasia | PTEN; ATM; ATR; EGFR; |
| | ERBB2; ERBB3; ERBB4; |
| | Notch1; Notch2; Notch3; Notch4; |
| | AKT; AKT2; AKT3; HIF; |
| | HIF1a; HIF3a; Met; HRG; Bcl2; |
| | PPAR alpha; PPAR |
| | gamma; WT1 (Wilms Tumor); |
| | FGF Receptor Family |
| | members (5 members: 1, 2, 3, 4, 5); |
| | CDKN2a; APC; RB |
| | (retinoblastoma); MEN1; VHL; |
| | BRCA1; BRCA2; AR |
| | (Androgen Receptor); TSG101; |
| | IGF; IGF Receptor; Igf1 (4 |
| | variants); Igf2 (3 variants); Igf 1 |
| | Receptor; Igf 2 Receptor; |
| | Bax; Bcl2; caspases family (9 members: |
| | 1, 2, 3, 4, 6, 7, 8, 9, 12); Kras; Apc |
| Age-related Macular | Aber; Ccl2; Cc2; cp (ceruloplasmin); |
| Degeneration | Timp3; cathepsinD; Vldlr; Ccr2 |
| Schizophrenia | Neuregulin1 (Nrg1); Erb4 |
| | (receptor for Neuregulin); |
| | Complexin1 (Cplx1); Tph1 |
| | Tryptophan hydroxylase; Tph2 |
| | Tryptophan hydroxylase 2; |
| | Neurexin 1; GSK3; GSK3a; |
| | GSK3b |
| Disorders | 5-HTT (Slc6a4); COMT; DRD |
| | (Drd1a); SLC6A3; DAOA; |
| | DTNBP1; Dao (Dao1) |
| Trinucleotide Repeat | HTT (Huntington's Dx); |
| Disorders | SBMA/SMAX1/AR (Kennedy's |
| | Dx); FWX25 (Friedrich's Ataxia); |
| | ATX3 (Machado- |
| | Joseph's Dx); ATXN1 and |
| | ATXN2 (spinocerebellar |
| | ataxias); DNIPK (myotonic |
| | dystrophy); Atrophin-1 and Atn1 |
| | (DRPLA Dx); CBP (Creb-BP-global |
| | instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin |
| Disorders | (Psen1); nicastrin (Ncstn); PEN-2 |
| Others | Nos1; Parp1; Nat1; Nat2 |
| Prion-related disorders | Prp |
| ALS | SOD1; ALS2; STEX; FUS; |
| | TARDBP; VEGF (VEGF-a; |
| | VEGF-b; VEGF-c) |
| Drug addiction | Prkce (alcohol); Drd2; Drd4; |
| | ABAT (alcohol); GRIA2; |
| | Grm5; Grinl; Htr1b; Grin2a; Drd3; |
| | Pdyn; Grial (alcohol) |
| Autism | Mecp2; BZRAP1; MDGA2; Sema5A; |
| | Neurexin 1; Fragile X |
| | (FMR2 (AFF2); FXR1; FXR2; Mglur5) |
| Alzheimer's Disease | E1; CHIP; UCH; UBB; Tau; LRP; |
| | PICALM; Clusterin; PS1; |
| | SORL1; CR1; Vldlr; Uba1; |
| | Uba3; CHIP28 (Aqp1, |
| | Aquaporin 1); Uchl1; Uchl3; APP |
| Inflammation | IL-10; IL-1 (IL-1a; IL-1b); IL-13; |
| | IL-17 (IL-17a (CTLA8); IL- |
| | 17b; IL-17c; IL-17d; IL-17f); 11-23; |
| | Cx3cr1; ptpn22; TNFa; |
| | NOD2/CARD15 for IBD; IL-6; |
| | IL-12 (IL-12a; IL-12b); |
| | CTLA4; Cx3cl1 |
| Parkinson's Disease | x-Synuclein; DJ-1; LRRK2; Parkin; PINK1 |

TABLE 5

| | |
|---|---|
| Blood and | Anemia (CDAN1, CDA1, RPS19, DBA, |
| coagulation | PKLR, PK1, NT5C3, ITMPH1, |
| diseases and | PSN1, RHAG, RH50A, NRAMP2, |
| disorders | SPTB, ALAS2, ANH1, ASB, |
| | ABCB7, ABC7, ASAT); Bare |
| | lymphocyte syndrome (TAPBP, TPSN, |
| | TAP2, ABCB3, PSF2, RING11, |
| | MHC2TA, C2TA, RFX5, RFXAP, |
| | RFX5), Bleeding disorders (TBXA2R, |
| | P2RX1, P2X1); Factor H and |
| | factor H-like 1 (HF1, CFH, HUS); |
| | Factor V and factor VIII (MCFD2); |
| | Factor VII deficiency (F7); Factor X |
| | deficiency (F10); Factor XI |
| | deficiency (F11); Factor XII |
| | deficiency (F12, HAF); Factor XIIIA |
| | deficiency (F13A1, F13A); Factor XIIIB |
| | deficiency (F13B); Fanconi |
| | anemia (FANCA, FACA, FA1, |
| | FA, FAA, FAAP95, FAAP90, |
| | FLJ34064, |
| | FANCB, FANCC, FACC, BRCA2, |
| | FANCD1, FANCD2, FANCD, |
| | FACD, FAD, FANCE, FACE, FANCF, |
| | XRCC9, FANCG, BRIP1, |
| | BACH1, FANCJ, PHF9, FANCL, |
| | FANCM, KIAA1596); |
| | Hemophagocytic lymphohistiocytosis |
| | disorders (PRF1, HPLH2, |
| | UNC13D, MUNC13-4, HPLH3, |
| | HLH3, FHL3); Hemophilia A (F8, |
| | F8C, |
| | HEMA); Hemophilia B (F9, HEMB), |
| | Hemorrhagic disorders (PI, ATT, |
| | F5); Leukocyde deficiencies and |
| | disorders (ITGB2, CD18, LCAMB, |
| | LAD, EIF2B1, EIF2BA, EIF2B2, |
| | EIF2B3, EIF2B5, LVWM, CACH, |
| | CLE, EIF2B4); Sickle cell anemia |
| | (HBB); Thalassemia (HBA2, HBB, |
| | HBD, LCRB, HBA1). |
| Cell | B-cell non-Hodgkin lymphoma |
| dysregulation | (BCL7A, BCL7); Leukemia (TAL1 |
| and oncology | TCL5, SCL, TAL2, FLT3, NBS1, |
| diseases and | NBS, ZNFN1A1, IK1, LYF1, |
| disorders | HOXD4, HOX4B, BCR, CML, PHL, |
| | ALL, ARNT, KRAS2, RASK2, |
| | GMPS, AF10, ARHGEF12, LARG, |
| | KIAA0382, CALM, CLTH, |
| | CEBPA, CEBP, CHIC2, BTL, FLT3, |
| | KIT, PBT, LPP, NPM1, NUP214, |
| | D9S46E, CAN, CAIN, RUNX1, CBFA2, |
| | AML1, WHSC1L1, NSD3, |
| | FLT3, AF1Q, NPM1, NUMA1, ZNF145, |
| | PLZF, PML, MYL, STAT5B, |
| | AF10, CALM, CLTH, ARL11, ARLTS1, |
| | P2RX7, P2X7, BCR, CML, |
| | PHL, ALL, GRAF, NF1, VRNF, WSS, |
| | NFNS, PTPN11, PTP2C, SHP2, |
| | NS1, BCL2, CCND1, PRAD1, BCL1, |
| | TCRA, GATA1, GF1, ERYF1, |
| | NFE1, ABL1, NQO1, DIA4, NMOR1, |
| | NUP214, D9546E, CAN, |
| | CAIN). |
| Inflammation | AIDS (KIR3DL1, NKAT3, NKB1, |
| and immune | AMB11, KIR3D51, IFNG, CXCL12, |
| related diseases | SDF1); Autoimmune lymphoproliferative |
| and disorders | syndrome (TNFRSF6, APT1, |
| | FAS, CD95, ALPS1A); Combined |
| | immunodeficiency, (IL2RG, |
| | SCIDX1, SCIDX, IMD4); HIV-1 |
| | (CCL5, SCYA5, D17S136E, |
| | TCP228), |
| | HIV susceptibility or infection (IL10, |
| | CSIF, CMKBR2, CCR2, |
| | CMKBR5, CCCKR5 (CCR5)); |
| | Immunodeficiencies (CD3E, CD3G, |
| | AICDA, AID, HIGM2, TNFRSF5, |
| | CD40, UNG, DGU, HIGM4, |
| | TNFSF5, CD40LG, HIGM1, IGM, |
| | FOXP3, IPEX, AID, XPID, PIDX, |
| | TNFRSF14B, TACT); Inflammation |
| | (IL-10, IL-1 (IL-1a, IL-1b), IL-13, |
| | IL-17 (IL-17a (CTLA8), IL-17b, IL-17c, |
| | IL-17d, IL-17f), 11-23, Cx3cr1, |
| | ptpn22, TNFa, NOD2/CARD15 for IBD, |
| | IL-6, IL-12 (IL-12a, IL-12b), |
| | CTLA4, Cx3cl1); Severe combined |
| | immunodeficiencies |
| | (SCIDs)(JAK3, |
| | JAKL, DCLRE1C, ARTEMIS, SCIDA, |
| | RAG1, RAG2, ADA, PTPRC, |
| | CD45, LCA, IL7R, CD3D, T3D, IL2RG, |
| | SCIDX1, SCIDX, IMD4). |
| Metabolic, liver, | Amyloid neuropathy (TTR, PALB); |
| kidney and | Amyloidosis (AP0A1, APP, AAA, |
| protein diseases | CVAP, AD1, GSN, FGA, LYZ, TTR, |
| and disorders | PALB); Cirrhosis (KRT18, KRT8, |
| | CIRH1A, NAIC, TEX292, KIAA1988); |
| | Cystic fibrosis (CFTR, ABCC7, |
| | CF, MRP7); Glycogen storage diseases |
| | (SLC2A2, GLUT2, G6PC, |
| | G6PT, G6PT1, GAA, LAMP2, LAMPB, |
| | AGL, GDE, GBE1, GYS2, |
| | PYGL, PFKM); Hepatic adenoma, |
| | 142330 (TCF1, HNF1A, MODY3), |
| | Hepatic failure, early onset, and neurologic |
| | disorder (SCOD1, SC01), |
| | Hepatic lipase deficiency (LIPC), |
| | Hepatoblastoma, cancer and |
| | carcinomas (CTNNB1, PDGFRL, |
| | PDGRL, PRLTS, AXIN1, AXIN, |
| | CTNNB1, TP53, P53, LFS1, IGF2R, |
| | MPRI, MET, CASP8, MCH5; |
| | Medullary cystic kidney disease |
| | (UMOD, HNFJ, FJHN, MCKD2, |
| | ADMCKD2); Phenylketonuria |
| | (PAH, PKU1, QDPR, DHPR, PTS); |
| | Polycystic kidney and hepatic disease |
| | (FCYT, PKHD1, ARPKD, |
| | PKD1, |
| | PKD2, PKD4, PKDTS, PRKCSH, |
| | G19P1, PCLD, SEC63). |
| Muscular/Skeletal | Becker muscular dystrophy (DMD, |
| diseases and | BMD, MYF6), Duchenne Muscular |
| disorders | Dystrophy (DMD, BMD); Emery-Dreifuss |
| | muscular dystrophy (LMNA, |
| | LMN1, EMD2, FPLD, CMD1A, HGPS, |
| | LGMD1B, LMNA, LMN1, |
| | EMD2, FPLD, CMD1A); |
| | Facioscapulohumeral muscular dystrophy |
| | (FSHMD1A, FSHD1A); Muscular |
| | dystrophy (FKRP, MDC1C, |
| | LGMD2I, LAMA2, LAMM, LARGE, |
| | KIAA0609, MDC1D, FCMD, |
| | TTID, MYOT, CAPN3, CANP3, DYSF, |
| | LGMD2B, SGCG, LGMD2C, |
| | DMDA1, SCG3, SGCA, ADL, DAG2, |
| | LGMD2D, DMDA2, SGCB, |
| | LGMD2E, SGCD, SGD, LGMD2F, |
| | CMD1L, TCAP, LGMD2G, |
| | CMD1N, TRIM32, HT2A, LGMD2H, |
| | FKRP, MDC1C, LGMD2I, TTN, |
| | CMD1G, TMD, LGMD2J, POMT1, |
| | CAV3, LGMD1C, SEPN1, SELN, |
| | RSMD1, PLEC1, PLTN, EBS1); |
| | Osteopetrosis (LRP5, BMND1, LRP7, |
| | LR3, OPPG, VBCH2, CLCN7, CLC7, |
| | OPTA2, OSTM1, GL, TCIRG1, |
| | TIRC7, 0C116, OPTB1); Muscular |
| | atrophy (VAPB, VAPC, ALS8, |
| | SMN1, SMA1, SMA2, SMA3, |
| | SMA4, BSCL2, SPG17, GARS, |
| | SMAD1, CMT2D, HEXB, |
| | IGHMBP2, SMUBP2, CATF1, SMARD1). |
| Neurological | ALS (SOD1, ALS2, STEX, FUS, |
| and neuronal | TARDBP, VEGF (VEGF-a, VEGF-b, |
| diseases and | VEGF-c); Alzheimer disease (APP, |
| disorders | AAA, CVAP, AD1, APOE, AD2, |
| | PSEN2, AD4, STM2, APBB2, FE65L1, |
| | NOS3, PLAU, URK, ACE, |
| | DCP1, ACE1, MPO, PACIP1, PAXIP1L, |
| | PTIP, A2M, BLMH, BMH, |
| | PSEN1, AD3); Autism (Mecp2, |
| | BZRAP1, MDGA2, Sema5A, Neurexin |
| | 1, GLO1, MECP2, RTT, PPMX, |
| | M1RX16, MRX79, NLGN3, NLGN4, |
| | KIAA1260, AUTSX2); Fragile X |
| | Syndrome (FMR2, FXR1, FXR2, |
| | mGLUR5); Huntington's disease and |
| | disease like disorders (HD, IT15, |
| | PRNP, PRIP, JPH3, JP3, HDL2, TBP, |
| | SCA17); Parkinson disease |
| | (NR4A2, NURR1, NOT, TINUR, |
| | SNCAIP, TBP, SCA17, SNCA, |
| | NACP, PARK1, PARK4, DJ1, PARK7, |
| | LRRK2, PARK8, PINK1, |
| | PARK6, UCHL1, PARKS, SNCA, |
| | NACP, PARK1, PARK4, PRKN, |
| | PARK2, PDJ, DBH, NDUFV2); Rett |
| | syndrome (MECP2, RTT, PPMX, |
| | MRX16, MRX79, CDKL5, STK9, |
| | MECP2, RTT, PPMX, MRX16, |
| | MRX79, x-Synuclein, DJ-1); Schizophrenia |
| | (Neuregulin1 (Nrgl), Erb4 |
| | (receptor for Neuregulin), Complexin1 |
| | (Cplx1), Tph1 Tryptophan |
| | hydroxylase, Tph2, Tryptophan |
| | hydroxylase 2, Neurexin 1, GSK3, |
| | GSK3a, GSK3b, 5-HTT (51c6a4), |
| | COMT, DRD (Drdla), SLC6A3, |
| | DAOA, DTNBP1, Dao (Dao1)); |
| | Secretase Related Disorders (APH-1 |
| | (alpha and beta), Presenilin (Psen1), |
| | nicastrin, (Ncstn), PEN-2, Nos1, |
| | Parp1, Nat1, Nat2); Trinucleotide |
| | Repeat Disorders (HTT (Huntington's |
| | Dx), SBMA/SMAX1/AR (Kennedy's |
| | Dx), FWX25 (Friedrich's |
| | Ataxia), ATX3 (Machado- Joseph's Dx), |
| | ATXN1 and ATXN2 |
| | (spinocerebellar ataxias), DMPK |
| | (myotonic dystrophy), Atrophin-1 and |
| | Atn1 (DRPLA Dx), CBP (Creb-BP-global |
| | instability), VLDLR |
| | (Alzheimer's), Atxn7, Atxn10). |
| Ocular diseases | Age-related macular degeneration |
| and disorders | (Abcr, Ccl2, Cc2, cp (ceruloplasmin), |
| | Timp3, cathepsinD, Vldlr, Ccr2); Cataract |
| | (CRYAA, CRYA1, CRYBB2, |
| | CRYB2, PITX3, BFSP2, CP49, CP47, |
| | CRYAA, CRYA1, PAX6, AN2, |
| | MGDA, CRYBA1, CRYB1, CRYGC, |
| | CRYG3, CCL, LIM2, MP19, |
| | CRYGD, CRYG4, BFSP2, CP49, CP47, |
| | HSF4, CTM, HSF4, CTM, |
| | MIP, AQPO, CRYAB, CRYA2, CTPP2, |
| | CRYBB1, CRYGD, CRYG4, |
| | CRYBB2, CRYB2, CRYGC, CRYG3, |
| | CCL, CRYAA, CRYA1, GJA8, |
| | CX50, CAE1, GJA3, CX46, CZP3, |
| | CAE3, CCM1, CAM, KRIT1); |
| | Corneal clouding and dystrophy |
| | (APOA1, TGFBI, CSD2, CDGG1, |
| | CSD, BIGH3, CDG2, TACSTD2, |
| | TROP2, M1S1, VSX1, RINX, PPCD, |
| | PPD, KTCN, COL8A2, FECD, PPCD2, |
| | PIP5K3, CFD); Cornea plana |
| | congenital (KERA, CNA2); Glaucoma |
| | (MYOC, TIGR, GLC1A, JOAG, |
| | GPOA, OPTN, GLC1E, FIP2, HYPL, |
| | NRP, CYP1B1, GLC3A, OPA1, |
| | NTG, NPG, CYP1B1, GLC3A); Leber |
| | congenital amaurosis (CRB1, |
| | RP12, CRX, CORD2, CRD, RPGRIP1, |
| | LCA6, CORD9, RPE65, RP20, |
| | AIPL1, LCA4, GUCY2D, GUC2D, |
| | LCA1, CORD6, RDH12, LCA3); |
| | Macular dystrophy (ELOVL4, ADMD, |
| | STGD2, STGD3, RDS, RP7, |
| | PRPH2, PRPH, AVMD, AOFMD, VMD2). |

In some embodiments, the target polynucleotide sequence can comprise a protospacer sequence (i.e. sequence recognized by the spacer region of a guide nucleic acid) of 20 nucleotides in length. The protospacer can be less than 20 nucleotides in length. The protospacer can be at least 5, 10, 15, 16, 17, 18, 19, 20, 21, 22, 23, 24, 25, 30 or more nucleotides in length. The protospacer sequence can be at most 5, 10, 15, 16, 17, 18, 19, 20, 21, 22, 23, 24, 25, 30 or more nucleotides in length. The protospacer sequence can be 16, 17, 18, 19, 20, 21, 22, or 23 bases immediately 5′ of the first nucleotide of the PAM. The protospacer sequence can be 16, 17, 18, 19, 20, 21, 22, or 23 bases immediately 3′ of the last nucleotide of the PAM sequence. The protospacer sequence can be 20 bases immediately 5′ of the first nucleotide of the PAM sequence. The protospacer sequence can be 20 bases immediately 3′ of the last nucleotide of the PAM. The target nucleic acid sequence can be 5′ or 3′ of the PAM.

A protospacer sequence can include a nucleic acid sequence present in a target polynucleotide to which a nucleic acid-targeting segment of a guide nucleic acid can bind. For example, a protospacer sequence can include a sequence to which a guide nucleic acid is designed to have complementarity. A protspacer sequence can comprise any polynucleotide, which can be located, for example, in the nucleus or cytoplasm of a cell or within an organelle of a cell, such as a mitochondrion or chloroplast. A protospacer sequence can include cleavage sites for Cas proteins. A protospacer sequence can be adjacent to cleavage sites for Cas proteins.

The Cas protein can bind the target polynucleotide at a site within or outside of the sequence to which the nucleic acid-targeting sequence of the guide nucleic acid can bind. The binding site can include the position of a nucleic acid at which a Cas protein can produce a single-strand break or a double-strand break.

Site-specific binding of a target nucleic acid by a Cas protein can occur at locations determined by base-pairing complementarity between the guide nucleic acid and the target nucleic acid. Site-specific binding of a target nucleic acid by a Cas protein can occur at locations determined by a short motif, called the protospacer adjacent motif (PAM), in the target nucleic acid. The PAM can flank the protospacer, for example at the 3′ end of the protospacer sequence. For example, the binding site of Cas9 can be about 1 to about 25, or about 2 to about 5, or about 19 to about 23 base pairs (e.g., 3 base pairs) upstream or downstream of the PAM sequence. The binding site of Cas (e.g., Cas9) can be 3 base pairs upstream of the PAM sequence. The binding site of Cas (e.g., Cpf1) can be 19 bases on the (+) strand and 23 base on the (−) strand.

Different organisms can comprise different PAM sequences. Different Cas proteins can recognize different PAM sequences. For example, in S. pyogenes, the PAM can comprise the sequence 5′-XRR-3′, where R can be either A or G, where X is any nucleotide and X is immediately 3′ of the target nucleic acid sequence targeted by the spacer sequence. The PAM sequence of S. pyogenes Cas9 (SpyCas9) can be 5′-XGG-3′, where X is any DNA nucleotide and is immediately 3′ of the protospacer sequence of the non-complementary strand of the target DNA. The PAM of Cpf1 can be 5′-TTX-3′, where X is any DNA nucleotide and is immediately 5′ of the CRISPR recognition sequence.

The target sequence for the guide nucleic acid can be identified by bioinformatics approaches, for example, locating sequences within the target sequence adjacent to a PAM sequence. The optimal target sequence for the guide nucleic acid can be identified by experimental approaches, for example, testing a number of guide nucleic acid sequences to identify the sequence with the highest on-target activity and lowest off-target activity. The location of a target sequence can be determined by the desired experimental outcome. For example, a target protospacer can be located in a promoter in order to activate or repress a target gene. A target protospacer can be within a coding sequence, such as a 5′ constitutively expressed exon or sequences encoding a known domain. A target protospacer can be a unique sequence within the genome in order to mitigate off-target effects. Many publicly available algorithms for determining and ranking potential target protospacers are known in the art and can be used.

A target nucleic acid can comprise one or more sequences that is at least partially complementary to one or more guide nucleic acids. A target nucleic acid can be part or all of a gene, a 5′ end of a gene, a 3′ end of a gene, a regulatory element (e.g. promoter, enhancer), a pseudogene, non-coding DNA, a microsatellite, an intron, an exon, chromosomal DNA, mitrochondrial DNA, sense DNA, antisense DNA, nucleoid DNA, chloroplast DNA, or RNA among other nucleic acid entities. The target nucleic acid can be part or all of a plasmid DNA. A plasmid DNA or a portion thereof can be negatively supercoiled. A target nucleic acid can be in vitro or in vivo.

A target nucleic acid can comprise a sequence within a low GC content region. A target nucleic acid can be negatively supercoiled. By non-limiting example, the target nucleic acid can comprise a GC content of at least about 5, 10, 15, 20, 25, 30, 35, 40, 45, 50, 55, 60, or 65% or more. The target nucleic acid can comprise a GC content of at most about 5, 10, 15, 20, 25, 30, 35, 40, 45, 50, 55, 60, or 65% or more.

A region comprising a particular GC content can be the length of the target nucleic acid that hybridizes with the guide nucleic acid. A region comprising the GC content can be longer or shorter than the length of the region that hybridizes with the guide nucleic acid. A region comprising the GC content can be at least 30, 40, 50, 60, 70, 80, 90 or 100 or more nucleotides longer or shorter than the length of the region that hybridizes with the guide nucleic acid. A region comprising the GC content can be at most 30, 40, 50, 60, 70, 80, 90 or 100 or more nucleotides longer or shorter than the length of the region that hybridizes with the guide nucleic acid.

In various embodiments of the aspects herein, subject systems can be used for selectively modulating transcription (e.g., reduction or increase) of a target nucleic acid in a host cell. Selective modulation of transcription of a target nucleic acid can reduce or increase transcription of the target nucleic acid, but may not substantially modulate transcription of a non-target nucleic acid or off-target nucleic acid, e.g., transcription of a non-target nucleic acid may be modulated by less than 1%, less than 5%, less than 10%, less than 20%, less than 30%, less than 40%, or less than 50% compared to the level of transcription of the non-target nucleic acid in the absence of an actuator moiety, such as a guide nucleic acid/enzymatically inactive or enzymatically reduced Cas protein complex. For example, selective modulation (e.g., reduction or increase) of transcription of a target nucleic acid can reduce or increase transcription of the target nucleic acid by at least about 10%, at least about 20%, at least about 30%, at least about 40%, at least about 50%, at least about 60%, at least about 70%, at least about 80%, at least about 90%, or greater than 90%, compared to the level of transcription of the target nucleic acid in the absence of an actuator moiety, such as a guide nucleic acid/enzymatically inactive or enzymatically reduced Cas protein complex.

In some embodiments, the disclosure provides methods for increasing transcription of a target nucleic acid. The transcription of a target nucleic acid can increase by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 50-fold, at least about 70-fold, or at least about 100-fold compared to the level of transcription of the target DNA in the absence of an actuator moiety, such as a guide nucleic acid/enzymatically inactive or enzymatically reduced Cas protein complex. Selective increase of transcription of a target nucleic acid increases transcription of the target nucleic acid, but may not substantially increase transcription of a non-target DNA, e.g., transcription of a non-target nucleic acid is increased, if at all, by less than about 5-fold, less than about 4-fold, less than about 3-fold, less than about 2-fold, less than about 1.8-fold, less than about 1.6-fold, less than about 1.4-fold, less than about 1.2-fold, or less than about 1.1-fold compared to the level of transcription of the non-targeted DNA in the absence of an actuator moiety, such as a guide nucleic acid/enzymatically inactive or enzymatically reduced Cas protein complex.

In some embodiments, the disclosure provides methods for decreasing transcription of a target nucleic acid. The transcription of a target nucleic acid can decrease by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 50-fold, at least about 70-fold, or at least about 100-fold compared to the level of transcription of the target DNA in the absence of an actuator moiety, such as a guide nucleic acid/enzymatically inactive or enzymatically reduced Cas protein complex. Selective decrease of transcription of a target nucleic acid decreases transcription of the target nucleic acid, but may not substantially decrease transcription of a non-target DNA, e.g., transcription of a non-target nucleic acid is decreased, if at all, by less than about 5-fold, less than about 4-fold, less than about 3-fold, less than about 2-fold, less than about 1.8-fold, less than about 1.6-fold, less than about 1.4-fold, less than about 1.2-fold, or less than about 1.1-fold compared to the level of transcription of the non-targeted DNA in the absence of an actuator moiety, such as a guide nucleic acid/enzymatically inactive or enzymatically reduced Cas protein complex.

Transcription modulation can be achieved by fusing the actuator moiety, such as an enzymatically inactive Cas protein, to a heterologous sequence. The heterologous sequence can be a suitable fusion partner, e.g., a polypeptide that provides an activity that indirectly increases, decreases, or otherwise modulates transcription by acting directly on the target nucleic acid or on a polypeptide (e.g., a histone or other DNA-binding protein) associated with the target nucleic acid. Non-limiting examples of suitable fusion partners include a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

A suitable fusion partner can include a polypeptide that directly provides for increased transcription of the target nucleic acid. For example, a transcription activator or a fragment thereof, a protein or fragment thereof that recruits a transcription activator, or a small molecule/drug-responsive transcription regulator. A suitable fusion partner can include a polypeptide that directly provides for decreased transcription of the target nucleic acid. For example, a transcription repressor or a fragment thereof, a protein or fragment thereof that recruits a transcription repressor, or a small molecule/drug-responsive transcription regulator.

The heterologous sequence or fusion partner can be fused to the C-terminus, N-terminus, or an internal portion (i.e., a portion other than the N- or C-terminus) of the actuator moiety, for example a dead Cas protein. Non-limiting examples of fusion partners include transcription activators, transcription repressors, histone lysine methyltransferases (KMT), Histone Lysine Demethylates, Histone lysine acetyltransferases (KAT), Histone lysine deacetylase, DNA methylases (adenosine or cytosine modification), CTCF, periphery recruitment elements (e.g., Lamin A, Lamin B), and protein docking elements (e.g., FKBP/FRB).

Non-limiting examples of transcription activators include GAL4, VP16, VP64, and p65 subdomain (NFkappaB).

Non-limiting examples of transcription repressors include Kruippel associated box (KRAB or SKD), the Mad mSIN3 interaction domain (SID), and the ERF repressor domain (ERD).

Non-limiting examples of histone lysine methyltransferases (KMT) include members from KMT1 family (e.g., SUV39H1, SUV39H2, G9A, ESET/SETDB1, Clr4, Su(var)3-9), KMT2 family members (e.g., hSET1A, hSET1B, MLL 1 to 5, ASH1, and homologs (Trx, Trr, Ashl)), KMT3 family (SYMD2, NSD1), KMT4 (DOT1L and homologs), KMT5 family (Pr-SET7/8, SUV4-20H1, and homologs), KMT6 (EZH2), and KMT8 (e.g., RIZ1).

Non-limiting examples of Histone Lysine Demethylates (KDM) include members from KDM1 family (LSD1/BHC110, Splsd1/Swm1/Saf110, Su(var)3-3), KDM3 family (JHDM2a/b), KDM4 family (JMJD2A/JHDM3A, JMJD2B, JMJD2C/GASC1, JMJD2D, and homologs (Rph1)), KDM5 family (JARID1A/RBP2, JARID1B/PLU-1, JARIDIC/SMCX, JARID1D/SMCY, and homologs (Lid, Jhn2, Jmj2)), and KDM6 family (e.g., UTX, JMJD3).

Non-limiting examples of KAT include members of KAT2 family (hGCN5, PCAF, and homologs (dGCN5/PCAF, Gcn5), KAT3 family (CBP, p300, and homologs (dCBP/NEJ)), KAT4, KAT5, KATE, KAT7, KATE, and KAT13.

In some embodiments, an actuator moiety comprising a dead Cas protein or dead Cas fusion protein is targeted by a guide nucleic acid to a specific location (i.e., sequence) in the target nucleic acid and exerts locus-specific regulation such as blocking RNA polymerase binding to a promoter (e.g., which can selectively inhibit transcription activator function), and/or modifying the local chromatin status (e.g., when a fusion sequence is used that can modify the target nucleic acid or modifies a polypeptide associated with the target nucleic acid). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some embodiments, a guide nucleic acid can comprise a protein binding segment to recruit a heterologous polypeptide to a target nucleic acid to modulate transcription of a target nucleic acid. Non-limiting examples of the heterologous polypeptide include a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. The guide nucleic acid can comprise a protein binding segment to recruit a transcriptional activator, transcriptional repressor, or fragments thereof.

In some embodiments, gene expression modulation is achieved by using a guide nucleic acid designed to target a regulatory element of a target nucleic acid, for example, transcription response element (e.g., promoters, enhancers), upstream activating sequences (UAS), and/or sequences of unknown or known function that are suspected of being able to control expression of the target DNA.

A subject system can be introduced into a variety of cells. A variety of cells can be utilized in the subject methods and systems. A cell can be in vitro. A cell can be in vivo. A cell can be ex vivo. A cell can be an isolated cell. A cell can be a cell inside of an organism. A cell can be an organism. A cell can be a cell in a cell culture. A cell can be one of a collection of cells. A cell can be a mammalian cell or derived from a mammalian cell. A cell can be a rodent cell or derived from a rodent cell. A cell can be a human cell or derived from a human cell. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a pluripotent stem cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a microbe cell or derived from a microbe cell. A cell can be a fungi cell or derived from a fungi cell. A cell can be from a specific organ or tissue.

A cell can be a stem cell or progenitor cell. Cells can include stem cells (e.g., adult stem cells, embryonic stem cells, iPS cells) and progenitor cells (e.g., cardiac progenitor cells, neural progenitor cells, etc.). Cells can include mammalian stem cells and progenitor cells, including rodent stem cells, rodent progenitor cells, human stem cells, human progenitor cells, etc. Clonal cells can comprise the progeny of a cell. A cell can comprise a target nucleic acid. A cell can be in a living organism. A cell can be a genetically modified cell. A cell can be a host cell.

A cell can be a totipotent stem cell, however, in some embodiments of this disclosure, the term “cell” may be used but may not refer to a totipotent stem cell. A cell can be a plant cell, but in some embodiments of this disclosure, the term “cell” may be used but may not refer to a plant cell. A cell can be a pluripotent cell. For example, a cell can be a pluripotent hematopoietic cell that can differentiate into other cells in the hematopoietic cell lineage but may not be able to differentiate into any other non-hematopoetic cell. A cell can be a hematopoietic progenitor cell. A cell can be a hematopoietic stem cell. A cell may be able to develop into a whole organism. A cell may or may not be able to develop into a whole organism. A cell may be a whole organism.

A cell can be a primary cell. For example, cultures of primary cells can be passaged 0 times, 1 time, 2 times, 4 times, 5 times, 10 times, 15 times or more. Cells can be unicellular organisms. Cells can be grown in culture.

A cell can be a diseased cell. A diseased cell can have altered metabolic, gene expression, and/or morphologic features. A diseased cell can be a cancer cell, a diabetic cell, and an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells with which a subject system can be utilized include, but are not limited to, lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In various embodiments of the aspects herein, a subject system is expressed in a cell or cell population. Cells, for example immune cells (e.g., lymphocytes including T cells and NK cells), can be obtained from a subject. Non-limiting examples of subjects include humans, dogs, cats, mice, rats, and transgenic species thereof. Examples of samples from a subject from which cells can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

In various embodiments of the aspects herein, an immune cell comprises a lymphocyte. In some embodiments, the lymphocyte is a natural killer cell (NK cell). In some embodiments, the lymphocyte is a T cell. T cells can be obtained from a number of sources, including peripheral blood mononuclear cells, bone marrow, lymph node tissue, spleen tissue, umbilical cord, and tumors. In some embodiments, any number of T cell lines available can be used. Immune cells such as lymphocytes (e.g., cytotoxic lymphocytes) can preferably be autologous cells, although heterologous cells can also be used. T cells can be obtained from a unit of blood collected from a subject using any number of techniques, such as Ficoll separation. Cells from the circulating blood of an individual can be obtained by apheresis or leukapheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media, such as phosphate buffered saline (PBS), for subsequent processing steps. After washing, the cells can be resuspended in a variety of biocompatible buffers, such as Ca-free, Mg-free PBS. Alternatively, the undesirable components of the apheresis sample can be removed and the cells directly resuspended in culture media. Samples can be provided directly by the subject, or indirectly through one or more intermediaries, such as a sample collection service provider or a medical provider (e.g. a physician or nurse). In some embodiments, isolating T cells from peripheral blood leukocytes can include lysing the red blood cells and separating peripheral blood leukocytes from monocytes by, for example, centrifugation through, e.g., a PERCOL™ gradient.

A specific subpopulation of T cells, such as CD4+ or CD8+ T cells, can be further isolated by positive or negative selection techniques. Negative selection of a T cell population can be accomplished, for example, with a combination of antibodies directed to surface markers unique to the cells negatively selected. One suitable technique includes cell sorting via negative magnetic immunoadherence, which utilizes a cocktail of monoclonal antibodies directed to cell surface markers present on the cells negatively selected. For example, to isolate CD4+ cells, a monoclonal antibody cocktail can include antibodies to CD14, CD20, CD11b, CD16, HLA-DR, and CD8. The process of negative selection can be used to produce a desired T cell population that is primarily homogeneous. In some embodiments, a composition comprises a mixture of two or more (e.g. 2, 3, 4, 5, or more) different kind of T-cells.

In some embodiments, the immune cell is a member of an enriched population of cells. One or more desired cell types can be enriched by any suitable method, non-limiting examples of which include treating a population of cells to trigger expansion and/or differentiation to a desired cell type, treatment to stop the growth of undesired cell type(s), treatment to kill or lyse undesired cell type(s), purification of a desired cell type (e.g. purification on an affinity column to retain desired or undesired cell types on the basis of one or more cell surface markers). In some embodiments, the enriched population of cells is a population of cells enriched in cytotoxic lymphocytes selected from cytotoxic T cells (also variously known as cytotoxic T lymphocytes, CTLs, T killer cells, cytolytic T cells, CD8+ T cells, and killer T cells), natural killer (NK) cells, and lymphokine-activated killer (LAK) cells.

For isolation of a desired population of cells by positive or negative selection, the concentration of cells and surface (e.g., particles such as beads) can be varied. In certain embodiments, it can be desirable to significantly decrease the volume in which beads and cells are mixed together (i.e., increase the concentration of cells), to ensure maximum contact of cells and beads. For example, a concentration of 2 billion cells/mL can be used. In some embodiments, a concentration of 1 billion cells/mL is used. In some embodiments, greater than 100 million cells/mL are used. A concentration of cells of 10, 15, 20, 25, 30, 35, 40, 45, or 50 million cells/mL can be used. In yet another embodiment, a concentration of cells from 75, 80, 85, 90, 95, or 100 million cells/mL can be used. In further embodiments, concentrations of 125 or 150 million cells/mL can be used. Using high concentrations can result in increased cell yield, cell activation, and cell expansion.

A cell, e.g., an immune cell, can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. In some embodiments, a cell transiently transfected with the various components of a subject system (such as by transient transfection of one or more vectors, or transfection with RNA), and modified through the activity of a CRISPR complex, is used to establish a new cell line comprising cells containing the modification but lacking any other exogenous sequence.

A subject system introduced into a cell can be used for regulating expression of a target polynucleotide (e.g., gene expression). The expressed GMP of various embodiments of the aspects herein are useful in regulating expression of a target gene. In an aspect, the disclosure provides methods of inducing expression of a gene modulating polypeptide (GMP). The method comprises (a) providing a cell expressing a transmembrane receptor having a ligand binding domain and a signaling domain; (b) binding a ligand to the ligand binding domain of the transmembrane receptor, wherein the binding activates a signaling pathway of the cell such that a promoter operably linked to a nucleic acid sequence encoding the GMP is in turn activated; and (c) expressing the GMP upon activation of the promoter.

Binding a ligand to the transmembrane receptor can occur in vitro and/or in vivo. Binding the ligand to the transmembrane receptor can comprise to bringing the receptor in contact with the ligand. The ligand can be a membrane-bound protein or non-membrane bound protein. The ligand is, in some cases, bound the membrane of a cell.

In some embodiments, the GMP is expressed preferentially when the ligand binds the transmembrane receptor. In some embodiments, the GMP is expressed primarily when the ligand binds the transmembrane receptor. In some embodiments, the GMP is expressed only when the ligand binds the transmembrane receptor.

The promoter operably linked to the GMP coding sequence can be present in the cell as part of a plasmid, for example a non-integrating vector. In some cases, the GMP coding sequence has been integrated into the genome. The GMP coding sequence can be integrated into the genome such that it is operably linked to an endogenous promoter. The GMP coding sequence can be integrated into the genome such that it is downstream of a gene encoding an endogenous protein that is regulated by an endogenous promoter. The GMP coding sequence may be joined in frame to the gene. Alternatively, the GMP coding sequence may be linked to the gene via a nucleic acid sequence comprising an IRES. In some cases, an expression cassette comprising a promoter operably linked to a nucleic acid sequence encoding the GMP is integrated into the genome. In some cases, this expression cassette is integrated randomly into the genome.

In an aspect, the present disclosure provides a method of regulating expression of a target gene in a cell, comprising (a) contacting a ligand to a transmembrane receptor comprising a ligand binding domain and a signaling domain, wherein upon the contacting, the signaling domain activates a signaling pathway of the cell; (b) expressing a gene modulating polypeptide (GMP) comprising an actuator moiety from an expression construct comprising a nucleic acid sequence encoding the GMP placed under control of a promoter, wherein the promoter is activated to drive expression of the GMP upon binding of the ligand to the ligand binding domain; and (c) increasing or decreasing expression of the target gene via binding of the expressed GMP, thereby regulating expression of the target gene.

In an aspect, the present disclosure provides a method of regulating expression of a target gene in a cell, comprising contacting a ligand to a transmembrane receptor comprising a ligand binding domain, a signaling domain, and a gene modulating polypeptide (GMP), the GMP comprising an actuator moiety linked to a cleavage recognition site, wherein upon contacting the ligand to the ligand binding domain, the signaling domain activates a signaling pathway of the cell; expressing a cleavage moiety from an expression cassette comprising a nucleic acid sequence encoding the cleavage moiety, wherein the nucleic acid sequence is placed under the control of a promoter activated by the signaling pathway to drive expression of the cleavage moiety upon binding of the ligand to the ligand binding domain; and cleaving, by the cleavage moiety, the cleavage recognition site to release the actuator moiety from the transmembrane receptor, wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane domain, the signaling domain, the cleavage recognition site, and the actuator moiety. The ligand binding domain can be located in the extracellular region of the cell. The signaling domain, the cleavage recognition site, and the actuator moiety can be located in the intracellular region of the cell.

In an aspect, the present disclosure provides a method of regulating expression of a target gene in a cell comprising contacting a ligand to a transmembrane receptor comprising a ligand binding domain, a signaling domain, and a cleavage moiety, wherein upon contacting the ligand to the ligand binding domain, the signaling domain activates a signaling pathway of the cell; expressing a fusion protein comprising a gene modulating polypeptide (GMP) linked to a nuclear export signal peptide from an expression cassette comprising a nucleic acid sequence encoding the fusion protein, the GMP comprising an actuator moiety linked to a cleavage recognition site, wherein the nucleic acid sequence is placed under the control of a promoter activated by the signaling pathway to drive expression of the fusion protein upon binding of the ligand to the ligand binding domain; and cleaving, by the cleavage moiety, the cleavage recognition site to release the actuator moiety, wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, the signaling domain, and the cleavage moiety. The ligand binding domain can be located in the extracellular region of the cell. The signaling domain, the cleavage recognition site, and the actuator moiety can be located in the intracellular region of the cell.

In an aspect, the present disclosure provides a method of regulating expression of a target gene in a cell, comprising contacting a ligand with a transmembrane receptor comprising a ligand binding domain and a signaling domain, wherein upon contacting the ligand to the ligand binding domain, the signaling domain activates a signaling pathway of the cell; expressing a cleavage moiety from an expression cassette comprising a nucleic acid sequence encoding the cleavage moiety, wherein the nucleic acid sequence is placed under the control of a promoter activated by the signaling pathway to drive expression of the cleavage moiety upon binding of the ligand to the ligand binding domain; and cleaving, by the cleavage moiety, a cleavage recognition site of a fusion protein comprising a gene modulating polypeptide (GMP) linked to a nuclear export signal peptide, wherein the GMP comprises an actuator moiety linked to the cleavage recognition site, wherein upon cleaving, the actuator moiety is released, and wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell.

In an aspect, the present disclosure provides a method of regulating expression of a target gene in a cell, comprising contacting a ligand to a transmembrane receptor comprising a ligand binding domain and a signaling domain, wherein upon contacting the ligand to the ligand binding domain, the signaling domain activates a signaling pathway of the cell; expressing a fusion protein comprising a gene modulating polypeptide (GMP) linked to a nuclear export signal peptide from an expression cassette comprising a nucleic acid sequence encoding the fusion protein, the GMP comprising an actuator moiety linked to a cleavage recognition sequence, wherein the nucleic acid sequence is placed under the control of a promoter activated by the signaling pathway to drive expression of the fusion protein upon binding of the ligand to the ligand binding domain; cleaving, by a cleavage moiety, the cleavage recognition site of the fusion protein to release the actuator moiety, wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell.

In an aspect, the present disclosure provides a method of regulating expression of a target gene in a cell, comprising contacting a ligand to a transmembrane receptor comprising a ligand binding domain and a signaling domain, wherein upon contacting the ligand to the ligand binding domain, the signaling domain activates a signaling pathway of the cell; expressing a fusion protein comprising a gene modulating polypeptide (GMP) linked to a nuclear export signal peptide from a first expression cassette comprising a first nucleic acid sequence encoding the fusion protein, the GMP comprising an actuator moiety linked to a cleavage recognition sequence, wherein the nucleic acid sequence is placed under the control of a first promoter activated by the signaling pathway to drive expression of the fusion protein upon binding of the ligand to the ligand binding domain; expressing a cleavage moiety from a second expression cassette comprising a nucleic acid sequence encoding the cleavage moiety, wherein the nucleic acid is placed under the control of a second promoter activated by the signaling pathway to drive expression of the cleavage moiety upon binding of the ligand to the ligand binding domain; and cleaving, by the expressed cleavage moiety, the cleavage recognition site of the expressed fusion protein to release the actuator moiety, wherein the released actuator moiety regulates expression of a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell.

In an aspect, the present disclosure provides a method of regulating expression of a target gene in a cell, comprising contacting a ligand to a transmembrane receptor comprising a ligand binding domain and a signaling domain, wherein upon contacting the ligand to the ligand binding domain, the signaling domain activates a signaling pathway of the cell; expressing a first partial gene modulating polypeptide (GMP) from a first expression cassette comprising a first nucleic acid sequence encoding the first partial GMP, the first partial GMP comprising a first portion of an actuator moiety, wherein the first nucleic acid sequence is placed under the control of a first promoter activated by the signaling pathway to drive expression of the first partial GMP upon binding of the ligand to the ligand binding domain; expressing a second partial gene modulating polypeptide (GMP) from a second expression cassette comprising a second nucleic acid sequence encoding the second partial GMP, the second partial GMP comprising a second portion of an actuator moiety, wherein the second nucleic acid sequence is placed under the control of a second promoter activated by the signaling pathway to drive expression of the second partial GMP upon binding of the ligand to the ligand binding domain; and forming a complex of the first partial GMP and second partial GMP to form a reconstituted actuator moiety, wherein the reconstituted actuator moiety regulates expression of a target polynucleotide, for example a target gene. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell.

In an aspect, the present disclosure provides a method of regulating expression of a target gene in a cell, comprising contacting a ligand to a transmembrane receptor comprising a ligand binding domain and a signaling domain, wherein upon binding of the ligand to the ligand binding domain, the signaling domain activates a signaling pathway of the cell; expressing a first partial cleavage moiety from a first expression cassette comprising a first nucleic acid sequence encoding the first partial cleavage moiety, wherein the first nucleic acid sequence is placed under the control of a first promoter activated by the signaling pathway to drive expression of the first partial cleavage moiety upon binding of the ligand to the ligand binding domain; expressing a second partial cleavage moiety from a second expression cassette comprising a second nucleic acid sequence encoding the second partial cleavage moiety, wherein the second nucleic acid sequence is placed under the control of a second promoter activated by the signaling pathway to drive expression of the second partial cleavage moiety upon binding of the ligand to the ligand binding domain; forming a complex of the first and second partial cleavage moiety to yield a reconstituted cleavage moiety; and cleaving, by the reconstituted cleavage moiety, a cleavage recognition site to release an actuator moiety from a nuclear export signal peptide, wherein the released actuator moiety regulates expression of a target polynucleotide, for example a target gene. In some embodiments, the cleavage moiety cleaves the cleavage recognition site when in proximity to the cleavage recognition site. In some cases, the transmembrane receptor comprises, from the N-terminus to the C-terminus, the ligand binding domain, a transmembrane region, and the signaling domain. The ligand binding domain can be located in the extracellular region of the cell. The signaling domain can be located in the intracellular region of the cell.

In an aspect, the present disclosure provides a method of regulating expression of a target gene in a cell, comprising contacting a ligand to a transmembrane receptor comprising a ligand binding domain and a signaling domain, wherein upon contacting the ligand to the ligand binding domain, the signaling domain activates a signaling pathway of the cell; expressing one or both of (i) a cleavage moiety and (ii) a fusion protein comprising a gene modulating polypeptide (GMP) linked to a nuclear export signal peptide, the GMP comprising an actuator moiety linked to a cleavage recognition site, from an expression cassette comprising a nucleic acid sequence encoding one or both of (i) and (ii), wherein the nucleic acid sequence is placed under the control of a promoter activated by the signaling pathway upon binding of a ligand to the ligand binding domain; and releasing the actuator moiety upon cleavage of the cleavage recognition site by the cleavage moiety, wherein the released actuator moiety regulates expression of a target polynucleotide, for example a target gene.

Contacting a ligand to the transmembrane receptor can be conducted in vitro and/or in vivo. Contacting the ligand to the transmembrane receptor can comprise to bringing the receptor in contact with the ligand. The ligand can be a membrane-bound protein or non-membrane bound protein. The ligand is, in some cases, bound the membrane of a cell. The ligand is, in some cases, not bound the membrane of a cell. Contacting a cell to a ligand can be conducted in vitro by culturing the cell expressing a subject system in the presence of the ligand. For example, a cell expressing subject system can be cultured as an adherent cell or in suspension, and the ligand can be added to the cell culture media. In some cases, the ligand is expressed by a target cell, and exposing can comprise co-culturing the cell expressing a subject system and the target cell expressing the ligand. Cells can be co-cultured in various suitable types of cell culture media, for example with supplements, growth factors, ions, etc. Exposing a cell expressing a subject system to a target cell (e.g., a target cell expressing an antigen) can be accomplished in vivo, in some cases, by administering the cells to a subject, for example a human subject, and allowing the cells to localize to the target cell via the circulatory system.

Contacting can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

In some embodiments, a GMP is expressed preferentially when the ligand binds the transmembrane receptor. In some embodiments, a GMP is expressed primarily when the ligand binds the transmembrane receptor. In some embodiments, a GMP is expressed only when the ligand binds the transmembrane receptor. In some embodiments, a first partial GMP is expressed preferentially when the ligand binds the transmembrane receptor. In some embodiments, a first GMP is expressed primarily when the ligand binds the transmembrane receptor. In some embodiments, a first partial GMP is expressed only when the ligand binds the transmembrane receptor. In some embodiments, a second partial GMP is expressed preferentially when the ligand binds the transmembrane receptor. In some embodiments, a second partial GMP is expressed primarily when the ligand binds the transmembrane receptor. In some embodiments, a second partial GMP is expressed only when the ligand binds the transmembrane receptor.

In some embodiments, a cleavage moiety is expressed preferentially when the ligand binds the transmembrane receptor. In some embodiments, a cleavage moiety is expressed primarily when the ligand binds the transmembrane receptor. In some embodiments, a cleavage moiety is expressed only when the ligand binds the transmembrane receptor. In some embodiments, a first partial cleavage moiety is expressed preferentially when the ligand binds the transmembrane receptor. In some embodiments, a first partial cleavage moiety is expressed primarily when the ligand binds the transmembrane receptor. In some embodiments, a first partial cleavage moiety is expressed only when the ligand binds the transmembrane receptor. In some embodiments, a second partial cleavage moiety is expressed preferentially when the ligand binds the transmembrane receptor. In some embodiments, a second partial cleavage moiety is expressed primarily when the ligand binds the transmembrane receptor. In some embodiments, a second partial cleavage moiety is expressed only when the ligand binds the transmembrane receptor.

Upon contacting the transmembrane receptor with the ligand, the promoter is activated to drive expression of the GMP. As previously described herein, the expressed GMP can regulate expression of the target gene by increasing or decreasing the expression of the target gene via the actuator moiety. The actuator moiety can regulate expression or activity of a gene and/or edit the sequence of a nucleic acid (e.g., a gene and/or gene product).

The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease, or a variant thereof. In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity. An actuator moiety having reduced or minimal nuclease activity can regulate expression and/or activity of a gene by physical obstruction of a target polynucleotide or recruitment of additional factors effective to suppress or enhance expression of the target polynucleotide. In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. An actuator moiety can regulate expression or activity of a gene and/or edit a nucleic acid sequence, whether exogenous or endogenous. For example, an actuator moiety can comprise a Cas protein which lacks cleavage activity.

The present disclosure also provides expression cassettes.

In an aspect, the present disclosure provides an expression cassette that comprises a promoter operably linked to a nucleic acid sequence encoding a gene modulating polypeptide (GMP) comprising an actuator moiety, wherein the expression cassette is characterized in that the promoter is activated to drive expression of the GMP from the expression cassette when the expression cassette is present in a cell that expresses a transmembrane receptor, wherein the transmembrane receptor has been activated by binding of a ligand to the transmembrane receptor.

In some embodiments, the expression cassette is supplied to the cell as part of a plasmid. The plasmid can be a non-integrating vector. The plasmid carrying the expression cassette can be replicating or non-replicating. The plasmid can be delivered to a cell by a variety of methods, including electroporation, microinjection, gene gun, hydrostatic pressure, and lipofection. The plasmid can also be delivered using polymeric carriers.

In some embodiments, the expression cassette is integrated into a cell genome. A variety of genome editing techniques can be used for the integration of an expression cassette. In some embodiments, the expression cassette is supplied to the cell as part of a viral vector. Viruses can insert genetic material into a cell genome. Viral mediated delivery of the expression cassette can facilitate insertion or integration of the expression cassette into the cell genome. Viruses, such as retroviruses, can utilize long terminal repeat (LTR) sequences and LTR specific integrases to integrate nucleic acid sequences into a cell genome. In some embodiments, an expression cassette provided herein comprises at least one long terminal repeat (LTR) useful for viral mediated nucleic acid integration.

In some embodiments, the expression cassette integrates into a region of the genome comprising a safe harbor site. Safe-harbor sites refer to regions of the genome which are generally transcriptionally active regions with an open chromatin configuration and transgene insertion has been previously demonstrated to have no or minimal effect on global and local gene expression. Exemplary safe-harbor sites include the AAVS1 site of chromosome 19 and the CCR5 site of chromosome 3. In some cases, integration of the expression cassette into the AAVS1 site disrupts the gene phosphatase 1 regulator subunit 12c (PPP1R12C).

In some embodiments, the expression cassette is inserted into a cell genome using an engineered nuclease. Nucleases for genome editing can create site-specific double-stranded breaks at untargeted or targeted (e.g., programmable) regions of the genome. Exemplary nucleases include meganucleases, zinc finger nucleases (ZFNs), transcription activator-like effector nucleases (TALENs), and the CRISPR-Cas system. Nuclease induced double-stranded breaks can then be repaired through nonhomologous end-joining (NHEJ) or homology directed repair (HDR) (e.g., homolgous recombination (HR)). In repairing these double-stranded breaks, nucleic acids sequences can be inserted or integrated in the genome.

In some embodiments, the expression cassette can be integrated into a cell genome via NHEJ or HDR following the generation of double-stranded breaks at targeted or untargeted regions of the genome. NHEJ uses a variety of enzymes to directly join the DNA ends in a double-stranded break. An expression cassette comprising a promoter operably linked to a GMP coding sequence can be integrated into the genome at the site of the double-stranded break during NHEJ. In HDR, a homologous sequence is utilized as a template for regeneration of missing DNA sequence at the break point. Nucleic acid sequences having sequences homologous to the site of the double-stranded break can be integrated into the genome during this repair process. In some embodiments, the expression cassette comprises homology sequences flanking the promoter and GMP coding sequence which effects homologous recombination at a site of interest in the genome.

Upon integration in the genome, the promoter of the expression cassette can be activated by one or multiple signaling pathways of the cell to drive expression of the GMP. The expressed GMP can then regulate expression of a target gene. In the case where the GMP is an RNA-guided actuator moiety, the expressed GMP is operable to complex with a guide-RNA and regulate expression of a target gene.

In an aspect, the present disclosure provides an expression cassette comprising (i) a nucleic acid sequence encoding a gene modulating polypeptide (GMP), and (ii) at least one integration sequence which facilitates integration of the expression cassette into a cell genome, wherein the GMP comprises an actuator moiety, and wherein the expression cassette is characterized in that activation of a transmembrane receptor by binding of a ligand to the transmembrane receptor activates a promoter to drive expression of the GMP from the expression cassette when the expression cassette has been integrated into the cell genome via the at least one integration sequence. In some embodiments, activation of a transmembrane receptor by binding of a ligand to the transmembrane receptor activates a promoter to drive expression of the GMP from the expression cassette only when the expression cassette has been integrated into the cell genome.

The at least one integration sequence of the expression cassette can mediate integration of the expression cassette in the cell genome.

In some cases, the integration sequence comprises a long terminal repeat (LTR) and the expression cassette is supplied to the cell as part of the viral vector. Viral mediated delivery of the expression cassette facilitates integration of the expression cassette into the cell genome (e.g., via LTR integrases).

In some embodiments, the integration sequence comprises a homology sequence which mediates integration through homology directed repair (HDR). In some cases, two homology sequences flank the GMP coding sequence and facilitate genome integration by homology directed repair. In some cases, integration is effected by a nuclease, e.g., programmable nuclease. Exemplary programmable nucleases include RNA-guided nucleases such as Cas proteins, zinc finger nucleases (ZFN) and transcription activator-like effector nucleases (TALENs). The homology sequences flanking GMP coding sequence can effect homologous recombination at a site downstream of an endogenous promoter. The GMP coding sequence, when integrated into the cell genome, can be operably linked to the endogenous promoter.

In some cases, the homology sequences flanking the GMP coding sequence can effect homologous recombination at a site downstream of a gene encoding an endogenous protein under the control of an endogenous promoter. As previously described herein, the GMP coding sequence can be joined to the gene by a nucleic acid sequence encoding a peptide linker. The peptide linker, in some cases, comprises a protease recognition sequence and can be cleaved by a protease. The peptide linker, in some cases, has a self-cleaving segment such as a 2A peptide (e.g., T2A, P2A, E2A, and F2A). In some cases, multiple self-cleaving segments are present. In some cases, the GMP coding sequence is joined to the gene by a nucleic acid sequence comprising an IRES.

Expression cassettes of the disclosure can be present in a cell as part of a plasmid (e.g., a non-integrating vector). In some embodiments, the expression cassette is integrated into the cell genome, for example via viral integration or genome editing using a programmable nuclease. The expression cassette may be integrated randomly into the cell genome, or is, in some cases, targeted to a specific region of the genome. An expression cassette comprising a GMP coding sequence operably linked to a promoter can be integrated into a region of the genome comprising a safe harbor site. The expression cassette can be integrated, for example, into the AAVS1 site of chromosome 19 or CCR5 site of chromosome 3.

Any suitable delivery method can be used for introducing the compositions and molecules (e.g., polypeptides and/or nucleic acid encoding polypeptides of the system) of the disclosure into a host cell. The compositions (e.g., expression cassette, GMP coding sequence, endogenous/exogenous promoter sequence, guide nucleic acid, etc) can be delivered simultaneously or temporally separated. The choice of delivery method of can be dependent on the type of cell being transformed and/or the circumstances under which the transformation is taking place (e.g., in vitro, ex vivo, or in vivo).

A method of delivery can involve contacting a target polynucleotide or introducing into a cell (or a population of cells) one or more nucleic acids comprising nucleotide sequences encoding the compositions of the disclosure (e.g., GMP coding sequence, exogenous promoter sequence, guide nucleic acid, etc). Suitable nucleic acids comprising nucleotide sequences encoding the compositions of the disclosure can include expression vectors, where an expression vector comprising a nucleotide sequence encoding one or more compositions of the disclosure (e.g., GMP coding sequence, exogenous promoter sequence, guide nucleic acid, etc) is a recombinant expression vector.

Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, use of cell permeable peptides, and nanoparticle-mediated nucleic acid delivery.

In some aspects, the present disclosure provides methods comprising delivering one or more polynucleotides, or one or more oligonucleotides as described herein, or vectors as described herein, or one or more transcripts thereof, and/or one or proteins transcribed therefrom, to a host cell. In some aspects, the disclosure further provides cells produced by such methods, and organisms (such as animals, plants, or fungi) comprising or produced from such cells.

A polynucleotide encoding any of the polypeptides disclosed herein can be codon-optimized. Codon optimization can entail the mutation of foreign-derived (e.g., recombinant) DNA to mimic the codon preferences of an intended host organism or cell while encoding the same protein. Thus, the codons can be changed, but the encoded protein remains unchanged. For example, if the intended target cell was a human cell, a human codon-optimized polynucleotide could be used for producing a suitable Cas protein. As another non-limiting example, if the intended host cell were a mouse cell, then a mouse codon-optimized polynucleotide encoding a Cas protein could be a suitable Cas protein. A polynucleotide encoding a polypeptide such as an actuator moiety (e.g., a Cas protein) can be codon optimized for many host cells of interest. A host cell can be a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. In some cases, codon optimization may not be required. In some instances, codon optimization can be preferable.

Conventional viral and non-viral based gene transfer methods can be used to introduce nucleic acids in mammalian cells or target tissues. Such methods can be used to administer nucleic acids encoding compositions of the disclosure to cells in culture, or in a host organism. Non-viral vector delivery systems can include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, and agent-enhanced uptake of DNA. Cationic and neutral lipids that are suitable for efficient receptor-recognition lipofection of polynucleotides can be used. Delivery can be to cells (e.g. in vitro or ex vivo administration) or target tissues (e.g. in vivo administration). The preparation of lipid:nucleic acid complexes, including targeted liposomes such as immunolipid complexes, can be used.

RNA or DNA viral based systems can be used to target specific cells in the body and trafficking the viral payload to the nucleus of the cell. Viral vectors can be administered directly (in vivo) or they can be used to treat cells in vitro, and the modified cells can optionally be administered (ex vivo). Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene. High transduction efficiencies can be observed in many different cell types and target tissues.

The tropism of a retrovirus can be altered by incorporating foreign envelope proteins, expanding the potential target population of target cells. Lentiviral vectors are retroviral vectors that can transduce or infect non-dividing cells and produce high viral titers. Selection of a retroviral gene transfer system can depend on the target tissue. Retroviral vectors can comprise cis-acting long terminal repeats with packaging capacity for up to 6-10 kb of foreign sequence. The minimum cis-acting LTRs can be sufficient for replication and packaging of the vectors, which can be used to integrate the therapeutic gene into the target cell to provide permanent transgene expression. Retroviral vectors can include those based upon murine leukemia virus (MuLV), gibbon ape leukemia virus (GaLV), Simian Immuno deficiency virus (SIV), human immuno deficiency virus (HIV), and combinations thereof.

An adenoviral-based systems can be used. Adenoviral-based systems can lead to transient expression of the transgene. Adenoviral based vectors can have high transduction efficiency in cells and may not require cell division. High titer and levels of expression can be obtained with adenoviral based vectors. Adeno-associated virus (“AAV”) vectors can be used to transduce cells with target nucleic acids, e.g., in the in vitro production of nucleic acids and peptides, and for in vivo and ex vivo gene therapy procedures.

Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV. Additional methods for the delivery of nucleic acids to cells can be used, for example, as described in US20030087817, incorporated herein by reference.

A host cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. In some embodiments, a cell transiently transfected with the compositions of the disclosure (such as by transient transfection of one or more vectors, or transfection with RNA), and modified through the activity of an actuator moiety such as a CRISPR complex, is used to establish a new cell line comprising cells containing the modification but lacking any other exogenous sequence.

Any suitable vector compatible with the host cell can be used with the methods of the disclosure. Non-limiting examples of vectors for eukaryotic host cells include pXT1, pSG5 (Stratagene™), pSVK3, pBPV, pMSG, and pSVLSV40 (Pharmacia™).

In some embodiments, a nucleotide sequence encoding a guide nucleic acid and/or Cas protein or chimera is operably linked to a control element, e.g., a transcriptional control element, such as a promoter. The transcriptional control element can be functional in either a eukaryotic cell, e.g., a mammalian cell, or a prokaryotic cell (e.g., bacterial or archaeal cell). In some embodiments, a nucleotide sequence encoding a guide nucleic acid and/or a Cas protein or chimera is operably linked to multiple control elements that allow expression of the nucleotide sequence encoding a guide nucleic acid and/or a Cas protein or chimera in prokaryotic and/or eukaryotic cells.

Depending on the host/vector system utilized, any of a number of suitable transcription and translation control elements, including constitutive and inducible promoters, transcription enhancer elements, transcription terminators, etc. may be used in the expression vector (e.g., U6 promoter, H1 promoter, etc.; see above) (see e.g., Bitter et al. (1987) Methods in Enzymology, 153:516-544).

In some embodiments, compositions of the disclosure (e.g., GMP, e.g., actuator moiety such as a Cas protein or Cas chimera, chimeric receptor, guide nucleic acid, etc) can be provided as RNA. In such cases, the compositions of the disclosure (e.g., GMP, e.g., actuator moiety such as a Cas protein or Cas chimera, chimeric receptor, guide nucleic acid, etc) can be produced by direct chemical synthesis or may be transcribed in vitro from a DNA. The compositions of the disclosure (e.g., GMP, e.g., actuator moiety such as a Cas protein or Cas chimera, chimeric receptor, guide nucleic acid, etc) can be synthesized in vitro using an RNA polymerase enzyme (e.g., T7 polymerase, T3 polymerase, SP6 polymerase, etc.). Once synthesized, the RNA can directly contact a target DNA or can be introduced into a cell using any suitable technique for introducing nucleic acids into cells (e.g., microinjection, electroporation, transfection, etc).

Nucleotides encoding a guide nucleic acid (introduced either as DNA or RNA) and/or a Cas protein or chimera (introduced as DNA or RNA) can be provided to the cells using a suitable transfection technique; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) Efficient gene targeting in Drosophila by direct embryo injection with zinc-finger nucleases. PNAS 105(50):19821-19826. Nucleic acids encoding the compositions of the disclosure (e.g., GMP, e.g., actuator moiety such as a Cas protein or Cas chimera, chimeric receptor, guide nucleic acid, etc) may be provided on DNA vectors or oligonucleotides. Many vectors, e.g. plasmids, cosmids, minicircles, phage, viruses, etc., useful for transferring nucleic acids into target cells are available. The vectors comprising the nucleic acid(s) can be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as MMLV, HIV-1, and ALV.

The compositions of the disclosure (e.g., GMP, e.g., an actuator moiety such as a Cas protein or Cas chimera, chimeric receptor, guide nucleic acid, etc), whether introduced as nucleic acids or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g. one time, twice, three times, or more than three times, and the cells allowed to incubate with the agent(s) for some amount of time following each contacting event e.g. 16-24 hours, after which time the media can be replaced with fresh media and the cells can be cultured further.

In cases in which two or more different targeting complexes are provided to the cell (e.g., two different guide nucleic acids that are complementary to different sequences within the same or different target DNA), the complexes may be provided simultaneously (e.g. as two polypeptides and/or nucleic acids), or delivered simultaneously. Alternatively, they may be provided consecutively, e.g. the targeting complex being provided first, followed by the second targeting complex, etc. or vice versa.

An effective amount of the compositions of the disclosure (e.g., GMP, e.g., actuator moiety such as Cas protein or Cas chimera, chimeric receptor, guide nucleic acid, etc) can be provided to the target DNA or cells. An effective amount can be the amount to induce, for example, at least about a 2-fold change (increase or decrease) or more in the amount of target regulation observed between two homologous sequences relative to a negative control, e.g. a cell contacted with an empty vector or irrelevant polypeptide. An effective amount or dose can induce, for example, about 2-fold change, about 3-fold change, about 4-fold change, about a 7-fold, about 8-fold increase, about 10-fold, about 50-fold, about 100-fold, about 200-fold, about 500-fold, about 700-fold, about 1000-fold, about 5000-fold, or about 10.000-fold change in target gene regulation. The amount of target gene regulation may be measured by any suitable method.

Contacting the cells with a composition of the can occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors can include polypeptides and non-polypeptide factors.

In numerous embodiments, the chosen delivery system is targeted to specific tissue or cell types. In some cases, tissue- or cell-targeting of the delivery system is achieved by binding the delivery system to tissue- or cell-specific markers, such as cell surface proteins. Viral and non-viral delivery systems can be customized to target tissue or cell-types of interest.

The present disclosure provides pharmaceutical compositions comprising a system or an expression cassette as described herein (e.g., nucleic acids, plasmids, polypeptides, guide RNA, etc, e.g., molecules). The pharmaceutical composition may further comprise one or more pharmaceutically acceptable excipients. Pharmaceutical compositions containing comprising a system or an expression cassette described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The molecules can be packed together or separately, in a single package or in a plurality of packages. One or all of the therapeutic agents can be given in multiple doses. If not simultaneous, the timing between the multiple doses may vary to as much as about a month.

Molecules described herein can be administered before, during, or after the occurrence of a disease or condition, and the timing of administering the composition containing a compound can vary. For example, the pharmaceutical compositions can be used as a prophylactic and can be administered continuously to subjects with a propensity to conditions or diseases in order to prevent the occurrence of the disease or condition. The molecules and pharmaceutical compositions can be administered to a subject during or as soon as possible after the onset of the symptoms. The administration of the molecules can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A molecule can be administered as soon as is practicable after the onset of a disease or condition is detected or suspected, and for a length of time necessary for the treatment of the disease, such as, for example, from about 1 month to about 3 months. The length of treatment can vary for each subject.

A molecule can be packaged into a biological compartment. A biological compartment comprising the molecule can be administered to a subject. Biological compartments can include, but are not limited to, viruses (lentivirus, adenovirus), nanospheres, liposomes, quantum dots, nanoparticles, microparticles, nanocapsules, vesicles, polyethylene glycol particles, hydrogels, and micelles.

For example, a biological compartment can comprise a liposome. A liposome can be a self-assembling structure comprising one or more lipid bilayers, each of which can comprise two monolayers containing oppositely oriented amphipathic lipid molecules. Amphipathic lipids can comprise a polar (hydrophilic) headgroup covalently linked to one or two or more non-polar (hydrophobic) acyl or alkyl chains. Energetically unfavorable contacts between the hydrophobic acyl chains and a surrounding aqueous medium induce amphipathic lipid molecules to arrange themselves such that polar headgroups can be oriented towards the bilayer's surface and acyl chains are oriented towards the interior of the bilayer, effectively shielding the acyl chains from contact with the aqueous environment.

Examples of preferred amphipathic compounds used in liposomes can include phosphoglycerides and sphingolipids, representative examples of which include phosphatidylcholine, phosphatidylethanolamine, phosphatidylserine, phosphatidylinositol, phosphatidic acid, phoasphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, di stearoylphosphatidylcholine (DSPC), dilinoleoylphosphatidylcholine and egg sphingomyelin, or any combination thereof.

A biological compartment can comprise a nanoparticle. A nanoparticle can comprise a diameter of from about 40 nanometers to about 1.5 micrometers, from about 50 nanometers to about 1.2 micrometers, from about 60 nanometers to about 1 micrometer, from about 70 nanometers to about 800 nanometers, from about 80 nanometers to about 600 nanometers, from about 90 nanometers to about 400 nanometers, from about 100 nanometers to about 200 nanometers.

In some instances, as the size of the nanoparticle increases, the release rate can be slowed or prolonged and as the size of the nanoparticle decreases, the release rate can be increased.

The amount of albumin in the nanoparticles can range from about 5% to about 85% albumin (v/v), from about 10% to about 80%, from about 15% to about 80%, from about 20% to about 70% albumin (v/v), from about 25% to about 60%, from about 30% to about 50%, or from about 35% to about 40%. The pharmaceutical composition can comprise up to 30, 40, 50, 60, 70 or 80% or more of the nanoparticle. In some instances, the nucleic acid molecules of the disclosure can be bound to the surface of the nanoparticle.

A biological compartment can comprise a virus. The virus can be a delivery system for the pharmaceutical compositions of the disclosure. Exemplary viruses can include lentivirus, retrovirus, adenovirus, herpes simplex virus I or II, parvovirus, reticuloendotheliosis virus, and adeno-associated virus (AAV).

The Pharmaceutical compositions of the disclosure can be delivered to a cell using a virus. The virus can infect and transduce the cell in vivo, ex vivo, or in vitro. In ex vivo and in vitro delivery, the transduced cells can be administered to a subject in need of therapy. Pharmaceutical compositions can be packaged into viral delivery systems. For example, the compositions can be packaged into virions by a HSV-1 helper virus-free packaging system.

Viral delivery systems (e.g., viruses comprising the pharmaceutical compositions of the disclosure) can be administered by direct injection, stereotaxic injection, intracerebroventricularly, by minipump infusion systems, by convection, catheters, intravenous, parenteral, intraperitoneal, and/or subcutaenous injection, to a cell, tissue, or organ of a subject in need. In some instances, cells can be transduced in vitro or ex vivo with viral delivery systems. The transduced cells can be administered to a subject having a disease. For example, a stem cell can be transduced with a viral delivery system comprising a pharmaceutical composition and the stem cell can be implanted in the patient to treat a disease. In some instances, the dose of transduced cells given to a subject can be about 1×105 cells/kg, about 5×105 cells/kg, about 1×106 cells/kg, about 2×106 cells/kg, about 3×106 cells/kg, about 4×106 cells/kg, about 5×106 cells/kg, about 6×106 cells/kg, about 7×106 cells/kg, about 8×106 cells/kg, about 9×106 cells/kg, about 1×107 cells/kg, about 5×107 cells/kg, about 1×108 cells/kg, or more in one single dose.

Introduction of the biological compartments into cells can occur by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In various embodiments of the aspects herein, methods of the disclosure are performed in a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician). A subject can be a plant or a crop.

### EXAMPLES

The following examples are given for the purpose of illustrating various embodiments of the invention and are not meant to limit the present invention in any fashion. Changes therein and other uses will occur to those skilled in the art.

### Example 1: System Comprising One Transmembrane Receptor

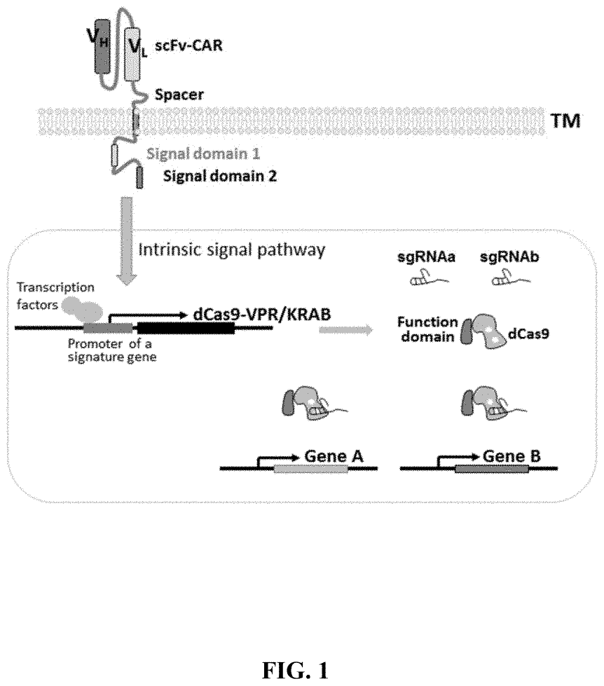

This example describes an illustrative system comprising a transmembrane receptor useful for regulating expression of at least one target gene. As illustrated in FIG. 1, upon binding of a ligand with a synthetic receptor comprising a chimeric antigen receptor (CAR, e.g., scFv-CAR), an intrinsic signal transduction pathway is activated, resulting in the recruitment of at least one cellular transcription factor to the promoter region of an endogenous gene (a signature gene) at its natural locus. A GMP coding sequence is integrated into the genome and is placed under the control of the promoter of the signature gene. Transcriptional activation of the promoter results in expression of the gene modulating polypeptide (GMP) comprising a dCas (e.g., dCas9) linked to VPR (e.g., transcriptional activator) or KRAB (e.g., transcription repressor). The expressed GMP, upon complexing with a guide RNA (e.g., sgRNAa, sgRNAb) which is constitutively expressed, can regulate (activate or suppress) the expression of a chosen target gene (e.g., Gene A, Gene B).

### Example 2: System Comprising Two Transmembrane Receptors

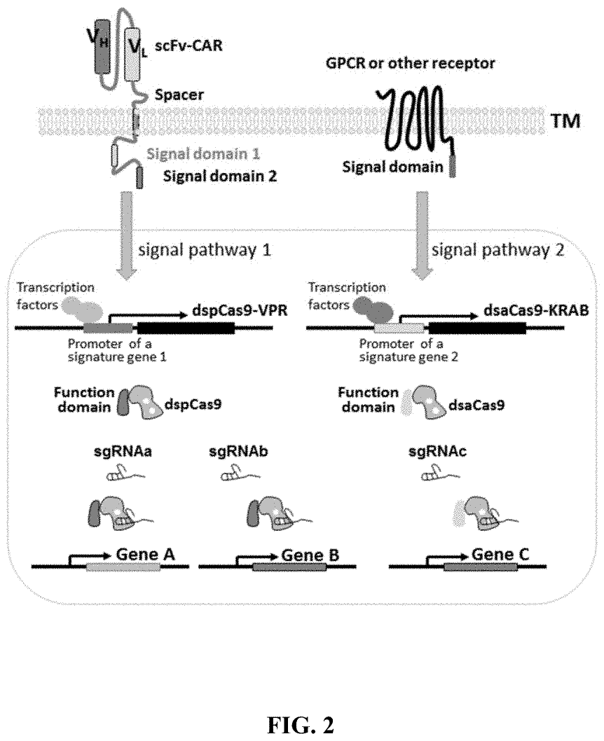

This example describes an illustrative system comprising two transmembrane receptors useful for regulating expression of at least one target gene. As illustrated in FIG. 2, binding of an antigen with a chimeric antigen receptor (CAR, e.g., scFv-CAR) activates an intrinsic signal transduction pathway 1, leading to the synthesis of dspCas9-VPR (dead S. pyogenes Cas9 linked to VPR) and subsequent activation of Gene A and B. Binding of a ligand with a GPCR receptor activates signal pathway 2, leading to the synthesis of dsaCas9-KRAB (dead S. aureus Cas9 linked to KRAB) and subsequent suppression of the expression of Gene C. Alternatively, signal pathway 2 can also be used to regulate CAR expression or the same target genes of signal pathway 1 for conditional control of signal output.

### Example 3: Conditional Expression of a GFP Reporter Gene by a Ligand-Dependent Signal Cascade

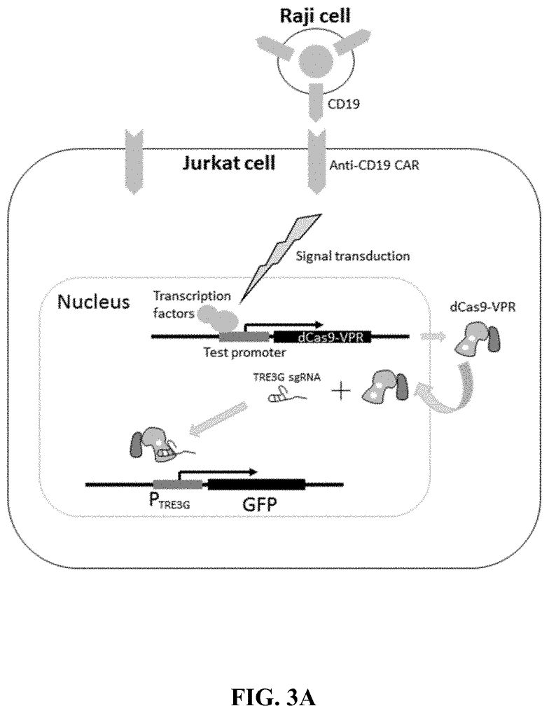

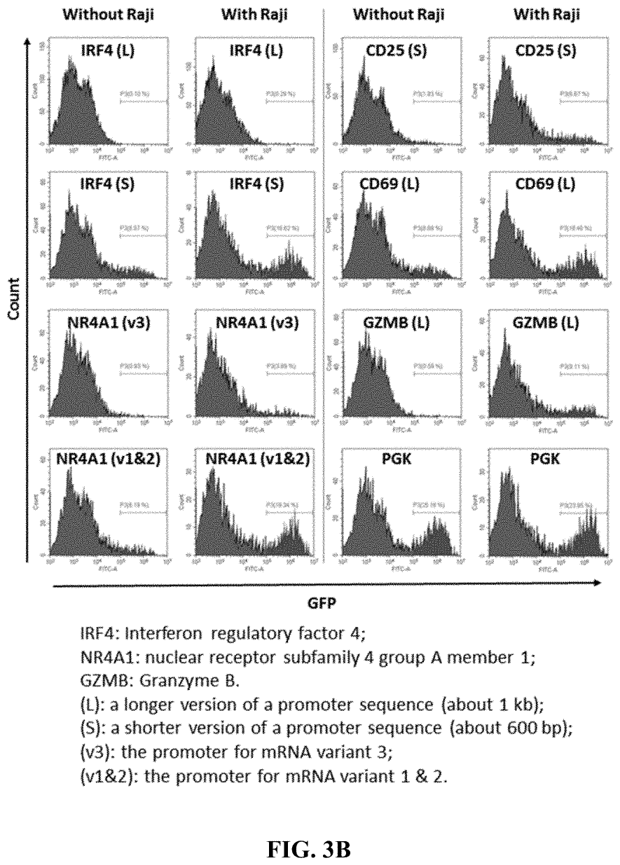

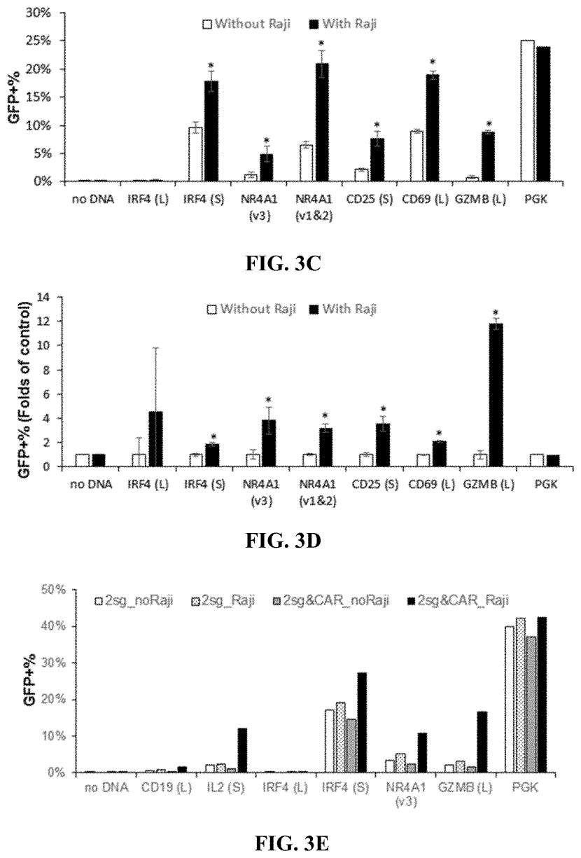

In this example, a stable Jurkat reporter cell line (‘2sg&CAR’) was generated by transduction with two lentiviral vectors encoding the following components: (1) an anti-CD19 CAR expression cassette; (2) a TRE3G promoter-driven GFP expression cassette (the promoter has 7 sgRNA binding sites); (3) a sgRNA targeting the TRE3G promoter; and (4) a sgRNA targeting the CXCR4 promoter. As illustrated in FIG. 3A, upon binding of anti-CD19 CAR present on the Jurkat cell surface with CD19 expressed on the surface of Raji cells, the intracellular signaling domain of anti-CD19 CAR is activated for signal transduction, resulting in transcriptional activation of the test promoter to drive dCas9-VPR expression. Newly synthesized dCas9-VPR protein can translocate into the cell nucleus and complex with a TRE3G sgRNA. The RNA-guided dCas9-VPR can activate the TRE3G promoter to drive GFP expression. In some cases, the dCas9-VPR can complex with the TRE3G sgRNA prior to translocation into the cell nucleus.

Jurkat reporter cells were transfected with a plasmid DNA encoding one of seven test promoters (Table 6) comprising endogenous promoter sequences.

TABLE 6

| | Name | Promoter | Description |
|---|---|---|---|
| Promoter1 | IRF4 (L) | Interferon | (L): long version of a |
| | | regulatory factor 4 | promoter sequence (~1 kb) |
| Promoter2 | IRF4 (S) | Interferon | (S): short version of a |
| | | regulatory factor 4 | promoter sequence (~600 bp) |
| Promoter3 | NR4A1 | Nuclear receptor | (v3): promoter for |
| | (v3) | subfamily 4 group | mRNA variant |
| | | A member 1 | 3 (~1 kb) |
| Promoter4 | NR4A1 | Nuclear receptor | (v1&2): promoter for mRNA |
| | (v1&2) | subfamily 4 group | variant 1 & 2 (~1 kb) |
| | | A member 1 | |
| Promoter5 | CD25 | | (S): short version of a |
| | (S) | | promoter sequence (~600 bp) |
| Promoter6 | CD69 | | (L): long version of a |
| | (L) | | promoter sequence (~1 kb) |
| Promoter7 | GZMB | GZMB: | (L): long version of a |
| | (L) | Granzyme B | promoter sequence (~1 kb) |

The test promoters were operably linked to a nucleic acid sequence encoding for dCas9-VPR. An hour after transfection, the cells were divided into equal parts and an equal number of Raji cells were added into one part of the transfected reporter cells. A day later, cells were evaluated for GFP expression in a flow cytometer. Jurkat reporter cells with Raji cells were stained for anti-CD19-PE and anti-CD3-APC before evaluating GFP expression. FIG. 3B shows GFP expression levels in unstimulated and Raji-stimulated Jurkat reporter cells. Plots shown are gated on alive (without Raji) or alive CD19-CD3+(with Raji) cells.

FIGS. 3C and 3D quantify the results of FIG. 3B. In FIG. 3C, average GFP+% values of two independent transfections are shown. Error bars represent standard deviation. * Student's t-test, p<0.05. For promoter 1 (IRF4 (L)), there is almost no GFP+ cells and no difference between cells treated with Raji or without Raji. The promoter1-dCas9-VPR construct can be regarded as a negative control construct in the experiment. For PGK promoter, nearly 25% of GFP+% cells were detected in both reporter cells treated either with Raji or without Raji, which is consistent with the notion that PGK promoter drives constitutive gene expression in T cells. For test promoters 2-7, significant increases in GFP+% cells were detected in Jurkat cells incubated with Raji cells compared to without Raji cell incubation, suggesting that ligand-dependent conditional upregulation of reporter gene expression is achieved using these 6 endogenous promoters.

In FIG. 3D, values of GFP+% cells in Raji-stimulated samples were divided by values in cells without Raji treatment. More than one log difference in GFP+% cells between reporter cells treated with Raji and without Raji were detected using one of the tested promoters, suggesting that the systems disclosed herein can amplify input signals.

For comparison, a control cell line generated by transduction with lentivirus encoding (1) a TRE3G promoter-driven GFP expression cassette (the promoter has 7 sgRNA binding sites) and (2) a sgRNA targeting the TRE3G promoter and another sgRNA targeting the CXCR4 gene were generated. The control cell line lacked CD19-CAR (2sg′). The control cell (2sg) and 2sg&CAR cell line were treated similar to above in this example. 2sg and 2sg&CAR cells were transfected with a plasmid DNA encoding one of seven test promoters—CD19 L (long version of promoter), IL2 S (short version of promoter), IRF4 L (long version of promoter), IRF4 S (short version of promoter), NR4A1 v3 (promoter for mRNA variant 3), GZMB L (long version of promoter), or PGK. As shown in FIG. 3E, inducible GFP expression was detected in the 2sg&CAR cell line treated with the dCas9VPR constructs driven by the short IL2, short IRF4, NR4A1v3, or long GZMB promoter but not in the 2sg cell line, suggesting that the conditional upregulation of the GFP reporter gene expression is dependent on the CD19 and CD19CAR interaction, e.g., ligand and receptor interaction (e.g., antigen and scFv interactions).

### Example 4: Promoters for Use in Systems and Methods Provided Herein

A list of potential promoters for use in systems disclosed herein for a TCR signaling pathway is provided in Table 7. Experimental evaluation of these promoters may identify at least one promoter with desired features for therapeutic and/or research purposes.

TABLE 7
Candidate promoters in TCR signaling pathway

| Gene name | Gene name | Gene name | Gene name | Gene name |
|---|---|---|---|---|
| A_23_P33103 | D12686 | MRPL12 | PLAGL2 | TXN |
| AB018273 | DDX18 | MRPL12 | PRDX1 | U97075 |
| AB023135 | EBNA1BP2 | MRPL13 | PRDX3 | UMPK |
| AB067484 | EDARADD | MRPL17 | PRDX4 | UMPS |
| ADRM1 | EGR2 | MRPL50 | PSAT1 | VIM |
| AF117229 | EIF2S1 | MRPS17 | PSMA3 | WDR3 |
| AF283301 | EIF3S1 | MTHFD2 | PSMC4 | WDR4 |
| AK000540 | EIF5B | MYC | PSMD12 | X66610 |
| AK074235 | ELL2 | NCBP1 | PWP2H | XCL2 |
| AK074970 | ENO1 | NCBP2 | PYCR1 | XCL2 |
| APOBEC3B | ERH | NFE2L3 | RBBP8 | ZBED2 |
| ATP1B3 | ETF1 | NM_006082 | RIOK2 | ZNF593 |
| AY033999 | EXOSC3 | NM_013285 | RUVBL2 | |
| AY423045 | F5 | NM_013332 | S40832 | |
| B3GALT6 | FABP5 | NM_014473 | S75463 | |
| BC001648 | GABPB2 | NM_016037 | SCO2 | |
| BC004856 | GART | NM_016077 | SFXN1 | |
| BC006201 | GBP1 | NM_016391 | SFXN1 | |
| BC017083 | GEMIN4 | NM_017858 | SHMT2 | |
| BC018929 | GEMIN6 | NM_018096 | SLC19A2 | |
| BC022522 | GTPBP4 | NM_018128 | SLC29A1 | |
| BC025376 | GZMB | NM_018405 | SLC43A3 | |
| BCL2A1 | HCCS | NM_018509 | SLCO4A1 | |
| BIRC3 | HOMER1 | NM_018664 | SNX1 | |
| BX648514 | HRB | NM_024096 | SNX9 | |
| BXDC1 | HSPA8 | NM_024098 | SPAG5 | |
| BYSL | HSPCB | NM_025115 | SRM | |
| C1orf33 | HSPE1 | NM_031216 | STIP1 | |
| C20orf53 | TARS | NM_032299 | TALDO1 | |
| CALM2 | ICSBP1 | NM_032346 | TARS | |
| CCL3 | IER3 | NM_138779 | THC1867539 | |
| CCL4 | IFNG | NM_152400 | THC1910362 | |
| CC T5 | IFRD2 | NM_152718 | THC1956109 | |
| CDK4 | IL2RA | NM_178014 | THC2002468 | |
| CLTC | IRF4 | NM_178834 | TNF | |
| CREM | LRP8 | NPTX1 | TNFRSF1B | |
| CSF2 | M90813 | NR4A3 | TNFRSF9 | |
| CTNNAL1 | MAT2A | PAK1IP1 | TNFSF6 | |
| CTPS | MCM6 | PBEF1 | TOMM40 | |
| CXCL9 | MCTS1 | PGAM1 | TRIT1 | |

### Example 5: Conditional Expression of a GFP Reporter Gene by a Ligand-Dependent Signal Cascade in Stable Cell Lines

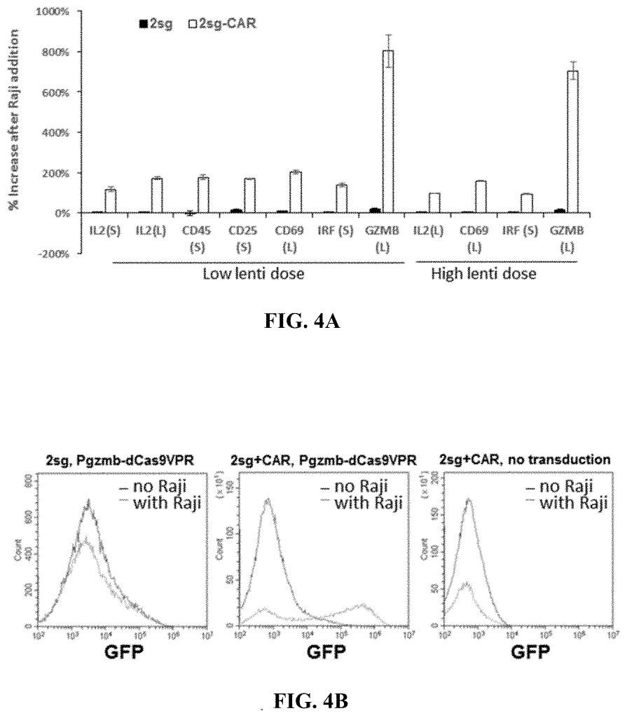

The Jurkat reporter cell line without CD19-CAR (2sg) or with CD19-CAR (2sg&CAR or 2sg+CAR or 2sg-CAR) as in FIG. 3E were transduced with lentiviral vectors at low or high lentivirus doses. The lentiviral vectors contain a dCas9-VPR transgene under the control of IL2 short promoter, IL2 long promoter, CD45 short promoter, CD25 short promoter, CD69 long promoter, IRF short promoter, or GZMB long promoter. After at least 2 weeks, the established stable cell lines were either untreated or stimulated by co-culture with Raji cells. After two days of co-culture, cells were evaluated for GFP expression by flow cytometry. Jurkat reporter cells stimulated with Raji cells were stained for anti-CD19-PE and anti-CD3-APC before evaluation. In FIG. 4A, plots shown are gated on live (without Raji) or live CD19-CD3+(with Raji) cells. % Increase in GFP high expression cells was calculated using the following formula: % increase=(GFP-hi %_with Raji−GFP-hi %_no Raji)/GFP-hi %_no Raji×100%. Average values of two treated samples are shown. Error bars represent standard deviation. The % increase in 2sg-CAR cell line for all the tested promoters shown is statistically significant compared to the 2sg cell line (p<0.05, student's t-test). CD19CAR-activation-dependent GFP expression was observed for various of the tested promoters. Among these promoters, the GZMB promoter showed the strongest induction regardless of the initial amount of lentivirus used.

FIG. 4B shows CAR-dependent signaling in sorted cells with stably integrated GZMB promoter-dCas9-VPR constructs. Induction of GFP reporter expression was observed in the cell line stably expressing both CD19-CAR and GZMB promoter-driven dCas9-VPR. Minimal expression was detected in the cell lines expressing either CD19CAR or GZMB promoter-driven dCAS9VPR. This data demonstrates the induction of reporter gene expression in a ligand-receptor interaction-specific manner in stable cell lines.

### Example 6: Simultaneously Induction of Expression of Multiple Genes, Including an Endogeneous Gene, by an Inducible Synthetic Promoter Through the CAR Signaling Pathway

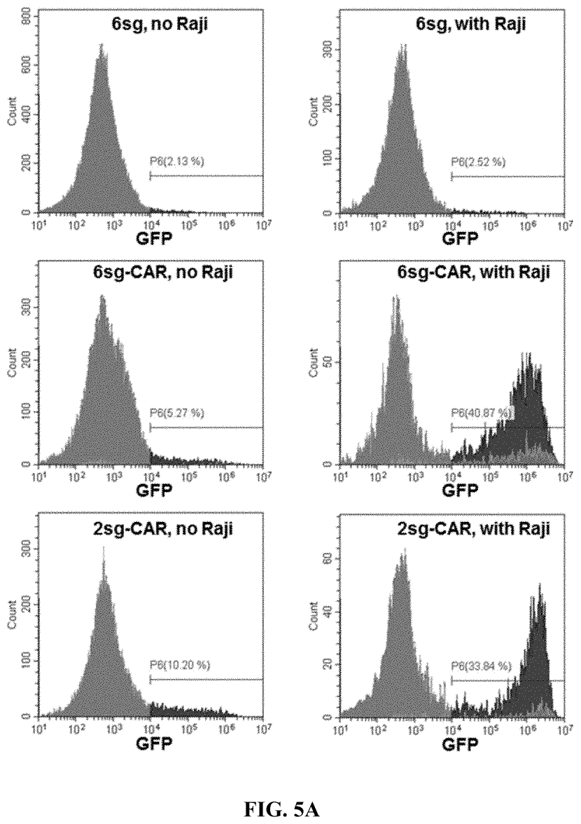

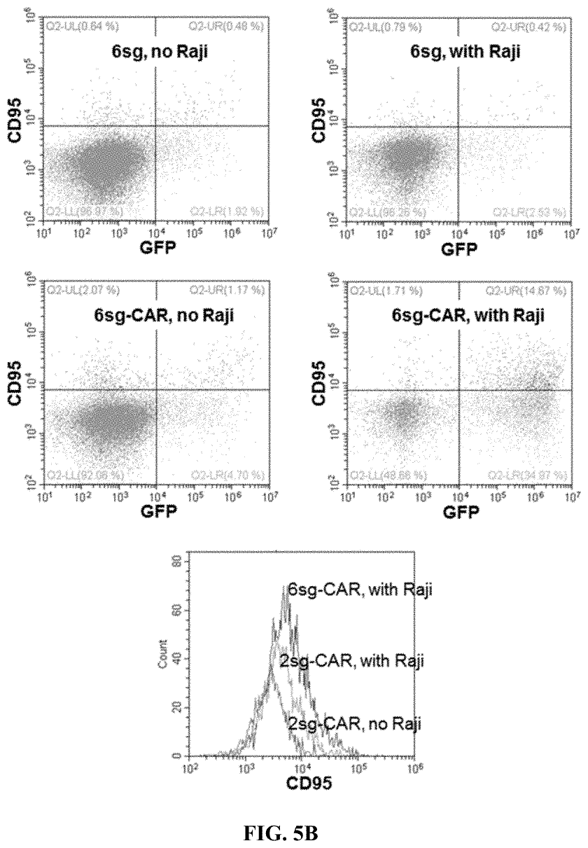

The 2sg-CAR Jurkat-derived cell line, a Jurkat-derived cell line (6sg) containing the GFP reporter gene and stably expressing 6 sgRNAs (3 sgRNAs targeting CD95 gene for upregulation, 2sgRNA targeting CXCR4 gene, and 1 sgRNA targeting the TRE3G promoter for the GFP reporter gene), and a 6sg-CAR cell line which is transduced with the CD19-CAR transgene and transiently transfected with a synthetic nuclear factor of activated T-cells responsive element (NFAT-RE) promoter-driven dCas9-VPR construct, were co-cultured with or without Raji cells (CD19+ and CD22+). After two days co-culture, cells were evaluated for GFP and CD95 expression by flow cytometry after staining with anti-CD95-PE and anti-CD22-APC. In FIG. 5A, plots shown are gated on live CD22⁻ Jurkat-derived cells. Induction of GFP expression was observed in cell lines with the CD19-CAR transgene, suggesting the induction of the synthetic NFAT-RE promoter-driven dCas9VPR can be used to control GFP expression in a ligand-receptor interaction-dependent manner.

FIG. 5B shows that the endogenous CD95 gene expression was also up-regulated simultaneously in the 6sg-CAR cell line by Raji stimulation. Compared with the 6sg-CAR cell line that was not treated with Raji, the Raji-treated cells had more CD95+% of cells (14.67% vs 1.17%). An upregulation of CD95 expression was also observed in the Raji-treated 2sg-CAR cell line (FIG. 5B, bottom), suggesting that endogeneous CD95 expression can be up-regulated in the CAR-activated T cell line. However, higher CD95 expression was observed in the 6sg-CAR cell line (FIG. 5B, bottom), suggesting that the additional upregulation of CD95 expression results from the presence of CD95-targeting sgRNA in the 6sg-CAR cell line. The data shows that multiple genes can be regulated simultaneously using an inducible promoter system.

### Example 7: CMV is an Inducible Promoter Through the CAR Signaling Pathway

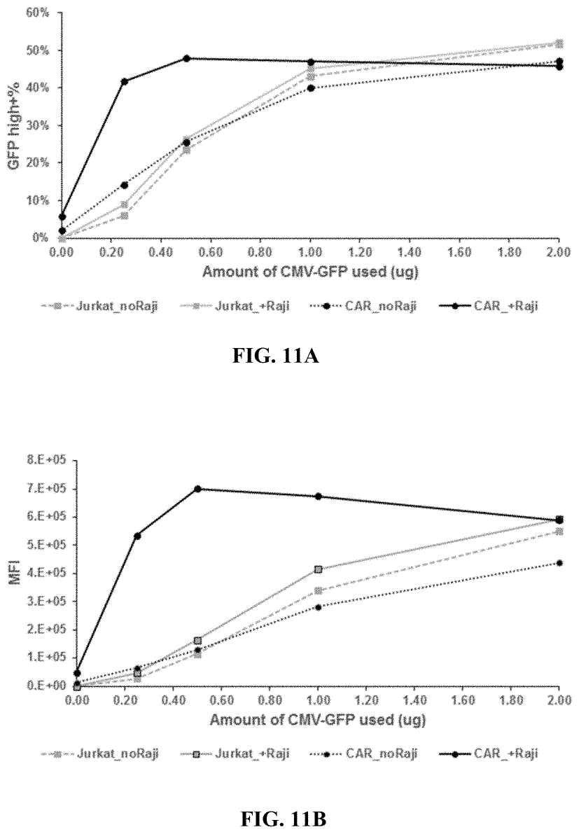

In FIGS. 11A and 11B, Jurkat cells or a Jurkat-derived cell line which contains the CD19-CAR transgene (CAR) were transiently transfected with various amount of a CMV promoter-driven GFP expression plasmid with a Neon-10 ul nucleofection kit (ThermoFisher Scientific). The cells were then stimulated with Raji (CD19+) cells. After one day, cells were evaluated for GFP expression by flow cytometry. More GFP-high % cells were detected in the Raji-stimulated Jurkat-CAR cell line at lower doses of the plasmid used (FIG. 11A) compared to without Raji-stimulation. To avoid bias introduced due to the selection of gate to define GFP-high cells, mean fluorescence intensity (MFI) of all live Jurkat-derived cells were also quantified (FIG. 11B). Again, GFP expression was induced by Raji-stimulation, suggesting that CMV promoter can be induced by the CAR signaling pathway, even though CMV promoter is usually considered to be a constitutive promoter.

### Example 8: Conditional Expression of a GFP Reporter Gene by Ligand-Dependent Signal Cascade

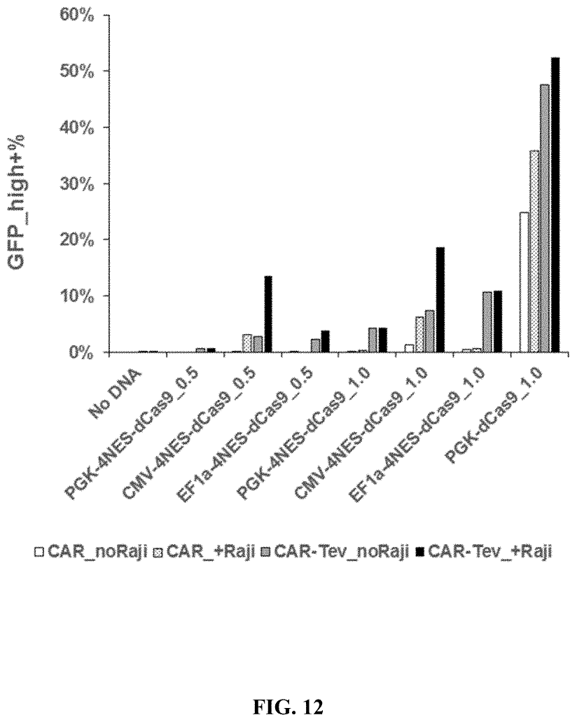

A Jurkat-derived cell line containing the CD19-CAR transgene (CAR) and a Jurkat-derived cell line containing the CD19-CAR-TEV transgene (CAR-Tev) were transiently transfected with the various promoter-driven 4NES-tcs-dCas9-VPR (4NES-dCas9 for short) constructs, with either 0.5 ug (0.5) or 1.0 ug (1.0) of plasmid with a Neon-10 ul nucleofection kit (ThermoFisher Scientific). CD19-CAR-TEV is a CD19-CAR fused to a Tobacco Etch Virus nuclear-inclusion-a endopeptidase (i.e. TEV protease). The 4NES indicates that 4 nuclear export signal sequences were incorporated into the constructs. Tcs is the Tev cleavage site/sequence. The cells were then stimulated with or without Raji (CD19+) cells. After two days of co-culture, cells were evaluated for GFP expression by flow cytometry (FIG. 12). More GFP-high % cells were detected in the Raji-stimulated CAR-Tev cell line. This result suggests that 4NES-tcs-dCas9-VPR expression was induced by Raji stimulation, the 4NES portion was then subsequently cleaved off by CAR-Tev to allow dCas9-VPR to translocate into cell nucleus to activate GFP reporter gene expression. The dCas9 can bind to a sgRNA prior to, concurrent with, or subsequent to cleavage by the protease.

FIG. 12 shows GFP expression levels regulated by systems described herein. As shown in FIG. 7, upon binding of a ligand with its receptor, a natural or synthetic receptor such as a chimeric antigen receptor fused with a protease such as TEV, intrinsic signal transduction pathway(s) can be activated, leading to the recruitment of cellular transcription factors to the promoter region. Such transcriptional activation of the promoter can result in expression of the transgene, a gene modulating polypeptide (GMP) such as a dCas9-VPR or dCas9-KRAB protein fused with nuclear export signal peptides (NES) through a TEV cleavage site (tcs). The NES-tcs-dCas9-VPR/KRAB protein can remain in the cytoplasm until being cleaved by TEV at the tcs. The cleaved dCas9-VPR or dCas9-KRAB protein can then translocate into to nucleus and regulate (activate or suppress) the expression of a target gene.

### Example 9: Conditional Expression of a GFP Reporter Gene by Ligand-Dependent Signal Cascade

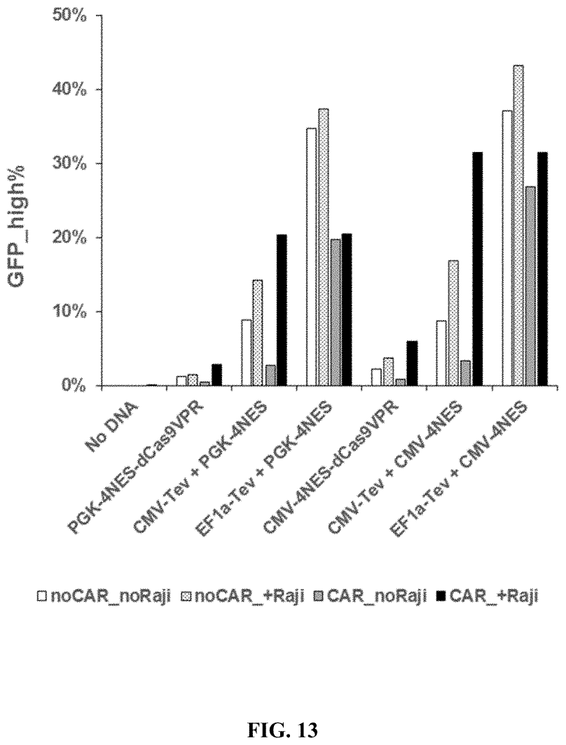

Jurkat cells (no CAR) and a Jurkat-derived cell line which contains the CD19-CAR transgene (CAR) were transiently transfected with the various promoter-driven 4NES-tcs-dCas9-VPR (4NES-dCas9 for short) constructs and the various promoter-driven TEV. The cells were then stimulated by co-culture with or without Raji (CD19+) cells. After two days, cells were evaluated for GFP expression by flow cytometry. More GFP-high % cells were detected in the Raji-stimulated CAR cell line transfected with (CMV-Tev+PGK-4NES-tcs-dCas9-VPR) or (CMV-Tev+CMV-4NES-tcs-dCas9-VPR) compared to without Raji-stimulation, suggesting that inducible expression of Tev alone or both Tev and 4NES-tcs-dCas9-VPR can regulate GFP gene expression (FIG. 13).

As shown in FIG. 6, upon binding of a ligand with its receptor, a natural or synthetic receptor such as a chimeric antigen receptor, intrinsic signal transduction pathway(s) can be activated, leading to the recruitment of cellular transcription factors to the promoter region. Such transcriptional activation of the promoter can lead to the expression of the transgene, a protease such as TEV. TEV can cleave a fusion protein, which is comprised of a gene modulating polypeptide (GMP) such as a dCas9-VPR or dCas9-KRAB protein fused with nuclear export signal peptides (NES) through a TEV cleavage site (TCS). The NES-tcs-dCas9-VPR/KRAB protein can stay in cytoplasm until being cleaved by TEV. The cleaved dCas9-VPR or dCas9-KRAB protein can then translocate into to the nucleus and regulate (activate or suppress) the expression of target genes.

As shown in FIG. 10, upon binding of a ligand with its receptor, a natural or synthetic receptor such as a chimeric antigen receptor, the intrinsic signal transduction pathway(s) can be activated, leading to the recruitment of cellular transcription factors to the promoter region. Such transcriptional activation of the promoter can result in the expression of the transgene a gene modulating polypeptide (GMP) such as a dCas9-VPR or dCas9-KRAB protein fused with nuclear export signal peptides (NES) through a TEV cleavage site (tcs). The expression of a protease transgene such as TEV can also be under the control of a promoter of the same or a different signature gene. The NES-tcs-dCas9-VPR/KRAB protein can stay in cytoplasm until being cleaved by free TEV. The cleaved dCas9-VPR or dCas9-KRAB protein can then translocate into to nucleus to regulate (activate or suppress) the expression of target genes.

### Example 10: Decreased PD-1 Expression by a Ligand-Dependent Signal Cascade

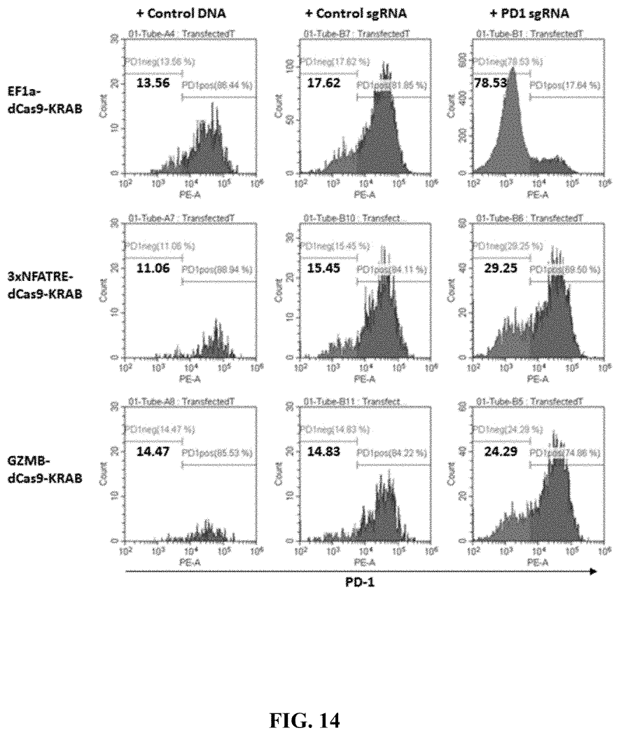

A Jurkat-derived cell line which contains the CD19-CAR transgene (CAR) was transiently transfected with (i) a PD-1 or control sgRNA and (ii) the various promoter-driven dCas9-KRAB. Either a constitutive promoter (e.g., elongation factor 1α (EF1a)) or an inducible promoter (e.g., NFATRE or GZMB P) was used. GZMB P may be a variant of the GZMB promotor that is shorter than the long version of the promoter, GZMB L, as discussed in FIG. 3E. The cells were cultured for 2 days and then stimulated by co-culture with Raji (CD19+) cells. After two more days, cells were stained with PE-conjugated anti-PD-1 and APC-conjugated anti-CD22 monoclonal antibody and evaluated for PD-1 surface expression by flow cytometry. CD22 was used as a Raji cell marker. A higher proportion of PD-1 negative (PD-1⁻) cells were detected in the Raji-stimulated CAR cell line when the CAR cell line was transfected with PD-1 sgRNA than a control sgRNA, suggesting that either constitutive expression or inducible expression of dCas9-KRAB together with a PD-1 sgRNA can down-regulate PD-1 gene expression (FIG. 14).

### Example 11: System Comprising One, Two, or Three Transmembrane Receptors and Multiple Nucleic Acid Binding Proteins

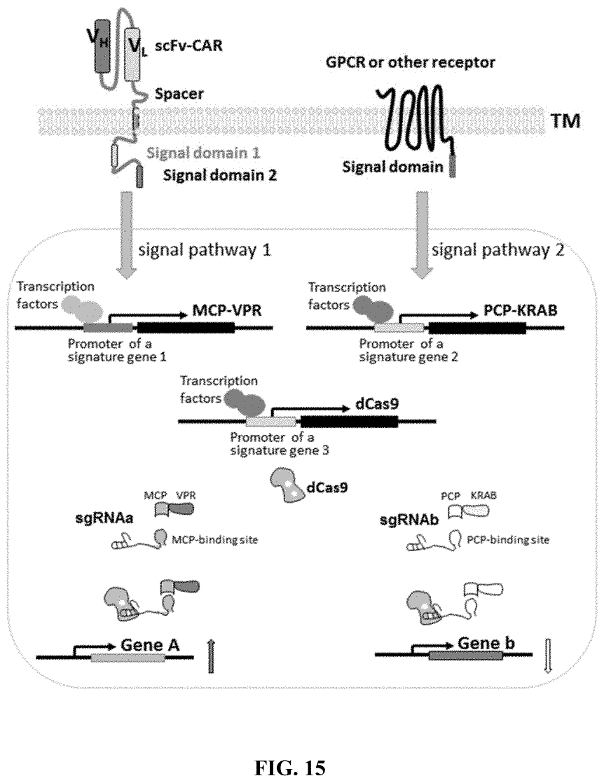

As shown in FIG. 15, upon binding of a ligand with its receptor, a natural receptor such as a G protein-coupled receptor (GPCR) or a synthetic receptor such as a chimeric antigen receptor (CAR, e.g., scFv-CAR), intrinsic signal transduction pathway(s) can be activated, leading to the recruitment of cellular transcription factors to the corresponding promoter regions. Such transcriptional activation of the promoters can lead to the expression of the corresponding transgene, such as (1) a gene modulating polypeptide (GMP) dCas9, (2) a fusion protein containing a gene activation domain MCP-VPR, or (3) a fusion protein containing a gene suppression domain PCP-KRAB. MCP may be a MS2 bacteriophage coat protein, and PCP may be a PP7 bacteriophage coat protein. In some cases, other RNA-binding proteins (RBPs) may be used. A sgRNA comprising a binding sequence for dCas9 and at least one binding sequence for an MCP or PCP can form a complex with (i) dCas9 and (ii) MCP-VPR or PCP-KRAB, respectively. The resulting dCas9-sgRNA-MCP-VPR or dCas9-sgRNA-PCP-KRAB complex can then up- or down-regulate expression of the corresponding target genes, respectively. Either the same or different (e.g., one, two, three, or more) receptors and promoters can be used. A MCP-KRAB/PCP-VPR combination or other combinations can also be used. Referring to FIG. 15, the scFv-CAR is a first receptor to induce signal pathway 1, and the GPCR (or other receptor) is a second receptor to induce signal pathway 2. Additionally, there may be a third receptor to induce signal pathway 3.

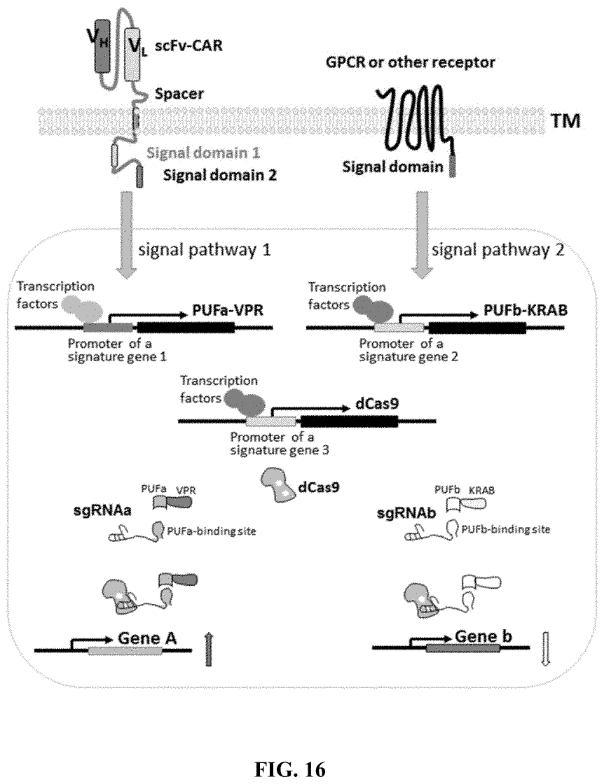

As shown in FIG. 16, upon binding of a ligand with its receptor, a natural receptor such as a G protein-coupled receptor (GPCR) or a synthetic receptor such as a chimeric antigen receptor (CAR, e.g., scFv-CAR), intrinsic signal transduction pathway(s) can be activated, leading to the recruitment of cellular transcription factors to the corresponding promoter regions. Such transcriptional activation of the promoters can lead to the expression of the corresponding transgene, such as (1) a gene modulating polypeptide (GMP) dCas9, (2) a fusion protein containing a gene activation domain PUFa-VPR, or (3) a fusion protein containing a gene suppression domain PUFb-KRAB. PUFa and PUFb may be engineered proteins containing the Pumilio/FBF (PUF) RNA-binding domain. In some cases, other variants of PUF protein such as wild-type PUF, PUF (3-2), PUF (6-2/7-2), PUFw, or PUFc may be used. A sgRNA comprising a binding sequence for dCas9 and at least one binding sequence for a different RBP (e.g., PUGa or PUFb) can form a complex with (i) dCas9 and (ii) PUFa-VPR or PUFb-KRAB. The resulting dCas9-sgRNA-FUFa-VPR or dCas9-sgRNA-PUFb-KRAB complex can then up- or down-regulate expression of the corresponding target genes, respectively. Either the same or different (e.g., one, two, three, or more) receptors and promoters can be used. In some cases, a PUFb-VPR/PUFa-KRAB combination or other combinations can also be used. Referring to FIG. 16, the scFv-CAR is a first receptor to induce signal pathway 1, and the GPCR (or other receptor) is a second receptor to induce signal pathway 2. Additionally, there may be a third receptor to induce signal pathway 3.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-191. (canceled)

192. A system for regulating expression of a target gene in a cell, comprising:
a transmembrane receptor comprising a ligand binding domain and a signaling domain, wherein the signaling domain activates a signaling pathway of the cell upon binding of a ligand to the ligand binding domain; and
a first expression cassette comprising a first nucleic acid sequence encoding a gene modulating polypeptide (GMP) placed under control of a first promoter, wherein the GMP comprises an actuator moiety, and wherein the first promoter is activated to drive expression of the GMP upon binding of the ligand to the ligand binding domain,
wherein the expressed GMP regulates expression of the target gene.

193. The system of claim 192, wherein the first expression cassette comprises a first gene encoding a first endogenous protein, wherein the first gene is located upstream or downstream of the first nucleic acid sequence encoding the GMP, and wherein expression of the first endogenous protein is driven by the first promoter.

194. The system of claim 193, wherein the first gene and the first nucleic acid sequence encoding the GMP are joined by a nucleic acid sequence encoding a peptide linker.

195. The system of claim 194, wherein the peptide linker is a protease recognition sequence.

196. The system of claim 194, wherein the peptide linker is a self-cleaving segment.

197. The system of claim 196, wherein the self-cleaving segment comprises a 2A peptide selected from the group consisting of: T2A, P2A, E2A, F2A, a variant thereof, and a combination thereof.

198. The system of claim 193, wherein the first gene and the first nucleic acid sequence encoding the GMP are joined by a nucleic acid sequence comprising a first internal ribosome entry site (IRES).

199. The system of claim 192, wherein the first promoter comprises an endogenous promoter or a fragment thereof or an exogenous promoter.

200. The system of claim 192, wherein the GMP further comprises a cleavage recognition sequence linked to the actuator moiety, wherein upon release of the actuator moiety via cleavage by a cleavage moiety at the cleavage recognition site, the released actuator moiety regulates expression of the target gene.

201. The system of claim 200, wherein the first nucleic acid sequence further encodes a nuclear export signal peptide linked to the cleavage recognition sequence, wherein the cleavage recognition sequence is flanked by the nuclear export signal peptide and the actuator moiety, and wherein the cleavage moiety is capable of releasing the actuator moiety from the nuclear export signal peptide via cleavage at the cleavage recognition site.

202. The system of claim 200, wherein the first expression cassette comprises a second nucleic acid sequence encoding the cleavage moiety under control of the first promoter, wherein the first promoter is activated to drive expression of the cleavage moiety upon binding of the ligand to the ligand binding domain.

203. The system of claim 200, wherein the system further comprises a second expression cassette comprising a second nucleic acid sequence encoding the cleavage moiety, wherein the second nucleic acid sequence is placed under the control of a second promoter activated by the signaling pathway to drive expression of the cleavage moiety upon binding of the ligand to the ligand binding domain.

204. The system of claim 200, wherein the transmembrane receptor further comprises the cleavage moiety.

205. The system of claim 192, wherein the transmembrane receptor comprises an endogenous receptor or a synthetic receptor.

206. The system of claim 205, wherein the endogenous receptor comprises a T cell receptor (TCR), G-protein coupled receptor (GPCR), integrin receptor, a Notch receptor, an integrin receptor, a cadherin receptor, or tumor necrosis factor receptor (TNFR).

207. The system of claim 205, wherein the synthetic receptor comprises a chimeric antigen receptor (CAR), a synthetic GPCR receptor, a synthetic integrin receptor, or a synthetic Notch receptor.

208. The system of claim 192, wherein the actuator moiety comprises a ribonucleic acid (RNA)-guided actuator moiety, and wherein the system further comprises a guide RNA that complexes with the actuator moiety.

209. The system of claim 208, wherein the RNA-guided actuator moiety substantially lacks DNA cleavage activity.

210. The system of claim 208, wherein the RNA-guided actuator moiety is a Cas protein or fragment thereof that substantially lacks DNA cleavage activity.

211. The system of claim 192, wherein the actuator moiety includes a fusion polypeptide conferring (i) a nuclease activity and (ii) an additional activity selected from the group consisting of: a methyltransferase activity, a demethylase activity, a depurination activity, a pyrimidine dimer forming activity, a polymerase activity, and a hydrolase activity.

212. The system of claim 192, wherein the actuator moiety is linked to a transcription activator or a transcription repressor.

213. The system of claim 192, wherein the first promoter is selected from the group consisting of: an IL-2 promoter, an IFN-γ promoter, an IRF4 promoter, a R4A1 promoter, a PRDM1 promoter, a TBX21 promoter, a CD69 promoter, a CD25 promoter, and a GZMB promoter.

214. The system of claim 192, wherein the cell is an immune cell, a hematopoietic progenitor cell, or a hematopoietic stem cell.

215. A method of regulating expression of a target gene in a cell, comprising:
contacting a ligand to a transmembrane receptor comprising a ligand binding domain and a signaling domain, wherein upon the contacting, the signaling domain activates a signaling pathway of the cell;
expressing a gene modulating polypeptide (GMP) comprising an actuator moiety from a first expression cassette comprising a first nucleic acid sequence encoding the GMP placed under control of a first promoter, wherein the first promoter is activated to drive expression of the GMP upon binding of the ligand to the ligand binding domain; and
increasing or decreasing expression of the target gene via binding of the expressed GMP, thereby regulating expression of the target gene.

216. The method of claim 215, wherein the GMP further comprises a cleavage recognition sequence linked to the actuator moiety, further comprising cleaving, by a cleavage moiety, the cleavage recognition site to release the actuator moiety, which released actuator moiety being capable of regulating expression of the target gene.

217. The method of claim 216, wherein the first nucleic acid sequence further encodes a nuclear export signal peptide linked to the cleavage recognition sequence, wherein the cleavage recognition sequence is flanked by the nuclear export signal peptide and the actuator moiety, further comprising cleaving, by the cleavage moiety, the cleavage recognition site to release the actuator moiety from the nuclear export signal peptide.

218. The method of claim 216, further comprising expressing the cleavage moiety from the first expression cassette comprising a second nucleic acid sequence encoding the cleavage moiety placed under control of the first promoter, wherein the first promoter is activated to drive expression of the cleavage moiety upon binding of the ligand to the ligand binding domain.

219. The method of claim 216, further comprising expressing the cleavage moiety from a second expression cassette comprising a second nucleic acid sequence encoding the cleavage moiety, wherein the second nucleic acid sequence is placed under the control of a second promoter activated by the signaling pathway to drive expression of the cleavage moiety upon binding of the ligand to the ligand binding domain.

220. The method of claim 216, wherein the transmembrane receptor further comprises the cleavage moiety.

221. The method of claim 215, wherein the first promoter is selected from the group consisting of: an IL-2 promoter, an IFN-γ promoter, an IRF4 promoter, a R4A1 promoter, a PRDM1 promoter, a TBX21 promoter, a CD69 promoter, a CD25 promoter, and a GZMB promoter.
